(19) United States
(12) Patent Application Publication
(10) Pub. No.: US 2004/0002482 A1
(43) Pub. Date: Jan. 1, 2004

(54) Androgen pharmaceutical composition and method for treating depression

(76) Inventors: Robert E. Dudley, Kenilworth, IL (US); S. George Kottayil, Long Grove, IL (US); Olivier Palatchi, L' Hay Les Roses (FR)

Correspondence Address: Josesph A. Mahoney, Mayer,, Brown, Rowe & Maw, P.O. Box 2828, Chicago, IL, 60690, US

(21) Appl. No.: 10/098,232

(22) Filed: Mar. 15, 2002

Related U.S. Application Data
(63) Continuation-in-part of application No. 09/703,753, filed on Nov. 1, 2000.
Application No. 09/703,753 is a continuation-in-part of application No. 09/651,777, filed on Aug. 30, 2000, now Pat. No. 6,503,894.
(60) Provisional application No. 60/292,398, filed on May 21, 2001.

(51) Int. Cl. A61K 31/56

(52) U.S. Cl. 514/169

(57) ABSTRACT

The present invention relates to methods, kits, combinations, and compositions for treating, preventing or reducing the risk of developing a depressive disorder, or the symptoms associated with, or related to a depressive disorder in a subject in need thereof. The present invention also relates to a method of administering a steroid in the testosterone synthetic pathway, for example testosterone, to a subject in need thereof. In addition, the methods, kits, combinations and compositions may be used in conjunction with other pharmaceutical agents including agents effective at treating, preventing, or reducing the risk of developing a depressive disorder in a subject.

This application is a continuation-in-part of U.S. patent application Ser. No. 09/703,753, filed Nov. 1, 2000, which is a continuation-in-part of U.S. patent application Ser. No. 09/651,777, filed Aug. 30, 2000. This application also claims priority to U.S. Provisional Application No. 60/292398, filed May 21, 2001. This application claims priority to all such previous applications, and such applications are hereby incorporated herein by reference.

### FIELD OF THE INVENTION

The present invention is related to methods, kits, combinations, and compositions for treating a depressive symptom in a subject by administering to the subject an effective amount of a steroid in the testosterone synthetic pathway.

### DESCRIPTION OF THE RELATED ART

In the 1940's several studies demonstrated that testosterone and other androgens may be successfully used to treat depressive syndromes in middle-aged men. But with increasing use of electroconvulsive therapy and the advent of tricyclic antidepressants and monoamine oxidase inhibitors in the 1950's, androgens lost favor as a treatment for depression. A few studies in the 1970's and 80's reconfirmed the efficacy of androgens such as mesterolone in depressed men, but androgens continued to arouse little interest, perhaps because of the steady introduction of newer classes of antidepressant agents, of which some could be administered to both sexes without concern for masculinizing effects.

In other studies some depressed men exhibited reduced testosterone levels, although this association is complex and probably affected by additional factors. Hypogonadal men also often exhibit depressive symptoms and testosterone replacement therapy generally improves these symptoms. This finding extends to men with HIV-induced hypogonadism, who also appear to show an antidepressant response to testosterone. Furthermore, men who ingest markedly supraphysiologic doses of testosterone and related androgens (such as illicit anabolic steroid abusers) may develop manic or hypomanic symptoms during androgen use and depressive symptoms on androgen withdrawal.

In more recent studies, the potential of testosterone as an antidepressant has been reconsidered. In one study by Seidman, et al. (Seidman S N, Rabkin J., J Affective Disord 1998;48:157-161), intramuscular testosterone enanthate, was administered at 400 mg every two weeks to five men who had remained depressed despite adequate treatment with selective serotonin reuptake inhibitors (SSRI's). These men's total testosterone levels were in the low or borderline range (200-350 ng/dl; reference range 300-990 ng/dl). All five subjects improved. Their mean depression scores on the Hamilton Rating Scale for Depression (HAM-D) declined from 19.2 at baseline to 4.0 at eight weeks. Subsequently, four of the five men were administered placebo injections, and three of these four relapsed within two weeks. Following this study Seidman, et al. (J Clin Psychiatry 2001;62:406-412), conducted a randomized, placebo-controlled trial of testosterone enanthate in men with major depressive disorder, again selecting subjects with testosterone levels of 350 ng/dl or less. However, this study differed from the prior open-label study in that subjects were not simultaneously taking an antidepressant medication, but received testosterone alone. After six weeks of treatment, the investigators found no significant difference between testosterone and placebo on the Hamilton Rating Scale for Depression or Beck Depression Inventory (BDI). About 40% of testosterone-treated subjects responded (as defined by a 50% or greater reduction in the Hamilton Rating Scale for Depression), but so did a comparable portion of subjects receiving placebo. Interestingly, of eight placebo non-responders offered open-label testosterone at the conclusion of the study, six responded. While admitting that these latter observations were subject to expectational bias, the authors speculated that testosterone possessed variable and possibly idiosyncratic antidepressant effects in some men, and that further research was justified.

Transdermal preparations of testosterone have provided a useful delivery system for normalizing serum testosterone levels in hypogonadal men and preventing the clinical symptoms and long term effects of androgen deficient men. Available transdermal preparations of testosterone include, for example, TESTODERM®, TESTODERM® TTS, and ANDRODERM®. Testosterone is also available in other formulations including those available as an injectable, for example, DEPO-TESTOSTERONE® (testosterone cypionate), and DELATESTRYL BTG® (testosterone enanthate), or as a gel, for example, ANDROGEL® marketed by Unimed Pharmaceuticals, Inc., Deerfield, Ill., the assignee of this application.

In men, transdermal patches are applied to the scrotal skin or other parts of the body. Recently, a one-percent testosterone gel has been approved for use in men, and provides dosing flexibility with minimal skin irritation. This gel is marketed under the name ANDROGEL®. However, all currently available testosterone transdermal products are specifically contraindicated for use in women in the United States. Furthermore, none of the currently available androgen treatment modalities for women, for example, oral methyltestosterone, intramuscular testosterone ester injections or subcutaneous testosterone implants can achieve reproducible testosterone serum levels on a consistent daily basis.

A. Androgens in Men

Testosterone, the major circulating androgen in men, is synthesized from cholesterol. The approximately 500 million Leydig cells in the testes secrete more than 95% of the 6-7 mg of testosterone produced per day. Two hormones produced by the pituitary gland, luteinizing hormone (“LH”) and follicle stimulating hormone (“FSH”), are required for the development and maintenance of testicular function and negatively regulate testosterone production. Circulating testosterone is metabolized to various 17-keto steroids through two different pathways. Testosterone can be metabolized to dihydrotestosterone (“DHT”) by the enzyme 5-alpha-reductase or to estradiol (“E₂”) by an aromatase enzyme complex.

Testosterone circulates in the blood 98% bound to protein. In men, approximately 40% of the binding is to the high-affinity sex hormone binding globulin (“SHBG”). The remaining 60% is bound weakly to albumin. Thus, a number of measurements for testosterone are available from clinical laboratories. The term “free” testosterone as used herein refers to the fraction of testosterone in the blood that is not bound to protein. The term “total testosterone” or “testosterone” as used herein means the free testosterone plus protein-bound testosterone. The term “bioavailable testosterone” as used herein refers to the non-sex hormone binding globulin bound testosterone and includes testosterone weakly bound to albumin.

The following table from the UCLA-Harbor Medical Center summarizes the hormone concentrations in normal adult men range:

TABLE 1
Hormone Levels in Normal Men

| Hormone | Normal Range | |
|---|---|---|
| Testosterone | 298 to 1043 | ng/dL |
| Free Testosterone | 3.5 to 17.9 | ng/dL |
| DHT | 31 to 193 | ng/dL |
| DHT/T Ratio | 0.052 to 0.33 | |
| DHT + T | 372 to 1349 | ng/dL |
| SHBG | 10.8 to 46.6 | nmol/L |
| FSH | 1.0 to 6.9 | mlU/mL |
| LH | 1.0 to 8.1 | mlU/mL |
| E₂ | 17.1 to 46.1 | pg/mL |

There is considerable variation in the half-life of testosterone reported in the literature, ranging from 10 to 100 minutes. Researchers do agree, however, that circulating testosterone has a diurnal variation in normal young men. Maximum levels occur at approximately 6:00 to 8:00 a.m. with levels declining throughout the day. Characteristic profiles have a maximum testosterone level of 720 ng/dL and a minimum level of 430 ng/dL. The physiological significance of this diurnal cycle, if any, however, is not clear.

Because increasing testosterone concentrations has been shown to alter sexual performance and libido, researchers have investigated methods of delivering testosterone to 10 men. These methods include intramuscular injections (43%), oral replacement (24%), pellet implants (23%), and transdermal patches (10%). A summary of these methods is shown in Table 2.

TABLE 2
Mode of Application and Dosage of Various Testosterone Preparations

| Preparation | Route Of Application | Full Substitution Dose |
|---|---|---|
| In Clinical Use | | |
| Testosterone enanthate | Intramuscular injection | 200-25.0 g every 2-3 weeks |
| Testosterone cypionate | Intramuscular injection | 200 mg every 2 weeks |
| Testosterone undecanoate | Oral | 2-4 capsules at 40 mg per day |
| Transdermal testosterone patch | Scrotal skin | 1 membrane per day |
| Transdermal testosterone patch | Non-scrotal skin | 1 or 2 systems per day |
| Testosterone implants | Implantation under the | 3-6 implants of 200 mg every |
| | abdominal skin | 6 months |
| Under Development | | |
| Testosterone cyclodextrin | Sublingual | 2.5-5.0 mg twice daily |
| Testosterone undecanoate | Intramuscular injection | 1000 mg every 8-10 weeks |
| Testosterone buciclate | Intramuscular injection | 1000 mg every 12-16 weeks |
| Testosterone microspheres | Intramuscular injection | 315 mg for 11 weeks |
| Obsolete | | |
| 17-Methyltestosterone | Oral | 25-5.0 g per day |
| Fluoxymesterone | Sublingual | 10-25 mg per day |
| | Oral | 10-20 mg per day |

All of the testosterone replacement methods currently employed, however, suffer from one or more drawbacks. For example, subdermal pellet implants and ester injections are painful and require doctor visits. Many of these methods, such as oral/sublingual/buccal preparations, suffer from undesirable pharmacokinetic profile-creating supra-physiologic testosterone concentrations followed a return to baseline. Transdermal patches provide less than optimal pharmacokinetic characteristics, are embarrassing for many subjects, and are associated with significant skin irritation. Thus, although the need for an effective testosterone replacement methodology has existed for decades, an alternative replacement therapy that overcomes these problems has never been developed.

B. Androgens in Women

The excretion of androgenic steroids in the urine of adult women was demonstrated more than 50 years ago. Since that time, physiologists and clinicians have explored the sources and biological functions of testosterone and other endogenous androgenic hormones in the human female, see, for example, Geist S. H., Androgen therapy in the human female, J. Clin. Endocrinol. 1941; 1:154-161. It is now known that androgens are secreted by both the ovaries and adrenal glands in women. Each source contributes about 50% (directly and through precursors) (see, for example, Abraham G. E., Ovarian and adrenal contribution to peripheral androgens during the menstrual cycle, J. Clin. Endocrinol. Metab. 1974; 39:340-346) to the approximately 300 μg of testosterone produced daily in healthy “cycling” women (see, for example, Southren A. L., et al., Further study of factors affecting the metabolic clearance rate of testosterone in man, J. Clin. Endocrinol. Metab. 1968; 28:1105-1112). While the adverse effects of excess androgen production, as occurs in the polycystic ovary syndrome and certain androgen producing tumors, have been well described (see, for example, Lobo R. A., Chapter 20: Androgen excess in Infertility, Contraception and Reproductive Endocrinology, Third Edition. D R Mishell, V. Davajan and R. Lobo, Editors. Blackwell Scientific Publications, Boston. pp 422-446, 1991), the normal physiological effects of androgens in women have been much less appreciated. As inferred from animal studies, male physiology, and the symptoms of women with deficient androgen production, the major physiological effects of androgens in normal women include, but are not limited to anabolic effects on muscle, skin, hair and bone; stimulatory effects on erythropoiesis; modulatory effects on immune function; and psychological effects on mood, well-being and sexual function.

In addition, endogenous androgens are important for the development of pubic hair and are thought to modulate the action of estrogens and progestins on a variety of reproductive target tissues. It is also believed that androgens play an important role in modulating the secretory function of the lacrimal gland.

Fifty percent of circulating testosterone is derived from direct ovarian secretion in the thecal cells under the control of luteinizing hormone. The other half is derived from peripheral conversion of adrenal androgen precursors dehydroepiandrosterone, androstenedione, and dehydroepiandrosterone sulfate. Testosterone can also be converted to dihydrotestosterone or estradiol. Thus, testosterone serves as both a hormone and as a pro-hormone.

Testosterone circulates in the blood 98% bound to protein. In women, approximately 66% of the binding is to the high-affinity sex hormone binding globulin. The remaining 34% is bound weakly to albumin. Thus, a number of measurements for testosterone are available from clinical laboratories. The term “free” testosterone as used herein refers to the fraction of testosterone in the blood that is not bound to protein. The term “total testosterone” or “testosterone” as used herein means the free testosterone plus protein-bound testosterone. The term “bioavailable testosterone” as used herein refers to the non-sex hormone binding globulin bound testosterone and includes that weakly bound to albumin. The order of affinity for the steroids most strongly bound by sex hormone binding globulin is dihydrotestosterone>testosterone>androstenedione>estrogen. Sex hormone binding globulin weakly binds dihydrotestosterone, but not dihydrotestosterone sulfate. Table 3 shows the approximate hormonal levels in normal pre-menopausal women.

TABLE 3
Hormone Levels in Normal Pre-Menopausal Women

| Hormone | Mean ± sd | Median | Range |
|---|---|---|---|
| Testosterone (nmol/L) | 1.20 ± 0.69 | 0.98 | 0.4-2.7 |
| Free testosterone (pmol/L) | 12.80 ± 5.59 | 12.53 | 4.1-24.2 |
| % Free testosterone of total | 1.4 ± 1.1 | 1.1 | 0.4-6.3 |
| testosterone | | | |
| Luteinizing hormone | 7.2 ± 3.3 | 6.7 | 3.0-18.7 |
| (IU/L) | | | |
| Follicle stimulating | 4.7 ± 3.6 | 4.2 | 1.5-21.4 |
| hormone (IU/L) | | | |
| Sex hormone binding | 66.1 ± 22.7 | 71.0 | 17.8-114.0 |
| globulin (nmol/L) | | | |

However, there is no general consensus on what constitutes “testosterone deficiency” in women because historically it has been impossible to develop assays capable of measuring such small hormonal levels. This is especially true when measuring free or bioavailable testosterone levels. Consequently, currently available laboratory evaluations, including measuring total, free, and bioavailable serum testosterone levels, have not been used extensively to identify hypoandrogenic women.

In comparison to other hormone deficiency states, testosterone deficiency in women has been largely ignored as a clinical entity. Nevertheless, there exist well-defined subject populations where androgen production is clearly deficient and where associated symptomatology has been described, including, for example, young oophorectomized/hysterectomized women, post-menopausal women on estrogen replacement therapy, women on oral contraceptives, women with adrenal dysfunction, women with corticosteroid-induced adrenal suppression, and human immunodeficiency virus-positive women.

Despite the clear benefits of administering testosterone to both normal and testosterone deficient women, almost all of the testosterone delivery preparations for human use are designed for hypogonadal men who require significantly greater amounts of testosterone than a testosterone deficient women. As a result, these formulations and devices are unsuitable for women requiring low doses of testosterone. Intramuscular injunction of testosterone esters, for example, is the popular form of androgen replacement for men but is unsatisfactory for women because of the very high levels of testosterone in the first 2-3 days after injection. Moreover, many women report increased acne and occasional cliteromegaly with this type of testosterone administration. Subjects receiving injection therapy often complain that the delivery mechanism is painful and causes local skin reactions.

None of the current testosterone replacement products available for use in women are approved in the United States for chronic treatment of the female testosterone deficiency states described herein. Also, currently available methyltestosterone products, which can be administered orally, are no longer recommended as a testosterone replacement method for hypogonadal men, see, for example, Gooren L J. G. and Polderman K. H., Safety aspects of androgens. In Testosterone: Action, Deficiency, Substitution. E. Nieschlag and H M. Behre, editors, Springer-Verlag, Heidelberg, p. 136 (1990). The long acting injectable testosterone-esters, such as enanthate or cypionate are formulated for high dose administration to men (for example 200-300 mg) and produce supra-physiological hormone levels, even when given at lower doses to women (for example 50-100 mg) (see, for example, Sherwin B. B. and Gelfand M. M., Differential symptom response to parenteral estrogen and/or androgen administration in the surgical menopause, Am. J. Obstet. Gynecol. 1985; 151:153-160). Testosterone implants, which have been used experimentally in the past, can likewise produce supra-physiological hormone levels in women, see, for example, Burger H. G. et al., The management of persistent menopausal symptoms with oestradiol-testosterone implants: clinical, lipid and hormonal results, Maturitas 1984; 6:351-358. The supra-physiological androgen levels associated with these products have produced virilizing side effects in some subjects, see for example, Burger H. G. et al., (1984). Also see, for example, Sherwin B. B, and Gelfand M. M., (1985). Also see, for example, Urman B., et al., Elevated serum testosterone, hirsutism and virilism associated with combined androgen-estrogen hormone replacement therapy, Obstet. Gynecol., 1991; 7:595-598.

Given the above, however, ESTRATEST®, which is a combination of methyltestosterone and esterified estrogens in oral tablet formulations, is the most commonly used androgen product used to treat women in the United States. At present, however, its only approved indication is for the treatment of moderate to severe vasomotor symptoms associated with menopause in those subjects not improved by estrogens alone. Pharmacological doses of methyltestosterone higher than those suggested for hypogonadal men have also been used to treat breast cancer in women. However, oral administration produces inappropriate testosterone levels and unpredictable absorption patters between subjects (Buckler 1998). Moreover, because the liver metabolizes the preparation, there is a risk of hepatoxicity not to mention first pass metabolism.

Testosterone pellet implants (50 mg or 100 mg of testosterone) inserted under local anesthesia in the abdominal wall have been used in conjunction with estrogen pellet implants for many years. Testosterone levels peak about one month after implantation and then return to baseline by month five or six. The testosterone levels are high and characterized by substantial rises and falls over several months and marked individual variation in this period. In addition, implants require a surgical procedure that many men and women simply do not wish to endure. In hypogonadal men, for example, implant therapy includes a risk of extrusion (8.5%), bleeding (2.3%), or infection (0.6%).

Given the problems associated with injected, orally administered and implant-based testosterone delivery methods, researchers have recently begun experimenting with more controlled release preparations that can deliver stable and physiological testosterone levels to women. In the past decade, the transdermal delivery of estradiol has become recognized as a safe, physiological and subject-friendly method for estrogen replacement therapy in women. Second generation estradiol patches that use adhesive matrix technology have recently become available in the United States and Europe. Matrix technology now exists to transdermally administer physiological amounts of testosterone alone for the treatment of androgen deficiency states in women. As the subject populations defined above are approximately 50% deficient in their testosterone production, the transdermal systems have been designed to deliver approximately half of the normal daily testosterone production rate or about 150 μg per day. Matrix technology-based transdermal testosterone administration has been used successfully in women to treat acquired immunodeficiency syndrome wasting and female sexual dysfunction after oophorectomy.

Two testosterone patches for women have been tested in clinical studies. Buckler and his associates have investigated a testosterone patch (Ethical Pharmaceuticals, UK) delivering either 840, 1100, 3000 μg testosterone per day applied twice weekly to the anterior abdominal wall, but did not disclose the composition of the patch (Buckler 1998). Another patch, the TMTDS patch (Watson Laboratories, Salt Lake City, Utah), is a translucent patch having a surface area of 18 cm² which uses sorbitan monooleate as a permeation enhancer and a hypoallergenic acrylic adhesive in an alcohol-free matrix. The average testosterone content of each patch is 4.1 mg. Each patch is designed to deliver testosterone at a nominal rate of 150 g of testosterone per day over an application period of three to four days. Thus, the TMTDS patch is applied twice per week (Javanbakht et al. 2000).

While clinical studies have reported that the testosterone-containing patch is capable of increasing testosterone concentrations in women via a controlled release mechanism, the patches do not provide dosing flexibility. Moreover, their visibility may be esthetically unappealing to some women and may have a tendency to fall off, especially during rigorous physical exercise.

For these and other reasons, therefore, it would be a difficult but much desired advance in the art to provide an effective percutaneously administered steroid in the testosterone synthetic pathway formulation to be applied directly to the skin of a subject in the form of, for example, a gel, an ointment, or a cream, to treat a depressive symptom, and in particular to treat a subject that has failed to respond to conventional antidepressants and/or who exhibited low or borderline testosterone levels.

### BRIEF DESCRIPTION OF THE FIGURES

FIG. No. 1(a) is a graph showing the 24-hour testosterone pharmacokinetic profile for hypogonadal men prior to receiving 5.0 g/day of AndroGel®, 10.0 g/day of AndroGel®, or the testosterone patch (by initial treatment group).

FIG. No. 1(b) is a graph showing the 24-hour testosterone pharmacokinetic profile for hypogonadal men on the first day of treatment with either 5.0 g/day of AndroGel®, 10.0 g/day of AndroGel®, or the testosterone patch (by initial treatment group).

FIG. No. 1(c) is a graph showing the 24-hour testosterone pharmacokinetic profile for hypogonadal men on day 30 of treatment with either 5.0 g/day of AndroGel®, 10.0 g/day of AndroGel, or the testosterone patch (by initial treatment group).

FIG. No. 1(d) is a graph showing the 24-hour testosterone pharmacokinetic profile for hypogonadal men on day 90 of treatment with either 5.0 g/day of AndroGel®, 10.0 g/day of AndroGel®, or the testosterone patch (by initial treatment group).

FIG. No. 1(e) is a graph showing the 24-hour testosterone pharmacokinetic profile for hypogonadal men on day 180 of treatment with either 5.0 g/day of AndroGel®, 10.0 g/day of AndroGel®, or the testosterone patch (by final treatment group).

FIG. No. 1(f) is a graph showing the 24-hour testosterone pharmacokinetic profile for hypogonadal men on day 0, 1, 30, 90, and 180 of treatment with 5.0 g/day of AndroGel®.

FIG. No. 1(g) is a graph showing the 24-hour testosterone pharmacokinetic profile for hypogonadal men on day 0, 1, 30, 90, and 180 of treatment with 10.0 g/day of AndroGel®.

FIG. No. 1(h) is a graph showing the 24-hour testosterone pharmacokinetic profile for hypogonadal men on day 0, 1, 30, 90, and 180 of treatment with the testosterone patch.

FIG. No. 2(a) is a graph showing the 24-hour free testosterone pharmacokinetic profile for hypogonadal men on day 1 of treatment with either 5.0 g/day of AndroGel®, 10.0 g/day of AndroGel®, or the testosterone patch (by initial treatment group).

FIG. No. 2(b) is a graph showing the 24-hour free testosterone pharmacokinetic profile for hypogonadal men on day 30 of treatment with either 5.0 g/day of AndroGel®, 10.0 g/day of AndroGel®, or the testosterone patch (by initial treatment group).

FIG. No. 2(c) is a graph showing the 24-hour free testosterone pharmacokinetic profile for hypogonadal men on day 90 of treatment with either 5.0 g/day of AndroGel®, 10.0 g/day of AndroGel®, or the testosterone patch (by initial treatment group).

FIG. No. 2(d) is a graph showing the 24-hour free testosterone pharmacokinetic profile for hypogonadal men on day 180 of treatment with either 5.0 g/day of AndroGel®, 10.0 g/day of AndroGel®, or the testosterone patch (by final treatment group).

FIG. No. 2(e) is a graph showing the 24-hour free testosterone pharmacokinetic profile for hypogonadal men on day 0, 1, 30, 90, and 180 of treatment with 5.0 g/day of AndroGel®.

FIG. No. 2(f) is a graph showing the 24-hour free testosterone pharmacokinetic profile for hypogonadal men on day 0, 1, 30, 90, and 180 of treatment with 10.0 g/day of AndroGel®.

FIG. No. 2(g) is a graph showing the 24-hour free testosterone pharmacokinetic profile for hypogonadal men on day 0, 1, 30, 90, and 180 of treatment with the testosterone patch.

FIG. No. 3 is a graph showing the DHT concentrations on days 0 through 180 for hypogonadal men receiving either 5.0 g/day of AndroGel®, 10.0 g/day of AndroGel®, or the testosterone patch (by initial treatment group).

FIG. No. 4 is a graph showing the DHT/T ratio on days 0 through 180 for hypogonadal men receiving either 5.0 g/day of AndroGel®, 10.0 g/day of AndroGel®, or the testosterone patch (by initial treatment group).

FIG. No. 5 is a graph showing the total androgen concentrations (DHT+T) on days 0 through 180 for hypogonadal men receiving either 5.0 g/day of AndroGel®, 10.0 g/day of AndroGel®, or the testosterone patch (by initial treatment group).

FIG. No. 6 is a graph showing the E₂ concentrations on days 0 through 180 for hypogonadal men receiving either 5.0 g/day of AndroGel®, 10.0 g/day of AndroGel®, or the testosterone patch (by initial treatment group).

FIG. No. 7 is a graph showing the SHBG concentrations on days 0 through 180 for hypogonadal men receiving either 5.0 g/day of AndroGel®, 10.0 g/day of AndroGel®, or the testosterone patch (by initial treatment group).

FIG. No. 8(a) is a graph showing the FSH concentrations on days 0 through 180 for men having primary hypogonadism and receiving either 5.0 g/day of AndroGel®, 10.0 g/day of AndroGel®, or the testosterone patch (by initial treatment group).

FIG. No. 8(b) is a graph showing the FSH concentrations on days 0 through 180 for men having secondary hypogonadism and receiving either 5.0 g/day of AndroGel®, 10.0 g/day of AndroGel®, or the testosterone patch (by initial treatment group).

FIG. No. 8(c) is a graph showing the FSH concentrations on days 0 through 180 for men having age-associated hypogonadism and receiving either 5.0 g/day of AndroGel®, 10.0 g/day of AndroGel®, or the testosterone patch (by initial treatment group).

FIG. No. 8(d) is a graph showing the FSH concentrations on days 0 through 180 for men having hypogonadism of an unknown origin and receiving either 5.0 g/day of AndroGel®, 10.0 g/day of AndroGel®, or the testosterone patch (by initial treatment group).

FIG. No. 9(a) is a graph showing the LH concentrations on days 0 through 180 for men having primary hypogonadism and receiving either 5.0 g/day of AndroGel®, 10.0 g/day of AndroGel®, or the testosterone patch (by initial treatment group).

FIG. No. 9(b) is a graph showing the LH concentrations on days 0 through 180 for men having secondary hypogonadism and receiving either 5.0 g/day of AndroGel®, 10.0 g/day of AndroGel®, or the testosterone patch (by initial treatment group).

FIG. No. 9(c) is a graph showing the LH concentrations on days 0 through 180 for men having age-associated hypogonadism and receiving either 5.0 g/day of AndroGel®, 10.0 g/day of AndroGel®, or the testosterone patch (by initial treatment group).

FIG. No. 9(d) is a graph showing the LH concentrations on days 0 through 180 for men having hypogonadism of an unknown origin and receiving either 5.0 g/day of AndroGel®, 10.0 g/day of AndroGel®, or the testosterone patch (by initial treatment group).

FIG. No. 10(a) is a graph showing sexual motivation scores on days 0 through 180 for hypogonadal men receiving either 5.0 g/day of AndroGel®, 7.5 g/day 10.0 g/day of AndroGel®, or the testosterone patch.

FIG. No. 10(b) is a graph showing overall sexual desire scores on days 0 through 180 for hypogonadal men receiving either 5.0 g/day of AndroGel®, 7.5 g/day 10.0 g/day of AndroGel®, or the testosterone patch.

FIG. No. 10(c) is a graph showing sexual enjoyment (with a partner) scores on days 0 through 180 for hypogonadal men receiving either 5.0 g/day of AndroGel®, 7.5 g/day 10.0 g/day of AndroGel®, or the testosterone patch.

FIG. No. 11(a) is a graph showing sexual performance scores on days 0 through 180 for hypogonadal men receiving either 5.0 g/day of AndroGel®, 7.5 g/day 10.0 g/day of AndroGel®, or the testosterone patch.

FIG. No. 11(b) is a graph showing erection satisfaction performance scores on days 0 through 180 for hypogonadal men receiving either 5.0 g/day of AndroGel®, 7.5 g/day 10.0 g/day of AndroGel®, or the testosterone patch.

FIG. No. 11(c) is a graph showing percent erection scores on days 0 through 180 for hypogonadal men receiving either 5.0 g/day of AndroGel®, 7.5 g/day 10.0 g/day of AndroGel®, or the testosterone patch.

FIG. No. 12(a) is a graph showing the 24-hour testosterone pharmacokinetic profile for hypogonadal men prior to receiving 5.0 g/day of AndroGel®, 10.0 g/day of AndroGel®, or the testosterone patch (by initial treatment group).

FIG. No. 12(b) is a graph showing the 24-hour testosterone pharmacokinetic profile for hypogonadal men on the first day of treatment with either 5.0 g/day of AndroGel®, 10.0 g/day of AndroGel®, or the testosterone patch (by initial treatment group).

FIG. No. 12(c) is a graph showing the 24-hour testosterone pharmacokinetic profile for hypogonadal men on day 30 of treatment with either 5.0 g/day of AndroGel®, 10.0 g/day of AndroGel, or the testosterone patch (by initial treatment group).

FIG. No. 12(d) is a graph showing the 24-hour testosterone pharmacokinetic profile for hypogonadal men on day 90 of treatment with either 5.0 g/day of AndroGel®, 10.0 g/day of AndroGel®, or the testosterone patch (by initial treatment group).

FIG. No. 12(e) is a graph showing the 24-hour testosterone pharmacokinetic profile for hypogonadal men on day 180 of treatment with either 5.0 g/day of AndroGel®, 10.0 g/day of AndroGel®, or the testosterone patch (by final treatment group).

FIG. No. 12(f) is a graph showing the 24-hour testosterone pharmacokinetic profile for hypogonadal men on day 0, 1, 30, 90, and 180 of treatment with 5.0 g/day of AndroGel®.

FIG. No. 12(g) is a graph showing the 24-hour testosterone pharmacokinetic profile for hypogonadal men on day 0, 1, 30, 90, and 180 of treatment with 10.0 g/day of AndroGel®.

FIG. No. 12(h) is a graph showing the 24-hour testosterone pharmacokinetic profile for hypogonadal men on day 0, 1, 30, 90, and 180 of treatment with the testosterone patch.

FIG. No. 13(a) is a graph showing the 24-hour free testosterone pharmacokinetic profile for hypogonadal men on day 1 of treatment with either 5.0 g/day of AndroGel®, 10.0 g/day of AndroGel®, or the testosterone patch (by initial treatment group).

FIG. No. 13(b) is a graph showing the 24-hour free testosterone pharmacokinetic profile for hypogonadal men on day 30 of treatment with either 5.0 g/day of AndroGel®, 10.0 g/day of AndroGel®, or the testosterone patch (by initial treatment group).

FIG. No. 13(c) is a graph showing the 24-hour free testosterone pharmacokinetic profile for hypogonadal men on day 90 of treatment with either 5.0 g/day of AndroGel®, 10.0 g/day of AndroGel®, or the testosterone patch (by initial treatment group).

FIG. No. 13(d) is a graph showing the 24-hour free testosterone pharmacokinetic profile for hypogonadal men on day 180 of treatment with either 5.0 g/day of AndroGel®, 10.0 g/day of AndroGel®, or the testosterone patch (by final treatment group).

FIG. No. 13(e) is a graph showing the 24-hour free testosterone pharmacokinetic profile for hypogonadal men on day 0, 1, 30, 90, and 180 of treatment with 5.0 g/day of AndroGel®.

FIG. No. 13(f) is a graph showing the 24-hour free testosterone pharmacokinetic profile for hypogonadal men on day 0, 1, 30, 90, and 180 of treatment with 10.0 g/day of AndroGel®.

FIG. No. 13(g) is a graph showing the 24-hour free testosterone pharmacokinetic profile for hypogonadal men on day 0, 1, 30, 90, and 180 of treatment with the testosterone patch.

FIG. No. 14 is a graph showing the DHT concentrations on days 0 through 180 for hypogonadal men receiving either 5.0 g/day of AndroGel®, 10.0 g/day of AndroGel®, or the testosterone patch (by initial treatment group).

FIG. No. 15 is a graph showing the DHT/T ratio on days 0 through 180 for hypogonadal men receiving either 5.0 g/day of AndroGel®, 10.0 g/day of AndroGel®, or the testosterone patch (by initial treatment group).

FIG. No. 16 is a graph showing the total androgen concentrations (DHT+T) on days 0 through 180 for hypogonadal men receiving either 5.0 g/day of AndroGel®, 10.0 g/day of AndroGel®, or the testosterone patch (by initial treatment group).

FIG. No. 17 is a graph showing the E₂ concentrations on days 0 through 180 for hypogonadal men receiving either 5.0 g/day of AndroGel®, 10.0 g/day of AndroGel®, or the testosterone patch (by initial treatment group).

FIG. No. 18 is a graph showing the SHBG concentrations on days 0 through 180 for hypogonadal men receiving either 5.0 g/day of AndroGel®, 10.0 g/day of AndroGel®, or the testosterone patch (by initial treatment group).

FIG. No. 19(a) is a graph showing the FSH concentrations on days 0 through 180 for men having primary hypogonadism and receiving either 5.0 g/day of AndroGel®, 10.0 g/day of AndroGel®, or the testosterone patch (by initial treatment group).

FIG. No. 19(b) is a graph showing the FSH concentrations on days 0 through 180 for men having secondary hypogonadism and receiving either 5.0 g/day of AndroGel®, 10.0 g/day of AndroGel®, or the testosterone patch (by initial treatment group).

FIG. No. 19(c) is a graph showing the FSH concentrations on days 0 through 180 for men having age-associated hypogonadism and receiving either 5.0 g/day of AndroGel®, 10.0 g/day of AndroGel®, or the testosterone patch (by initial treatment group).

FIG. No. 19(d) is a graph showing the FSH concentrations on days 0 through 180 for men having hypogonadism of an unknown origin and receiving either 5.0 g/day of AndroGel®, 10.0 g/day of AndroGel®, or the testosterone patch (by initial treatment group).

FIG. No. 20(a) is a graph showing the LH concentrations on days 0 through 180 for men having primary hypogonadism and receiving either 5.0 g/day of AndroGel®, 10.0 g/day of AndroGel®, or the testosterone patch (by initial treatment group).

FIG. No. 20(b) is a graph showing the LH concentrations on days 0 through 180 for men having secondary hypogonadism and receiving either 5.0 g/day of AndroGel®, 10.0 g/day of AndroGel®, or the testosterone patch (by initial treatment group).

FIG. No. 20(c) is a graph showing the LH concentrations on days 0 through 180 for men having age-associated hypogonadism and receiving either 5.0 g/day of AndroGel®, 10.0 g/day of AndroGel®, or the testosterone patch (by initial treatment group).

FIG. No. 20(d) is a graph showing the LH concentrations on days 0 through 180 for men having hypogonadism of an unknown origin and receiving either 5.0 g/day of AndroGel®, 10.0 g/day of AndroGel®, or the testosterone patch (by initial treatment group).

FIG. No. 21(a) is a bar graph showing the change in hip BMD for hypogonadal men after 180 days of treatment with 5.0 g/day of AndroGel®, 7.5 g/day of AndroGel®, 10.0 g/day of AndroGel®, or the testosterone patch.

FIG. No. 21(b) is a bar graph showing the change in spine BMD for hypogonadal men after 180 days of treatment with 5.0 g/day of AndroGel®, 7.5 g/day of AndroGel®, 10.0 g/day of AndroGel®, or the testosterone patch

FIG. No. 22 is a graph showing PTH concentrations on days 0 through 180 for hypogonadal men receiving either 5.0 g/day of AndroGel®, 10.0 g/day of AndroGel®, or the testosterone patch (by initial treatment group).

FIG. No. 23 is a graph showing SALP concentrations on days 0 through 180 for hypogonadal men receiving either 5.0 g/day of AndroGel®, 10.0 g/day of AndroGel®, or the testosterone patch (by initial treatment group).

FIG. No. 24 is a graph showing the osteocalcin concentrations on days 0 through 180 for hypogonadal men receiving either 5.0 g/day of AndroGel®, 10.0 g/day of AndroGel®, or the testosterone patch (by initial treatment group).

FIG. No. 25 is a graph showing the type I procollagen concentrations on days 0 through 180 for hypogonadal men receiving either 5.0 g/day of AndroGel®, 10.0 g/day of AndroGel®, or the testosterone patch (by initial treatment group).

FIG. No. 25 is a graph showing the N-telopeptide/Cr ratio on days 0 through 180 for hypogonadal men receiving either 5.0 g/day of AndroGel®, 10.0 g/day of AndroGel®, or the testosterone patch (by initial treatment group).

FIG. No. 27 is a graph showing the Ca/Cr ratio on days 0 through 180 for hypogonadal men receiving either 5.0 g/day of AndroGel®, 10.0 g/day of AndroGel®, or the testosterone patch (by initial treatment group).

FIG. No. 28(a) is a graph showing sexual motivation scores on days 0 through 180 for hypogonadal men receiving either 5.0 g/day of AndroGel®, 7.5 g/day of Androgel®, 10.0 g/day of AndroGel®, or the testosterone patch.

FIG. No. 28(b) is a graph showing overall sexual desire scores on days 0 through 180 for hypogonadal men receiving either 5.0 g/day of AndroGel®, 7.5 g/day of Androgel®, 10.0 g/day of AndroGel®, or the testosterone patch.

FIG. No. 28(c) is a graph showing sexual enjoyment (with a partner) scores on days 0 through 180 for hypogonadal men receiving either 5.0 g/day of AndroGel®, 7.5 g/day of Androgel®, 10.0 g/day of AndroGel®, or the testosterone patch.

FIG. No. 29(a) is a graph showing sexual performance scores on days 0 through 180 for hypogonadal men receiving either 5.0 g/day of AndroGel®, 7.5 g/day of Androgel®, 10.0 g/day of AndroGel®, or the testosterone patch.

FIG. No. 29(b) is a graph showing erection satisfaction performance scores on days 0 through 180 for hypogonadal men receiving either 5.0 g/day of AndroGel®, 7.5 g/day of Androgel®, 10.0 g/day of AndroGel®, or the testosterone patch.

FIG. No. 29(c) is a graph showing percent erection scores on days 0 through 180 for hypogonadal men receiving either 5.0 g/day of AndroGel®, 7.5 g/day of Androgel®, 10.0 g/day of AndroGel®, or the testosterone patch.

FIG. No. 30(a) is a graph showing positive mood scores on days 0 through 180 for hypogonadal men receiving either 5.0 g/day of AndroGel®, 7.5 g/day of Androgel®, 10.0 g/day of AndroGel®, or the testosterone patch.

FIG. No. 30(b) is a graph showing negative mood scores on days 0 through 180 for hypogonadal men receiving either 5.0 g/day of AndroGel®, 7.5 g/day of Androgel®, 10.0 g/day of AndroGel®, or the testosterone patch.

FIG. No. 31(a) is a bar graph showing the change in leg strength on days 90 and 180 for hypogonadal men receiving either 5.0 g/day of AndroGel®, 7.5 g/day of Androgel®, 10.0 g/day of AndroGel®, or the testosterone patch.

FIG. No. 31(b) is a bar graph showing the change in arm strength on days 90 and 180 for hypogonadal men receiving either 5.0 g/day of AndroGel®, 7.5 g/day of Androgel®, 10.0 g/day of AndroGel®, or the testosterone patch.

FIG. No. 32(a) is a bar graph showing the change in total body mass on days 90 and 180 for hypogonadal men receiving either 5.0 g/day of AndroGel®, 7.5 g/day of Androgel®, 10.0 g/day of AndroGel®, or the testosterone patch.

FIG. No. 32(b) is a bar graph showing the change in lean body mass on days 90 and 180 for hypogonadal men receiving either 5.0 g/day of AndroGel®, 7.5 g/day of Androgel®, 10.0 g/day of AndroGel®, or the testosterone patch.

FIG. No. 32(c) is a bar graph showing the change in fat mass on days 90 and 180 for hypogonadal men receiving either 5.0 g/day of AndroGel®, 7.5 g/day of Androgel®, 10.0 g/day of AndroGel®, or the testosterone patch.—

FIG. No. 32(d) is a bar graph showing the change in percent body fat on days 90 and 180 for hypogonadal men receiving either 5.0 g/day of AndroGel®, 7.5 g/day of Androgel®, 10.0 g/day of AndroGel®, or the testosterone patch.

FIG. No. 33 is a flow diagram showing subject progress through an eight-week randomized placebo-controlled depression trial of testosterone transdermal gel.

FIG. No. 34 is a line graph showing the Hamilton Depression Rating Scale scores in an eight-week randomized placebo-controlled depression trial of testosterone transdermal gel.

FIG. No. 35 is a line graph showing the Clinical Impression scores in eight-week randomized placebo-controlled depression trial of testosterone transdermal gel.

FIG. No. 36 is a line graph showing the Beck Depression Inventory scores an eight-week randomized placebo-controlled depression trial of testosterone transdermal gel.

### DETAILED DESCRIPTION OF THE PRESENT INVENTION

While the present invention may be embodied in many different forms, several specific embodiments are discussed herein with the understanding that the present disclosure is to be considered only as an exemplification of the principles of the invention, and it is not intended to limit the invention to the embodiments illustrated. Where the invention is illustrated herein with particular reference to testosterone, it will be understood that any other steroid in the testosterone synthetic pathway can, if desired, be substituted in whole or in part for testosterone in the methods, kits, combinations, and compositions herein described. Where the invention is illustrated herein with particular reference to methyltestosterone, it will be understood that any other agent that inhibits the synthesis of sex hormone binding globulin (SHBG) can, if desired, be substituted in whole or in part for methyltestosterone in the methods, kits, combinations, and compositions herein described. Where the invention is illustrated herein with particular reference to estrogen, it will be understood that any other estrogenic hormone can, if desired, be substituted in whole or in part for estrogen in the methods, kits, combinations, and compositions herein described.

The present invention is directed to methods, kits, combinations, and compositions for treating, preventing or reducing the risk of developing a depressive disorder, or the symptoms associated with, or related to a depressive disorder in a subject in need thereof. The method comprises administering, for example, percutaneously, to a subject a depressive-disorder-effective amount of a steroid in the testosterone synthetic pathway, for example, testosterone. The present invention includes methods of reversing, halting or slowing the progression of a depressive disorder once it becomes clinically evident, or treating the symptoms associated with, or related to the depressive disorder. The subject may already have a depressive disorder at the time of administration, or be at risk of developing a depressive disorder.

Also included in the present invention is a method of administering to a subject in need thereof a steroid in the testosterone synthetic pathway, for example testosterone. In one embodiment, the method comprises administering to the subject a depressive-disorder-effective amount of a percutaneously deliverable composition comprised of a pharmaceutically-acceptable steroid in the testosterone synthetic pathway, for example testosterone, one or more lower alcohols, such as ethanol or isopropanol, a penetration enhancing agent, a thickening agent, and water.

Also included in the methods, kits, combinations, and compositions of the present invention is a pharmaceutical composition comprising a depressive-disorder-effective amount of testosterone. In one embodiment the testosterone composition is formulated as a gel, ointment, cream, or patch. In yet another embodiment the testosterone composition is a hydroalcoholic gel. In another embodiment, the composition is a gel comprising testosterone, one or more lower alcohols, such as ethanol or isopropanol, a penetration enhancing agent, a thickening agent, and water.

The present invention also includes kits comprising percutaneously deliverable testosterone. The kits also contain a set of instructions for the subject. In another embodiment, the methods, kits, combinations, and compositions are used in conjunction with another steroid or a pharmaceutical agent effective at treating, preventing, or reducing the risk of developing a depressive disorder. A pharmaceutical agent effective at treating, preventing, or reducing the risk of developing a depressive disorder include, but are not limited to, an estrogenic hormone, an agent that inhibit the synthesis of sex hormone binding globulin, and an antidepressant agent.

In one embodiment, the composition of the present invention is administered once, twice, or three times a day, or as many times necessary to achieve the desired therapeutic effect. In another embodiment the composition of the present invention is administered once, twice, or three times a day on alternate days. In another embodiment the composition of the present invention is administered once, twice, or three times a day on a weekly, biweekly, or monthly basis.

In one embodiment, the present invention employs testosterone in conjunction with a pharmacologically-effective amount of an estrogenic hormone, an agent that inhibits the synthesis of sex hormone binding globulin, or an antidepressant agent in the same dosage form or in a separate dosage form.

In another embodiment, the methods, kits, combinations, and compositions are used with another steroid or pharmaceutical agent that increases testosterone levels in a subject, for example, an agent that inhibits the synthesis of sex hormone binding globulin, for example, methyltestosterone or fluoxymesterone.

In yet another embodiment, the present invention employs a packet having a polyethylene liner compatible with the components of the gel. In another embodiment, the methods, kits, combinations, and compositions employ a composition that is dispensed from a rigid multi-dose container (for example, with a hand pump) having a larger foil packet of the composition inside the container. Such larger packets can also comprise a polyethylene liner as above.

Additionally, the methods, kits, combinations, and compositions of the present invention optionally include a salt, an ester, an amide, an enantiomer, an isomer, a tautomer, a prodrug, or a derivative of an agent of the present invention, as well as an emollient, a stabilizer, an antimicrobial, a fragrance, or a propellant.

The methods, kits, combinations, and compositions of the present invention provide enhanced treatment options for treating a depressive disorder in a subject, for example, a man or a women, as compared to those currently available.

Besides being useful for human treatment, the present invention is also useful for veterinary treatment of companion mammals, exotic animals and farm animals, including mammals, rodents, and the like. In one embodiment, the mammal includes a horse, a dog, or a cat.

A class of steroids in the testosterone synthetic pathway useful in the methods, kits, combinations, and compositions of the present invention include steroids in the testosterone anabolic or catabolic pathway. In a broad aspect of the invention, the active ingredients employed in the present invention may include anabolic steroids such as androisoxazole, bolasterone, clostebol, ethylestrenol, formyldienolone, 4-hydroxy-19-nortestosterone, methenolone, methyltrienolone, nandrolone, oxymesterone, quinbolone, stenbolone, trenbolone; androgenic steroids such as boldenone, fluoxymesterone, mestanolone, mesterolone, methandrostenolone, 17 ∝ methyltestosterone, 17 alpha-methyl-testosterone 3-cyclopentyl enol ether, norethandrolone, normethandrone, oxandrolone, oxymetholone, prasterone, stanlolone, stanozolol, dihydrotestosterone, testosterone; and progestogens such as anagestone, chlormadinone acetate, delmadinone acetate, demegestone, dimethisterone, dihydrogesterone, ethinylestrenol, ethisterone, ethynodiol, ethynodiol diacetate, flurogestone acetate, gestodene, gestonorone caproate, haloprogesterone, 17-hydroxy-16-methylene-progesterone, 17 alpha-hydroxyprogesterone, 17 alpha-hydroxyprogesterone caproate, medrogestone, medroxyprogesterone, megestrol acetate, melengestrol, norethindrone, norethindrone acetate, norethynodrel, norgesterone, norgestimate, norgestrel, norgestrienone, 19-norprogesterone, norvinisterone, pentagestrone, progesterone, promegestone, quingestrone, and trengestone; and all salts, esters, amides, enantiomers, isomers, tautomers, prodrugs and derivatives of these compounds. (Based in part upon the list provided in The Merck Index, Merck & Co. Rahway, N.J. (1998)). Combinations of the above mentioned steroids can be used in the methods, kits, combinations, and compositions herein described.

Antidepressant agents useful in the methods, kits, combinations, and compositions of the present invention include, for example, bicyclics, such as binedaline, caroxazone, citalopram, dimethazan, fencamine, indalpine, indeloxzine hydrochloride, nefopam, nomifensine, oxitriptan, oxypertine, paroxetine, sertraline, thiazesim, and trazodone; hydrazides/hydrazines, such as benmoxine, iproclozide, iproniazid, isocarboxazid, nialamide, octamoxin, and phenelzine; pyrrolidones, such as cotinine, rolicyprine, or rolipram; tetracyclics, such as maprotiline, metralindole, mianserin, and mitrazepine; tricyclics, such as adinazolam, amitriptyline, amitriptylinoxide, amoxaprine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, and trimipramine; and others, such as adrafinil, amoxapine, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, maprotline, mirtazapine, nefazodone, oxaflozane, phenelzine, piberaline, prolintane, pyrisuccideanol, ritanserin, roxindole, rubidium chloride, sulpride, tandospirone, thozalinone, tofenacin, toloxatone, tranylcypromine, trazodone, L-tryptophan, venlafaxine, viloxazine, and zimeldine; and all salts, esters, amides, enantiomers, isomers, tautomers, prodrugs and derivatives of these compounds. (Based in part upon the list provided in The Merck Index, Merck & Co. Rahway, N.J. (1998)). Combinations of the above mentioned antidepressant agents can be used in the methods, kits, combinations, and compositions herein described.

Other classes of antidepressant agents useful in the methods, kits, combinations, and compositions of the present invention include, for example, anitparkinsonian agents, such as amantadine, benserazide, bietanautine, biperiden, bromocriptine, budipine, carbidopa, dexetimide, diethazine, droxidopa, ethopropazine, ethylbenzhydramine, lazabemide, levodopa, mofegiline, pergolide, piroheptine, pramipexole, pridinol, prodipine, ropinirole, selegiline, talipexole, terguride, and trihexyphenidyl hydrochloride; antipsychotic agents such as benzamides: alizapride, amisulpride, nemoapride, remoxipride, sulpiride, and sultopride; benzisoxazoles, such as risperidone; butyrophenones, such as benperidol, bromperidol, droperidol, fluanisone, haloperidol, melperone, moperone, pipamperone, spiperone, timiperone, and trifluperidol; phenothiazines, such as acetophenazine, butaperazine, carphenazine, chlorproetbazine, chlorpromazine, clospirazine, cyamemazine, dixyranzine, fluphenazine, imiclopazine, mepazine, mesoridazine, methoxypromazine, metofenazate, oxaflumazine, perazine, pericyazine, perimethazine, perphenazine, piperacetazine, pipotiazine, prochlorperazine, promazine, sulforidazine, thiopropazate, thioproperazine, thioridazine, trifluoperazine, and triflupromazine; thioxanthenes, such as chlorprothixene, clopenthixol, flupentixol, thiothixene; other tricyclics, such as benzquinamide, carpipramine, clocapramine, clomacran, clothiapine, clozapine, mosapramine, olanzapine, opipramol, prothipendyl, seroquel®, tetrabenazine, and zotepine;,and other anitparkinsonian agents, such as buramate, fluspirilene, molindone, penfluridol, pimozide, ziprasidone; dopamine receptor angonists, such as bromocriptine, cabergoline, carmoxirole, dopexamine, fenoldopam, ibopamine, lisuride, pergolide, pramipexole, quinagolide, ropinrole, roxindole, and talipexole; dopamine receptor antagonist, such as amisuipride, clebopride, domperidone, metoclopramide, mosapramine, nemonapride, remoxipride, risperidone, sulpiride, sultopride, and ziprasidone; monoamine oxidase inhibiting agents, such as iproclozide, iproniazid, isocarboxazid, lazabemide, mofegiline, moclobemide, octamoxin, pargyline, pheneizine, phenoxypropazine, pivalylbenzhydrazine, prodipine, selegiline, and toloxatone, tranylcypromine; and selective serotonin reuptake inhibitors, such as, citalopram, fluoxetine, fluvoxamine, venlafaxine, sertraline, paroxetine; and all salts, esters, amides, enantiomers, isomers, tautomers, prodrugs and derivatives of these compounds. (Based in part upon the list provided in The Merck Index, Merck & Co. Rahway, N.J. (1998)). Combinations of the above mentioned antidepressant agents can be used in the methods, kits, combinations, and compositions herein described.

Illustratively, antidepressant agents of particular interest that can be used in the methods, kits, combinations, and compositions of the present invention include, but are not limited to Ativan®, Librium®, Limbitrol®, Tranxene®, Valium®, Xanax®, Atarax®, BuSpar®, Effexor®, Mebaral®, Miltown®, Paxil®, Sinequan®, Triavil®, Vistaril®, Remeron®, Serzone®, Wellbutrin®, Nardil®, Parnate®, Celexa®, Prozac®, Zoloft®, Elavil®, Etrafon®, Norpramin®, Surmontil®, Vivactil®, Depakote®, Eskalith®, lithium, Lithobid®, Klonopin®, Clozaril®, Haldol®, Loxitane®, Moban®, Navane®, Orap®, Risperdal®, Seroquel®, Zyprexa®, Compazine®, Serentil®, Stelazine®, Thioridazine®, Trilafon®, and Luvox®. Combinations of the above mentioned antidepressant agents can be used in the methods, kits, combinations, and compositions herein described.

A class of steroids or pharmaceutical agents that increases testosterone levels in a subject useful in the methods, kits, combinations, and compositions of the present invention include compounds that inhibit the synthesis of the sex hormone binding globulin. Sex hormone binding globulin is a serum protein, and is known to bind to testosterone and estradiol, effecting the biological activity of these hormones. Specific compounds of interest that inhibit the synthesis the sex hormone binding globulin include but are not limited to methyltestosterone and fluoxymesterone, and all salts, esters, amides, enantiomers, isomers, tautomers, prodrugs and derivatives of these compounds. Combinations of the above these compounds can be used in the methods, kits, combinations, and compositions herein described. Methyltestosterone is currently available in various formulations including those available orally, for example, ANDROID® and TESTRED®. Fluoxymesterone is also currently available in various formulations including those available orally, for example, HALOSTESTIN®.

While not wishing to be bound by theory, it is believed that methyltestosterone decreases hepatic synthesis of endogenous proteins like sex hormone binding globulin. This decrease in synthesis produces a decline in blood concentrations of sex hormone binding globulin, which is the primary means of endogenous hormone transport. The decrease in sex hormone binding globulin subsequently causes an increase in free-hormone concentration for binding at the receptor. Transdermal application of an androgen, for example, testosterone, or an estrogen, for example, estradiol, bypasses first-pass metabolism and can provide a means of increasing hormone concentrations in the bloodstream. Thus, when used in combination, methyltestosterone and percutaneously administered testosterone (and optionally estradiol) produce a greater therapeutic effect and provide a means of increasing hormone concentrations in the bloodstream. Methyltestosterone and testosterone (and optionally estradiol) produce a greater therapeutic effect than either entity alone because the decrease in hormone binding ability is coupled with an increased hormone bioavailability, producing higher free-hormone concentrations that would be produced by testosterone alone.

In another embodiment of the present invention, the estrogenic hormone that can be used in conjunction with the methods, kits, combinations, and composition is the naturally occurring estrogen 17 beta-estradiol (beta-estradiol; 1, 3, 5(10)-estratriene-3, 17 beta-diol). Other estrogenic steroid hormones can be used in partial or complete replacement of 17 beta-estradiol, for example, an ester which is biologically compatible and can be absorbed effectively transdermally. The estradiol esters can be, illustratively estradiol-3,17-diacetate; estradiol-3-acetate; estradiol-17-acetate; estradiol-3,17-divalerate; estradiol-3-valerate; estradiol-17-valerate; 3-mono, 17-mono and 3,17-dipropionate esters, corresponding cypionate, heptanoate, benzoate and the like esters; ethynyl estradiol; estrone and other estrogenic steroids and salts, enantiomers, isomers, tautomers, prodrugs and derivatives thereof that are possible to administer by transdermal route. Other estrogen-related compounds that may be used in the methods, kits, combinations, and compositions of the present invention include, but are not limited to conjugated estrogens (including estrone sulfate, equilin, and 17-.alpha.-dihydroequilin), estradiol valerate, estriol, estrone, estrone sulfate, estropipate, ethinyl estradiol, mestranol, and all salts, esters, amides, enantiomers, isomers, tautomers, prodrugs and derivatives of these compounds.

Estrogenic hormones are currently available in various formulations including, but not limited to those available as a cream, pessary, vaginal ring, vaginal tablet, transdermal preparation, gel, and oral tablet. Examples of vaginal creams include PREMARIN® (conjugated estrogen), ORTHO DIENOSTEROL® (dienosterol), and OVESTIN® (estriol). Available pessary formulations include ORTHO-GYNEST® (estriol), and TAMPOVAGAN® (stilbestrol). An example of a vaginal ring formulation is ESTRING® (estradiol), and an example of a vaginal tablet is VAGIFEM® (estradiol). Available transdermal estrogen preparations containing estradiol include ERC ALORA®, CLIMARA®, DERMESTRIL®, ESTRADERM®, ESTRADERM® TTS, ESTRADERM® MX, EVOREL®, FEMATRIX®, FEMPATCH®, FEMSEVEN®, MENOREST®, PROGYNOVA® TS, and VIVELLE®. Estrogen gels containing estradiol include ESTRAGEL (under development by Applicant), and SANDRENA®. Estradiol is also available formulated as an implant pellet, for example, ESTRADIOL IMPLANT®. Tablet formulations include PREMARIN® (conjugated estrogen), ESTRATAB® (esterified estrogen), ESTRATEST® (esterified estrogen, methyltestosterone), MENEST® (esterified estrogen), CLIMAGEST®, (estradiol), CLIMAVAL® (estradiol), ELLESTE SOLO® (estradiol), ESTRACE® (estradiol), PROGYNOVA® (estradiol), ZUMENON® (estradiol), HORMONIN® (estradiol, estrone, estriol), HARMOEN® (estrone), OGEN® (estropipate), and ORTHO-EST® (estropipate).

Combinations of the above mentioned estrogenic hormones can be used in the methods, kits, combinations, and compositions herein described.

In one embodiment, testosterone is formulated as a hydroalcoholic gel. In another embodiment, the gel comprises testosterone, one or more lower alcohols, such as ethanol or isopropanol, a penetration enhancing agent, a thickening agent, and water. Additionally, the gel optionally includes the a salt, an ester, an amide, an enantiomer, an isomer, a tautomer, a prodrug, or a derivative of testosterone, as well as an emollient, a stabilizer, an antimicrobial, a fragrance, or a propellant.

Illustratively, certain formulations of the present invention deliver about 0.01 g to about 100 g testosterone, or the equivalent thereof, to a subject per dosage unit. In another embodiment of the present invention, the formulations deliver from about 0.1 g to about 10 g testosterone, or the equivalent thereof, to a subject per dosage unit. In yet another embodiment of the present invention, the formulations of the present invention deliver from about 0.17 g to about 5 g testosterone, or the equivalent thereof, to a subject per dosage unit. In another embodiment of the present invention, the formulations of the present invention deliver about 1 g testosterone, or the equivalent thereof, to a subject per dosage unit. In still another embodiment of the present invention, the formulations of the present invention deliver about 0.25 g testosterone, or the equivalent thereof, to a subject per dosage unit. Thus, for example, a testosterone gel, ointment, cream or patch is formulated as a single dosage unit for once a day administration contains about 0.17 g, or about 0.25 g, or about 0.5 g testosterone, or about 1.0 g testosterone, while a gel, ointment, cream or patch formulated as a single dosage unit for once a week administration contains about 1.19 g, or about 1.75 g, or about 3.50 g, or about 7.0 g testosterone, respectfully.

In one embodiment, the formulation is a gel, an ointment, a cream or a patch and is comprised of testosterone; a penetration enhancing agent, such as isopropyl myristate; a thickening agent, such as Carbopol; a lower alcohol, such as ethanol or isopropanol; and water. In another embodiment the formulation is a gel, an ointment, a cream or a patch and is comprised of the following substances in approximate percentages:

TABLE 4
Composition of Testosterone
Formulation

| SUBSTANCE | AMOUNT (w/w) |
|---|---|
| Testosterone | 0.01-70% |
| Penetration | 0.01-50% |
| enhancing agent | |
| Thickening agent | 0.01-50% |
| Lower alcohol | 30-98% |
| Purified water (qsf) | 100% |

Illustratively, in a 100 g composition, the gel, ointment, cream, or patch may contain about 0.01 g to about 70 g of testosterone, about 0.01 g to about 50 g penetration enhancing agent, about 0.1 g to about 50 g thickening agent, and about 30 g to about 98 g lower alcohol. In another embodiment, in a 100 g composition, the gel, ointment, cream, or patch may contain about 0.1 g to 10 g of testosterone, about 0.1 g to about 5 g of penetration enhancing agent, about 0.1 g to about 5 g of thickening agent, an about 45 g to about 90 g lower alcohol.

In yet another embodiment, the composition is a gel, ointment, cream, or patch that further comprises a hydroxide releasing agent, such as sodium hydroxide (fore example, 0.1 N NaOH), in an amount of about 0.1% to about 10% w/w of the composition.

In one embodiment, the formulation is a gel and is comprised of the following substances in approximate weights:

TABLE 5
Composition of AndroGel ®

| | AMOUNT (w/w) |
|---|---|
| | PER 100 g OF |
| SUBSTANCE | GEL |
| Testosterone | 1.0 g |
| Isopropyl myristate | 0.50 g |
| Carbopol 980 | 0.90 g |
| 0.1 N NaOH | 4.72 g |
| Ethanol (95% w/w) | 72.5 g* |
| Purified water | q.s. |

*Corresponding to 67 g of ethanol.

In another embodiment, the formulation is a gel and is comprised of the following substances in approximate weights:

TABLE 6
Composition of Relibra ®

| | AMOUNT (w/w) |
|---|---|
| | PER 100 g OF |
| SUBSTANCE | GEL |
| Testosterone | 0.1 g |
| Isopropyl myristate | 0.50 g |
| Carbopol 980 | 0.90 g |
| 0.1 N NaOH | 4.72 g |
| Ethanol (95% w/w) | 72.5 g* |
| Purified water | q.s. |

*Corresponding to 67 g of ethanol.

In still another embodiment, the composition comprises testosterone in an amount greater than 0.01%, a penetration enhancing agent in an amount greater than about 0.1%, a thickening agent in an amount greater than about 0.1%, and a lower alcohol in an amount greater than about 30% w/w of the composition.

The gel, ointment, cream, or patch is rubbed or placed onto an area of skin of the subject and allowed to dry. Illustratively, the gel, ointment, or cream is rubbed onto an area of skin, for example, on the upper outer thigh and/or hip once daily. Following application the subject washes his or her hands. Application of the gel results in an increased testosterone level having a desirable pharmacokinetic profile effective to treat, prevent or reduce the risk of developing a depressive disorder, or the symptoms associated with, or related to a depressive disorder in the subject. The composition is thus useful for treating a number of disorders, conditions or diseases in both men and women.

In one embodiment of the present invention a method is provided for treating, preventing or reducing the risk of developing a depressive disorder in a subject in need thereof, that is, a subject indicated for having, or at risk of developing a depressive disorder. The method comprises administering a depressive-disorder-effective amount of a composition to an area of skin of the subject for delivery of a steroid in the testosterone synthetic pathway to blood serum of the subject. The composition comprises:

(a) about 0.01% to about 70% (w/w) steroid in the testosterone synthetic pathway;

(b) about 0.01% to about 50% (w/w) penetration enhancing agent;

(c) about 0.01% to about 50% (w/w) thickening agent; and

(d) about 30% to about 98% (w/w) lower alcohol.

The composition is capable of releasing the steroid after applying the composition to the skin at a rate and duration that delivers at least about 10 μg per day of the steroid to the blood serum of the subject.

In one embodiment of the present invention the steroid in the testosterone synthetic pathway is testosterone.

In another embodiment of the methods, kits, combinations, and compositions of the present invention, the composition is capable of releasing the testosterone after applying the composition to the skin of a subject at a rate and duration that achieves a circulating serum concentration of testosterone greater than about 400 ng per dl serum during a time period beginning about 2 hours after administration and ending about 24 hours after administration.

In another embodiment of the methods, kits, combinations, and compositions of the present invention, the composition is capable of releasing the testosterone after applying the composition to the skin of a subject at a rate and duration that achieves a circulating serum concentration of the testosterone between about 400 ng testosterone per dl serum to about 1050 ng testosterone per dl serum.

In another embodiment of the methods, kits, combinations, and compositions of the present invention, for each about 0.1 gram per day application of the composition of the present invention to the skin of a subject, an increase of at least about 5 ng/dl in serum testosterone concentration results in the subject.

In another embodiment of the methods, kits, combinations, and compositions of the present invention, the composition of the present invention is provided to a subject for daily administration in about a 0.1 g to about a 10 g dose.

In yet another embodiment of the methods, kits, combinations, and compositions of the present invention, the subject in need of treatment has a serum testosterone level before the first application (pretreatment) of the composition of the present invention of less than about 300 ng/dl.

In another embodiment of the methods, kits, combinations, and compositions of the present invention, where after at least about 30 days of daily administration of the composition of the present invention the serum testosterone concentration in a subject is at least about 490 ng/dl to about 860 ng/dl.

In still another embodiment of the methods, kits, combinations, and compositions of the present invention, where after at least about 30 days of daily administration of the composition of the present invention the total serum androgen concentration in a subject is greater than about 372 ng/dl.

In another embodiment of the methods, kits, combinations, and compositions of the present invention, the composition of the present invention is administered once, twice, or three times daily to a subject for at least about 7 days.

The present invention also provides a method of treating, preventing or reducing the risk of developing a depressive disorder in a subject in need thereof, that is, a subject indicated for having, or at risk of developing a depressive disorder, by administering to the subject:

(a) an amount of a composition comprising:

(i) about 0.01% to about 70% (w/w) steroid in the testosterone synthetic pathway;

(ii) about 0.01% to about 50% (w/w) penetration enhancing agent;

(iii) about 0.01% to about 50% (w/w) thickening agent; and

(iv) about 30% to about 98% (w/w) lower alcohol; and

(b) an amount of a therapeutic agent comprising an antidepressant, an inhibitor of the synthesis of sex hormone binding globulin, or an estrogenic hormone.

The composition is administered to an area of skin of the subject for delivery of the steroid in the testosterone synthetic pathway to the blood serum of the subject, and is capable of releasing the steroid after applying the composition to the skin at a rate and duration that delivers at least about 10 μg per day of the steroid to the blood serum of the subject. The amount of the composition and the amount of the therapeutic agent together make a depressive-disorder-effective amount.

In one embodiment of the methods, kits, combinations, and compositions of the present invention, the composition and the therapeutic agent are provided as separate components to a kit.

In another embodiment of the methods, kits, combinations, and compositions of the present invention, the composition and the therapeutic agent are administered substantially simultaneously, or sequentially.

In still another embodiment of the methods, kits, combinations, and compositions of the present invention, the therapeutic agent is administered orally, percutaneously, intravenously, intramuscularly, or by direct absorption through mucous membrane tissue.

The present invention also provides a pharmaceutical composition, comprising:

(i) about 0.01% to about 70% (w/w) steroid in the testosterone synthetic pathway;

(ii) about 0.01% to about 50% (w/w) penetration enhancing agent;

(iii) about 0.01% to about 50% (w/w) thickening agent;

(iv) about 30% to about 98% (w/w) lower alcohol; and

(v) a therapeutic agent comprising an antidepressant, an inhibitor of the synthesis of sex hormone binding globulin, or an estrogenic hormone.

The composition is administered to an area of skin of the subject for delivery of the testosterone and the therapeutic agent to the blood serum of the subject, and is capable of releasing the steroid after applying the composition to the skin at a rate and duration that delivers at least about 10 μg per day of the steroid to the blood serum of the subject. The amount of the testosterone and the amount of the therapeutic agent together make a depressive-disorder-effective amount.

Achieving target delivery rates demonstrated by testosterone gel can be estimated from the pharmacokinetics in testosterone gel in men. The mean serum concentration (Cavg) values in men after applying of varying amounts of gel to the upper body is given below in Table 7.

TABLE 7
Mean Average Serum Testosterone Concentrations and Daily Delivery
Rate after Administration of Testosterone Gel 1% in Men

| Dose (μL) | Mean Cavg | Daily Delivery Rate |
|---|---|---|
| (gram) | (ng/dL) | (μg/day)^{a} |
| 5.0 | 555 (±225) | 3330 |
| 7.5 | 601 (±309) | 3606 |
| 10 | 713 (±209) | 4278 |

^{a}Metabolic Clearance Rate of Daily Testosterone = 600 L/day

Based on the results obtained in men, a testosterone gel dose of 0.5 grams delivers approximately 300 μg of testosterone per day.

Illustratively, for an adult woman, a depressive-disorder-effective amount of testosterone per daily dose delivers to the blood serum typically greater than about 10 μg of testosterone per day, or to about 25 μg to about 150 μg to about 300 μg of testosterone per day. Thus, to achieve a serum blood level of about 100 μg testosterone, the composition is administered at about 0.17 g/day, which delivers about 1.7 mg/day of testosterone to the skin of which about 0.1 mg, is absorbed; or to achieve a serum blood level of about 150 μg testosterone, the composition is administered at about 0.25 g/day, which delivers about 2.5 mg/day of testosterone to the skin of which about 0.15 mg, is absorbed; or to achieve a serum blood level of about 300 μg testosterone, the composition is administered at about 0.5 g/day, which delivers 5.0 mg/day of testosterone to the skin of which about 0.3 mg, is absorbed.

The phrase “depressive disorder” refers to a condition, disorder, or disease such as a mood disorder, decreased libido, melancholia, reactive depression, endogenous depression, endogenomorphic depression, anaclitic depression, or any depressive symptom sufficient to meet one or more of the DSM-IV criteria for current major depressive disorder, or any depressive symptom that increases a depression score on the Hamilton Rating Scale or the Depression Beck Depression Inventory.

The term “treat” or “treatment” as used herein refers to any treatment of a mammalian condition, disorder, or disease associated with a depressive disorder, and includes, but is not limited to, preventing the condition, disorder, or disease from occurring in a subject which may be predisposed to the condition, disorder, or disease, but has not yet been diagnosed as having the condition, disorder, or disease; inhibiting the condition, disorder, or disease, for example, arresting the development of the condition, disorder, or disease; relieving the condition, disorder, or disease, for example, causing regression of the condition, disorder, or disease; or relieving the condition caused by the disease or disorder, for example, stopping the symptoms of the disease or disorder. In one embodiment “treat” or “treatment” includes, for example, improving or alleviating a mood disorder, increasing libido, improving or alleviating one or more symptoms of melancholia, improving or alleviating one or more symptoms of reactive depression, improving or alleviating one or more symptoms of endogenous depression, improving or alleviating one or more symptoms of endogenomorphic depression, improving or alleviating one or more symptoms of anaclitic depression, or improving or alleviating any depressive symptom that meets the DSM-IV criteria for current major depressive disorder, or improving or alleviating any depressive symptom that increases a depression score on the Hamilton Rating Scale or the Depression Beck Depression Inventory.

The term “prevent” or “prevention,” in relation to a depressive condition, disorder, or disease, means no depressive condition, disorder, or disease development if none had occurred, or no further depressive condition, disorder, or disease development if there had already been development of the depressive condition, disorder, or disease.

A “depressive-disorder effect” or “depressive-disorder-effective amount” is intended to qualify the amount of an agent required to treat or prevent a depressive disorder in a subject, or relieve to some extent one or more of the symptoms associated with, or related to, a depressive disorder in a subject. In a mammal, this includes, but is not limited to, improving or alleviating a mood disorder, increasing libido, improving or alleviating one or more symptoms of melancholia, improving or alleviating one or more symptoms of reactive depression, improving or alleviating one or more symptoms of endogenous depression, improving or alleviating one or more symptoms of endogenomorphic depression, improving or alleviating one or more symptoms of anaclitic depression, or improving or alleviating any depressive symptom that meets the DSM-IV criteria for current major depressive disorder, or improving or alleviating any depressive symptom that increases a depression score on the Hamilton Rating Scale or the Depression Beck Depression Inventory. Treatment of a subject with the methods, kits, combinations, and compositions of the present invention also include, for example, normalizing hypogonadism; improving sexual dysfunction; normalizing cholesterol levels; normalizing abnormal electrocardiograms of subjects and improving vasomotor symptoms; improving diabetic retinopathy as well as lowering the insulin requirements of diabetic subjects; decreasing the percentage of body fat; normalizing glucose levels; decreasing the risk factors for cardiovascular disease, including normalizing hypertension, and treating obesity; preventing osteoporosis, osteopenia, vaginal dryness, and thinning of the vaginal wall; relieving menopausal symptoms and hot flashes; improving cognitive dysfunction; treating, preventing or reducing the onset of cardiovascular disease, Alzheimer's disease, dementia, and cataracts; and treating, preventing or reducing the risk of cervical, uterine or breast cancer.

When the compositions of the present invention are used in a “depressive-disorder effective amount” this means that the concentration of the therapeutic agent is such that a therapeutic level of agent is delivered over the term that the composition is to be used. Such delivery is dependent on a number of variables including the time period for which the individual dosage unit is to be used, the flux rate of the therapeutic agent, for example, testosterone, from the gel, surface area of application site, etc. The amount of therapeutic agent necessary can be experimentally determined based on the flux rate of the drug through the gel, for example, and through the skin when used with and without enhancers. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of menopause in accordance with the present invention. In terms of treatment protocols, it should be appreciated that the dosage to be administered will depend on several factors, including the particular agent that is administered, the route administered the condition of the particular subject, etc. Generally speaking, one will desire to administer an amount of the agent that is effective to achieve a serum level commensurate with the concentrations found to be effective in vitro. Thus, where an agent is found to demonstrate in vitro activity at, for example, 10 ng/ml, one will desire to administer an amount of the agent that is effective to provide about a 10 ng/ml concentration in vivo. Determination of these parameters is well within the skill of the art. These considerations, as well as effective formulations and administration procedures are well known in the art and are described in standard textbooks.

In order to measure and determine the amount of testosterone to be delivered to a subject to administer a depressive-disorder effective amount to the subject, serum testosterone concentrations can be measured using standard assay techniques. For example, free serum testosterone levels are measured by the recently validated and highly sensitive equilibrium dialysis method discussed in Sinha-Hikim et al., The Use of a Sensitive Equilibrium Dialysis Method for the Measurement of Free Testosterone Levels in Healthy, Cycling Women and in HIV-Infected Women, 83 J. CLINICAL ENDOCRINOLOGY & METABOLISM 1312-18. (1998), and is herein fully incorporated by reference.

As used herein, the phrases “androgen deficiency” or “testosterone deficiency” are used interchangeably, and refer to lower serum levels of free testosterone in a subject as compared to the median serum levels for healthy subject of the same age. For example, normal cycling women produce approximately 300 μg of testosterone per day. Their total serum testosterone levels generally range from about 20 ng/dL to about 80 ng/dL averaging about 40 ng/dL. In healthy young women, for example, mean free testosterone levels are generally about 3.6 pg/mL. However, several factors may influence both total and free testosterone serum levels. For example, in regularly ovulating women, there is a small but significant increase in plasma testosterone levels during the middle third of the menstrual cycle. However, mean testosterone levels (1.2 nmol/L or 33 ng/dL) and mean free testosterone levels (12.8 pmo/L or 3.6 pg/mL) during the luteal and follicular phases are not significantly different. Additionally, testosterone production declines continuously after age 30 so that serum testosterone levels in a 60-year-old woman are only 50% of the levels in a young 30-year-old woman. Although the percentage of free testosterone generally does not vary with age, an absolute decline in free testosterone has been observed. This decline does not occur abruptly at menopause but instead occurs gradually and continuously as a result of the age-related decrease in both the adrenal and ovarian androgen production. Thus, women begin to experience symptoms associated with menopause in the immediate pre-menopausal years. The decline in testosterone following menopause results from the combination of ovarian failure, decreasing renal secretion, and peripheral conversion. Also, for example, after ovariectomy, testosterone concentrations decrease by about 50%. Diagnosis of a testosterone deficiency is known to the average physician practicing in the relevant field of medicine.

The use of the term “about” in the present disclosure means “approximately,” and use of the term “about” indicates that dosages slightly outside the cited ranges may also be effective and safe, and such dosages are also encompassed by the scope of the present claims.

The term “prodrug” refers to a drug or compound in which the pharmacological action (active curative agent) results from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak, et al., Am. J. Physiol, 269:G210-218 (1995), describe dexamethasone-beta-D-glucuronide. McLoed, et al., Gastroenterol., 106:405-413 (1994), describe dexamethasone-succinate-dextrans. Hochhaus, et al., Biomed. Chrom., 6:283-286 (1992), describe dexamethasone-21-sulphobenzoate sodium and dexamethasone-21-isonicotinate. Additionally, J. Larsen and H. Bundgaard [Int. J. Pharmaceutics, 37, 87 (1987)] describe the evaluation of N-acylsulfonamides as potential prodrug derivatives. J. Larsen et al., [Int. J. Pharmaceutics, 47, 103 (1988)] describe the evaluation of N-methylsulfonamides as potential prodrug derivatives. Prodrugs are also described in, for example, Sinkula et al., J. Pharm. Sci., 64:181-210 (1975).

The term “derivative” refers to a compound that is produced from another compound of similar structure by the replacement of substitution of one atom, molecule or group by another. For example, a hydrogen atom of a compound may be substituted by alkyl, acyl, amino, etc., to produce a derivative of that compound.

The phrase “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The phrase “penetration enhancing agent” refers to an agent known to accelerate the delivery of the drug through the skin. These agents also have been referred to as accelerants, adjuvants, and absorption promoters, and are collectively referred to herein as “enhancers.” This class of agents includes those with diverse mechanisms of action including those which have the function of improving the solubility and diffusibility of the drug, and those which improve percutaneous absorption by changing the ability of the stratum corneum to retain moisture, softening the skin, improving the skin's permeability, acting as penetration assistants or hair-follicle openers or changing the state of the skin such as the boundary layer. The penetration enhancing agent of the present invention is a functional derivative of a fatty acid, which includes isosteric modifications of fatty acids or non-acidic derivatives of the carboxylic functional group of a fatty acid or isosteric modifications thereof. In one embodiment, the functional derivative of a fatty acid is an unsaturated alkanoic acid in which the —COOH group is substituted with a functional derivative thereof, such as alcohols, polyols, amides and substituted derivatives thereof. The term “fatty acid” means a fatty acid that has four (4) to twenty-four (24) carbon atoms.

Non-limiting examples of penetration enhancing agents include C₈-C₂₂ fatty acids such as isostearic acid, octanoic acid, and oleic acid; C₈-C₂₂ fatty alcohols such as oleyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

The thickening agents used herein may include anionic polymers such as polyacrylic acid (CARBOPOL® by B. F. Goodrich Specialty Polymers and Chemicals Division of Cleveland, Ohio), carboxypolymethylene, carboxymethylcellulose and the like, including derivatives of Carbopol® polymers, such as Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3, and other polymers such as Pemulen® polymeric emulsifiers, and Noveon® polycarbophils. Additional thickening agents, enhancers and adjuvants may generally be found in Remington's The Science and Practice of Pharmacy, Meade Publishing Co., United States Pharmacopeia/National Formulary.

As used herein, the term “lower alcohol,” alone or in combination, means a straight-chain or branched-chain alcohol moiety containing one to about six carbon atoms. In one embodiment, the lower alcohol contains one to about 4 carbon atoms, and in another embodiment the lower alcohol contains two to about 3 carbon atoms. Examples of such alcohol moieties include methanol, ethanol, n-propanol, isopropanol, n-butanol, isobutanol, sec-butanol, and tert-butanol.

As used herein, the term “lower alkyl”, alone or in combination, means a straight-chain or branched-chain alkyl radical containing one to about six carbon atoms. In one embodiment, the lower alkyl contains one to about four carbon atoms. Examples of such radicals include methyl, ethyl, n-propyl, isopropyl, n-butyl, isobutyl, sec-butyl, and tert-butyl.

Decreased production of testosterone in a subject can be caused by several factors well known to those skilled in the relevant field of medicine. For example, in a woman decreased testosterone production can be caused by use of oral contraceptives; surgery, for example, removal of the uterus (hysterectomy), or removal of one of both ovaries (oophorecty/ovaricctomy); estrogen replacement therapy in post-menopausal women; premature ovarian failure; adrenal dysfunction, for example primary adrenal insufficiency; corticosteroid-induced adrenal suppression; panhypopituitarism; and chronic illness, such as systemic lupus erythematosis, rheumatoid arthritis, human immunodeficiency virus (HIV) infection, chronic obstructive lung disease, and end stage renal disease.

Physiological and psychological disorders associated with testosterone deficiency in a subject include, for example, decreased mood, libido and sexual performance, decreased bone mineral density and related markers, diminished body composition, human immunodeficiency virus wasting syndrome, decreased cognition, diminished mood and self-esteem, decreased muscle mass and perfornmance, premenstrual syndrome, and autoimmune disease.

Nevertheless, there exist well-defined subject populations where testosterone production is clearly deficient and where associated symptomatology has been described, and such populations are contemplated as falling within the scope of the present invention.

Subjects to be treated with the present invention include those at risk of developing a depressive disorder, or subjects currently experiencing a depressive disorder event. Standard depressive disorder risk factors are known to the average physician practicing in the relevant field of medicine. Subjects who are identified as having one or more risk factors known in the art to be at risk of developing a depressive disorder, as well as people who already have a depressive disorder, are intended to be included within the group of people considered to be at risk for having a depressive disorder event.

In addition, contemplated methods, kits, combinations, and compositions of the present invention are useful to treat testosterone deficiency in a subject, which includes a subject where testosterone production is deficient, or where the associated symptomatology related to deficient testosterone production is clinically evident. In men, this includes age, for example. In women, this includes, for example, a oophorectomized/hysterectomized woman, a post-menopausal woman on estrogen replacement therapy, a woman on oral contraceptives, a woman with an ovariectomy, a woman with premature ovarian failure, a woman with adrenal dysfunction, a woman with corticosteroid-induced adrenal suppression, a woman with panhypopituitarism, a woman with primary adrenal insufficiency, and a woman experiencing chronic illness, such as systemic lupus erythematosis, rheumatoid arthritis, human immunodeficiency virus (HIV) infection, chronic obstructive lung disease, and end stage renal disease.

In one embodiment of the present invention, the methods, kits, combinations, and composition are usefuil in treating a woman who have undergone surgery, including, for example, bilateral oophorectomy with hysterectomy, and particularly a woman whose surgery was performed at a younger age, prior to her natural menopause. In the U.S. alone, more than 250,000 women undergo combined oophorectomy/hysterectomy procedures annually and are clearly deficient in testosterone production. Serum testosterone levels typically decrease by 50% in a oophorectomized woman compared to their pre-operative levels, however, in some cases the levels may still remain within the normal reference range (approximately 20-80 ng/dL). Estrogen and progesterone levels, which are primarily dependent on ovarian secretion, are also markedly reduced after oophorectomy. The resulting multiple hormone deficiency state is associated with vasomotor symptoms, high-turnover osteoporosis, and female sexual dysfunction. While estrogen replacement therapy is standard for the treatment of vasomotor symptoms and osteoporosis in the oophorectomized/hysterectomized female, concomitant testosterone therapy has not been indicated for treatment of female sexual dysfunction or for its effects with estrogen replacement therapy on bone metabolism. Such women are contemplated as falling within the scope of the present invention.

In another embodiment of the present invention, the methods, kits, combinations, and composition are useful in treating a post-menopausal woman. In contrast to the oophorectomized state, the post-menopausal ovary may continue to synthesize testosterones in the stromal tissue at rates that are not necessarily lower than the premenopausal period. In some post-menopausal women, testosterone levels increase as a consequence of the stromal response to elevated luteinizing hormone levels, while in others testosterone levels decrease or remain the same. Since estrogen replacement therapy lowers luteinizing hormone levels, ovarian testosterone secretion would be expected to decrease in post-menopausal women who receive estrogen replacement therapy. With oral estrogen replacement therapy preparations, the fall in testosterone levels may be obscured by the concomitant rise in sex hormone binding globulin levels, which reduces testosterone clearance. However, free and/or bioavailable testosterone levels are found to be lower in a post-menopausal woman receiving oral estrogen replacement therapy. While the effects of transdermal estrogen replacement therapy on the androgen/luteinizing hormone status of post-menopausal women has not been studied, a reduction in total and free testosterone levels, associated with a decrease in luteinizing hormone levels, would also be expected. As many post-menopausal women experience symptoms of female sexual dysfunction that are not ameliorated by estrogen replacement therapy, it is believed that testosterone deficiency is a contributing factor, and this group of women would fall within the scope of the present invention.

In yet another embodiment of the present invention, the methods, kits, combinations, and composition are useful in treating a woman who uses oral contraception. Oral contraception is the most common method of contraception among adolescents, and overall about 46% of the sexually active population use oral contraception. The most common type of oral contraceptive contains both estrogen and progestin and has proven to be about 99% effective. Thus, almost half of all premenopausal women (<44 years old) are potentially taking oral contraceptives. In comparison to healthy “cycling” women, the testosterone levels in women treated with estrogen-containing oral contraceptives are markedly lower, particularly when compared at the pre-ovulatory phase of the normal cycle, when testosterone levels are highest. This effect result from the luteinizing hormone suppression produced by oral contraceptives and is analogous to the effect of estrogen replacement therapy described above. Psychosexual aspects of perception are affected by the lower testosterone levels and may be related to the clinical observation of decreased libido in some women using oral contraceptives.

In yet another embodiment of the present invention, the methods, kits, combinations, and composition are useful in treating a woman who have an undergone an ovariectomy by, for example, surgery, chemical means, irradiation, or gonadotropin-releasing hormone antagonists. Such surgery leads to decreased ovarian androgen product.

In another embodiment of the present invention, the methods, kits, combinations, and composition are useful in treating a woman with premature ovarian failure. Premature ovarian failure, such as that associated with Turner's Syndrome or the autoimmune or idiopathic destruction of the ovary, is associated with impaired testosterone production.

In still another embodiment of the present invention, the methods, kits, combinations, and composition are useful in treating a subject who has decreased adrenal function. Decrease adrenal function, which may result from a variety of causes, represents another category of subjects where testosterone production may be reduced by approximately 50%. Primary adrenocortical deficiency, or Addison's disease, is a rare endocrine disorder with multiple etiologies, including tuberculosis and fungal infections. The estimated prevalence in women is approximately 5 per 100,000. Due to the lack of gluco- and mineral corticoid secretion, Addison's disease can be life threatening. While some researchers have noted the associated testosterone deficiency, replacement therapy is often ignored. As the adrenocorticotropic hormone appears to be the primary stimulator of adrenal androgen production, deficient adrenocorticotropic hormone secretion can also lead to testosterone deficiency in women. This can result from pituitary disease or surgery, for example, secondary adrenocortical deficiency, or as a pharmacological effect of exogenous corticosteroid administration that can suppress adrenocorticotropic hormone secretion.

In one embodiment of the present invention, the methods, kits, combinations, and composition are useful in treating a subject where chronic corticosteroid therapy is administered. Chronic corticosteroid therapy is used for a variety of conditions, which include rheumatoid arthritis, systemic lupus erythematosus, Sjogren's syndrome, immunosuppression for transplants, asthma, etc. Corticosteroid-induced adrenal suppression may thus represent the largest group of subjects with deficient adrenal androgen production. Androgen deficiency is recognized as a contributory factor to corticosteroid-induced osteoporosis. By stimulating bone formation (osteoblast activity), testosterone replacement is beneficial in the treatment of corticosteroid-induced osteoporosis in premenopausal women, and is beneficial in estrogen replacement therapy where treating post-menopausal women. In a subject with autoimmune disorders, such as rheumatoid arthritis and systemic lupus erythematosus, testosterone deficiency can contribute to the underlying tendency to produce autoantibodies, as has been seen in a variety of animal models of autoimmune disease. Testosterone replacement can thus help to ameliorate the autoimmune disease process, itself. Despite these considerations, the potential therapeutic benefits of testosterone replacement in treating corticosteroid suppressed subjects have largely been ignored.

In another embodiment of the present invention, the methods, kits, combinations, and composition are useful in treating a panhypopituitarism woman. Panhypopituitarism from any cause is attended by a severe testosterone deficiency because of derangement of androgen secretion by both the ovaries and the adrenal glands.

In yet another embodiment of the present invention, the methods, kits, combinations, and composition are useful in treating a subject with primary adrenal insufficiency. Primary adrenal insufficiency is associated with testosterone deficiency.

In one embodiment of the present invention, the methods, kits, combinations, and composition are useful in treating a subject with chronic illnesses. Chronic illnesses in a subject are attended by decreased circulating testosterone concentrations. Glucocorticoid administration inhibits adrenal androgen production by their inhibitory effects on adrenocorticotropic hormone secretion. In addition, glucocorticoids also have inhibitory effects at all levels of the hypothalamic-pituitary-ovarian axis.

In still another embodiment of the present invention, the methods, kits, combinations, and composition are useful in treating a human immunodeficiency virus-positive man or women. In contrast to human immunodeficiency virus-positive men, where testosterone deficiency is common, it is not known whether human immunodeficiency virus-positive women are deficient in testosterone. Amenorrhea, which appears to be increased in women with acquired immunodeficiency syndrome (AIDS), may be an indication that ovarian steroid production is diminished. Adrenal function can also be deficient in acquired immunodeficiency syndrome subjects due to cytomegaloviris infection, tuberculosis and/or fungal infections. Megestrol acetate, a progestational agent used to stimulate appetite in human immunodeficiency virus infected persons, suppresses gonadotropins and is it believed to lower testosterone levels in women, similar to its effects in men. In addition, the use of oral contraceptives by a human immunodeficiency virus-positive woman also reduces testosterone levels, as described above in normal women. Physiological testosterone replacement can be used as an anabolic agent for treating/preventing the wasting syndrome and for enhancing quality of life in a woman.

The methods, kits, combinations, and compositions of the present invention are also useful to treat a number of physiological and psychological parameters associated with testosterone deficiency in a man or a woman, and include, for example, increasing libido and improving sexual performance and dysfunction, increasing bone mineral density and related markers, improving body composition, preventing human immunodeficiency virus wasting syndrome, improving cognition, improving mood and self-esteem, improving muscle mass and performance, treating premenstrual syndrome, and treating autoimmune diseases.

In one embodiment of the present invention, the methods, kits, combinations, and composition are useful in treating the libido of a subject. Testosterone concentrations clearly affect male and female libido. Over the past few decades, several correlational studies found that higher testosterone levels were associated with less sexual avoidance, more sexual gratification, more sexual thoughts, more initiation of sexual activity, higher levels of sexual interest and desire, and more anticipation of sexual activity. More recently, found a correlation between sexual desire and testosterone in a subset of women, those who were human immunodeficiency virus-positive.

In one embodiment of the present invention, the methods, kits, combinations, and composition are useful in treating sexual performance in a subject. Studies have shown that testosterone influences sexual performance in men and women. In women, for example, correlational studies have found that testosterone is associated with higher sexual arousability as measured by vasocongestive responses to erotic films, increased frequency of masturbation, increased frequency of coitus, and a higher number of sexual partners. Another correlational study also showed that testosterone is associated with decreased vaginal atrophy.

In another embodiment of the present invention, the methods, kits, combinations, and composition are useful in treating female sexual dysfunction in a woman. Surgical menopause, that is, total abdominal hysterectomy and bilateral salpingo-oophorectomy, performed prior to the natural menopause causes a syndrome of female sexual dysfunction in a significant number of women that is unrelieved by conventional estrogen replacement therapy. The sexual components of this syndrome include decreased libido, decreased arousal and a diminished ability to attain orgasm. The psychological components include decreased energy, depressed mood, and a general decrease in well-being. These are generally distinguishable from the classic estrogen deficiency symptoms of vaginal atrophy, diminished lubrication, hot flushes and emotional liability that can adversely affect sexual function and psychological well-being in menopausal women who do not receive adequate estrogen replacement therapy. Rather than estrogen deficiency, the hormonal basis for this syndrome is attributed to a testosterone deficiency state resulting from the absent ovarian production of testosterone and its precursors.

In one study, the effects of testosterone in women with impaired sexual function after surgically induced menopause were evaluated using a transdermal patch. Seventy-five women, 31 to 56 years old, who had undergone oophorectomy and hysterectomy received conjugated equine estrogens (at least 0.625 mg per day orally) and, in random order, 150 μg of testosterone, and 300 μg of testosterone per day transdermally for 12 weeks each. Outcome measures included scores on the Brief Index of Sexual Functioning for Women (BISF), the Psychological Well-Being Index (PGWI), and a sexual function diary completed over the telephone. The mean (±SD) serum free testosterone concentration increased from 1.2±0.8 pg/mL during placebo treatment to 3.9±2.4 pg/mL and 4.9±4.8 pg/mL during treatment with 160 and 300 μg of testosterone per day, respectively (normal range, 1.3 to 6.8 pg/mL. Despite an appreciable placebo response, the higher testosterone dose resulted in further increases in scores for frequency of sexual activity and pleasure-orgasm in the Brief Index of Sexual Functioning for Women (P=0.03 for both comparisons with placebo). At the higher dose, the percentages of women who had sexual fantasies, masturbated, or engaged in sexual intercourse at Icast once a week increased two to three times from base line. The positive-well-being, depressed-mood, and composite scores of the Psychological Well-Being Index also improved at the higher dose (P=0.04, P=0.04, respectively, for the comparison with placebo), but the scores on the telephone-based diary did not increase significantly.

In another embodiment of the present invention, testosterone therapy is used in conjunction with estrogen therapy. Studies have shown that testosterone and estrogen replacement resulted in increased sexual desire, frequency of sexual fantasies, sexual arousal, and coital or orgasmic frequency compared to those given estrogen alone or a placebo reported that women receiving estrogen plus testosterone experienced more increased libido, activity, satisfaction, pleasure, fantasy, orgasm, and relevancy as compared to women receiving estrogen alone. Treatment with Premarin and methyltestosterone resulted in significantly increased reports of pleasure from masturbation. Treatment with estrogen and methyltestosterone similarly results in increased sexual interest. Most recently, it has been found that transdermal testosterone treatment in women after oophorectomy improved sexual function and psychological well-being. It is contemplated that testosterone administration alone will have therapeutic benefits if given without estrogen. For example, women with hypothalamic amenorrhea show increased vaginal vasocongestion with testosterone treatment compared to a placebo.

In still another embodiment of the present invention, the methods, kits, combinations, and composition are useful in treating decreased bone density in a subject, for example, a women. Another physiologic parameter linked to testosterone administration in women is decreased bone mineral density. Several correlational studies have shown that increased testosterone concentrations are associated with increased bone mineral density. It has been found that higher bioavailable testosterone levels were associated with higher bone mineral density in the ultradistal radius in women. Women having polycystic ovary syndrome had neck bone mineral density positively correlated to free testosterone levels. Upper body bone mineral density had significant correlation with testosterone. A cross-sectional analysis of sex hormone concentrations and bone mineral density in women recruited for a prospective study of risk factors for osteoporosis and found a significant positive correlation between testosterone and bone mineral density. Another study involved an age-stratified sample of 304 women and found a correlation coefficient between bone mineral density and testosterone as shown below in Table 8:

TABLE 8
Correlational Coefficients between
Testosterone and Bone Mineral Density*

| | Total | Bioavailable |
|---|---|---|
| | Testosterone | Testosterone |
| Total body | 0.22 | 0.22 |
| Lateral spine | 0.27 | 0.29 |
| Proximal femur | 0.25 | 0.30 |
| Radius | 0.27 | 0.28 |

*Khosla S. et al., J Clin Endocrinol Metab. 1998 Jul; 83(7): 2266-74.

As with libido and sexual performance, testosterone is often given in conjunction with estrogen in order to prevent bone loss or increase bone mineral density. For example, in a cross sectional study, it was found that subcutaneous estradiol (75 mg) and testosterone (100 mg) prevented osteoporosis and maintained normal bone mineral density in post-menopausal women. In another study the effects of estrogen given alone to those of estrogen plus androgen therapy in post-menopausal women. While the estrogen-only group had a reduction in serum markers of bone formation, women treated with combined estrogen and testosterone had increased bone formation markers. Similarly, it has been shown that estrogen and testosterone replacement with implant pellets increases bone mass more than estrogen implants alone, increased bone mineral density by 5.7% in the spine and 5.2% in the neck femur region. Treatment with estrogen and methyltestosterone similarly results in increased spine and hip bone mineral density. Also, it has been reported that orally given estrogens and methyltestosterone prevented bone loss and increased bone mineral density in the spine and hip.

In another embodiment of the present invention, the methods, kits, combinations, and composition are useful in treating body composition of a subject. For example, testosterone has been linked to improved body composition in women. Testosterone is positively correlated to body mass index and exogenous androgens influenced body composition and regional body fat distribution in obese post-menopausal women. Other researchers have found an increase in fat-free mass and a reduced fat mass to fat free mass ratio in postmenopausal women treated with concurrent estrogen-testosterone therapy. Thus, administration of testosterone to normal women or those having testosterone deficiencies may have a therapeutic improvement in body composition.

In still another embodiment of the present invention, the methods, kits, combinations, and composition are useful in treating or preventing human immunodeficiency virus wasting syndrome in a subject. For example, in recent years, researchers have found that testosterone administration to women infected with human immunodeficiency virus may treat or prevent human immunodeficiency virus wasting syndrome. It has been found that lower free testosterone levels in human immunodeficiency virus-infected women using a tracer analog method. For example, testosterone replacement in a patch delivering 150 ug/day of testosterone to human immunodeficiency virus-infected women had a 4% increase in body weight over 12 weeks. In addition, the subjects had an improved quality of life. Thus, testosterone administration can be used as a method of preventing wasting in a subject suffering from acquired immunodeficiency syndrome or related disorders.

In yet another embodiment of the present invention, the methods, kits, combinations, and composition are useful in treating or preventing short-term and long-term memory and other higher-order cognitive functions in a subject. Sex steroids are important for short-term and long-term memory and other higher-order cognitive functions. For example, postmenopausal women receiving estrogen plus testosterone following oophorectomy had higher scores on two tests of short-term memory, a test of long-term memory, and a test of logical reasoning. It has been reported that the administration of testosterone is associated with better visio-spacial function and verbal skills. Women with high testosterone levels scored higher on special/mathematical tasks than women with low testosterone concentrations. Women with higher Mini-Mental State Examination scores had significantly higher mean total and bioavailable testosterone concentrations. Testosterone levels are also related to verbal fluency. Again, the benefits of testosterone administration on cognitive parameters may be optimized by concurrent estrogen administration. For example, subcutaneous implants of oestradiol (40 mg) and testosterone (100 mg) have shown increases in concentration.

In one embodiment of the present invention, the methods, kits, combinations, and compositions are useful in treating or preventing a mood or self-esteem disorder in a subject. Parameters associated with testosterone serum levels in a subject are mood and self-esteem. For example, menopausal women who received both estrogen and testosterone felt more composed, elated, and energetic than those who were given estrogen alone. Similarly, testosterone concentrations are positively correlated to self-esteem. Thus, it is contemplated that testosterone therapy will improve mood when used alone or in the case of a woman, when used in conjunction with estrogen.

In another embodiment of the present invention, the methods, kits, combinations, and composition are useful in increasing muscle size and performance in a subject. Androgens and anabolic steroids have long since been used to increase muscle size and performance in men. Researchers have recently also found that testosterone is an important determinant of greater muscle size in women with polycystic ovary syndrome. Thus, administration of testosterone to a normal or testosterone deficient woman may be useful for improving muscle mass and performance.

Many of the symptoms for women described above fall under the umbrella of what is commonly considered to be premenstrual syndrome (PMS). In general, lower levels of testosterone throughout the menstrual cycle have been reported in women who suffer from premenstrual syndrome compared with controls. Testosterone replacement is currently used as a management of premenstrual syndrome in the United Kingdom and Australia. Managing premenstrual syndrome with oestradiol/testosterone implants resulted in improvements in libido, enjoyment of sex, and tiredness. Thus, it is contemplated that the methods, kits, combinations, and compositions of the present invention can be useful in treating premenstrual syndrome in a woman, especially in conjunction with administration of an estrogenic hormone.

In one embodiment, the estrogenic hormone is formulated for percutaneous administration in a hydroalcoholic gel. The gel comprises one or more lower alcohols, a penetration enhancing agent, a thickening agent, and water. Additionally, the estrogenic gel optionally includes salts, emollients, stabilizers, antimicrobials, fragrances, and propellants.

Illustratively, the estrogenic gel is comprised of the following substances as shown below in Table 9, in approximate amounts.

TABLE 9
Composition of ESTRAGEL

| | AMOUNT (w/w) |
|---|---|
| SUBSTANCE | PER 100 g OF GEL |
| 17-beta-oestradiol | 0.06 g |
| Carbopol 980 | 1.0 g |
| Triethanolamine | 1.35 g |
| Ethanol (95% w/w) | (59 ml) |
| Purified water (qsf) | 100 g |

One skilled in the art will appreciate that the constituents of this formulation may be varied in amounts yet continue to be within the spirit and scope of the present invention. For example, the composition may contain about 0.1 to about 10 g of estradiol, about 0.1 to about 5.0 g CARBOPOL, about 0.1 to about 5.0 g triethanolamine, and about 30.0 to about 98.0 g ethanol.

In one embodiment of the present invention, the methods, kits, combinations, and composition are useful in suppressing both cell-mediated and humoral immune responses in a subject. Androgens appear to suppress both cell-mediated and humoral immune responses. Many researchers have advocated increasing testosterone levels in a subject as protective against autoimmune disease, such as rheumatoid arthritis. Testosterone administration therefore is contemplated to be effective in treating a subject with such disorders.

Toxicity and therapeutic efficacy of the therapeutic agents of the present invention can be determined by standard pharmaceutical procedures, for example, for determining LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The active agents of the present invention may be administered, if desired, in the form of a salt, an ester, an amide, an enantiomer, an isomer, a tautomers, a prodrug, a derivative or the like, provided the salt, ester, amide, enantiomer, isomer, tautomer, prodrug, or derivative is suitable pharmacologically, that is, effective in the present methods, kits, combinations, and compositions. Salts, esters, amides, enantiomers, isomers, tautomers, prodrugs and other derivatives of the active agents may be prepared using standard procedures known to those skilled in the art of synthetic organic chemistry and described, for example, by J. March, Advanced Organic Chemistry; Reactions, Mechanisms and Structure, 4th Ed. (New York: Wiley-Interscience, 1992). For example, acid addition salts are prepared from the free base using conventional methodology, and involves reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, for example, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, for example, hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Particularly preferred basic salts here are alkali metal salts, for example, the sodium salt, and copper salts. Preparation of esters involves functionalization of hydroxyl and/or carboxyl groups which may be present within the molecular structure of the drug. The esters are typically acyl-substituted derivatives of free alcohol groups, that is, moieties that are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

The therapeutic agents of the present invention can be formulated as a single pharmaceutical composition containing at least one therapeutic agent, for example, testosterone alone or with an antidepressant agent, or as independent multiple pharmaceutical compositions where each composition contains at least one therapeutic agent. Pharmaceutical compositions according to the present invention include those compositions with at least one therapeutic agent formulated for percutaneous administration. Percutaneous administration includes transdermal delivery systems that include patches, gels, tapes and creams, and can contain excipients such as alcohols, penetration enhancing agents, hydroxide releasing agents, and thickening agents, as well as solubilizers (for example propylene glycol, bile salts, and amino acids), hydrophilic polymers (for example, polycarbophil and polyvinylpyrolidone), and adhesives and tackifiers (for example, polyisobutylenes, silicone-based adhesives, acrylates and polybutene).

The therapeutic agents of the present invention can then be administered percutaneously in dosage unit formulations containing conventional nontoxic pharmaceutically acceptable carriers, adjuvants, and vehicles as desired.

The compounds of the present invention can be administered by any conventional means available for use in conjunction with pharmaceuticals, either as individual therapeutic compounds or as a combination of therapeutic compounds.

The compositions of the present invention can be administered for treating, preventing, or reducing the risk of developing a testosterone deficiency in a subject by any means that produce contact of these compounds with their site of action in the body, for example in the ileum, the plasma, or the liver of a subject. For example the compositions can be administered, for example, orally, rectally, topically, bucally, or parenterally, Additionally, the methods, kits, combinations, and compositions of the present invention may optionally include salts, emollients, stabilizers, antimicrobials, fragrances, and propellants.

In another embodiment of the present invention, the therapeutic agents come in the form of kits or packages containing testosterone. Illustratively, the kits or packages contain testosterone in a dosage form suitable for percutaneous administration, for example, a gel, a cream, an ointment, or a patch, in amounts for the proper dosing of the drugs. The therapeutic agents of the present invention can be packaged in the form of kits or packages in which the daily (or other periodic) dosages are arranged for proper sequential or simultaneous administration. The present invention further provides a kit or package containing a plurality of dosage units, adapted for successive daily administration, each dosage unit comprising at least one of the therapeutic agents of the present invention. This drug delivery system can be used to facilitate administering any of the various embodiments of the therapeutic compositions. In one embodiment, the system contains a plurality of dosages to be to be administered daily or weekly where at least one of the dosages is administered via percutaneous administration. In another embodiment, the system contains a plurality of dosages to be to be administered daily or weekly where at least one of the dosages is administered via percutaneous administration, and at least one of the dosages is administered orally. The kits or packages also contain a set of instructions for the subject.

The present methods, kits, combinations, and compositions can also be used in “combination therapy” with another steroid, or a pharmaceutical agent that increases testosterone levels in a subject, or an estrogenic hormone, or another pharmaceutical agent such as, for example, an antidepressant agent.

The phrase “combination therapy” embraces the administration of a steroid in the testosterone synthesis pathway in conjunction with another steroid, or a pharmaceutical agent that increases testosterone levels in a subject, or an estrogenic hormone, or another pharmaceutical agent such as, for example, an antidepressant agent, as part of a specific treatment regimen intended to provide a beneficial effect from the co-action of these therapeutic agents for the treatment of a depressive disorder in a subject. The beneficial effect of the combination includes, but is not limited to, pharmacokinetic or pharmacodynamic co-action resulting from the combination of therapeutic agents. Administration of these therapeutic agents in combination typically is carried out over a defined time period (usually simultaneously, minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single gel having a fixed ratio of each therapeutic agent or in multiple, single capsules, tablets, or gels for each of the therapeutic agents. Sequential or substantially simultaneous administration of each therapeutic agent can be effected by any appropriate route including, but not limited to, an oral route, a percutaneous route, an intravenous route, an intramuscular route, or by direct absorption through mucous membrane tissues. The therapeutic agents can be administered by the same route or by different routes. For example, a first therapeutic agent of the combination selected may be administered orally, while the other therapeutic agents of the combination may be administered percutaneously. Alternatively, for example, all therapeutic agents may be administered percutaneously, or all therapeutic agents may be administered intravenously, or all therapeutic agents may be administered intramuscularly, or all therapeutic agents can be administered by direct absorption through mucous membrane tissues. The sequence in which the therapeutic agents are administered is not narrowly critical. “Combination therapy” also can embrace the administration of the therapeutic agents as described above in further combination with other biologically active ingredients, such as, but not limited to, agents for improving sexual performance, such as, for example, an agent effective at inhibiting the activity of a phosphodiesterase, and non-drug therapies, such as, but not limited to, surgery.

The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by percutaneous route. Whether the therapeutic compounds of the combined therapy are administered orally, by inhalation spray, rectally, topically, buccally (e.g., sublingual), or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components. Examples of suitable pharmaceutically-acceptable formulations containing the therapeutic compounds are given above. Additionally, drug formulations are discussed in, for example, Hoover, John E., Remington's Pharmaceutical Sciences, Mack Publishing Co., Easton, Pa. 1975. Another discussion of drug formulations can be found in Liberman, H. A. and Lachman, L., Eds., Pharmaceutical Dosage Forms, Marcel Decker, New York, N.Y., 1980.

The present invention is further illustrated by the following examples, which should not be construed as limiting in any way. In the below example, it is assumed that normal cycling women produce approximately 300 μg of testosterone per day, and their serum testosterone levels generally range from about 20 ng/dL to about 80 ng/dL averaging about 40 ng/dL. Bilateral oophorectomy in pre-menopausal women reduces testosterone production by approximately 50%, resulting in an average total serum level of approximately 20 ng/dL. From a physiological perspective, testosterone therapy in surgically menopausal women who, for example, experience female sexual dysfunction, is to replace the missing ovarian testosterone production of approximately 150 μg per day and restore the levels of testosterone and its active androgenic metabolite dihydrotestosterone (DHT) to their previous levels within the normal physiological range.

The following examples are provided for exemplification of the present invention and are not intended to be limiting in any way.

### EXAMPLES

### Example 1

### Dosage of Testosterone in a Female after Bilateral Oophorectomy

In one embodiment of the present invention, the methods, kits, combinations, and compositions are comprised of a percutaneously deliverable testosterone formulation. In this example, testosterone is formulated as a gel for transdermal administration as described above in Table 5a (Relibra®).

In a prophetic example, 24 pre-menopausal women who have undergone bilateral oophorectomy are randomized to receive: (a) 1.7 g/day of Relibra®, which delivers 1.7 mg/day of testosterone to the skin of which about 0.1 mg, is absorbed, for 30 days; or (b) 2.5 g/day of Relibra®, which delivers 2.5 mg/day of testosterone to the skin of which about 0.15 mg is absorbed, for 30 days; or (c) 5 g/day of Relibra®, which delivers 5.0 mg/day of testosterone to the skin of which about 0.3 mg is absorbed, for 30 days; or (d) a gel containing a placebo for 30 days. The gel is rubbed onto the clean dry skin of the upper outer thigh and hip once daily. Following application, the gel is allowed to air dry. The subject washes her hands

Applicants expect that from a physiological perspective, all test parameters will show an improvement in female sexual dysfunction and an improvement in overall depressive symptoms over the placebo. Accordingly, Applicant expects that the composition can be applied to improve female sexual dysfunction and a depressive disorder as compared to placebo in pre-menopausal women who have undergone a bilateral oophorectomy.

### Example 2

### Dosage of Testosterone and Methyltestosterone in a Female after Bilateral Oophorectomy

In one embodiment of the present invention, the methods, kits, combinations, and compositions are comprised of a percutaneously deliverable testosterone formulation, and an orally deliverable methyltestosterone formulation. In this example, testosterone is formulated as a gel for transdermal administration as described above in Table 5a (Relibra®), and mcthyltestosterone is formulated as a capsule for oral administration and each dosage unit contains 10 mg of methyltestosterone.

In a prophetic example, 24 pre-menopausal women who have undergone bilateral oophorectomy are randomized to receive a daily oral dose of 10 mg or 50 mg methyltestosterone for 30 days, plus: (a) 1.7 g/day of Relibrae, which delivers 1.7 mg/day of testosterone to the skin of which about 0.1 mg, is absorbed, for 30 days; or (b) 2.5 g/day of Relibra®, which delivers 2.5 mg/day of testosterone to the skin of which about 0.15 mg is absorbed, for 30 days; or (c) 5 g/day of Relibra®, which delivers 5.0 mg/day of testosterone to the skin of which about 0.3 mg is absorbed, for 30 days; or (d) a gel containing a placebo for 30 days. The gel is rubbed onto the clean dry skin of the upper outer thigh and hip once daily. Following application, the gel is allowed to air dry. The subject washes her hands.

Applicants expect that from a physiological perspective, all test parameters will show an improvement in female sexual dysfunction and an improvement in overall depressive symptoms over the placebo. Accordingly, Applicant expects that Relibra® can be administered in conjunction with methyltestosterone to improve female sexual dysfunction and a depressive disorder as compared to placebo in pre-menopausal women who have undergone a bilateral oophorectomy.

### Example 3

### Dosage of Testosterone and Estrogen in a Female after Bilateral Oophorectomy

In one embodiment of the present invention, the methods, kits, combinations, and compositions are comprised of a percutaneously deliverable testosterone formulation, and a non-orally deliverable estrogen. In this example, testosterone is formulated as a gel for transdermal administration as described above in Table 5a (Relibra®), and estradiol is formulated as a gel for transdermal administration as described above in Table 9 (ESTRAGEL).

In a prophetic example, 24 pre-menopausal women who have undergone bilateral oophorectomy are randomized to receive a daily dose of 5 g or 10 g ESTRAGEL for 30 days, plus: (a) 1.7 g/day of Relibra®, which delivers 1.7 mg/day of testosterone to the skin of which about 0.1 mg, is absorbed, for 30 days; or (b) 2.5 g/day of Relibra®, which delivers 2.5 mg/day of testosterone to the skin of which about 0.15 mg is absorbed, for 30 days; or (c) 5 g/day of Relibra®, which delivers 5.0 mg/day of testosterone to the skin of which about 0.3 mg is absorbed, for 30 days; or (d) a gel containing a placebo for 30 days. The gel is rubbed onto the clean dry skin of the upper outer thigh and hip once daily. Following application, the gel is allowed to air dry. The subject washes her hands.

Applicants expect that from a physiological perspective, all test parameters will show an improvement in female sexual dysfunction and depressive disorders over the placebo. Accordingly, Applicant expects that the composition can be administered in conjunction with estradiol to improve female sexual dysfunction as compared to placebo in pre-menopausal women who have undergone a bilateral oophorectomy.

Example 4
Combination Testosterone and Estrogen Gel

| Substance | Amount (w/w) per 100 g of Gel |
|---|---|
| Testosterone | 1.0 g (or about 0.5 g) |
| 17-beta-oestradiol | 0.06 g (or about 0.10 g) |
| Carbopol 980 | 1.0 g |
| Triethanolamine | 1.35 g |
| Isopropyl myristate | 0.50 g |
| 0.1 N NaOH | 4.72 g |
| Ethanol (95% w/w) | 72.5 g |
| Purified Water (qsf) | 100 g |

The gel is rubbed onto the clean dry skin of the upper outer thigh and hip once daily. Following application, the gel is allowed to air dry. The subject washes her hands. Application of the gel results in an increased testosterone level having a desirable pharmacokinetic profile similar to that in normal women. The gel is thus useful for treating a number of conditions or diseases in women, such as a depressive disorder.

### Example 5

### Method of Improving Sexual Performance and Increasing Libido in Hypogonadal Men

One embodiment of the present invention involves the transdermal application of AndroGel® as a method of increasing sexual performance and libido in hypogonadal men without causing significant skin irritation.

In this example, hypogonadal men were recruited and studied in 16 centers in the United States. The patients were between 19 and 68 years and had single morning serum testosterone levels at screening of less than or equal to 300 ng/dL (10.4 nmol/L ). A total of 227 patients were enrolled: 73, 78, and 76 were randomized to receive 5.0 g/day of AndroGel® (delivering 50 mg/day of testosterone to the skin of which about 10% or 5 mg is absorbed), 10 g/day of AndroGel® (delivering 100 mg/day of testosterone to the skin of which about 10% or 10 mg is absorbed), or the ANDRODERM® testosterone patch (“T patch”; delivering 50 mg/day of testosterone), respectively.

As shown in the Table 10, there were no significant group-associated differences of the patients' characteristics at baseline.

TABLE 10
Baseline Characteristics of the Hypogonadal Men

| | | AndroGel ® | AndroGel ® |
|---|---|---|---|
| Treatment Group | T patch | (5.0 g/day) | (10.0 g/day) |
| No of subjects enrolled | 76 | 73 | 78 |
| Age (years) | 51.1 | 51.3 | 51.0 |
| Range (years) | 28-67 | 23-67 | 19-68 |
| Height (cm) | 179.3 ± 0.9 | 175.8 ± 0.8 | 178.6 ± 0.8 |
| Weight (kg) | 92.7 ± 1.6 | 90.5 ± 1.8 | 91.6 ± 1.5 |
| Serum testosterone (nmol/L) | 6.40 ± 0.41 | 6.44 ± 0.39 | 6.49 ± 0.37 |
| Causes of hypogonadism | | | |
| Primary hypogonadism | 34 | 26 | 34 |
| Klinefelter's Syndrome | 9 | 5 | 8 |
| Post Orchidectomy/Anorchia | 2 | 1 | 3 |
| Primary Testicular Failure | 23 | 20 | 23 |
| Secondary hypogonadism | 15 | 17 | 12 |
| Kallman's Syndrome | 2 | 2 | 0 |
| Hypothalimic Pituitary Disorder | 6 | 6 | 3 |
| Pituitary Tumor | 7 | 9 | 9 |
| Aging | 6 | 13 | 6 |
| Not classified | 21 | 17 | 26 |
| Years diagnosed | 5.8 ± 1.1 | 4.4 ± 0.9 | 5.7 ± 1.24 |
| Number previously treated with | 50 (65.8%) | 38 (52.1%) | 46 (59.0%) |
| testosterone | | | |
| Type of Previous Hormonal Treatment | | | |
| Intramuscular injections | 26 | 20 | 28 |
| Transdermal patch | 12 | 7 | 8 |
| All others | 12 | 11 | 10 |
| Duration of treatment (years) | 5.8 ± 1.0 | 5.4 ± 0.8 | 4.6 ± 80.7 |

Forty-one percent (93/227) of the subjects had not received prior testosterone replacement therapy. Previously treated hypogonadal men were withdrawn from testosterone ester injection for at least six weeks and oral or transdermal androgens for four weeks before the screening visit. Aside from the hypogonadism, the subjects were in good health as evidenced by medical history, physical examination, complete blood count, urinalysis, and serum biochemistry. If the subjects were on lipid-lowering agents or tranquilizers, the doses were stabilized for at least three months prior to enrollment. Less than 5% of the subjects were taking supplemental calcium or vitamin D during the study. The subjects had no history of chronic medical illness, alcohol or drug abuse. They had a normal rectal examination, a PSA level of less than 4 ng/mL, and a urine flow rate of 12 mL/s or greater. Patients were excluded if they had a generalized skin disease that might affect the testosterone absorption or prior history of skin irritability with ANDRODERM® patch. Subjects weighing less than 80% or over 140% of their ideal body weight were also excluded.

The randomized, multi-center, parallel study compared two doses of AndroGel® with the ANDRODERM® testosterone patch. The study was double-blind with respect to the AndroGel® dose and open-labeled for the testosterone patch group. For the first three months of the study (days 1 to 90), the subjects were randomized to receive 5.0 g/day of AndroGel®, 10.0 g/day of AndroGel®, or two non-scrotal patches. In the following three months (days 91 to 180), the subjects were administered one of the following treatments: 5.0 g/day of AndroGel®, 10.0 g/day of AndroGel®, 7.5 g/day of AndroGel®, or two non-scrotal patches. Patients who were applying AndroGel® had a single, pre-application serum testosterone measured on day 60 and, if the levels were within the normal range of 300 to 1,000 ng/dL (10.4 to 34.7 nmol/L ), then they remained on their original dose. Patients with testosterone levels less than 300 ng/dL and who were originally assigned to apply 5.0 g/day of AndroGel® and those with testosterone levels more than 1,000 ng/dL who had received 10.0 g/day of AndroGel® were then reassigned to administer 7.5 g/day of AndroGel® for days 91 to 180.

Accordingly, at 90 days, dose adjustments were made in the AndroGel® groups based on the pre-application serum testosterone levels on day 60. Twenty subjects in the 5.0 g/day AndroGel® group had the dose increased to 7.5 g/day. Twenty patients in the 10.0 g/day AndroGel® group had the AndroGel® dose reduced to 7.5 g/day. There were three patients in the testosterone patch group who were switched to 5.0 g/day AndroGel® because of patch intolerance. One 10.0 g/day AndroGel® subject was adjusted to receive 5.0 g/day and one 5.0 g/day AndroGel® subject had the dose adjusted to 2.5 g/day. The number of subjects enrolled into day 91 to 180 of the study thus consisted of 51 receiving 5.0 g/day of AndroGel®, 40 receiving 7.5 g/day of AndroGel®, 52 receiving 10.0 g/day of AndroGel®, and 52 continuing on the ANDRODERM® patch. The treatment groups in this example may thus be characterized in two ways, either by “initial” or by the “final” treatment group. Subjects returned to the study center on days 0, 30, 60, 90, 120, 150, and 180 for a clinical examination, skin irritation and adverse event assessments.

AndroGel® and ANDRODERM® Patch

Approximately 250 g of AndroGel® was packaged in multidose glass bottles that delivered 2.25 g of the gel for each actuation of the pump. Patients assigned to apply 5.0 g/day of Androgel® testosterone were given one bottle of AndroGel® and one bottle of placebo gel (containing vehicle but no testosterone), while those assigned to receive 10.0 g/day of AndroGel® were dispensed two bottles of the active AndroGel®. The patients were then instructed to apply the bottle contents to the right and left upper arms/shoulders and to the right and left sides of the abdomen on an alternate basis. For example, on the first day of the study, patients applied two actuations from one bottle, one each to the left and right upper arm/shoulder, and two actuations from the second bottle, one each to the left and right abdomen. On the following day of treatment, the applications were reversed. Alternate application sites continued throughout the study. After application of the gel to the skin, the gel dried within a few minutes. Patients washed their hands thoroughly with soap and water immediately after gel application.

The 7.5 g/day AndroGel® group received their dose in an open-label fashion. After 90 days, for the subjects titrated to the AndroGel® 7.5 g/day dose, the patients were supplied with three bottles, one containing placebo and the other two AndroGel®. The subjects were instructed to apply one actuation from the placebo bottle and three actuations from a AndroGel® bottle to four different sites of the body as above. The sites were rotated each day taking the same sequence as described above.

ANDRODERM® testosterone patches each delivering 2.5 mg/day of testosterone were provided to about one-third of the patients in the study. These patients were instructed to apply two testosterone patches to a clean, dry area of skin on the back, abdomen, upper arms, or thighs once per day. Application sites were rotated with approximately seven days interval between applications to the same site.

On study days when the patients were evaluated, the gel/patches were applied following pre-dose evaluations. On the remaining days, the testosterone gel or patches were applied at approximately 8:00 a.m. for 180 days.

Study Method and Results

Hormone Pharmacokinetics

On days 0, 1, 30, 90, and 180, the patients had multiple blood samples for testosterone and free testosterone measurements at 30, 15 and 0 minutes before and 2, 4, 8, 12, 16, and 24 hours after AndroGel® or patch application. In addition, subjects returned on days 60, 120, and 150 for a single blood sampling prior to application of the gel or patch. Serum DHT, E₂, FSH, LH and SHBG were measured on samples collected before gel application on days 0, 30, 60, 90, 120, 150, and 180. Sera for all hormones were stored frozen at −20 ° C. until assay. All samples for a patient for each hormone were measured in the same assay whenever possible. The hormone assays were then measured at the Endocrine Research Laboratory of the UCLA-Harbor Medical Center.

Table 11 summarizes the pharmacokinetic parameters were measured for each patient:

TABLE 11
Pharmacokinetic Parameters

| | |
|---|---|
| AUC₀₋₂₄ | area under the curve from 0 to 24 hours, |
| | determined using the linear trapezoidal rule. |
| C_{base} or Cₒ | Baseline concentration |
| C_{avg} | time-averaged concentration over the 24-hour dosing |
| | interval determined by AUC₀₋₂₄/24 |
| Cₘₐₓ | maximum concentration during the 24-hour dosing |
| | interval |
| Cₘᵢₙ | minimum concentration during the 24-hour dosing |
| | interval |
| Tₘₐₓ | time at which Cₘₐₓ occurred |
| Tₘᵢₙ | time at which Cₘᵢₙ occurred |
| Fluctuation | extent of variation in the serum concentration over the |
| Index | course of a single day, calculated as (Cₘₐₓ − Cₘᵢₙ)/C_{avg} |
| Accumulation | increase in the daily drug exposure with continued |
| ratio | dosing, calculated as the ratio of the AUC at steady on |
| | a particular day over the AUC on day 1 (e.g., AUC_{day 30}/ |
| | AUC_{day 1}) |
| Net AUC₀₋₂₄ | AUC₀₋₂₄ on days 30, 90, 180 - AUC₀₋₂₄ on day 0 |

Testosterone Pharmacokinetics

Methods

Serum testosterone levels were measured after extraction with ethylacetate and hexane by a specific radioimmunoassay (“RIA”) using reagents from ICN (Costa Mesa, Calif.). The cross reactivities of the antiserum used in the testosterone RIA were 2.0% for DHT, 2.3% for androstenedione, 0.8% for 3-β-androstanediol, 0.6% for etiocholanolone and less than 0.01% for all other steroids tested. The lower limit of quantitation (“LLQ”) for serum testosterone measured by this assay was 25 ng/dL (0.87 nmol/L). The mean accuracy of the testosterone assay, determined by spiking steroid free serum with varying amounts of testosterone (0.9 nmol/L to 52 nmol/L), was 104% and ranged from 92% to 117%. The intra-assay and inter-assay coefficients of the testosterone assay were 7.3 and 11.1%, respectively, at the normal adult male range. In normal adult men, testosterone concentrations range from 298 to 1,043 ng/dL (10.33 to 36.17 nmol/L) as determined at the UCLA-Harbor Medical Center.

Baseline Concentration

As shown in Table 12(a)-6(b) and FIG. No. 1 (a), at baseline, the average serum testosterone concentrations over 24 hours (C_{avg}) were similar in the groups and below the adult normal range. Moreover the variations of the serum concentration (based on maximum and minimum concentrations during the 24-hour period, Cₘₐₓ and Cₘᵢₙ, respectively) during the day were also similar in the three groups. FIG. No. 1(a) shows that the mean testosterone levels had a the maximum level between 8 to 10 a.m. (i.e., at 0 to 2 hours) and the minimum 8 to 12 hours later, demonstrating a mild diurnal variation of serum testosterone. About one-third of the patients in each group had C_{avg} within the lower normal adult male range on day 0 (24/73 for the 5.0 g/day AndroGel® group, 26/78 for the 10.0 g/day AndroGel® group, and 25/76 for testosterone patch group). All except three of the subjects met the enrollment criterion of serum testosterone less than 300 ng/dL (10.4 nmol/L) on admission.

TABLE 12(a)
Baseline Phamacokinetic Parameters
by Initial Treatment Group (Mean ± SD)

| | 5.0 g/day T-Gel | 10.0 g/day T-gel | T-patch |
|---|---|---|---|
| N | 73 | 78 | 76 |
| C_{avg} (ng/dL) | 237 ± 130 | 248 ± 140 | 237 ± 139 |
| Cₘₐₓ (ng/dL) | 328 ± 178 | 333 ± 194 | 314 ± 179 |
| Tₘₐₓ* (hr) | 4.0 (0.0-24.5) | 7.9 (0.0-24.7) | 4.0 (0.0-24.3) |
| Cₘᵢₙ (ng/dL) | 175 ± 104 | 188 ± 112 | 181 ± 112 |
| Tₘᵢₙ* (hr) | 8.01 (0.0-24.1) | 8.0 (0.0-24.0) | 8.0 (0.0-23.9) |
| Fluc Index (ratio) | 0.627 ± 0.479 | 0.556 ± 0.384 | 0.576 ± 0.341 |

*Median (Range*)

TABLE 12(b)
Baseline Testosterone Pharmacokinetic Parameters
by Final Treatment Group (Mean ± SD)

| | Doses Received During Initial => Extended Treatment Phases | | | | |
|---|---|---|---|---|---|
| | 5.0 g/day | 5.0 => 7.5 g/day | 10.0 => 7.5 g/day | 10.0 g/day | |
| | T-gel | T-gel | T-gel | T-gel | T-patch |
| N | 53 | 20 | 20 | 58 | 76 |
| C_{avg} (ng/dL) | 247 ± 137 | 212 ± 109 | 282 ± 157 | 236 ± 133 | 237 ± 140 |
| Cₘₐₓ (ng/dL) | 333 ± 180 | 313 ± 174 | 408 ± 241 | 307 ± 170 | 314 ± 179 |
| Tₘₐₓ* (hr) | 4.0 (0.0-24.5) | 4.0 (0.0-24.0) | 19.7 (0.0-24.3) | 4.0 (0.0-24.7) | 4.0 (0.0-24.3) |
| Cₘᵢₙ (ng/dL) | 185 ± 111 | 150 ± 80 | 206 ± 130 | 182 ± 106 | 181 ± 112 |
| Tₘᵢₙ* (hr) | 8.0 (0.0-24.1) | 11.9 (0.0-24.0) | 8.0 (0.0-23.3) | 8.0 (0.0-24.0) | 8.0 (0.0-23.9) |
| Fluc Index (ratio) | 0.600 ± 0.471 | 0.699 ± 0.503 | 0.678 ± 0.580 | 0.514 ± 0.284 | 0.576 ± 0.341 |

*Median (range)

Day 1

FIG. No. 1(b) and Tables 12(c)-(d) show the pharmacokinetic profile for all three initial treatment groups after the first application of transdermal testosterone. In general, treatment with AndroGel® and the testosterone patch produced increases in testosterone concentrations sufficiently large to bring the patients into the normal range in just a few hours. However, even on day 1, the pharmacokinetic profiles were markedly different in the AndroGel® and patch groups. Serum testosterone rose most rapidly in the testosterone patch group reaching a maximum concentration (Cₘₐₓ) at about 12 hours (Tₘₐₓ). In contrast, serum testosterone rose steadily to the normal range after AndroGel® application with Cₘₐₓ levels achieved by 22 and 16 hours in the 5.0 g/day AndroGel® group and the 10.0 g/day AndroGel® group, respectively.

TABLE 12(c)
Testosterone Pharmacokinetic Parameters on Day 1
by Initial Treatment Group (Mean ± SD)

| | | |
|---|---|---|
| 5.0 g/day T-Gel | 10.0 g/day T-gel | T-patch |

| | | | |
|---|---|---|---|
| N | 73 | 76 | 74 |
| C_{avg} (ng/dL) | 398 ± 156 | 514 ± 227 | 482 ± 204 |
| Cₘₐₓ (ng/dL) | 560 ± 269 | 748 ± 349 | 645 ± 280 |
| Tₘₐₓ* (hr) | 22.1 (0.0-25.3) | 16.0 (0.0-24.3) | 11.8 (1.8-24.0) |
| Cₘᵢₙ (ng/dL) | 228 ± 122 | 250 ± 143 | 232 ± 132 |
| Tₘᵢₙ* (hr) | 1.9 (0.0-24.0) | 0.0 (0.0-24.2) | 1.5 (0.0-24.0) |

*Median (Range)

TABLE 12(d)
Testosterone Phamacokinetic Parameters on Day 1
by Final Treatment Group (Mean ± SD)

| | Doses Received During Initial => Extended Treatment Phases | | | | |
|---|---|---|---|---|---|
| | 5.0 g/day | 5.0 => 7.5 g/day | 10.0 => 7.5 g/day | 10.0 g/day | |
| | T-gel | T-gel | T-gel | T-gel | T-patch |
| N | 53 | 20 | 19 | 57 | 74 |
| C_{avg} (ng/dL) | 411 ± 160 | 363 ± 143 | 554 ± 243 | 500 ± 223 | 482 ± 204 |
| Cₘₐₓ (ng/dL) | 573 ± 285 | 525 ± 223 | 819 ± 359 | 724 ± 346 | 645 ± 280 |
| Tₘₐₓ* (hr) | 22.1 (0.0-25.3) | 19.5 (1.8-24.3) | 15.7 (3.9-24.0) | 23.0 (0.0-24.3) | 11.8 (1.8-24.0) |
| Cₘᵢₙ (ng/dL) | 237 ± 125 | 204 ± 112 | 265 ± 154 | 245 ± 140 | 232 ± 132 |
| Tₘᵢₙ* (hr) | 1.8 (0.0-24.0) | 3.5 (0.0-24.0) | 1.9 (0.0-24.2) | 0.0 (0.0-23.8) | 1.5 (0.0-24.0) |
| Fluc lndex (ratio) | 0.600 ± 0.471 | 0.699 ± 0.503 | 0.678 ± 0.580 | 0.514 ± 0.284 | 0.576 ± 0.341 |

*Median (range)

Days 30, 90, and 180

FIG. Nos. 1(c) and 1(d) show the unique 24-hour pharmacokinetic profile of AndroGel®-treated patients on days 30 and 90. In the AndroGel® groups, serum testosterone levels showed small and variable increases shortly after dosing. The levels then returned to a relatively constant level. In contrast, in the testosterone patch group, patients exhibited a rise over the first 8 to 12 hours, a plateau for another 8 hours, and then a decline to the baseline of the prior day. Further, after gel application on both days 30 and 90, the C_{avg} in the 10.0 g/day AndroGel® group was 1.4 fold higher than in the 5.0 g/day AndroGel® group and 1.9 fold higher than the testosterone patch group. The testosterone patch group also had a Cₘᵢₙ substantially below the lower limit of the normal range. On day 30, the accumulation ratio was 0.94 for testosterone patch group, showing no accumulation. The accumulation ratios at 1.54 and 1.9 were significantly higher in the 5.0 g/day AndroGel® group and 10.0 g/day AndroGel® group, respectively. The differences in accumulation ratio among the groups persisted on day 90. This data indicates that the AndroGel® preparations had a longer effective half-life than testosterone patch.

FIG. No. 1(e) shows the 24-hour pharmacokinetic profile for the treatment groups on day 180. In general, as Table 12(e) shows the 24-hour pharmacokinetic profile for the treatment groups on day 180. In general, as Table 8(e) shows, the serum testosterone concentrations achieved and the pharmacokinetic parameters were similar to those on days 30 and 90 in those patients who continued on their initial randomized treatment groups. Table 8(f) shows that the patients titrated to the 7.5 g/day AndroGel® group were not homogeneous. The patients that were previously in the 10.0 g/day group tended to have higher serum testosterone levels than those previously receiving 5.0 g/day. On day 180, the C_{avg} in the patients in the 10.0 g/day group who converted to 7.5 g/day on day 90 was 744 ng/dL, which was 1.7 fold higher than the C_{avg} of 450 ng/dL in the patients titrated to 7.5 g/day from 5.0 g/day. Despite adjusting the dose up by 2.5 g/day in the 5.0 to 7.5 g/day group, the C_{avg} remained lower than those remaining in the 5.0 g/day group. In the 10.0 to 7.5 g/day group, the C_{avg} became similar to those achieved by patients remaining in the 10.0 g/day group without dose titration. These results suggest that many of the under-responders may actually be poorly compliant patients. For example, if a patient does not apply AndroGel® properly (e.g., preferentially from the placebo container or shortly before bathing), then increasing the dose will not provide any added benefit.

FIG. Nos. 1(f)-(h) compare the pharmacokinetic profiles for the 5.0 g/day AndroGel® group, the 10.0 AndroGel® g/day group, and the testosterone patch group at days 0, 1, 30, 90, and 180. In general, the mean serum testosterone levels in the testosterone patch group remained at the lower limit of the normal range throughout the treatment period. In contrast, the mean serum testosterone levels remained at about 490-570 ng/dL for the 5.0 g/day AndroGel® group and about 630-860 ng/dL AndroGel® for the 10.0 g/day group.

TABLE 12(e)
Testosterone Phamacokinetic Parameters on Day 1
by Initial Treatment Group (Mean ± SD)

| | 5.0 g/day T-Gel | 10.0 g/day T-gel | T-patch |
|---|---|---|---|
| Day 30 | N = 66 | N = 74 | N = 70 |
| C_{avg} (ng/dL) | 566 ± 262 | 792 ± 294 | 419 ± 163 |
| Cₘₐₓ (ng/dL) | 876 ± 466 | 1200 ± 482 | 576 ± 223 |
| Tₘₐₓ* (hr) | 7.9 (0.0-24.0) | 7.8 (0.0-24.3) | 11.3 (0.0-24.0) |
| Cₘᵢₙ (ng/dL) | 361 ± 149 | 505 ± 233 | 235 ± 122 |
| Tₘᵢₙ* (hr) | 8.0 (0.0-24.1) | 8.0 (0.0-25.8) | 2.0 (0.0-24.2) |
| Fluc Index | 0.857 ± 0.331 | 0.895 ± 0.434 | 0.823 ± 0.289 |
| (ratio) | | | |
| Accum Ratio | 1.529 ± 0.726 | 1.911 ± 1.588 | 0.937 ± 0.354 |
| (ratio) | | | |
| Day 90 | N = 65 | N = 73 | N = 64 |
| C_{avg} (ng/dL) | 553 ± 247 | 792 ± 276 | 417 ± 157 |
| Cₘₐₓ (ng/dL) | 846 ± 444 | 1204 ± 570 | 597 ± 242 |
| Tₘₐₓ* (hr) | 4.0 (0.0-24.1) | 7.9 (0.0-25.2) | 8.1 (0.0-25.0) |
| Cₘᵢₙ (ng/dL) | 354 ± 147 | 501 ± 193 | 213 ± 105 |
| Tₘᵢₙ* (hr) | 4.0 (0.0-25.3) | 8.0 (0.0-24.8) | 2.0 (0.0-24.0) |
| Fluc Index | 0.851 ± 0.402 | 0.859 ± 0.399 | 0.937 ± 0.442 |
| (ratio) | | | |
| Accum Ratio | 1.615 ± 0.859 | 1.927 ± 1.310 | 0.971 ± 0.453 |
| (ratio) | | | |
| Day 180 | N = 63 | N = 68 | N = 45 |
| C_{avg} (ng/dL) | 520 ± 227 | 722 ± 242 | 403 ± 163 |
| Cₘₐₓ (ng/dL) | 779 ± 359 | 1091 ± 437 | 580 ± 240 |
| Tₘₐₓ* (hr) | 4.0 (0.0-24.0) | 7.9 (0.0-24.0) | 10.0 (0.0-24.0) |
| Cₘᵢₙ (ng/dL) | 348 ± 164 | 485 ± 184 | 223 ± 114 |
| Tₘᵢₙ* (hr) | 11.9 (0.0-24.0) | 11.8 (0.0-27.4) | 2.0 (0.0-25.7) |
| Fluc Index | 0.845 ± 0.379 | 0.829 ± 0.392 | 0.891 ± 0.319 |
| (ration) | | | |
| Accum Ratio | 1.523 ± 1.024 | 1.897 ± 2.123 | 0.954 ± 0.4105 |
| (ratio) | | | |

*Median (Range)

TABLE 12(f)
Testosterone Phamacokinetic Parameters on Days 30, 90, 180
by Final Treatment Group (Mean ± SD)

| | Doses Received During Initial => Extended Treatment Phases | | | | |
|---|---|---|---|---|---|
| | 5.0 g/day | 5.0 => 7.5 g/day | 10.0 => 7.5 g/day | 10.0 g/day | |
| | T-gel | T-gel | T-gel | T-gel | T-patch |
| Day 30 | N = 47 | N = 19 | N = 19 | N = 55 | N = 70 |
| C_{avg} (ng/dL) | 604 ± 288 | 472 ± 148 | 946 ± 399 | 739 ± 230 | 419 ± 163 |
| Cₘₐₓ (ng/dL) | 941 ± 509 | 716 ± 294 | 1409 ± 556 | 1128 ± 436 | 576 ± 223 |
| Tₘₐₓ* (hr) | 7.9 (0.0-24.0) | 8.0 (0.0-24.0) | 8.0 (0.0-24.3) | 7.8 (0.0-24.3) | 11.3 (0.0-24.0) |
| Cₘᵢₙ (ng/dL) | 387 ± 159 | 296 ± 97 | 600 ± 339 | 471 ± 175 | 235 ± 122 |
| Tₘᵢₙ* (hr) | 8.1 (0.0-24.1) | 1.7 (0.0-24.1) | 11.4 (0.0-24.1) | 8.0 (0.0-25.8) | 2.0 (0.0-24.2) |
| Fluc Index (ratio) | 0.861 ± 0.341 | 0.846 ± 0.315 | 0.927 ± 0.409 | 0.884 ± 0.445 | 0.823 ± 0.289 |
| Accum Ratio (ratio) | 1.543 ± 0.747 | 1.494 ± 0.691 | 2.053 ± 1.393 | 1.864 ± 1.657 | 0.937 ± 0.354 |
| Day 90 | N = 45 | N = 20 | N = 18 | N = 55 | N = 64 |
| C_{avg} (ng/dL) | 596 ± 266 | 455 ± 164 | 859 ± 298 | 771 ± 268 | 417 ± 157 |
| Cₘₐₓ (ng/dL) | 931 ± 455 | 654 ± 359 | 1398 ± 733 | 1141 ± 498 | 597 ± 242 |
| Tₘₐₓ* (hr) | 3.8 (0.0-24.1) | 7.7 (0.0-24.0) | 7.9 (0.0-24.0) | 7.9 (0.0-25.2) | 8.1 (0.0-25.0) |
| Cₘᵢₙ (ng/dL) | 384 ± 147 | 286 ± 125 | 532 ± 181 | 492 ± 197 | 213 ± 105 |
| Tₘᵢₙ* (hr) | 7.9 (0.0-25.3) | 0.0 (0.0-24.0) | 12.0 (0.0-24.1) | 4.0 (0.0-24.8) | 2.0 (0.0-24.0) |
| Fluc Index (ratio) | 0.886 ± 0.391 | 0.771 ± 0.425 | 0.959 ± 0.490 | 0.826 ± 0.363 | 0.937 ± 0.442 |
| Accum Ratio (ratio) | 1.593 ± 0.813 | 1.737 ± 1.145 | 1.752 ± 0.700 | 1.952 ± 1.380 | 0.971 ± 0.453 |
| Day 180 | N = 44 | N = 18 | N = 19 | N = 48 | N = 41 |
| C_{avg} (ng/dL) | 555 ± 225 | 450 ± 219 | 744 ± 320 | 713 ± 209 | 408 ± 165 |
| Cₘₐₓ (ng/dL) | 803 ± 347 | 680 ± 369 | 1110 ± 468 | 1083 ± 434 | 578 ± 245 |
| Tₘₐₓ* (hr) | 5.8 (0.0-24.0) | 2.0 (0.0-24.0) | 7.8 (0.0-24.0) | 7.7 (0.0-24.0) | 10.6 (0.0-24.0) |
| Cₘᵢₙ (ng/dL) | 371 ± 165 | 302 ± 150 | 505 ± 233 | 485 ± 156 | 222 ± 116 |
| Tₘᵢₙ* (hr) | 11.9 (0.0-24.0) | 9.9 (0.0-24.0) | 12.0 (0.0-24.0) | 8.0 (0.0-27.4) | 2.0 (0.0-25.7) |
| Fluc Index (ratio) | 0.853 ± 0.402 | 0.833 ± 0.335 | 0.824 ± 0.298 | 0.818 ± 0.421 | 0.866 ± 0.311 |
| Accum Ratio (ratio) | 1.541 ± 0.917 | NA | NA | 2.061 ± 2.445 | 0.969 ± 0.415 |

*Median (range)

Dose Proportionality for AndroGel®

Table 12(g) shows the increase in AUC₀₋₂₄ on days 30, 90, and 180 from the pretreatment baseline (net AUC₀₋₂₄) as calculated using an arithmetic mean. In order to assess dose-proportionality, the bioequivalence assessment was performed on the log-transformed AUCs using “treatment” as the only factor. The AUCs were compared after subtracting away the AUC contribution from the endogenous secretion of testosterone (the AUC on day 0) and adjusting for the two-fold difference in applied doses. The AUC ratio on day 30 was 0.95 (90% C.I.:0.75-1.19) and on day 90 was 0.92 (90% C.I.:0.73-1.17). the day 30 and day 90 data was combined, the AUC ratio was 0.93 (90% C.I.:0.79-1.10).

The data shows dose proportionality for AndroGel® treatment. The geometric mean for the increase in AUC₀₋₂₄ from day 0 to day 30 or day 90 was twice as great for the 10.0 g/day group as for the 5.0 g/day group. A 125 ng/dL mean increase in serum testosterone C_{avg} level was produced by each 2.5 g/day of AndroGel®. In other words, the data shows that 0.1 g/day of AndroGel® produced, on the average, a 5 ng/dL increase in serum testosterone concentration. This dose proportionality aids dosing adjustment by the physician. Because AndroGel® is provided in 2.5 g packets (containing 25 mg of testosterone), each 2.5 g packet will produce, on average, a 125 ng/dL increase in the C_{avg} for serum total testosterone.

TABLE 12(g)
Net AUC₀₋₂₄ (nmol * h/L) on Days 30, 90, and 180
after Transdermal Testosterone Application

| | T Patch | T gel 5.0 g/day | T gel 10.0 g/day |
|---|---|---|---|
| Day 30 | 154 ± 18 | 268 ± 28 | 446 ± 30 |
| Day 90 | 157 ± 20 | 263 ± 29 | 461 ± 28 |
| Day 180 | 160 ± 25 | 250 ± 32 | 401 ± 27 |

The increase in AUC₀₋₂₄ from pretreatment baseline achieved by the 10.0 g/day and the 5.0 g/day groups were approximately 2.7 and 1.7 fold higher than that resulting from application of the testosterone patch. These figures also indicate that an ANDRODERM® patch, which produces an approximately 180 ng/dL increase in C_{avg}, is equivalent to approximately 3.5 g/day of AndroGel®.

Pharmacokinetics of Serum Free Testosterone Concentration

Methods

Serum free testosterone was measured by RIA of the dialysate, after an overnight equilibrium dialysis, using the same RIA reagents as the testosterone assay. The LLQ of serum free testosterone, using the equilibrium dialysis method, was estimated to be 22 pmol/L. When steroid free serum was spiked with increasing doses of testosterone in the adult male range, increasing amounts of free testosterone were recovered with a coefficient of variation that ranged from 11.0-18.5%. The intra- and interassay coefficients of free testosterone were 15% and 16.8% for adult normal male values, respectively. As estimated by the UCLA-Harbor Medical Center, free testosterone concentrations range from 3.48-17.9 ng/dL (121-620 pmol/L) in normal adult men.

Pharmacokinetic Results

In general, as shown in Table 13, the pharmacokinetic parameters of serum free testosterone mirrored that of serum total testosterone as described above. At baseline (day 0), the mean serum free testosterone concentrations (C_{avg}) were similar in all three groups which were at the lower limit of the adult male range. The maximum serum free testosterone concentration occurred between 8 and 10 a.m., and the minimum about 8 to 16 hours later. This data is consistent with the mild diurnal variation of serum testosterone.

FIG. No. 2(a) shows the 24-hour pharmacokinetic profiles for the three treatment groups on day 1. After application of the testosterone patch, the serum free testosterone levels peaked at 12 hours about 4 hours earlier than those achieved by the AndroGel® groups The serum free testosterone levels then declined in the testosterone patch group whereas in the AndroGel® groups, the serum free testosterone levels continued to rise.

FIG. Nos. 2(b) and 2(c) show the pharmacokinetic profiles of free testosterone in the AndroGel®-treated groups resembled the unique testosterone profiles on days 30 and 90. After AndroGel® application, the mean serum free testosterone levels in the three groups were within normal range. Similar to the total testosterone results, the free testosterone C_{avg} achieved by the 10.0 g/day group was 1.4 fold higher than the 5.0 g/day group and 1.7 fold higher than the testosterone patch group. Moreover, the accumulation ratio for the testosterone patch was significantly less than that of the 5.0 g/day AndroGel® group and the 10.0 g/day AndroGel® group.

FIG. No. 2(d) shows the free testosterone concentrations by final treatment groups on day 180. In general, the free testosterone concentrations exhibited a similar pattern as serum testosterone. The 24-hour pharmacokinetic parameters were similar to those on days 30 and 90 in those subjects who remained in the three original randomized groups. Again, in the subjects titrated to receive 7.5 g/day of AndroGel®, the group was not homogenous. The free testosterone C_{avg} in the patients with doses adjusted upwards from 5.0 to 7.5 g/day remained 29% lower than those of subjects remaining in the 5.0 g/day group. The free testosterone C_{avg} in the patients whose doses were decreased from 10.0 to 7.5 g/day was 11% higher than those in remaining in the 10.0 g/day group.

FIG. Nos. 2(e)-(g) show the free testosterone concentrations in the three groups of subjects throughout the 180-day treatment period. Again, the free testosterone levels followed that of testosterone. The mean free testosterone levels in all three groups were within the normal range with the 10.0 g/day group maintaining higher free testosterone levels than both the 5.0 g/day and the testosterone patch groups.

TABLE 13
Free Testosterone Pharmacokinetic Parameters
by Final Treatment (Mean ± SD)

| | Doses Received During Initial => Extended Treatment Phases | | | | |
|---|---|---|---|---|---|
| | 5.0 g/day | 5.0 => 7.5 g/day | 10.0 => 7.5 g/day | 10.0 g/day | |
| | T-gel | T-gel | T-gel | T-gel | T-patch |
| Day 0 | N = 53 | N = 20 | N = 20 | N = 58 | N = 76 |
| C_{avg} (ng/dL) | 4.52 ± 3.35 | 4.27 ± 3.45 | 4.64 ± 3.10 | 4.20 ± 3.33 | 4.82 ± 3.64 |
| Cₘₐₓ (ng/dL) | 5.98 ± 4.25 | 6.06 ± 5.05 | 6.91 ± 4.66 | 5.84 ± 4.36 | 6.57 ± 4.90 |
| Tₘₐₓ* (hr) | 4.0 (0.0-24.5) | 2.0 (0.0-24.0) | 13.5 (0.0-24.2) | 2.1 (0.0-24.1) | 3.8 (0.0-24.0) |
| Cₘᵢₙ (ng/dL) | 3.23 ± 2.74 | 3.10 ± 2.62 | 3.14 ± 2.14 | 3.12 ± 2.68 | 3.56 ± 2.88 |
| Tₘᵢₙ* (hr) | 8.0 (0.0-24.2) | 9.9 (0.0-16.0) | 4.0 (0.0-23.3) | 8.0 (0.0-24.0) | 7.9 (0.0-24.0) |
| Fluc Index (ratio) | 0.604 ± 0.342 | 0.674 ± 0.512 | 0.756 ± 0.597 | 0.634 ± 0.420 | 0.614 ± 0.362 |
| Day 1 | N = 53 | N = 20 | N = 19 | N = 57 | N = 74 |
| C_{avg} (ng/dL) | 7.50 ± 4.83 | 6.80 ± 4.82 | 9.94 ± 5.04 | 8.93 ± 6.09 | 9.04 ± 4.81 |
| Cₘₐₓ (ng/dL) | 10.86 ± 7.45 | 10.10 ± 7.79 | 15.36 ± 7.31 | 13.20 ± 8.61 | 12.02 ± 6.14 |
| Tₘₐₓ* (hr) | 16.0 (0.0-25.3) | 13.9 (0.0-24.3) | 15.7 (2.0-24.0) | 23.5 (1.8-24.3) | 12.0 (1.8-24.0) |
| Cₘᵢₙ (ng/dL) | 4.30 ± 3.33 | 3.69 ± 3.24 | 3.88 ± 2.73 | 4.40 ± 3.94 | 4.67 ± 3.52 |
| Tₘᵢₙ* (hr) | 0.0 (0.0-24.1) | 1.8 (0.0-24.0) | 0.0 (0.0-24.2) | 0.0 (0.0-23.9) | 0.0 (0.0-24.0) |
| Day 30 | N = 47 | N = 19 | N = 19 | N = 55 | N = 70 |
| C_{avg} (ng/dL) | 11.12 ± 6.22 | 7.81 ± 3.94 | 16.18 ± 8.18 | 13.37 ± 7.13 | 8.12 ± 4.15 |
| Cₘₐₓ (ng/dL) | 16.93 ± 10.47 | 11.62 ± 6.34 | 25.14 ± 10.80 | 19.36 ± 9.75 | 11.48 ± 5.78 |
| Tₘₐₓ* (hr) | 8.0 (0.0-27.8) | 8.0 (0.0-26.3) | 8.0 (0.0-24.3) | 8.0 (0.0-24.3) | 8.0 (0.0-24.0) |
| Cₘᵢₙ (ng/dL) | 6.99 ± 3.82 | 4.78 ± 3.10 | 9.99 ± 7.19 | 8.25 ± 5.22 | 4.31 ± 3.20 |
| Tₘᵢₙ* (hr) | 4.0 (0.0-24.1) | 3.5 (0.0-24.1) | 11.4 (0.0-24.1) | 7.8 (0.0-25.8) | 2.0 (0.0-24.8) |
| Fluc Index (ratio) | 0.853 ± 0.331 | 0.872 ± 0.510 | 1.051 ± 0.449 | 0.861 ± 0.412 | 0.929 ± 0.311 |
| Accum Ratio (ratio) | 1.635 ± 0.820 | 1.479 ± 0.925 | 2.065 ± 1.523 | 1.953 ± 1.626 | 0.980 ± 0.387 |
| Day 90 | N = 45 | N = 20 | N = 18 | N = 55 | N = 64 |
| C_{avg} (ng/dL) | 12.12 ± 7.78 | 8.06 ± 3.78 | 17.65 ± 8.62 | 13.11 ± 5.97 | 8.50 ± 5.04 |
| Cₘₐₓ (ng/dL) | 18.75 ± 12.90 | 10.76 ± 4.48 | 25.29 ± 12.42 | 18.61 ± 8.20 | 12.04 ± 6.81 |
| Tₘₐₓ* (hr) | 4.0 (0.0-24.0) | 9.7 (0.0-24.0) | 8.0 (0.0-24.0) | 8.0 (0.0-25.2) | 11.6 (0.0-25.0) |
| Cₘᵢₙ (ng/dL) | 7.65 ± 4.74 | 4.75 ± 2.86 | 10.56 ± 6.07 | 8.40 ± 4.57 | 4.38 ± 3.70 |
| Tₘᵢₙ* (hr) | 8.0 (0.0-24.0) | 1.9 (0.0-24.0) | 5.9 (0.0-24.1) | 4.0 (0.0-24.8) | 2.0 (0.0-24.1) |
| Fluc Index (ratio) | 0.913 ± 0.492 | 0.815 ± 0.292 | 0.870 ± 0.401 | 0.812 ± 0.335 | 0.968 ± 0.402 |
| Accum Ratio (ratio) | 1.755 ± 0.983 | 1.916 ± 1.816 | 1.843 ± 0.742 | 2.075 ± 1.866 | 1.054 ± 0.498 |
| Day 180 | N = 44 | N = 18 | N = 19 | N = 48 | N = 41 |
| C_{avg} (ng/dL) | 11.01 ± 5.24 | 7.80 ± 4.63 | 14.14 ± 7.73 | 12.77 ± 5.70 | 7.25 ± 4.90 |
| Cₘₐₓ (ng/dL) | 16.21 ± 7.32 | 11.36 ± 6.36 | 22.56 ± 12.62 | 18.58 ± 9.31 | 10.17 ± 5.90 |
| Tₘₐₓ* (hr) | 7.9 (0.0-24.0) | 2.0 (0.0-23.9) | 7.8 (0.0-24.0) | 8.0 (0.0-24.0) | 11.1 (0.0-24.0) |
| Cₘᵢₙ (ng/dL) | 7.18 ± 3.96 | 5.32 ± 4.06 | 9.54 ± 6.45 | 8.23 ± 4.01 | 3.90 ± 4.20 |
| Tₘᵢₙ* (hr) | 9.9 (0.0-24.2) | 7.9 (0.0-24.0) | 8.0 (0.0-23.2) | 11.8 (0.0-27.4) | 2.5 (0.0-25.7) |
| Fluc Index (ratio) | 0.897 ± 0.502 | 0.838 ± 0.378 | 0.950 ± 0.501 | 0.815 ± 0.397 | 0.967 ± 0.370 |
| Accum Ratio (ratio) | 1.712 ± 1.071 | NA | NA | 2.134 ± 1.989 | 1.001 ± 0.580 |

*Median (Range)

Serum DHT Concentrations

Serum DHT was measured by RIA after potassium permanganate treatment of the sample followed by extraction. The methods and reagents of the DHT assay were provided by DSL (Webster, Tex.). The cross reactivities of the antiserum used in the RIA for DHT were 6.5% for 3-β-androstanediol, 1.2% for 3-α-androstanediol, 0.4% for 3-α-androstanediol glucuronide, and 0.4% for testosterone (after potassium permanganate treatment and extraction), and less than 0.01% for other steroids tested. This low cross-reactivity against testosterone was further confirmed by spiking steroid free serum with 35 nmol/L (1,000 pg/dL) of testosterone and taking the samples through the DHT assay. The results even on spiking with over 35 nmol/L of testosterone was measured as less than 0.1 nmol/L of DHT. The LLQ of serum DHT in the assay was 0.43 nmol/L. The mean accuracy (recovery) of the DHT assay determined by spiking steroid free serum with varying amounts of DHT from 0.43 nmol/L to 9 nmol/L was 101% and ranged from 83 to 114%. The intra-assay and inter-assay coefficients of variation for the DHT assay were 7.8 and 16.6%, respectively, for the normal adult male range. The normal adult male range of DHT was 30.7-193.2 ng/dL (1.06 to 6.66 nmol/L ) as determined by the UCLA-Harbor Medical Center.

As shown in Table 14, the pretreatment mean serum DHT concentrations were between 36 and 42 ng/dL, which were near the lower limit of the normal range in all three initial treatment groups. None of the patients had DHT concentrations above the upper limit of the normal range on the pretreatment day, although almost half (103 patients) had concentrations less than the lower limit.

FIG. No. 3 shows that after treatment, the differences between the mean DHT concentrations associated with the different treatment groups were statistically significant, with patients receiving AndroGel® having a higher mean DHT concentration than the patients using the patch and showing dose-dependenice in the mean serum DHT concentrations. Specifically, after testosterone patch application mean serum DHT levels rose to about 1.3 fold above the baseline. In contrast, serum DHT increased to 3.6 and 4.8 fold above baseline after application of 5.0 g/day and 10.0 g/day of AndroGel®, respectively.

TABLE 14
DHT Concentrations (ng/dL)
on Each of the Observation Days
By Initial Treatment (Mean ± SD)

| | Day 0 | Day 30 | Day 60 | Day 90 | Day 120 | Day 150 | Day 180 |
|---|---|---|---|---|---|---|---|
| 5.0 g/day | N = 73 | N = 69 | N = 70 | N = 67 | N = 65 | N = 63 | N = 65 |
| T-gel | 36.0 ± 19.9 | 117.6 ± 74.9 | 122.4 ± 99.4 | 130.1 ± 99.2 | 121.8 ± 89.2 | 144.7 ± 110.5 | 143.7 ± 105.9 |
| 10.0 g/day | N = 78 | N = 78 | N = 74 | N = 75 | N = 68 | N = 67 | N = 71 |
| T-gel | 42.0 ± 29.4 | 200.4 ± 127.8 | 222.0 ± 126.6 | 207.7 ± 111.0 | 187.3 ± 97.3 | 189.1 ± 102.4 | 206.1 ± 105.9 |
| T-Patch | N = 76 | N = 73 | N = 68 | N = 66 | N = 49 | N = 46 | N = 49 |
| | 37.4 ± 21.4 | 50.8 ± 34.6 | 49.3 ± 27.2 | 43.6 ± 26.9 | 53.0 ± 52.8 | 54.0 ± 42.5 | 52.1 ± 34.3 |
| Across RX | 0.6041 | 0.0001 | 0.0001 | 0.0001 | 0.0001 | 0.0001 | 0.0001 |

The increase in DHT concentrations are likely attributed to the concentration and location of 5α-reductase in the skin. For example, the large amounts of 5α-reductase in the scrotal skin presumably causes an increase in DHT concentrations in the TESTODERM® patch. In contrast, the ANDRODERM® and TESTODERM TTS® patches create little change in DTH levels because the surface area of the patch is small and little 5α-reductase is located in nonscrotal skin. AndroGel® presumably causes an increase in DHT levels because the gel is applied to a relatively large skin area and thus exposes testosterone to greater amounts of the enzyme.

To date, elevated DHT levels have not been reported to have any adverse clinical effects. Moreover, there is evidence to suggest that increased DHT levels may inhibit prostate cancer.

DHT/T Ratio

The UCLA-Harbor Medical Center reports a DHT/T ratio of 0.052-0.328 for normal adult men. In this example, the mean ratios for all three treatments were within the normal range on day 0. As shown in FIG. No. 4 and Table 15, there were treatment and concentration-dependent increases observed over the 180-day period. Specifically, the AndroGel® treatment groups showed the largest increase in DHT/T ratio. However, the mean ratios for all of the treatment groups remained within the normal range on all observation days.

TABLE 15
DHT/T Ratio
on Each of the Observation Days
By Initial Treatment (Mean ± SD)

| | Day 0 | Day 30 | Day 60 | Day 90 | Day 120 | Day 150 | Day 180 |
|---|---|---|---|---|---|---|---|
| 5.0 g/day | N = 73 | N = 68 | N = 70 | N = 67 | N = 65 | N = 62 | N = 64 |
| T-gel | 0.198 ± 0.137 | 0.230 ± 0.104 | 0.256 ± 0.132 | 0.248 ± 0.121 | 0.266 ± 0.119 | 0.290 ± 0.145 | 0.273 ± 0.160 |
| 10.0 g/day | N = 78 | N = 77 | N = 74 | N = 74 | N = 68 | N = 67 | N = 71 |
| T-gel | 0.206 ± 0.163 | 0.266 ± 0.124 | 0.313 ± 0.160 | 0.300 ± 0.131 | 0.308 ± 0.145 | 0.325 ± 0.142 | 0.291 ± 0.124 |
| T-Patch | N = 76 | N = 73 | N = 68 | N = 65 | N = 49 | N = 46 | N = 46 |
| | 0.204 ± 0.135 | 0.192 ± 0.182 | 0.175 ± 0.102 | 0.175 ± 0.092 | 0.186 ± 0.134 | 0.223 ± 0.147 | 0.212 ± 0.160 |
| Across RX | 0.7922 | 0.0001 | 0.0001 | 0.0001 | 0.0001 | 0.0001 | 0.0002 |

Total Androgen (DHT+T)

The UCLA-Harbor Medical Center has determined that the normal total androgen concentration is 372 to 1,350 ng/dL. As shown in FIG. No. 5 and Table 16, the mean pre-dose total androgen concentrations for all three treatments were below the lower limit of the normal range on pretreatment day 0. The total androgen concentrations for both AndroGel® groups were within the normal range on all treatment observation days. In contrast, the mean concentrations for patients receiving the testosterone patch was barely within the normal range on day 60 and 120, but were below the lower normal limit on days 30, 90, 150, and 180.

TABLE 16
Total Androgen (DHT + T) (ng/dL)
on Each of the Observation Days
By Initial Treatment (Mean ± SD)

| | Day 0 | Day 30 | Day 60 | Day 90 | Day 120 | Day 150 | Day 180 |
|---|---|---|---|---|---|---|---|
| 5.0 g/day | N = 73 | N = 68 | N = 70 | N = 67 | N = 65 | N = 62 | N = 64 |
| T-gel | 281 ± 150 | 659 ± 398 | 617 ± 429 | 690 ± 431 | 574 ± 331 | 631 ± 384 | 694 ± 412 |
| 10.0 g/day | N = 78 | N = 77 | N = 74 | N = 74 | N = 68 | N = 67 | N = 71 |
| T-gel | 307 ± 180 | 974 ± 532 | 1052 ± 806 | 921 ± 420 | 827 ± 361 | 805 ± 383 | 944 ± 432 |
| T-Patch | N = 76 | N = 73 | N = 68 | N = 65 | N = 49 | N = 46 | N = 46 |
| | 282 ± 159 | 369 ± 206 | 392 ± 229 | 330 ± 173 | 378 ± 250 | 364 ± 220 | 355 ± 202 |
| Across RX | 0.7395 | 0.0001 | 0.0001 | 0.0001 | 0.0001 | 0.0001 | 0.0001 |

E₂ Concentrations

Serum E₂ levels were measured by a direct assay without extraction with reagents from ICN (Costa Mesa, Calif.). The intra-assay and inter-assay coefficients of variation of E₂ were 6.5 and 7.1% respectively. The UCLA-Harbor Medical Center reported an average E₂ concentration ranging from 7.1 to 46.1 pg/mL (63 to 169 pmol/L) for normal adult male range. The LLQ of the E₂ was 18 pmol/L. The cross reactivities of the E₂ antibody were 6.9% for estrone, 0.4% for equilenin, and less than 0.01% for all other steroids tested. The accuracy of the E₂ assay was assessed by spiking steroid free serum with increasing amount of E₂ (18 to 275 pmol/L). The mean recovery of E₂ compared to the amount added was 99.1% and ranged from 95 to 101%.

FIG. No. 6 depicts the E₂ concentrations throughout the 180-day study. The pretreatment mean E₂ concentrations for all three treatment groups were 23-24 pg/mL. During the study, the E₂ levels increased by an average 9.2% in the testosterone patch during the treatment period, 30.9% in the 5.0 g/day AndroGel® group, and 45.5% in the 10.0 g/day AndroGel® group. All of the mean concentrations fell within the normal range.

TABLE 17
Estradiol Concentration (pg/mL)
on Each of the Observation Days
By Initial Treatment (Mean ± SD)

| | Day 0 | Day 30 | Day 60 | Day 90 | Day 120 | Day 150 | Day 180 |
|---|---|---|---|---|---|---|---|
| 5.0 g/day T-gel | N = 73 | N = 69 | N = 68 | N = 67 | N = 64 | N = 65 | N = 65 |
| | 23.0 ± 9.2 | 29.2 ± 11.0 | 28.1 ± 10.0 | 31.4 ± 11.9 | 28.8 ± 9.9 | 30.8 ± 12.5 | 32.3 ± 13.8 |
| 10.0 g/day T-gel | N = 78 | N = 78 | N = 74 | N = 75 | N = 71 | N = 66 | N = 71 |
| | 24.5 ± 9.5 | 33.7 ± 11.5 | 36.5 ± 13.5 | 37.8 ± 13.3 | 34.6 ± 10.4 | 35.0 ± 11.1 | 36.3 ± 13.9 |
| T-Patch | N = 76 | N = 72 | N = 68 | N = 66 | N = 50 | N = 49 | N = 49 |
| | 23.8 ± 8.2 | 25.8 ± 9.8 | 24.8 ± 8.0 | 25.7 ± 9.8 | 25.7 ± 9.4 | 27.0 ± 9.2 | 26.9 ± 9.5 |
| Across RX | 0.6259 | 0.0001 | 0.0001 | 0.0001 | 0.0001 | 0.0009 | 0.0006 |

E₂ is believed to be important for the maintenance of normal bone. In addition, E₂ has a positive effect on serum lipid profiles.

Serum SHBG Concentrations

Serum SHBG levels were measured with a fluoroimmunometric assay (“FIA”) obtained from Delfia (Wallac, Gaithersberg, Md.). The intra- and interassay coefficients were 5% and 12% respectively. The LLQ was 0.5 nmol/L. The UCLA-Harbor Medical Center determined that the adult normal male range for the SHBG assay is 0.8 to 46.6 nmol/L.

As shown in FIG. No. 7 and Table 18, the serum SHBG levels were similar and within the normal adult male range in the three treatment groups at baseline. None of the treatment groups showed major changes from these the baseline on any of the treatment visit days. After testosterone replacement serum SHBG levels showed a small decrease in all three groups. The most marked change occurred in the 10.0 g/day AndroGel® group.

TABLE 18
SHBG Concentration (nmol/L)
on Each of the Observation Days
By Initial Treatment (Mean ± SD)

| | Day 0 | Day 30 | Day 60 | Day 90 | Day 120 | Day 150 | Day 180 |
|---|---|---|---|---|---|---|---|
| 5.0 g/day | N = 73 | N = 69 | N = 69 | N = 67 | N = 66 | N = 65 | N = 65 |
| T-gel | 26.2 ± 14.9 | 24.9 ± 14.0 | 25.9 ± 14.4 | 25.5 ± 14.7 | 25.2 ± 14.1 | 24.9 ± 12.9 | 24.2 ± 13.6 |
| 10.0 g/day | N = 78 | N = 78 | N = 75 | N = 75 | N = 72 | N = 68 | N = 71 |
| T-gel | 26.6 ± 17.8 | 24.8 ± 14.5 | 25.2 ± 15.5 | 23.6 ± 14.7 | 25.5 ± 16.5 | 23.8 ± 12.5 | 24.0 ± 14.5 |
| T-Patch | N = 76 | N = 72 | N = 68 | N = 66 | N = 50 | N = 49 | N = 49 |
| | 30.2 ± 22.6 | 28.4 ± 21.3 | 28.2 ± 23.8 | 28.0 ± 23.6 | 26.7 ± 16.0 | 26.7 ± 16.4 | 25.8 ± 15.1 |
| Across RX | 0.3565 | 0.3434 | 0.5933 | 0.3459 | 0.8578 | 0.5280 | 0.7668 |

Gonadotropins

Serum FSH and LH were measured by highly sensitive and specific solid-phase FIA assays with reagents provided by Delfia (Wallac, Gaithersburg, Md.). The intra-assay coefficient of variations for LH and FSH fluroimmunometric assays were 4.3 and 5.2%, respectively; and the interassay variations for LH and FSH were 11.0% and 12.0%, respectively. For both LH and FSH assays, the LLQ was determined to be 0.2 IU/L. All samples obtained from the same subject were measured in the same assay. The UCLA-Harbor Medical Center reports that the adult normal male range for LH is 1.0-8.1 U/L and for FSH is 1.0-6.9U/L.

FSH

Table 19(a)-(d) shows the concentrations of FSH throughout the 180-day treatment depending on the cause of hypogonadism: (1) primary, (2) secondary, (3) age-associated, or (4) unknown.

Patients with primary hypogonadism show an intact feedback mechanism in that the low serum testosterone concentrations are associated with high FSH and LH concentrations. However, because of testicular or other failures, the high LH concentrations are not effective at stimulating testosterone production.

Secondary hypogonadism involves an idiopathic gonadotropin or LH-releasing hormone deficiency. Because patients with secondary hypogonadism do not demonstrate an intact feedback pathway, the lower testosterone concentrations are not associated with increased LH or FSH levels. Thus, these men have low testosterone serum levels but have gonadotropins in the normal to low range.

Hypogonadism may be age-related. Men experience a slow but continuous decline in average serum testosterone after approximately age 20 to 30 years. These untreated testosterone deficiencies in older men may lead to a variety of physiological changes. The net result is geriatric hypogonadism, or what is commonly referred to as “male menopause.”

As discussed above, patients with primary hypogonadism have an intact feedback inhibition pathway, but the testes do not secrete testosterone. As a result, increasing serum testosterone levels should lead to a decrease in the serum FSH concentrations. In this example, a total of 94 patients were identified as having primary hypogonadism. For these patients, the mean FSH concentrations in the three treatment groups on day 0 were 21-26 mlU/mL, above the upper limit of the normal range. As shown in FIG. No. 8(a) and Table 19(a), the mean FSH concentrations decreased during treatment in all three treatment regimens. However, only the 10.0 g/day AndroGel® group reduced the mean concentrations to within the normal range during the first 90 days of treatment. Treatment with the 10.0 g/day AndroGel® group required approximately 120 days to reach steady state. The mean FSH concentration in patients applying 5.0 g/day of AndroGel® showed an initial decline that was completed by day 30 and another declining phase at day 120 and continuing until the end of treatment. Mean FSH concentrations in the patients receiving the testosterone patch appeared to reached steady state after 30 days but were significantly higher than the normal range.

TABLE 19(a)
FSH Concentrations (mlU/mL) on Each of the
Observation Days by Initial Treatment Group for Patients
Having Primary Hypogonadism (Mean ± SD)

| | N | 5 g/day | N | 10 g/day | N | T-patch |
|---|---|---|---|---|---|---|
| Day 0 | 26 | 21.6 ± 21.0 | 33 | 20.9 ± 15.9 | 34 | 25.5 ± 25.5 |
| Day 30 | 23 | 10.6 ± 15.0 | 34 | 10.6 ± 14.1 | 31 | 21.4 ± 24.6 |
| Day 60 | 24 | 10.8 ± 16.9 | 32 | 7.2 ± 12.6 | 31 | 21.7 ± 23.4 |
| Day 90 | 24 | 10.4 ± 19.7 | 31 | 5.7 ± 10.1 | 30 | 19.5 ± 20.0 |
| Day 120 | 24 | 8.1 ± 15.2 | 28 | 4.6 ± 10.2 | 21 | 25.3 ± 28.4 |
| Day 150 | 22 | 6.7 ± 15.0 | 29 | 5.3 ± 11.0 | 21 | 18.6 ± 24.0 |
| Day 180 | 24 | 6.2 ± 11.3 | 28 | 5.3 ± 11.2 | 22 | 24.5 ± 27.4 |

Patients with secondary hypogonadism have a deficient testosterone negative feedback system. As shown in FIG. No. 8(b), of 44 patients identified as having secondary hypogonadism, the mean FSH concentrations decreased during treatment, although the decrease over time was not statistically significant for the testosterone patch. The patients in the 5.0 g/day AndroGel® group showed a decrease in the mean FSH concentration by about 35% by day 30, with no further decrease evident by day 60. Beyond day 90, the mean FSH concentration in the patients appeared to slowly return toward the pretreatment value. By day 30, all of the 10.0 g/day AndroGel® group had FSH concentrations less than the lower limit.

TABLE 19(b)
FSH Concentrations (mlU/mL) on Each of the
Observation Days by Initial Treatment Group for Patients
Having Secondary Hypogonadism (Mean ± SD)

| | N | 5 g/day | N | 10 g/day | N | T-patch |
|---|---|---|---|---|---|---|
| Day 0 | 17 | 4.2 ± 6.6 | 12 | 2.1 ± 1.9 | 15 | 5.1 ± 9.0 |
| Day 30 | 16 | 2.8 ± 5.9 | 12 | 0.2 ± 0.1 | 14 | 4.2 ± 8.0 |
| Day 60 | 17 | 2.8 ± 6.1 | 12 | 0.2 ± 0.1 | 13 | 4.2 ± 7.4 |
| Day 90 | 15 | 2.9 ± 5.6 | 12 | 0.2 ± 0.1 | 14 | 4.9 ± 9.0 |
| Day 120 | 14 | 3.0 ± 6.1 | 12 | 0.1 ± 0.1 | 12 | 6.1 ± 10.7 |
| Day 150 | 14 | 3.5 ± 7.5 | 12 | 0.2 ± 0.2 | 11 | 4.6 ± 6.5 |
| Day 180 | 14 | 3.7 ± 8.6 | 12 | 0.1 ± 0.1 | 12 | 4.9 ± 7.4 |

Twenty-five patients were diagnosed with age-associated hypogonadism. As shown in FIG. No. 8(c), the 5.0 g/day AndroGel® group had a mean pretreatment FSH concentration above the normal range. The mean concentration for this group was within the normal range by day 30 and had decreased more than 50% on days 90 and 180. The decrease in FSH mean concentration in the 10.0 g/day AndroGel® group showed a more rapid response. The concentrations in all six patients decreased to below the lower normal limit by day 30 and remained there for the duration of the study. The six patients who received the testosterone patch exhibited no consistent pattern in the mean FSH level; however, there was an overall trend towards lower FHS levels with continued treatment.

TABLE 19(c)
FSH Concentrations (mlU/mL) on Each of the
Observation Days by Initial Treatment Group for Patients
Having Age-Related Hypogonadism (Mean ± SD)

| | N | 5 g/day | N | 10 g/day | N | T-patch |
|---|---|---|---|---|---|---|
| Day 0 | 13 | 8.0 ± 9.1 | 6 | 5.2 ± 1.9 | 6 | 4.7 ± 1.7 |
| Day 30 | 12 | 4.6 ± 7.4 | 6 | 0.4 ± 0.3 | 6 | 3.7 ± 2.0 |
| Day 60 | 12 | 3.9 ± 6.6 | 6 | 0.3 ± 0.3 | 4 | 4.3 ± 3.3 |
| Day 90 | 11 | 3.8 ± 7.0 | 6 | 0.4 ± 0.7 | 4 | 3.5 ± 1.9 |
| Day 120 | 11 | 4.2 ± 8.3 | 6 | 0.4 ± 0.7 | 4 | 4.2 ± 3.3 |
| Day 150 | 11 | 4.3 ± 8.1 | 5 | 0.2 ± 0.2 | 4 | 3.4 ± 2.7 |
| Day 180 | 11 | 4.0 ± 7.2 | 6 | 0.2 ± 0.2 | 4 | 2.7 ± 2.1 |

Sixty-four patients in the study suffered from unclassified hypogonadism. As shown in FIG. No. 8(d), the patients showed a marked and comparatively rapid FSH concentration decrease in all three groups, with the greatest decrease being in the 10.0 g/day AndroGel® group. The 10.0 g/day AndroGel® group produced nearly a 90% decrease in the mean FSH concentration by day 30 and maintained the effect to day 180. The 5.0 g/day AndroGel® group produced about a 75% drop in mean FSH concentration by day 30 and stayed at that level for the remainder of treatment. The 21 patients receiving the testosterone patch had a 50% decrease in the mean FSH concentration by day 30, a trend that continued to day 90 when the concentration was about one-third of its pretreatment value.

TABLE 19(d)
Concentrations (mlU/mL) for FSH on Each of
the Observation Days by Initial Treatment Group for
Patients Having Unknown-Related Hypogonadism (Mean ± SD)

| | N | 5 g/day | N | 10 g/day | N | T-patch |
|---|---|---|---|---|---|---|
| Day 0 | 17 | 4.0 ± 1.8 | 26 | 4.1 ± 1.6 | 21 | 3.7 ± 1.4 |
| Day 30 | 17 | 1.1 ± 1.0 | 26 | 0.5 ± 0.5 | 21 | 1.8 ± 0.8 |
| Day 60 | 16 | 1.1 ± 1.1 | 26 | 0.3 ± 0.3 | 18 | 1.6 ± 1.0 |
| Day 90 | 17 | 1.1 ± 1.1 | 25 | 0.4 ± 0.7 | 18 | 1.2 ± 0.9 |
| Day 120 | 16 | 1.2 ± 1.4 | 26 | 0.4 ± 0.6 | 12 | 1.4 ± 1.0 |
| Day 150 | 17 | 1.4 ± 1.4 | 23 | 0.3 ± 0.5 | 13 | 1.4 ± 1.2 |
| Day 180 | 16 | 1.0 ± 0.9 | 24 | 0.4 ± 0.4 | 11 | 1.3 ± 0.9 |

This data shows that feedback inhibition of FSH secretion functioned to some extent in all four subpopulations. The primary hypogonadal population showed a dose-dependency in both the extent and rate of the decline in FSH levels. The sensitivity of the feedback process appeared to be reduced in the secondary and age-associated groups in that only the highest testosterone doses had a significant and prolonged impact on FSH secretion. In contrast, the feedback inhibition pathway in the patients in the unclassified group was quite responsive at even the lowest dose of exogenous testosterone.

LH

The response of LH to testosterone was also examined separately for the same four subpopulations. Table 20(a)-(d) shows the LH concentrations throughout the treatment period.

As shown in FIG. No. 9(a) and Table 20(a), the LH concentrations prior to treatment were about 175% of the upper limit of the normal range in primary hypogonadal patients. The mean LH concentrations decreased during treatment in all groups. However, only the AndroGel® groups decreased the mean LH concentrations enough to fall within the normal range. As with FSH, the primary hypogonadal men receiving AndroGel® showed dose-dependence in both the rate and extent of the LH response.

TABLE 20(a)
Concentrations for LH (mlU/mL) on Each of
the Observation Days for Patients Having Primary
Hypogonadism (Summary of Mean ± SD)

| | N | 5 g/day | N | 10 g/day | N | T-patch |
|---|---|---|---|---|---|---|
| Day 0 | 26 | 12.2 ± 12.1 | 33 | 13.9 ± 14.9 | 33 | 13.3 ± 14.3 |
| Day 30 | 23 | 5.6 ± 7.6 | 34 | 5.9 ± 8.1 | 31 | 10.9 ± 12.9 |
| Day 60 | 24 | 6.8 ± 9.0 | 32 | 4.8 ± 10.0 | 31 | 10.8 ± 11.8 |
| Day 90 | 24 | 5.9 ± 9.5 | 31 | 4.2 ± 11.0 | 30 | 10.0 ± 11.7 |
| Day 120 | 24 | 6.4 ± 11.9 | 28 | 3.8 ± 10.4 | 21 | 11.5 ± 11.5 |
| Day 150 | 22 | 4.4 ± 8.5 | 29 | 4.0 ± 11.3 | 21 | 7.4 ± 6.0 |
| Day 180 | 24 | 4.8 ± 6.8 | 28 | 4.0 ± 11.9 | 22 | 11.2 ± 10.5 |

The secondary hypogonadal men were less sensitive to exogenous testosterone. For the 44 patients identified as having secondary hypogonadism, the pretreatment mean concentrations were all within the lower limit normal range. The mean LH concentrations decreased during treatment with all three regimens as shown in FIG. No. 9(b) and Table 20(b).

TABLE 20(b)
Concentrations for LH (mlU/mL) on Each of
the Observation Days for Patients Having Secondary
Hypogonadism (Summary of Mean ± SD)

| | N | 5 g/day | N | 10 g/day | N | T-patch |
|---|---|---|---|---|---|---|
| Day 0 | 17 | 1.8 ± 2.6 | 12 | 1.4 ± 1.8 | 15 | 1.6 ± 3.1 |
| Day 30 | 16 | 1.1 ± 2.2 | 12 | 0.2 ± 0.2 | 14 | 0.4 ± 0.4 |
| Day 60 | 17 | 1.4 ± 3.8 | 12 | 0.2 ± 0.2 | 13 | 0.6 ± 0.5 |
| Day 90 | 15 | 1.2 ± 2.4 | 12 | 0.2 ± 0.2 | 14 | 0.7 ± 1.0 |
| Day 120 | 14 | 1.6 ± 4.0 | 12 | 0.2 ± 0.2 | 12 | 0.8 ± 0.8 |
| Day 150 | 14 | 1.6 ± 3.5 | 12 | 0.2 ± 0.2 | 11 | 1.2 ± 2.0 |
| Day 180 | 14 | 1.5 ± 3.7 | 12 | 0.2 ± 0.2 | 12 | 1.4 ± 2.1 |

None of the 25 patients suffering from age-associated hypogonadism had pretreatment LH concentrations outside of the normal range as shown in FIG. No. 9(c) and Table 20(c). The overall time and treatment effects were significant for the AndroGel® patients but not those patients using the testosterone patch.

TABLE 20(c)
Concentrations for LH (mlU/mL) on Each of
the Observation Days for Patients Having Age-Related
Hypogonadism (Summary of Mean ± SD)

| | N | 5 g/day | N | 10 g/day | N | T-patch |
|---|---|---|---|---|---|---|
| Day 0 | 13 | 3.2 ± 1.1 | 6 | 2.4 ± 1.8 | 6 | 2.9 ± 0.6 |
| Day 30 | 12 | 1.1 ± 1.0 | 6 | 0.1 ± 0.0 | 6 | 1.8 ± 1.1 |
| Day 60 | 12 | 0.8 ± 0.7 | 6 | 0.2 ± 0.3 | 5 | 3.4 ± 2.8 |
| Day 90 | 11 | 0.9 ± 1.2 | 6 | 0.1 ± 0.0 | 4 | 2.3 ± 1.4 |
| Day 120 | 11 | 1.0 ± 1.4 | 6 | 0.1 ± 0.0 | 4 | 2.2 ± 1.4 |
| Day 150 | 11 | 1.3 ± 1.5 | 5 | 0.1 ± 0.0 | 4 | 1.9 ± 1.2 |
| Day 180 | 11 | 1.8 ± 2.1 | 6 | 0.1 ± 0.0 | 4 | 1.4 ± 1.0 |

Of the 64 patients suffering from an unclassified hypogonadism, none of the patients had a pretreatment LH concentration above the upper limit. Fifteen percent, however, had pretreatment concentrations below the normal limit. The unclassified patients showed comparatively rapid LH concentration decreases in all treatment groups as shown in FIG. No. 9(d) and Table 20(d).

TABLE 20(d)
Concentrations for LH (mlU/mL) on Each of
the Observation Days for Patients Having Unknown-
Related Hypogonadism (Summary of Mean ± SD)

| | N | 5 g/day | N | 10 g/day | N | T-patch |
|---|---|---|---|---|---|---|
| Day 0 | 17 | 1.8 ± 1.2 | 26 | 2.5 ± 1.5 | 21 | 2.5 ± 1.5 |
| Day 30 | 17 | 0.3 ± 0.3 | 26 | 0.3 ± 0.3 | 21 | 1.3 ± 1.3 |
| Day 60 | 17 | 0.4 ± 0.5 | 26 | 0.3 ± 0.3 | 18 | 1.2 ± 1.4 |
| Day 90 | 17 | 0.5 ± 0.5 | 26 | 0.3 ± 0.4 | 18 | 1.0 ± 1.4 |
| Day 120 | 17 | 0.4 ± 0.4 | 26 | 0.4 ± 0.5 | 12 | 1.2 ± 1.1 |
| Day 150 | 17 | 0.8 ± 1.1 | 23 | 0.3 ± 0.4 | 13 | 1.1 ± 1.1 |
| Day 180 | 15 | 0.3 ± 0.4 | 25 | 0.4 ± 0.4 | 11 | 1.5 ± 1.3 |

Summary: LH and FSH

Patients receiving AndroGel® or the testosterone patch achieve “hormonal steady state” only after long-term treatment. Specifically, data involving FSH and LH show that these hormones do not achieve steady-state until many weeks after treatment. Because testosterone concentrations are negatively inhibited by FSH and LH, testosterone levels do not achieve true steady state until these other hormones also achieve steady state. However, because these hormones regulate only endogenous testosterone (which is small to begin with in hypogonadal men) in an intact feedback mechanism (which may not be present depending on the cause of hypogonadism), the level of FSH and/or LH may have little effect on the actual testosterone levels achieved. The net result is that the patients do not achieve a “hormonal steady state” for testosterone even though the C_{avg} , Cₘᵢₙ, and Cₘₐₓ for testosterone remains relatively constant after a few days of treatment.

Libido and Sexual Performance

Libido and sexual function were assessed by questionnaires the patients answered daily for seven consecutive days before clinic visits on day 0 and on days 30, 60, 90, 120, 150, and 180 days during gel and patch application. The subjects recorded whether they had sexual day dreams, anticipation of sex, flirting, sexual interaction (e.g., sexual motivation parameters) and orgasm, erection, masturbation, ejaculation, intercourse (e.g., sexual performance parameters) on each of the seven days. The value was recorded as 0 (none) or 1 (any) for analyses and the number of days the subjects noted a parameter was summed for the seven-day period. The average of the four sexual motivation parameters was taken as the sexual motivation mean score and that of the five sexual performance parameters as the sexual performance mean score (0 to 7).

The subjects also assessed their level of sexual desire, sexual enjoyment, and satisfaction of erection using a seven-point Likert-type scale (0 to 7) and the percent of full erection from 0 to 100%. The subjects rated their mood using a 0 to 7 score. Weekly average scores were calculated. The details of this questionnaire had been described previously and are fully incorporated by reference. See Wang et al., Testosterone Replacement Therapy Improves Mood in Hypogonadal Men—A Clinical Research Center Study, 81 J. CLINICAL ENDOCRINOLOGY& METABOLISM 3578-3583 (1996).

Libido

As shown in FIG. No. 10(a), at baseline, sexual motivation was the same in all treatment groups. After transdermal testosterone treatment, overall sexual motivation showed significant improvement. The change in the summary score from baseline, however, was not different among the three treatment groups.

Libido was also assessed from responses on a linear scale of: (1) overall sexual desire, (2) enjoyment of sexual activity without a partner, and (3) enjoyment of sexual activity with a partner. As shown in FIG. No. 10(b) and Table 21, as a group, overall sexual desire increased after transdermal testosterone treatment without inter-group difference. Sexual enjoyment with and without a partner (FIG. No. 10(c) and Tables 22 and 23) also increased as a group.

TABLE 21
Overall Sexual Desire
Changes From Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gcl | 69 | 2.1 ± 1.6 | 63 | 3.5 ± 1.6 | 60 | 1.4 ± 1.9 | 0.0001 |
| 100 g/ay T-gel | 77 | 2.0 ± 1.4 | 68 | 3.6 ± 1.6 | 67 | 1.5 ± 1.9 | 0.0001 |
| T-Patch | 72 | 2.0 ± 1.6 | 47 | 3.1 ± 1.9 | 45 | 1.6 ± 2.1 | 0.0001 |
| Across-Groups | | 0.8955 | | 0.2247 | | 0.8579 | |
| p-value | | | | | | | |

TABLE 22
Level of Sexual Enjoyment Without a Partner
Changes From Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gel | 60 | 1.5 ± 1.9 | 51 | 1.9 ± 1.9 | 44 | 0.8 ± 1.4 | 0.0051 |
| 10.0 g/day T-gel | 63 | 1.2 ± 1.4 | 53 | 2.2 ± 1.9 | 48 | 1.1 ± 1.6 | 0.0001 |
| T-Patch | 66 | 1.4 ± 1.8 | 44 | 2.2 ± 2.3 | 40 | 1.0 ± 1.9 | 0.0026 |
| Across-Groups | | 0.6506 | | 0.7461 | | 0.6126 | |
| p-value | | | | | | | |

TABLE 23
Level of Sexual Enjoyment With a Partner
Change from Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gel | 64 | 2.1 ± 2.1 | 55 | 2.6 ± 2.2 | 48 | 0.4 ± 2.2 | 0.0148 |
| 10.0 g/day T-gel | 66 | 1.8 ± 1.7 | 58 | 3.0 ± 2.2 | 52 | 1.0 ± 2.3 | 0.0053 |
| T-Patch | 61 | 1.5 ± 1.7 | 40 | 2.2 ± 2.4 | 35 | 0.7 ± 2.3 | 0.1170 |
| Across-Groups | | 02914 | | 01738 | | 0.3911 | |
| p-value | | | | | | | |

Sexual Performance

FIG. No. 11(a) shows that while all treatment groups had the same baseline sexual performance rating, the rating improved with transdermal testosterone treatment in all groups. In addition, as a group, the subjects' self-assessment of satisfaction of erection (FIG. No. 11(b) and Table 24) and percent full erection (FIG. No. 11(c) and Table 25) were also increased with testosterone replacement without significant differences between groups. The improvement in sexual function was not related to the dose or the delivery method of testosterone. Nor was the improvement related to the serum testosterone levels achieved by the various testosterone preparations. The data suggest that once a threshold (serum testosterone level probably at the low normal range) is achieved, normalization of sexual function occurs. Increasing serum testosterone levels higher to the upper normal range does not further improve sexual motivation or performance.

TABLE 24
Satisfaction with Duration of Erection
Change from Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gel | 55 | 2.5 ± 2.1 | 57 | 4.3 ± 1.8 | 44 | 1.9 ± 2.0 | 0.0001 |
| 10.0 g/day T-gel | 64 | 2.9 ± 1.9 | 58 | 4.5 ± 1.7 | 53 | 1.5 ± 2.0 | 0.0001 |
| T-Patch | 45 | 3.4 ± 2.1 | 34 | 4.5 ± 2.0 | 20 | 1.3 ± 2.1 | 0.0524 |
| Across-Groups | | 0.1117 | | 0.7093 | | 0.5090 | |
| p-value | | | | | | | |

TABLE 25
Percentage of Full Erection
Change from Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gel | 53 | 53.1 ± 24.1 | 57 | 67.4 ± 22.5 | 43 | 18.7 ± 22.1 | 0.0001 |
| 10.0 g/day T-gel | 62 | 59.6 ± 22.1 | 59 | 72.0 ± 20.2 | 52 | 10.4 ± 23.4 | 0.0001 |
| T-Patch | 47 | 56.5 ± 24.7 | 33 | 66.7 ± 26.7 | 19 | 12.7 ± 20.3 | 0.0064 |
| Across-Groups | | 0.3360 | | 0.4360 | | 0.1947 | |
| p-value | | | | | | | |

### Example 6

### Method of Increasing Libido in Eugonadal Men Having a Diminished Libido

As discussed above, transdermal application of testosterone using AndroGel® to hypogonadal men results in improved libido and sexual performance. Researchers have found that eugonadal men having a diminished libido have a significant increase in sexual interest after receiving testosterone injections. See O'Carrol & Bancroft, Testosterone Therapy for Low Sexual Interest and Erectile Dysfunction in Men: A Controlled Study, Brit. J. Psychiatry 145:146-151 (1984). Thus, the present example is directed to a method of treating a diminished libido in eugonadal men by transdermal application of a hydroalcoholic testosterone gel to such men. In one embodiment, AndroGel® is applied to the body in accordance with the protocol summarized in Example 1. Libido is measured as in Example 1. Men receiving AndroGel are expected to show a increase in their libido.

### Example 7

### Method of Increasing Libido in Eugonadal Men Having a Normal Libido

As discussed above, transdermal application of testosterone using AndroGel® to hypogonadal men results in improved libido and sexual performance. Studies have shown that supra-physiological doses of testosterone administered to eugonadal men having a normal libido resulted in a significant increase in libido. See Anderson et al., The Effect of Exogenous Tstoserone on Sexuality and Mood of Normal Men, J. CLINICAL ENDOCRINOLOGY & METABOLISM 75:1505-1507 (1992); Bagatel et al., Metabolic & Behavioral Effects of High-Dose, Exogenous Testosterone in Healthy Men, J. CLINICAL METABOLISM & ENDOCRINOLOGY 79:561-567 (1994). Thus, this example is directed to a method of increasing the libido of normal eugonadal men by application of a transdermal hydroalcoholic testosterone gel. In one embodiment, AndroGel® is applied to the body in accordance with the protocol summarized in Example 1. Libido is measured as in Example 1. Men receiving AndroGel are expected to show a increase in their libido.

### Example 8

### Method of Improving Sexual Performance in Eugonadal Men Having Erectile Dysfunction

In a prophetic example, 10 eugonadal males age 18 and older having erectile dysfunction will be randomized to receive: (a) 5.0 g/day of AndroGel® (delivering 50 mg/day of testosterone to the skin of which about 10% or 5 mg is absorbed) for 30 days or (b) 10.0 g/day of AndroGel® (delivering 100 mg/day of testosterone to the skin of which about 10% or 10 mg is absorbed) for 30 days ; or (c) nothing. The effectiveness of AndroGel® in improving sexual performance and treating erecile dysfunction will be evaluated using several assessment instruments. The primary measure will be a sexual function questionnaire, the International Index of Erectile Function (“IIEF”). Two of the questions from the IIEF will serve as primary study endpoints; categorical responses shall be elicited to questions about (1) the ability to achieve erections sufficient for sexual intercourse and (2) the maintenance of erections after penetration. The possible categorical responses to these questions will be (0) no attempted intercourse, (1) never or almost never, (2) a few times, (3) sometimes, (4) most times, and (5) almost always or always. Also collected as part of the IIEF will be information about other aspects of sexual function, including information on erectile function, orgasm, desire, satisfaction with intercourse, and overall sexual satisfaction. Sexual function data shall also be recorded by patients in a daily diary. In addition, patients shall be asked a global efficacy question and an optional partner questionnaire was administered. In addition, the improvement in erectile dysfunction shall be assessed by an objective measurement of hardness and duration of erections (RigiScan®) with AndroGel treatment compared with placebo. Applicant expects that all test parameters will show improvement over the placebo.

### Example 9

### Method of Improving Sexual Performance in Eugonadal Men Having Normal Erections

In a prophetic example, 10 eugonadal males age 18 and older having normal erections (i.e. not diagnosed with erectile dysfunction) will be randomized to receive: (a) 5.0 g/day of AndroGel® (delivering 50 mg/day of testosterone to the skin of which about 10% or 5 mg is absorbed) for 30 days or (b) 10.0 g/day of AndroGel® (delivering 100 mg/day of testosterone to the skin of which about 10% or 10 mg is absorbed) for 30 days ; or (c) nothing. The effectiveness of AndroGel® will be evaluated using several assessment instrument as discussed in Example 4. Applicant expects that all test parameters will show an increase in sexual performance over the placebo. Accordingly, Applicant expects that AndroGel® can be applied to normal men in order to increase the sexual performance above their normal baseline.

### Example 10

### Treatment of Hypogonadism in Male Subjects

One embodiment of the present invention involves the transdermal application of AndroGel® as a method of treating male hypogonadism. As demonstrated below, application of the gel results in a unique pharmacokinetic profile for testosterone, as well as concomitant modulation of several other sex hormones. Application of the testosterone gel to hypogonadal male subjects also results in: (1) increased bone mineral density, (2) enhanced libido, (3) enhanced erectile capability and satisfaction, (4) increased positive mood, (5) increased muscle strength, and (6) better body composition, such increased total body lean mass and decreased total body fat mass. Moreover, the gel is not associated with significant skin irritation.

Methods

In this example, hypogonadal men were recruited and studied in 16 centers in the United States. The patients were between 19 and 68 years and had single morning serum testosterone levels at screening of less than or equal to 300 ng/dL (10.4 nmol/L ). A total of 227 patients were enrolled: 73, 78, and 76 were randomized to receive 5.0 g/day of AndroGel® (delivering 50 mg/day of testosterone to the skin of which about 10% or 5 mg is absorbed), 10.0 g/day of AndroGel® (delivering 100 mg/day of testosterone to the skin of which about 10% or 10 mg is absorbed), or the ANDRODERM® testosterone patch (“T patch”) (delivering 50 mg/day of testosterone), respectively.

As shown in the Table 26, there were no significant group-associated differences of the patients' characteristics at baseline.

TABLE 26
Baseline Characteristics of the Hypogonadal Men

| | | AndroGel ® | AndroGel ® |
|---|---|---|---|
| Treatment Group | T patch | (5.0 g/day) | (10.0 g/day) |
| No of subjects enrolled | 76 | 73 | 78 |
| Age (years) | 51.1 | 51.3 | 51.0 |
| Range (years) | 28-67 | 23-67 | 19-68 |
| Height (cm) | 179.3 ± 0.9 | 175.8 ± 0.8 | 178.6 ± 0.8 |
| Weight (kg) | 92.7 ± 1.6 | 90.5 ± 1.8 | 91.6 ± 1.5 |
| Serum testosterone (nmol/L) | 6.40 ± 0.41 | 6.44 ± 0.39 | 6.49 ± 0.37 |
| Causes of hypogonadism | | | |
| Primary hypogonadism | 34 | 26 | 34 |
| Klinefelter's Syndrome | 9 | 5 | 8 |
| Post Orchidectomy/Anorchia | 2 | 1 | 3 |
| Primary Testicular Failure | 23 | 20 | 23 |
| Secondary hypogonadism | 15 | 17 | 12 |
| Kallman's Syndrome | 2 | 2 | 0 |
| Hypothalimic Pituitary Disorder | 6 | 6 | 3 |
| Pituitary Tumor | 7 | 9 | 9 |
| Aging | 6 | 13 | 6 |
| Not classified | 21 | 17 | 26 |
| Years diagnosed | 5.8 ± 1.1 | 4.4 ± 0.9 | 5.7 ± 1.24 |
| Number previously treated with | 50 (65.8%) | 38 (52.1%) | 46 (59.0%) |
| testosterone | | | |
| Type of Previous Hormonal Treatment | | | |
| Intramuscular injections | 26 | 20 | 28 |
| Transdermal patch | 12 | 7 | 8 |
| All others | 12 | 11 | 10 |
| Duration of treatment (years) | 5.8 ± 1.0 | 5.4 ± 0.8 | 4.6 ± 80.7 |

Forty-one percent (93/227) of the subjects had not received prior testosterone replacement therapy. Previously treated hypogonadal men were withdrawn from testosterone ester injection for at least six weeks and oral or transdermal androgens for four weeks before the screening visit. Aside from the hypogonadism, the subjects were in good health as evidenced by medical history, physical examination, complete blood count, urinalysis, and serum biochemistry. If the subjects were on lipid-lowering agents or tranquilizers, the doses were stabilized for at least three months prior to enrollment. Less than 5% of the subjects were taking supplemental calcium or vitamin D during the study. The subjects had no history of chronic medical illness, alcohol or drug abuse. They had a normal rectal examination, a PSA level of less than 4 ng/mL, and a urine flow rate of 12 mL/s or greater. Patients were excluded if they had a generalized skin disease that might affect the testosterone absorption or prior history of skin irritability with ANDRODERM® patch. Subjects weighing less than 80% or over 140% of their ideal body weight were also excluded.

The randomized, multi-center, parallel study compared two doses of AndroGel® with the ANDRODERM® testosterone patch. The study was double-blind with respect to the AndroGel® dose and open-labeled for the testosterone patch group. For the first three months of the study (days 1 to 90), the subjects were randomized to receive 5.0 g/day of AndroGel®, 10.0 g/day of AndroGel®, or two non-scrotal patches. In the following three months (days 91 to 180), the subjects were administered one of the following treatments: 5.0 g/day of AndroGel®, 10.0 g/day of AndroGel®, 7.5 g/day of AndroGel®, or two non-scrotal patches. Patients who were applying AndroGel® had a single, pre-application serum testosterone measured on day 60 and, if the levels were within the normal range of 300 to 1,000 ng/dL (10.4 to 34.7 nmol/L ), then they remained on their original dose. Patients with testosterone levels less than 300 ng/dL and who were originally assigned to apply 5.0 g/day of AndroGel® and those with testosterone levels more than 1,000 ng/dL who had received 10.0 g/day of AndroGel® were then reassigned to administer 7.5 g/day of AndroGel® for days 91 to 180.

Accordingly, at 90 days, dose adjustments were made in the AndroGel® groups based on the pre-application serum testosterone levels on day 60. Twenty subjects in the 5.0 g/day AndroGel® group had the dose increased to 7.5 g/day. Twenty patients in the 10.0 g/day AndroGel® group had the AndroGel® dose reduced to 7.5 g/day. There were three patients in the testosterone patch group who were switched to 5.0 g/day AndroGel® because of patch intolerance. One 10.0 g/day AndroGel® subject was adjusted to receive 5.0 g/day and one 5.0 g/day AndroGel® subject had the dose adjusted to 2.5 g/day. The number of subjects enrolled into day 91 to 180 of the study thus consisted of 51 receiving 5.0 g/day of AndroGel®, 40 receiving 7.5 g/day of AndroGel®, 52 receiving 10.0 g/day of AndroGel®, and 52 continuing on the ANDRODERM® patch. The treatment groups in this example may thus be characterized in two ways, either by “initial” or by the “final” treatment group.

Subjects returned to the study center on days 0, 30, 60, 90, 120, 150, and 180 for a clinical examination, skin irritation and adverse event assessments. Fasting blood samples for calcium, inorganic phosphorus, parathyroid hormone (“PTH”), osteocalcin, type I procollagen, and skeletal specific alkaline phosphatase (“SALP”) were collected on days 0, 30, 90, 120, and 180. In addition, a fasting two-hour timed urine collection for urine creatinine, calcium, and type I collagen cross-linked N-telopeptides (“N-telopeptide”) were collected on days 0, 30, 90, 120, and 180. Other tests performed were as follows:

(1) Hematology: hemoglobin, hematocrit, red blood cell count, platelets, white blood cell counts with differential analysis (neutrophils, lymphocytes, monocytes, eosinophils, and basophils);

(2) Chemistry: alkaline phosphatase, alanine aminotransferase, serum glutamic pyruvic transaminase (“ALT/SGPT”), asparate aminotransferase/serum glutamin axaloacetic transaminase (“AST/SGOT”), total bilirubin, creatinine, glucose, and elecrolytes (sodium, potassium, choride, bicarbonate, calcium, and inorganic phosphorus);

(3) Lipids: total cholesterol, high-density lipoprotein (“HDL”), low-density lipoprotein (“LDL”), and triglycerides;

(4) Urinalysis: color, appearance, specific gravity, pH, protein, glucose, ketones, blood, bilirubin, and nitrites; and

(5) Other: PSA (screening days 90-180), prolactin (screening), and testosterone (screening) including electrolytes, glucose, renal, and liver function tests and lipid profile, were performed at all clinic visits. Bone mineral density (“BMD”) was analyzed at day 0 and day 180.

A. AndroGel® and ANDRODERM® Patch

Approximately 250 g of AndroGel® was packaged in multidose glass bottles that delivered 2.25 g of the gel for each actuation of the pump. Patients assigned to apply 5.0 g/day of AndroGel® testosterone were given one bottle of AndroGel® and one bottle of placebo gel (containing vehicle but no testosterone), while those assigned to receive 10.0 g/day of AndroGel® were dispensed two bottles of the active AndroGel®. The patients were then instructed to apply the bottle contents to the right and left upper arms/shoulders and to the right and left sides of the abdomen on an alternate basis. For example, on the first day of the study, patients applied two actuations from one bottle, one each to the left and right upper arm/shoulder, and two actuations from the second bottle, one each to the left and right abdomen. On the following day of treatment, the applications were reversed. Alternate application sites continued throughout the study. After application of the gel to the skin, the gel dried within a few minutes. Patients washed their hands thoroughly with soap and water immediately after gel application.

The 7.5 g/day AndroGel® group received their dose in an open-label fashion. After 90 days, for the subjects titrated to the AndroGel® 7.5 g/day dose, the patients were supplied with three bottles, one containing placebo and the other two AndroGel®. The subjects were instructed to apply one actuation from the placebo bottle and three actuations from a AndroGel® bottle to four different sites of the body as above. The sites were rotated each day taking the same sequence as described above.

ANDRODERM® testosterone patches each delivering 2.5 mg/day of testosterone were provided to about one-third of the patients in the study. These patients were instructed to apply two testosterone patches to a clean, dry area of skin on the back, abdomen, upper arms, or thighs once per day. Application sites were rotated with approximately seven days interval between applications to the same site.

On study days when the patients were evaluated, the gel/patches were applied following pre-dose evaluations. On the remaining days, the testosterone gel or patches were applied at approximately 8:00 a.m. for 180 days.

Study Method and Results

Hormone Pharmacokinetics

On days 0, 1, 30, 90, and 180, the patients had multiple blood samples for testosterone and free testosterone measurements at 30, 15 and 0 minutes before and 2, 4, 8, 12, 16, and 24 hours after AndroGel® or patch application. In addition, subjects returned on days 60, 120, and 150 for a single blood sampling prior to application of the gel or patch. Serum DHT, E₂, FSH, LH and SHBG were measured on samples collected before gel application on days 0, 30, 60, 90, 120, 150, and 180. Sera for all hormones were stored frozen at −20° C. until assay. All samples for a patient for each hormone were measured in the same assay whenever possible. The hormone assays were then measured at the Endocrine Research Laboratory of the UCLA-Harbor Medical Center.

The following table summarizes the pharmacokinetic parameters were measured for each patient:

TABLE 27
Pharmacokinetic Parameters

| | |
|---|---|
| AUC₀₋₂₄ | area under the curve from 0 to 24 hours, determined |
| | using the linear trapezoidal rule. |
| C_{base} or Cₒ | Baseline concentration |
| C_{avg} | time-averaged concentration over the 24-hour dosing |
| | interval determined by AUC₀₋₂₄/24 |
| Cₘₐₓ | maximum concentration during the 24-hour dosing |
| | interval |
| Cₘᵢₙ | minimum concentration during the 24-hour dosing |
| | interval |
| Tₘₐₓ | time at which Cₘₐₓ occurred |
| Tₘᵢₙ | time at which Cₘᵢₙ occurred |
| Fluctuation | extent of variation in the serum concentration over the |
| Index | course of a single day, calculated as (Cₘₐₓ − Cₘᵢₙ)/C_{avg} |
| Accumulation | increase in the daily drug exposure with continued |
| ratio | dosing, calculated as the ratio of the AUC at steady on |
| | a particular day over the AUC on day 1 (e.g., AUC_{day 30}/ |
| | AUC_{day 1}) |
| Net AUC₀₋₂₄ | AUC₀₋₂₄ on days 30, 90, 180 - AUC₀₋₂₄ on day 0 |

Testosterone Pharmacokinetics

Methods

Serum testosterone levels were measured after extraction with ethylacetate and hexane by a specific radioimmunoassay (“RIA”) using reagents from ICN (Costa Mesa, Calif.). The cross reactivities of the antiserum used in the testosterone RIA were 2.0% for DHT, 2.3% for androstenedione, 0.8% for 3- [symbol] -androstanediol, 0.6% for etiocholanolone and less than 0.01% for all other steroids tested. The lower limit of quantitation (“LLQ”) for serum testosterone measured by this assay was 25 ng/dL (0.87 nmol/L). The mean accuracy of the testosterone assay, determined by spiking steroid free serum with varying amounts of testosterone (0.9 nmol/L to 52 nmol/L), was 104% and ranged from 92% to 117%. The intra-assay and inter-assay coefficients of the testosterone assay were 7.3 and 11.1%, respectively, at the normal adult male range. In normal adult men, testosterone concentrations range from 298 to 1,043 ng/dL (10.33 to 36.17 nmol/L) as determined at the UCLA-Harbor Medical Center.

Baseline Concentration

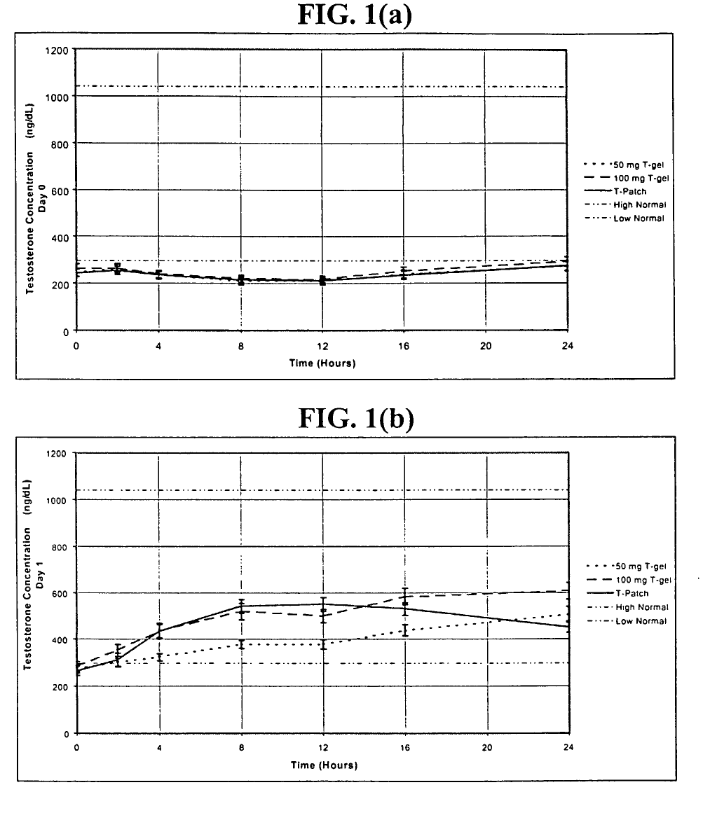

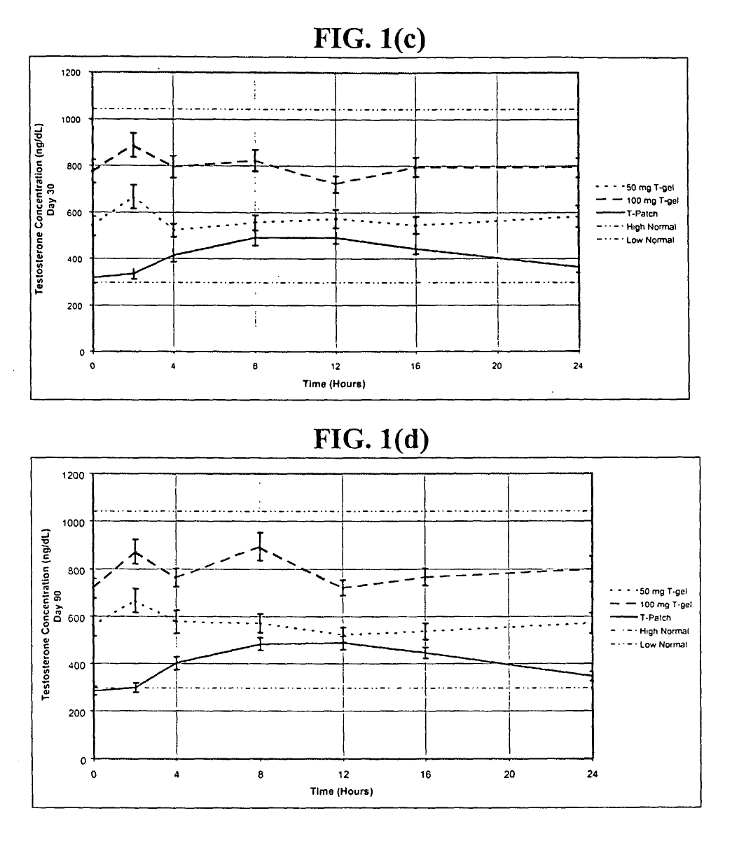

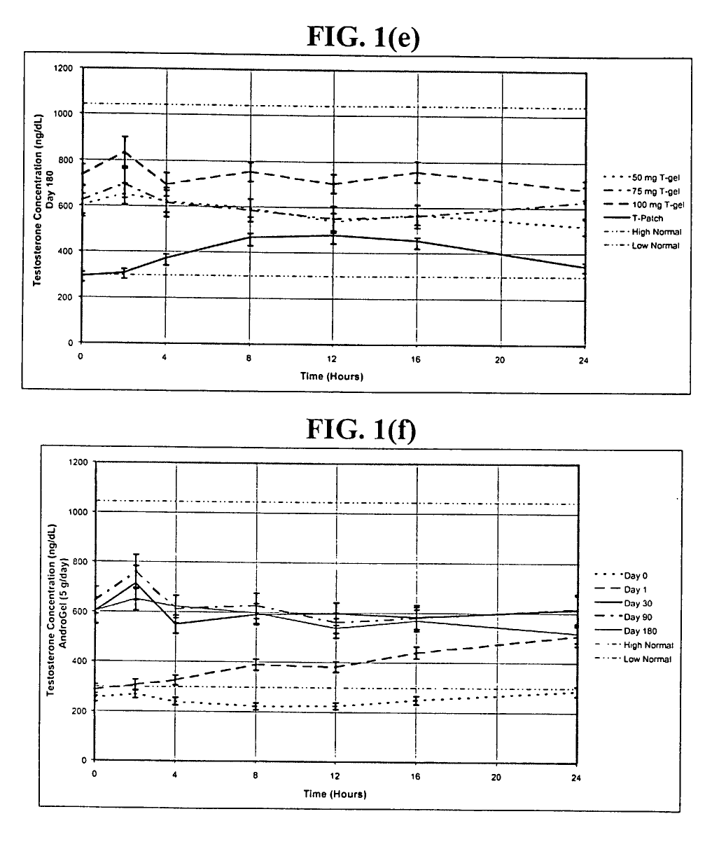

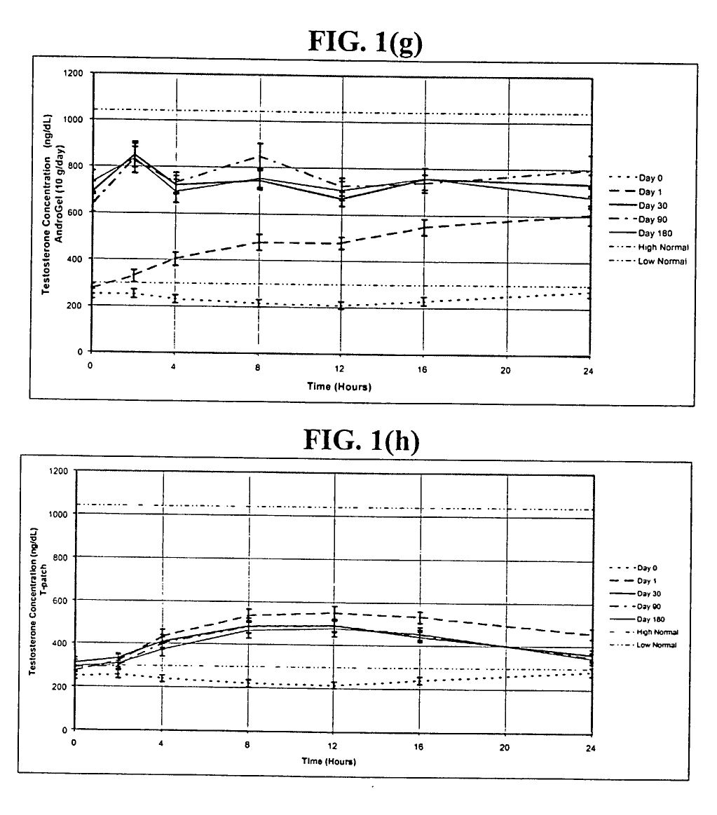

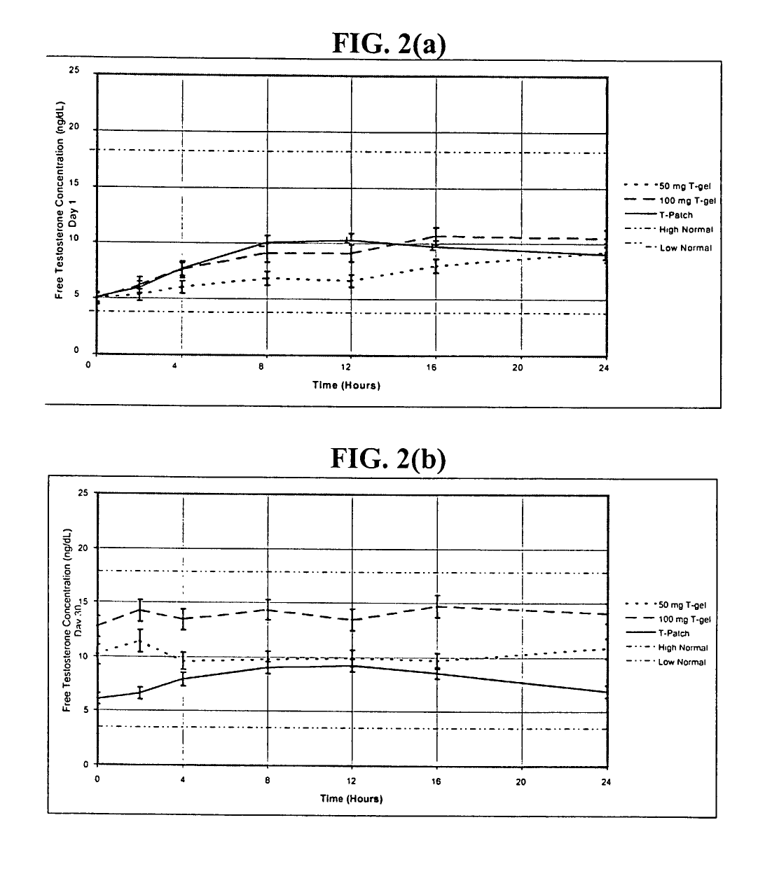

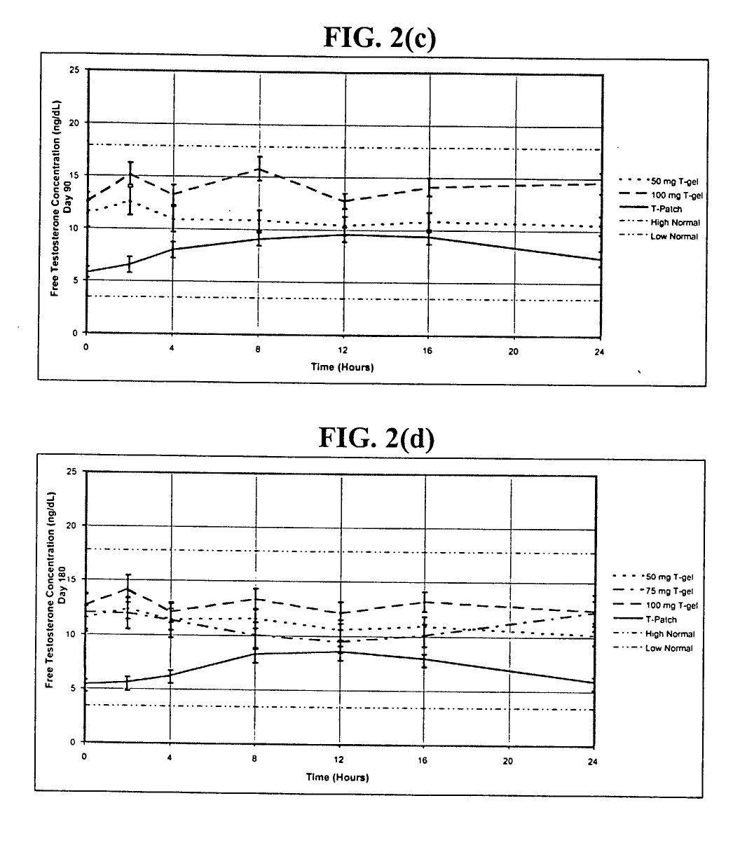

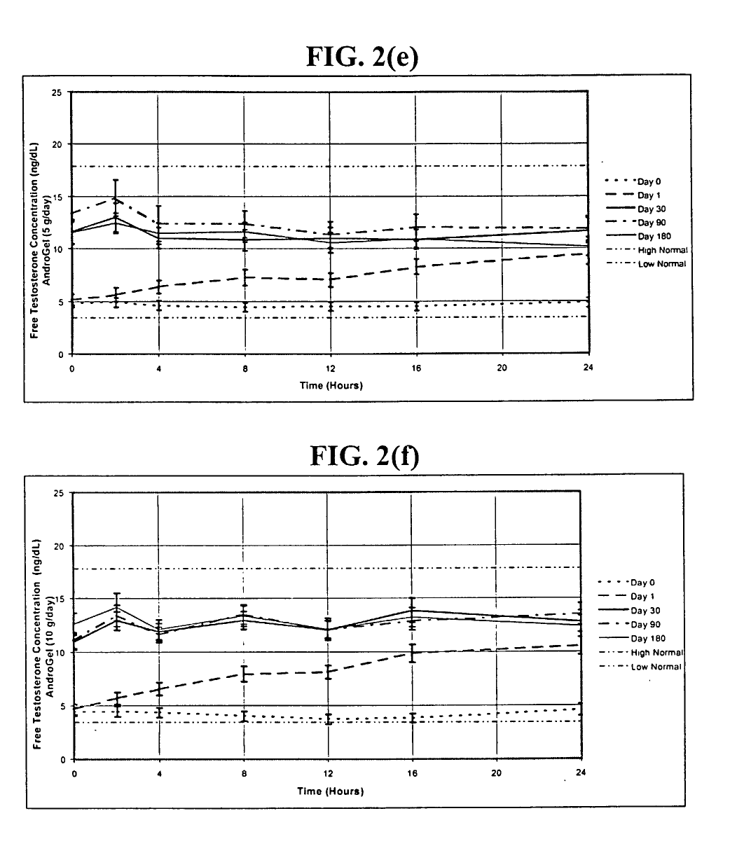

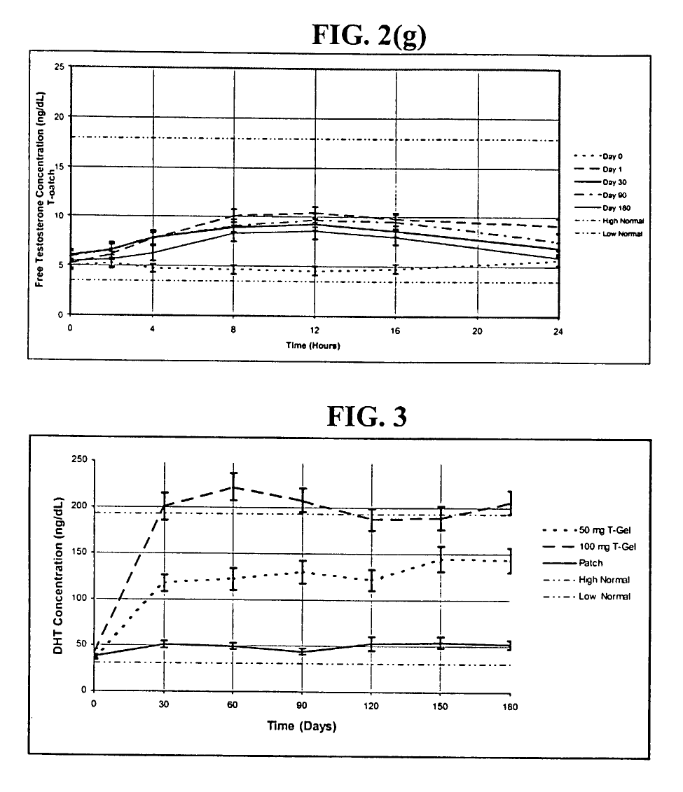

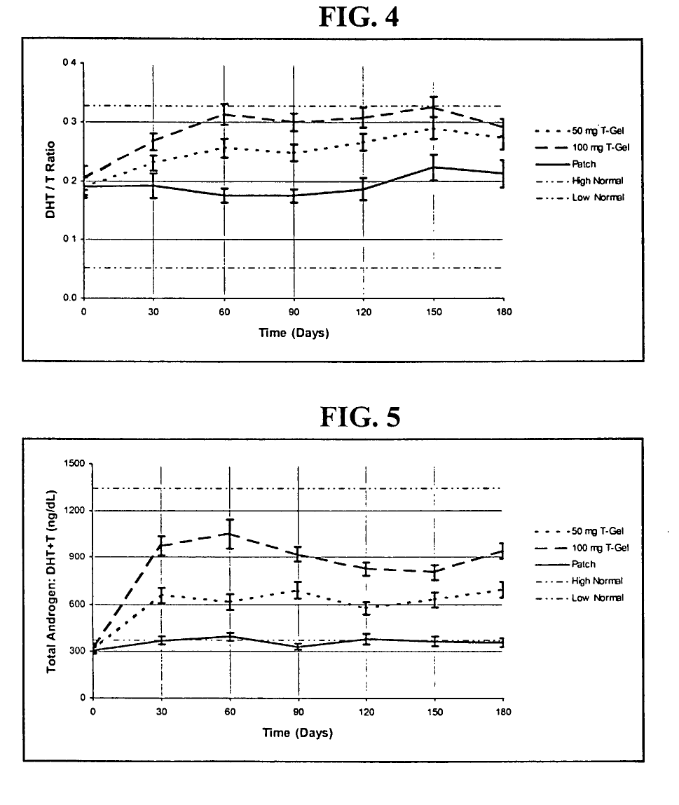

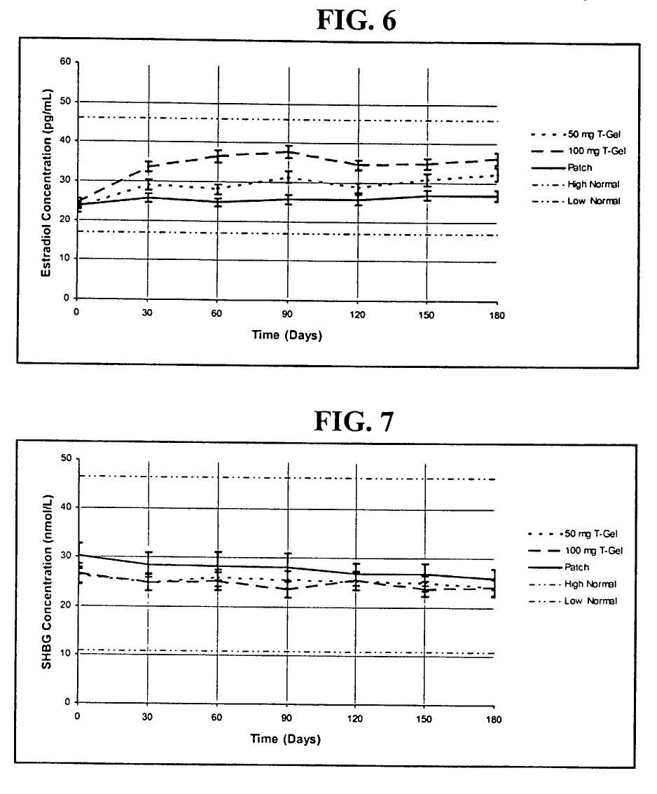

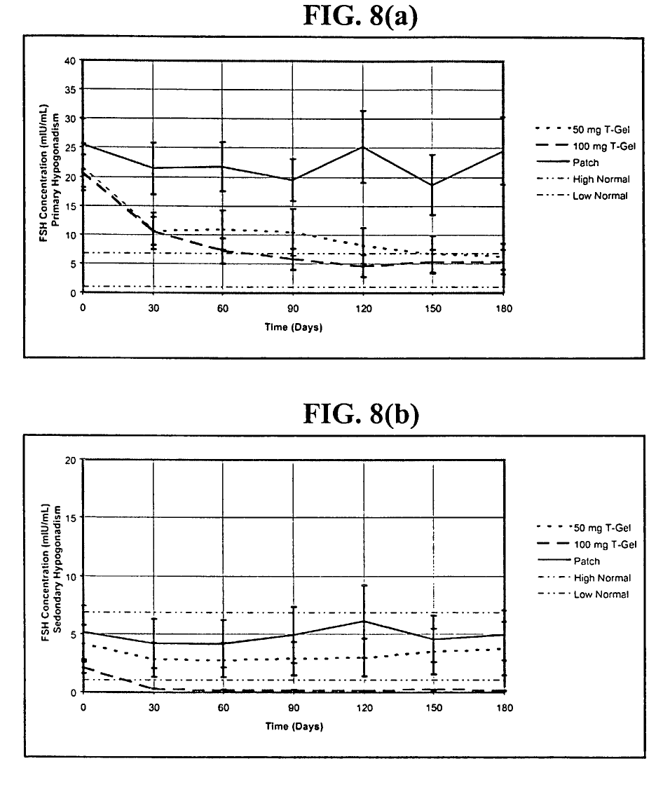

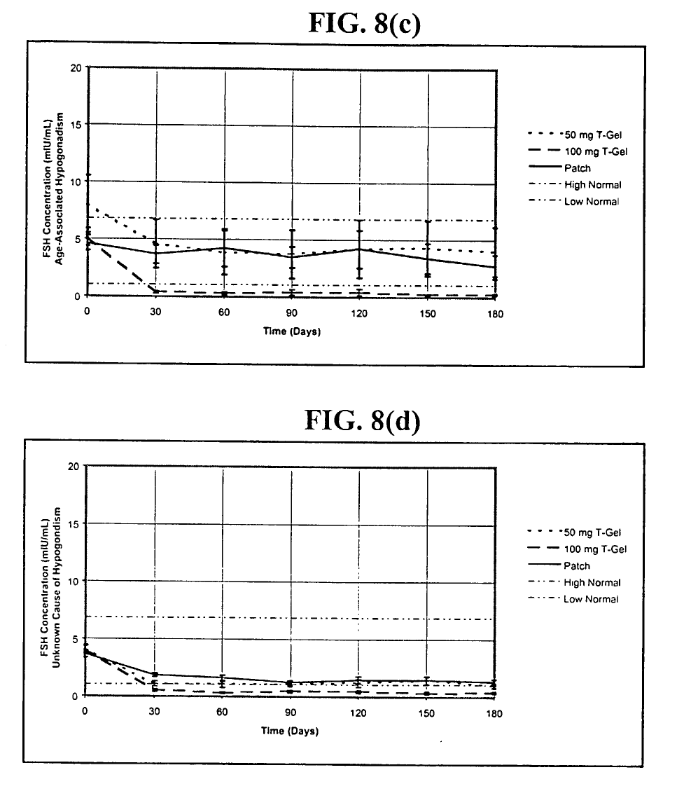

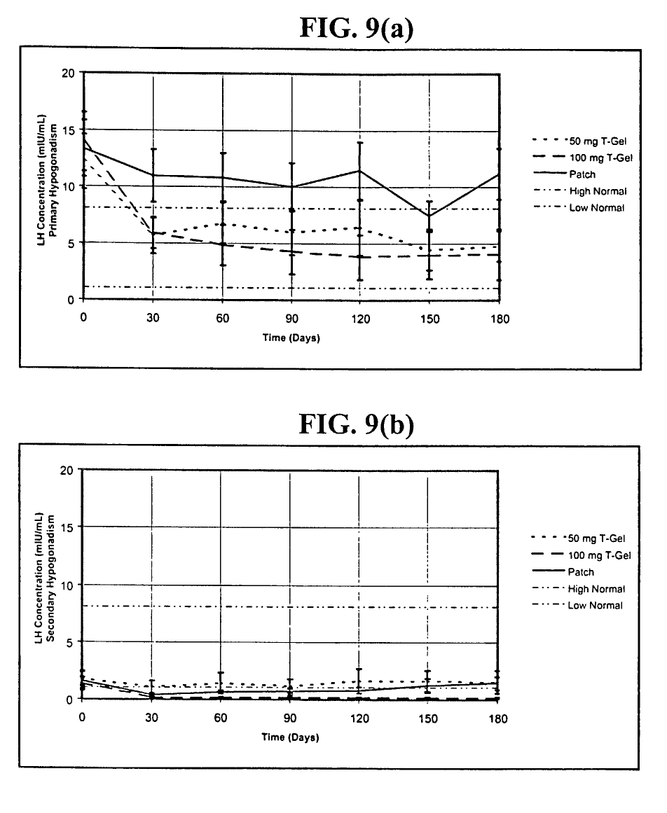

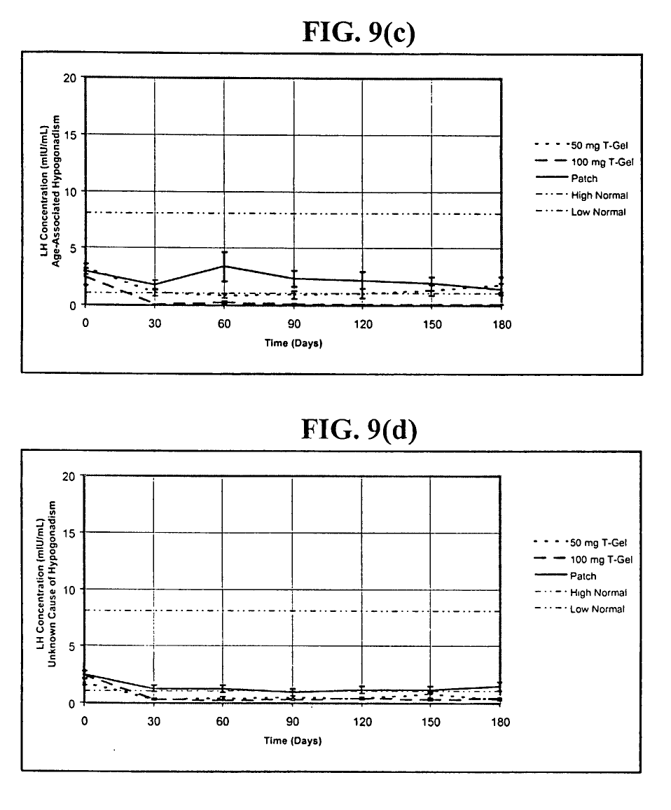

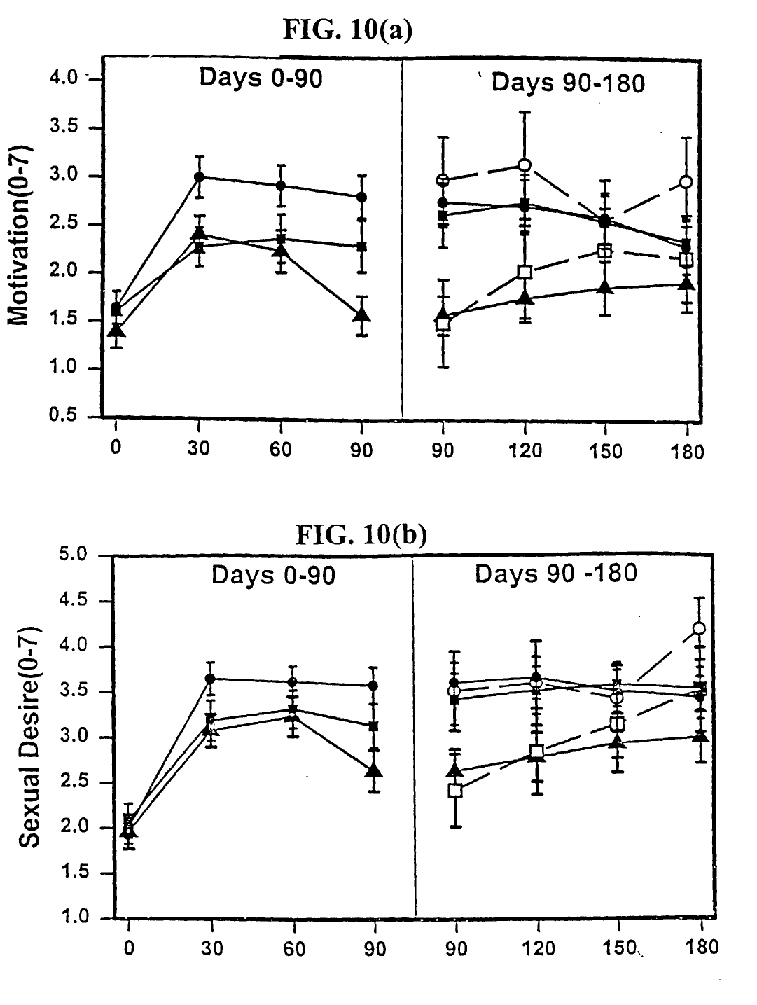

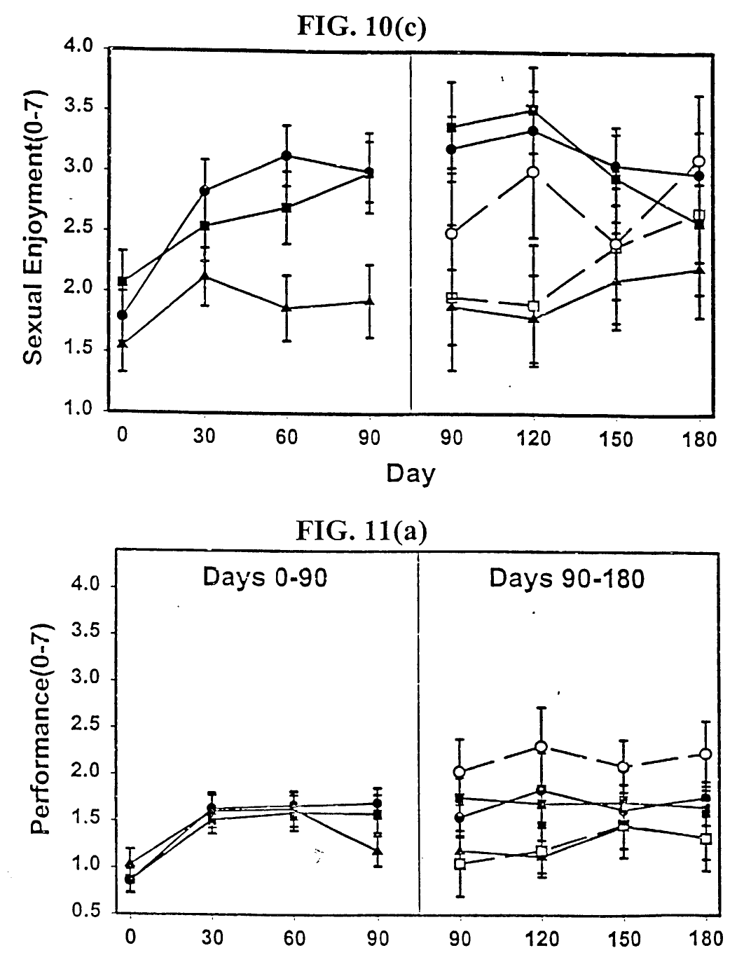

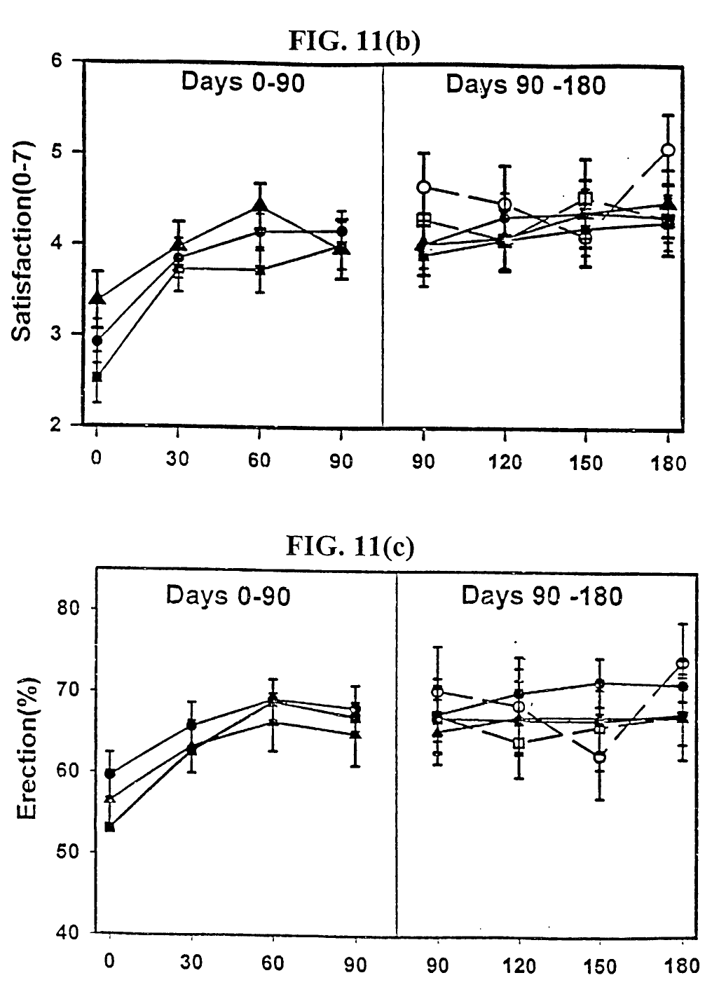

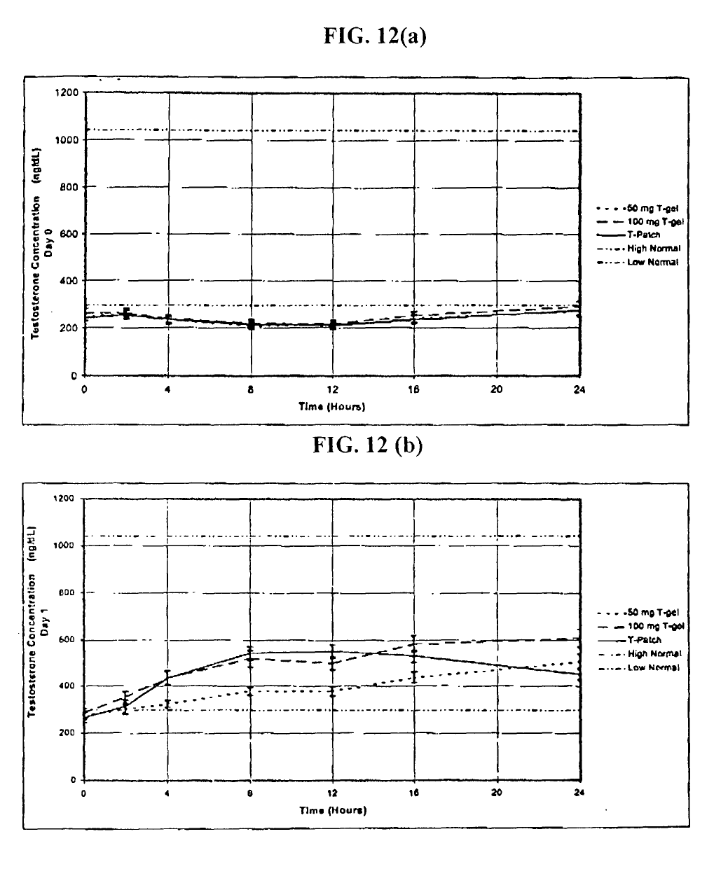

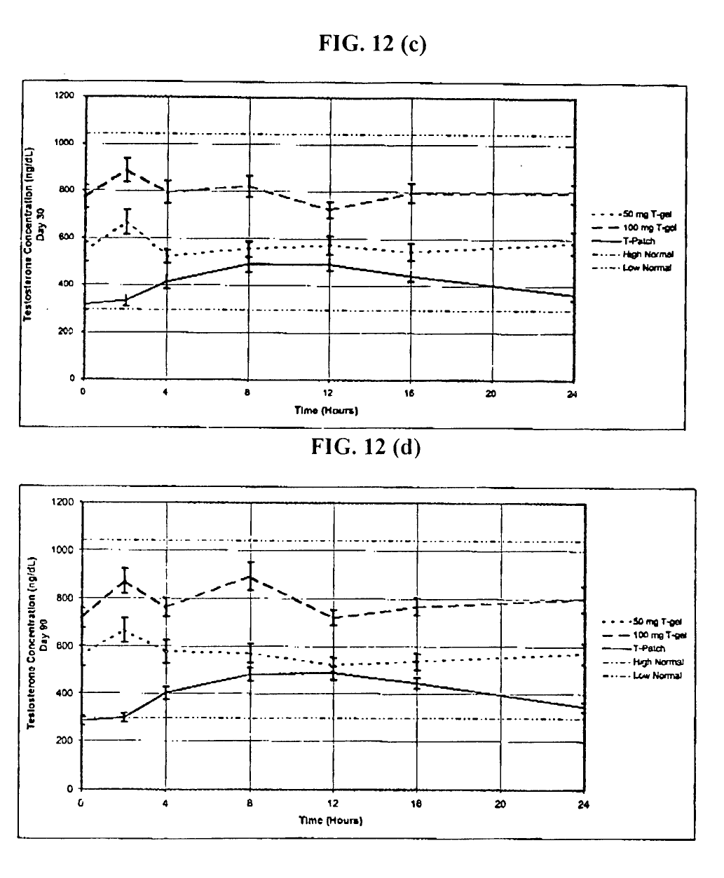

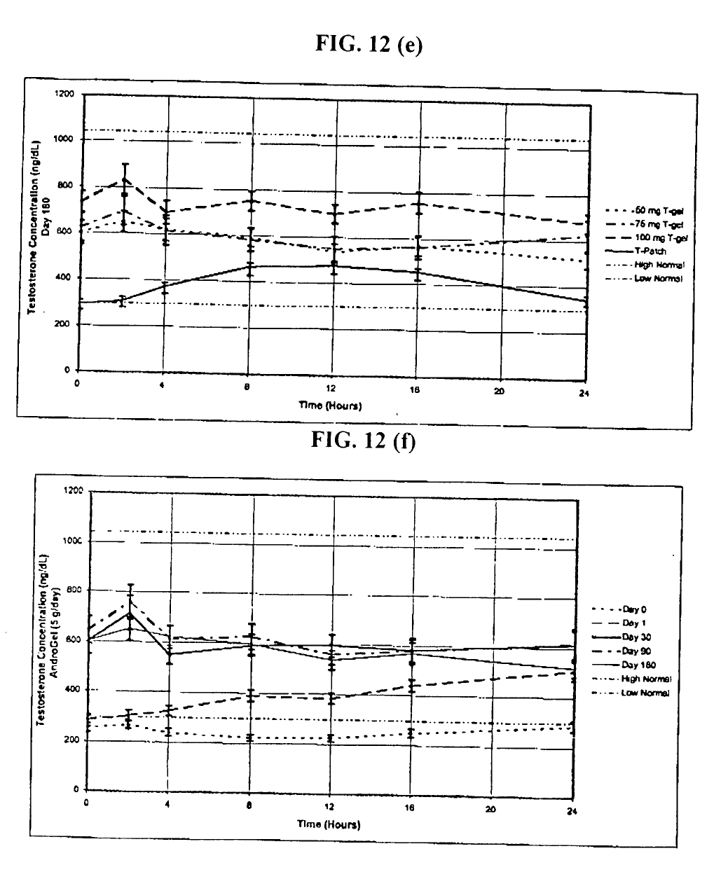

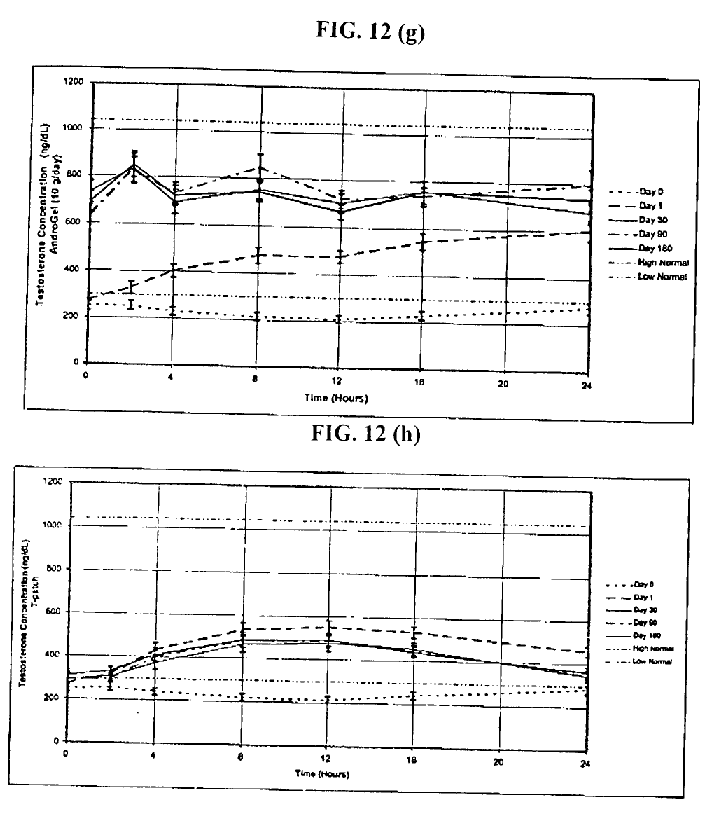

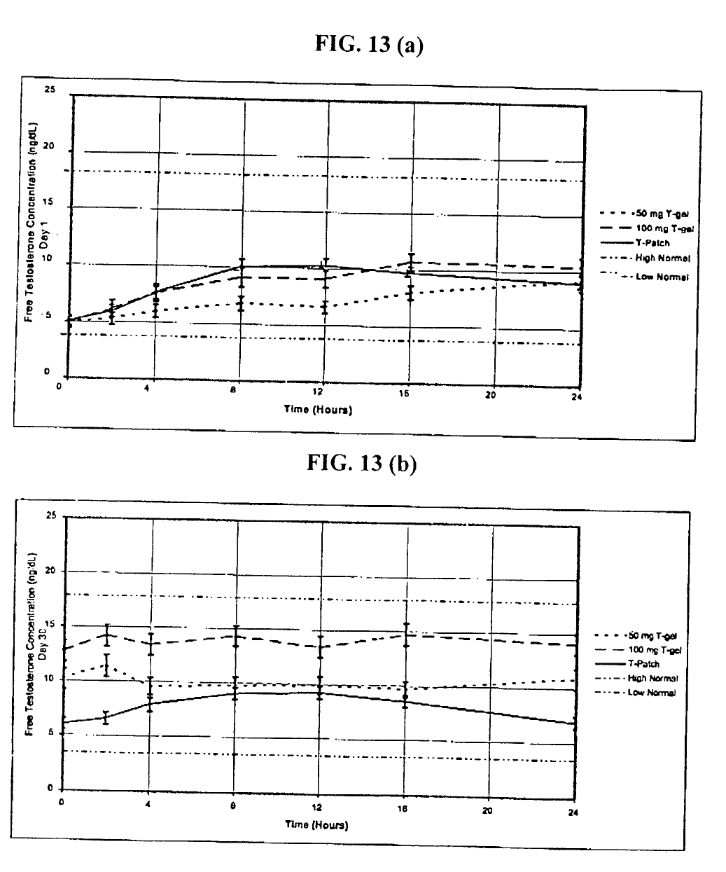

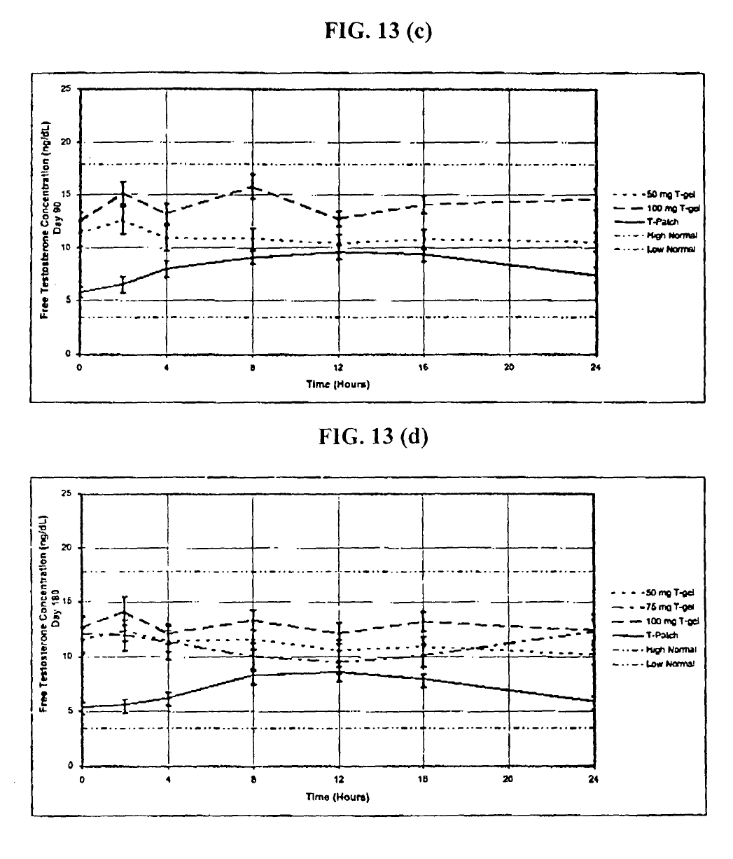

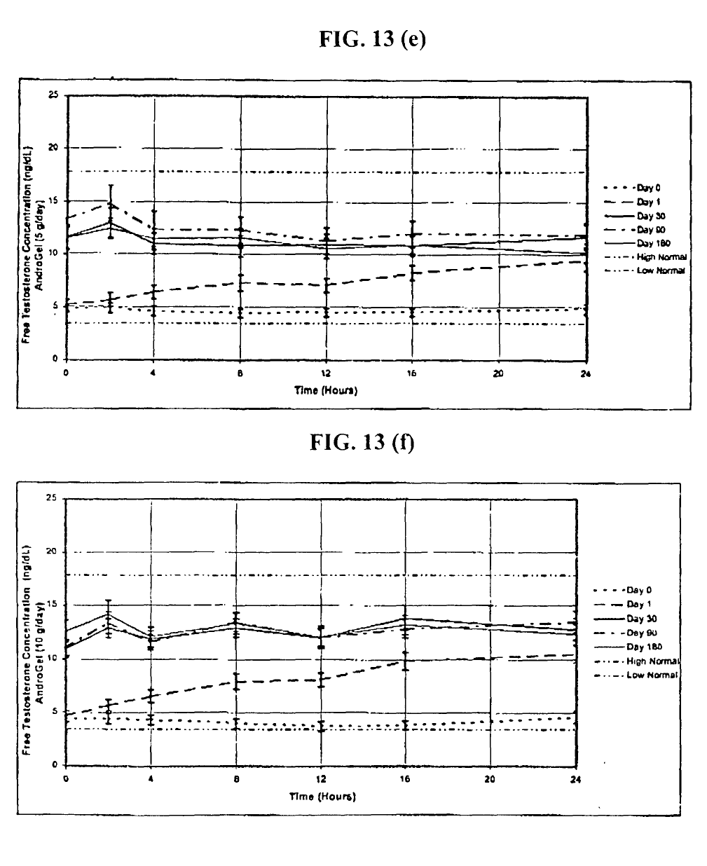

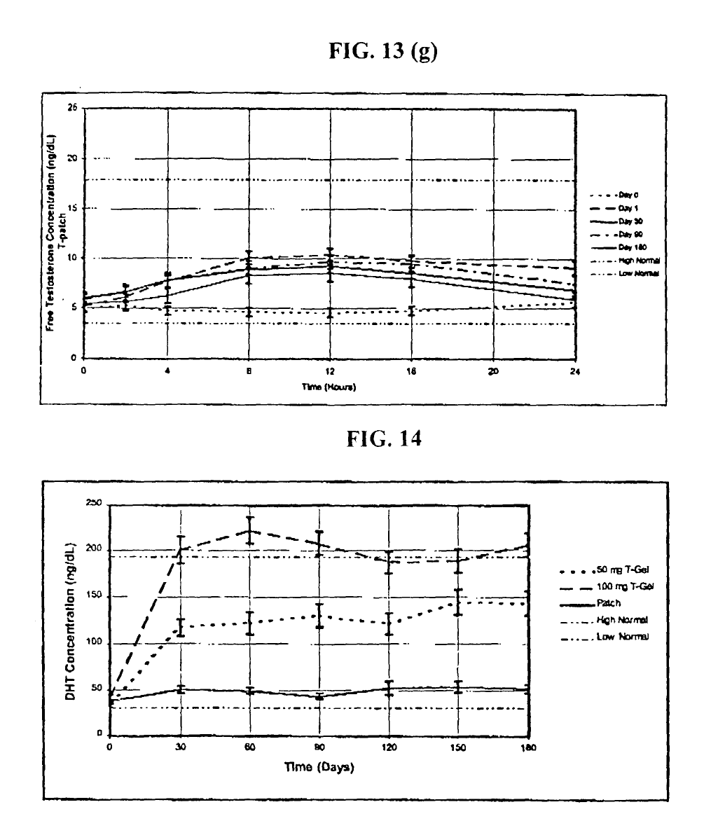

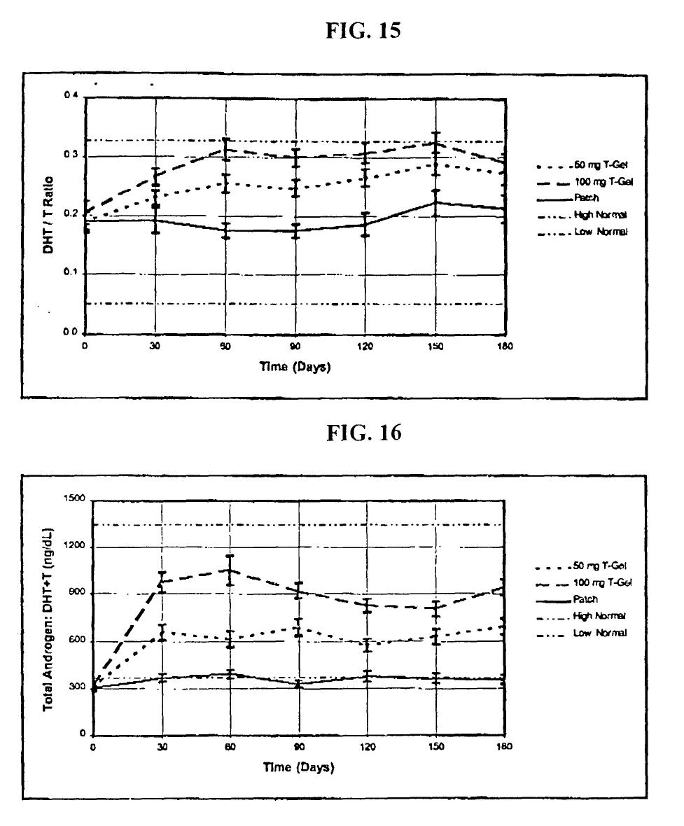

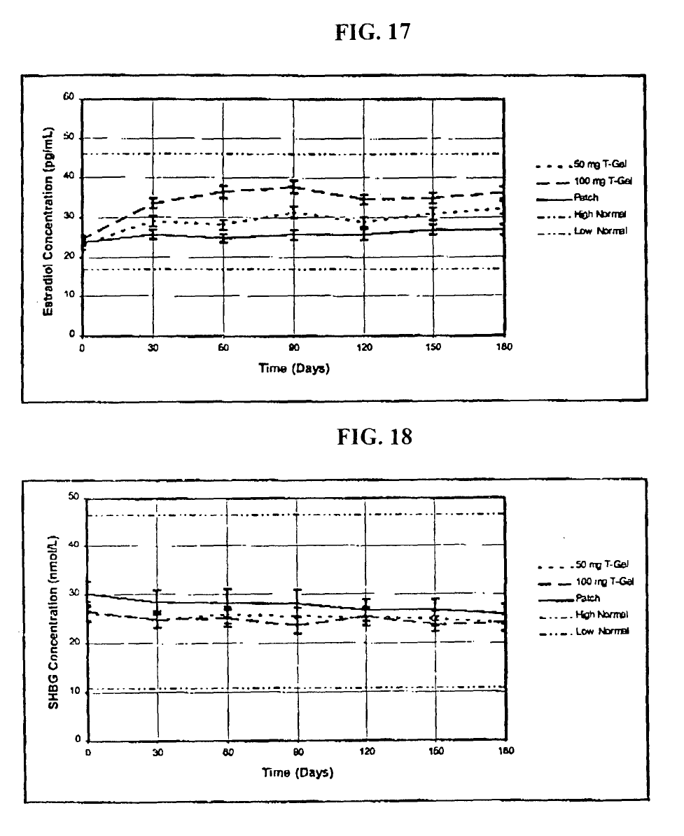

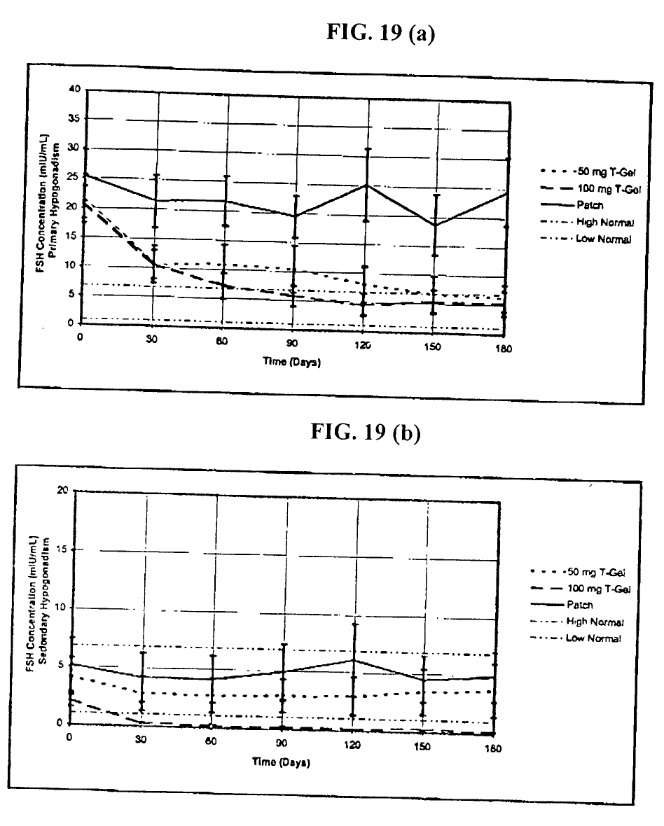

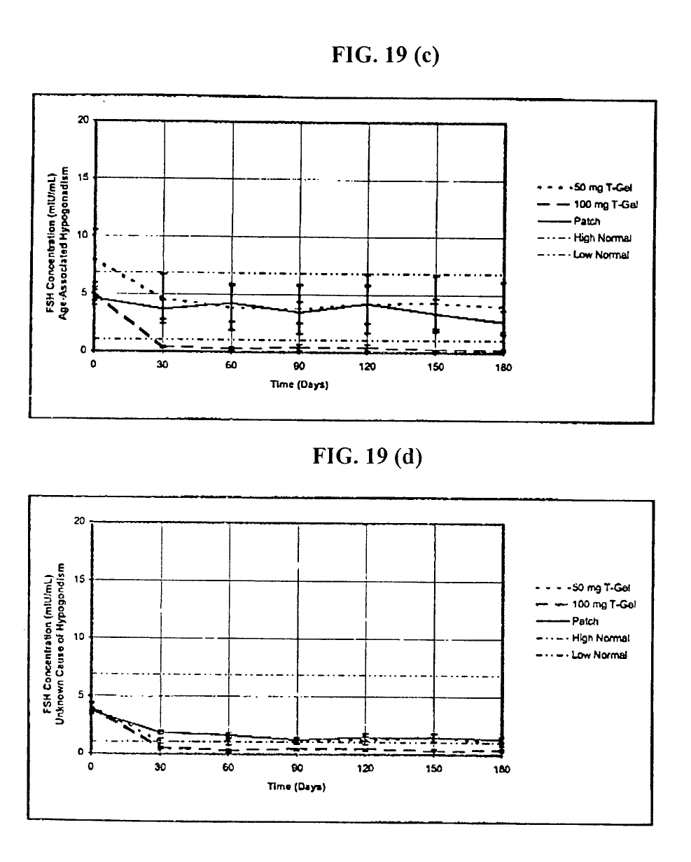

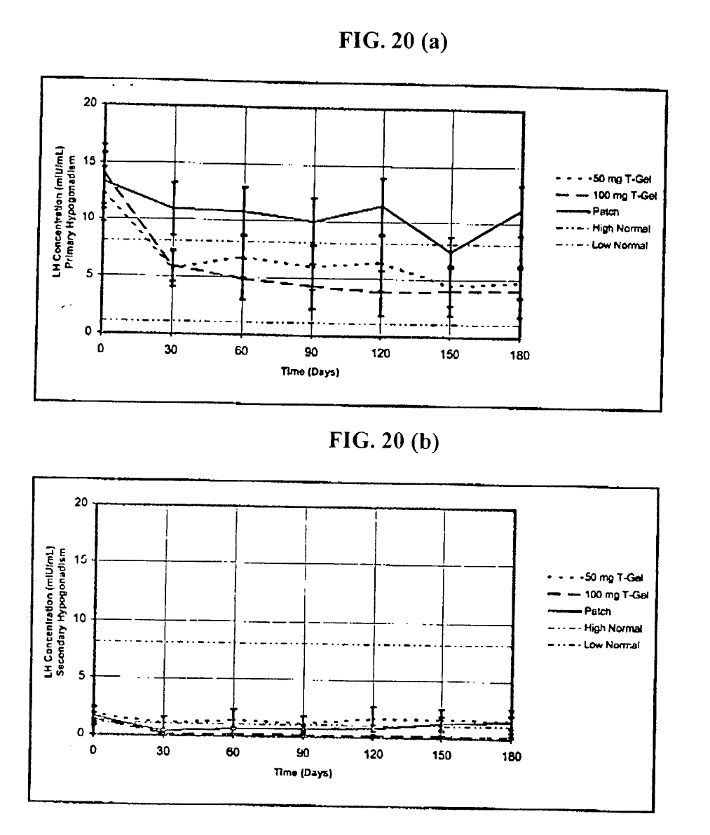

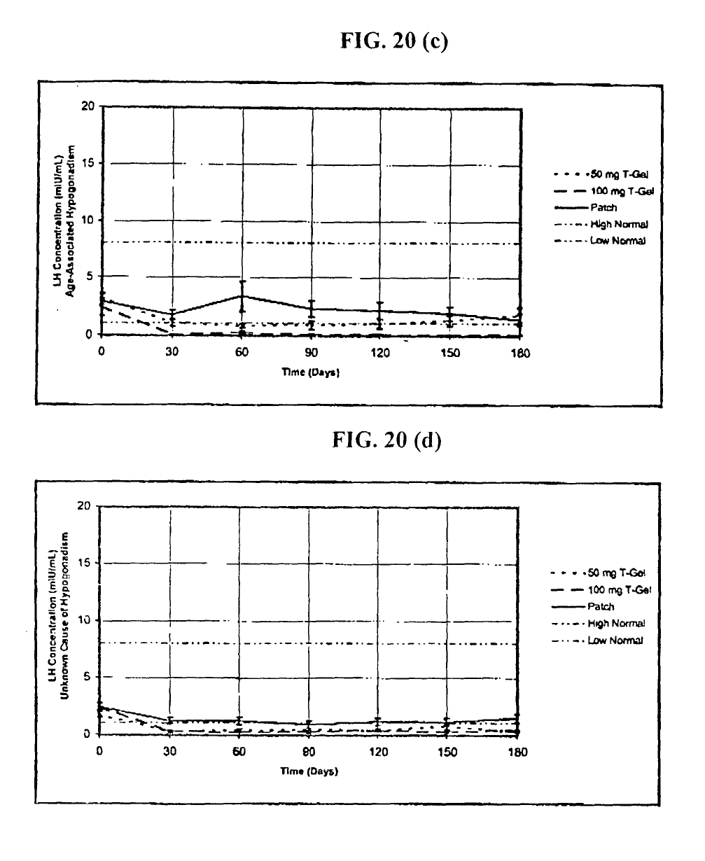

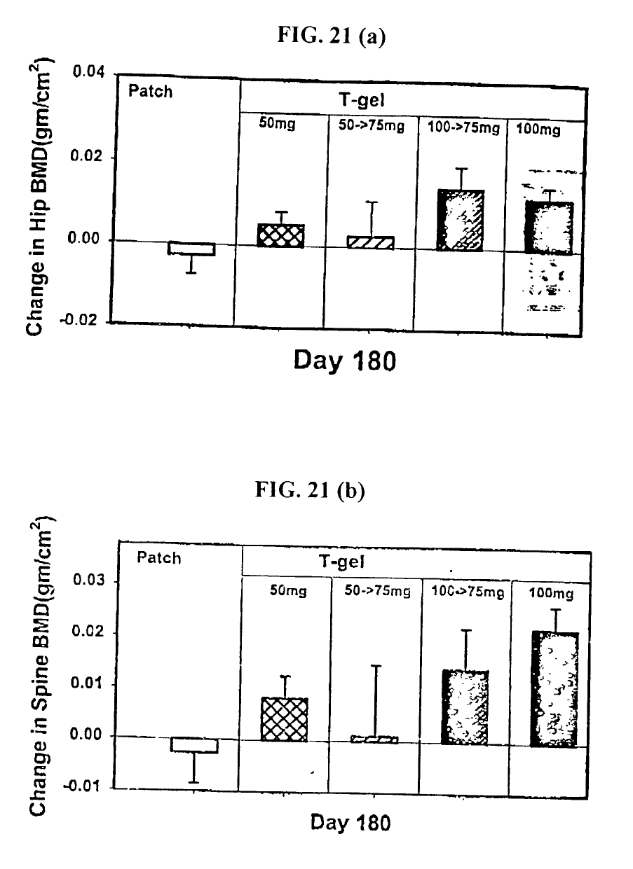

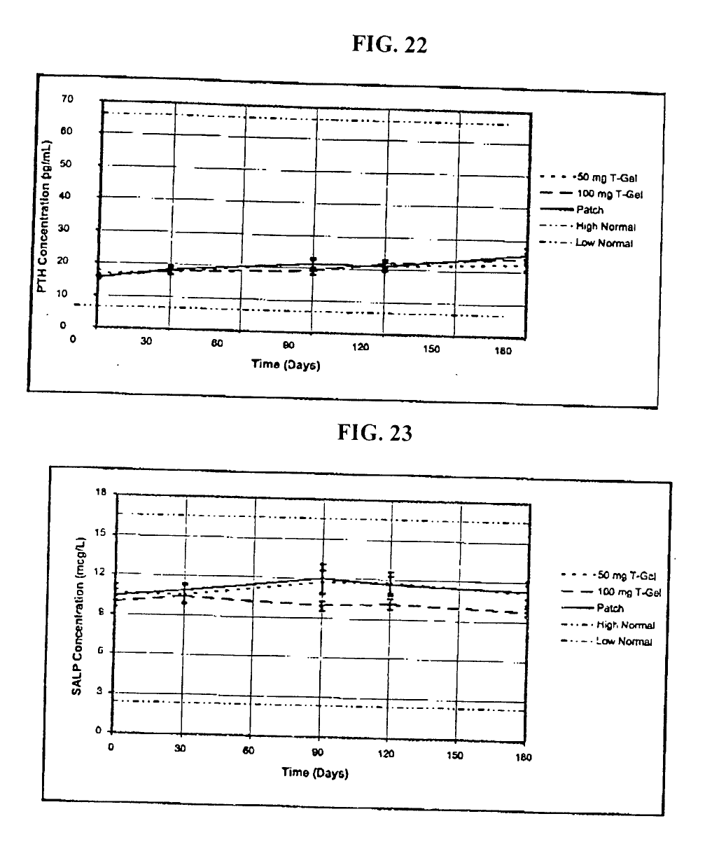

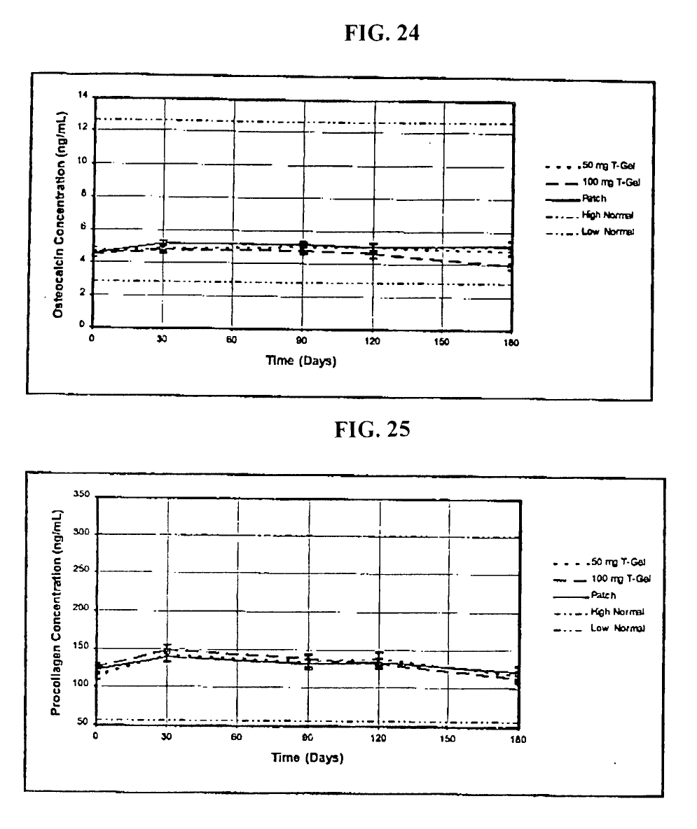

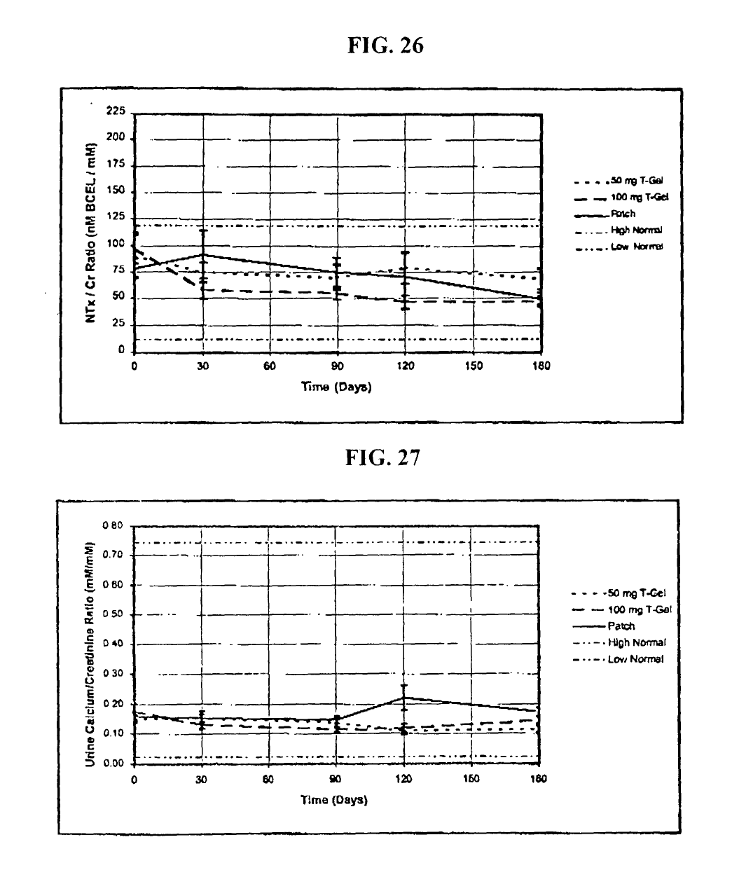

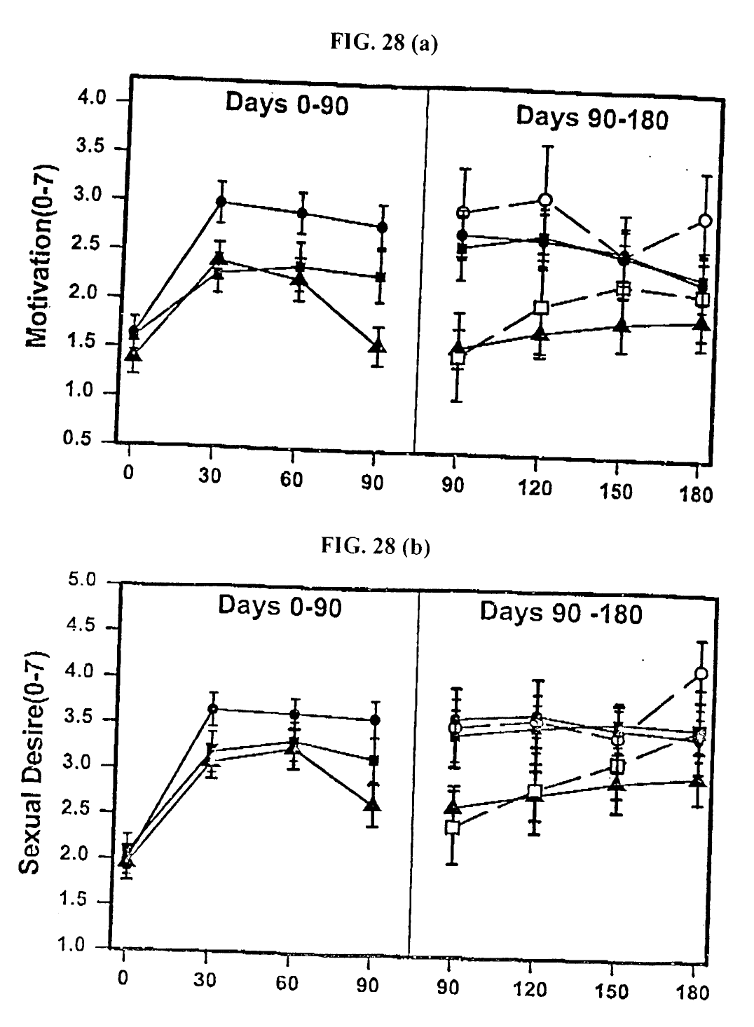

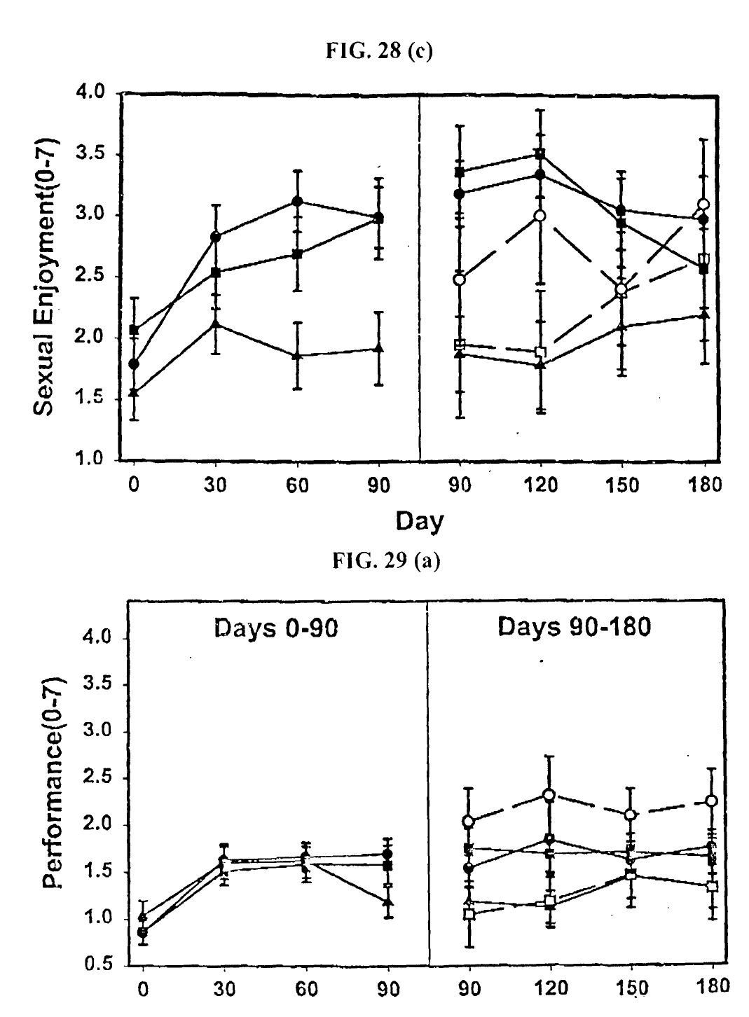

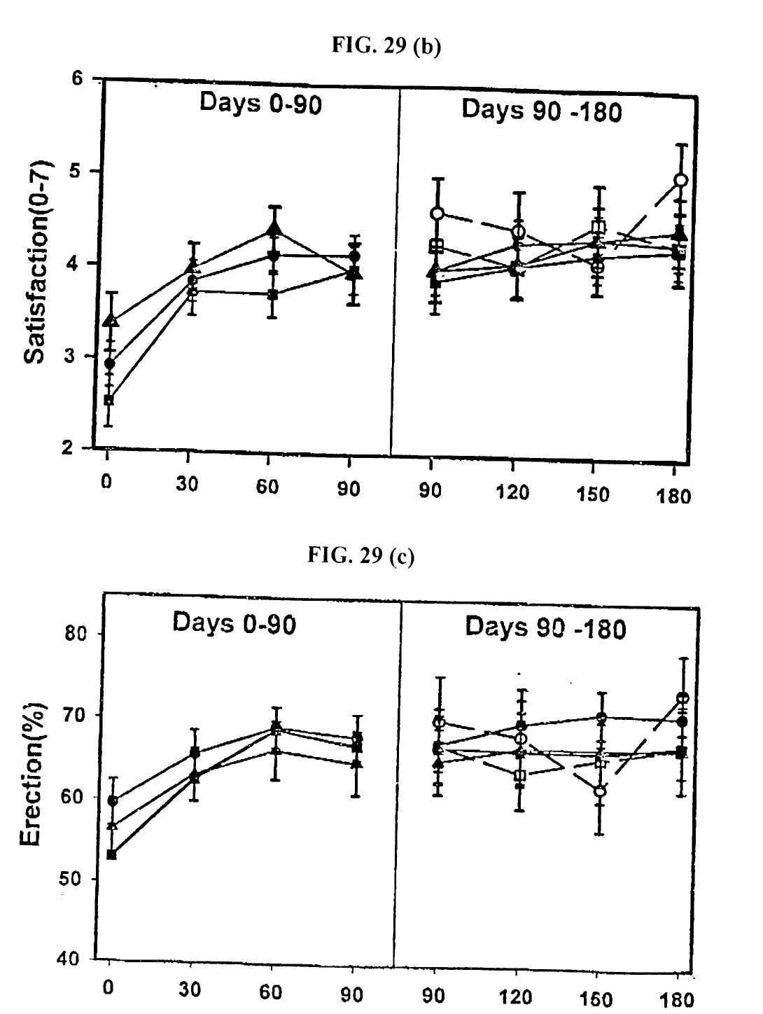

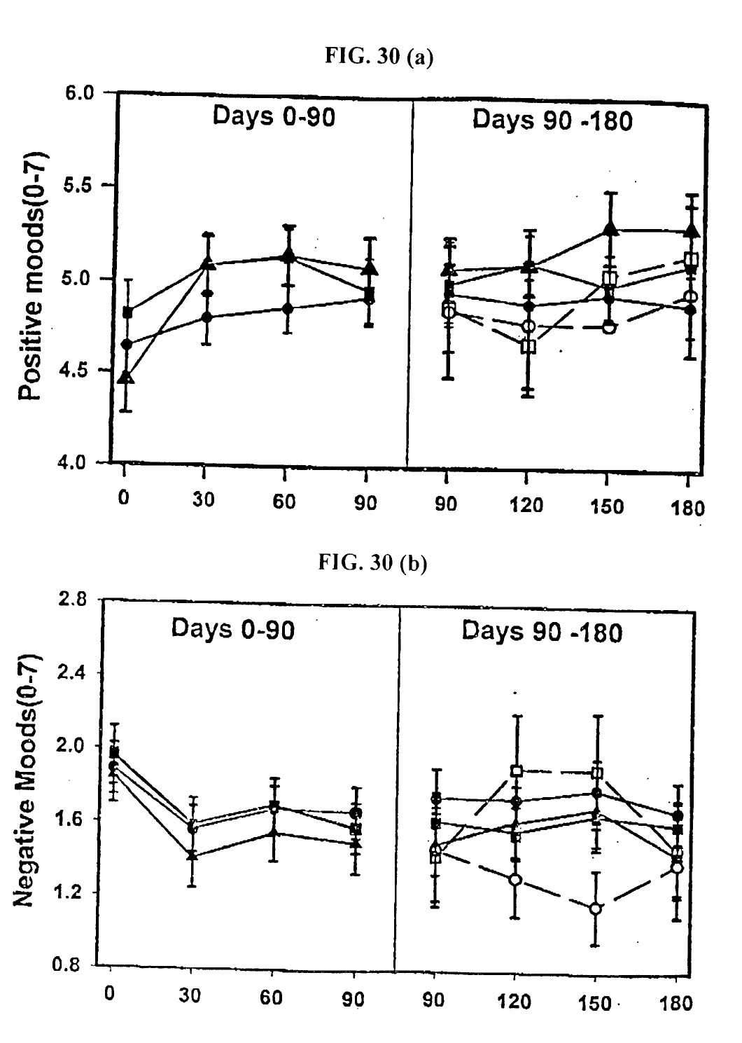

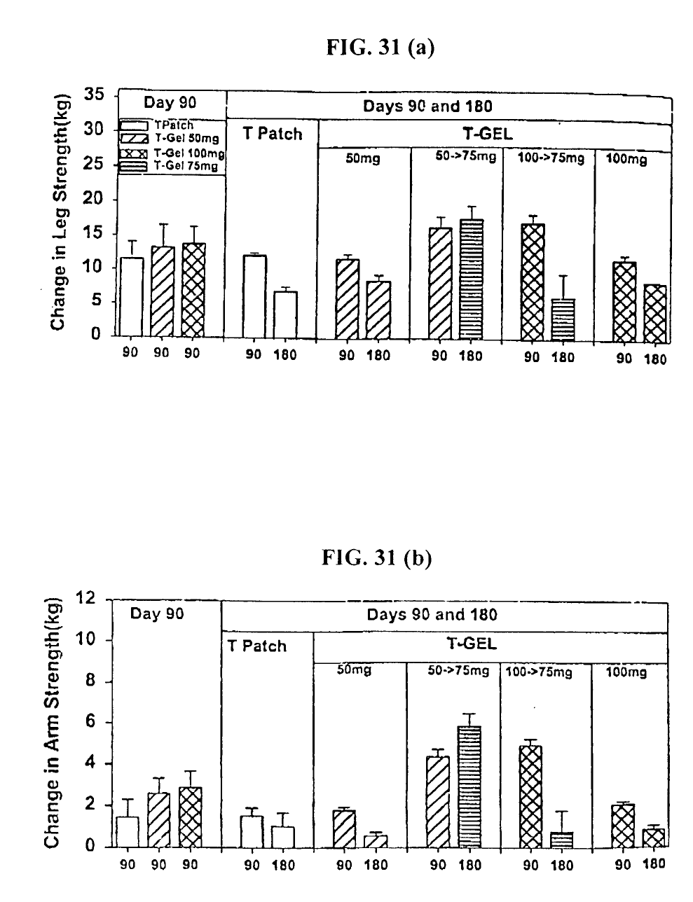

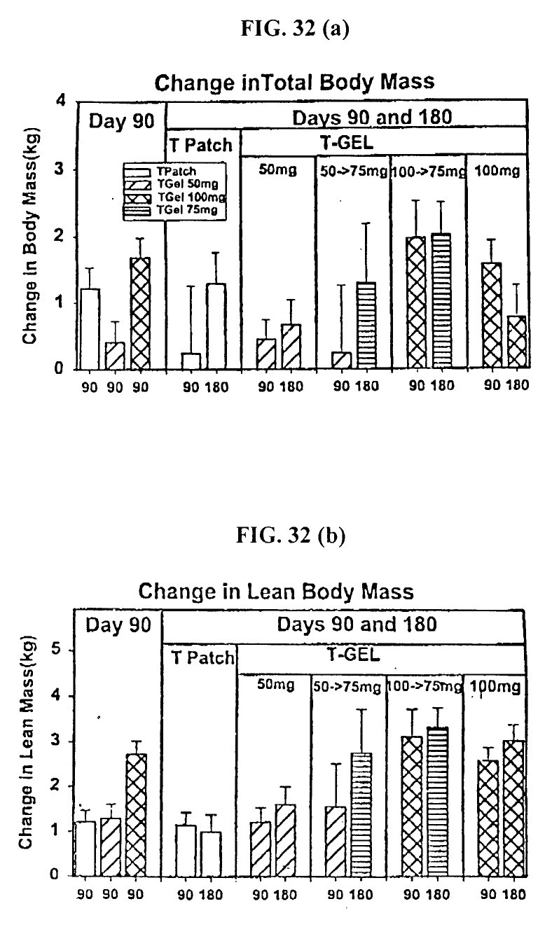

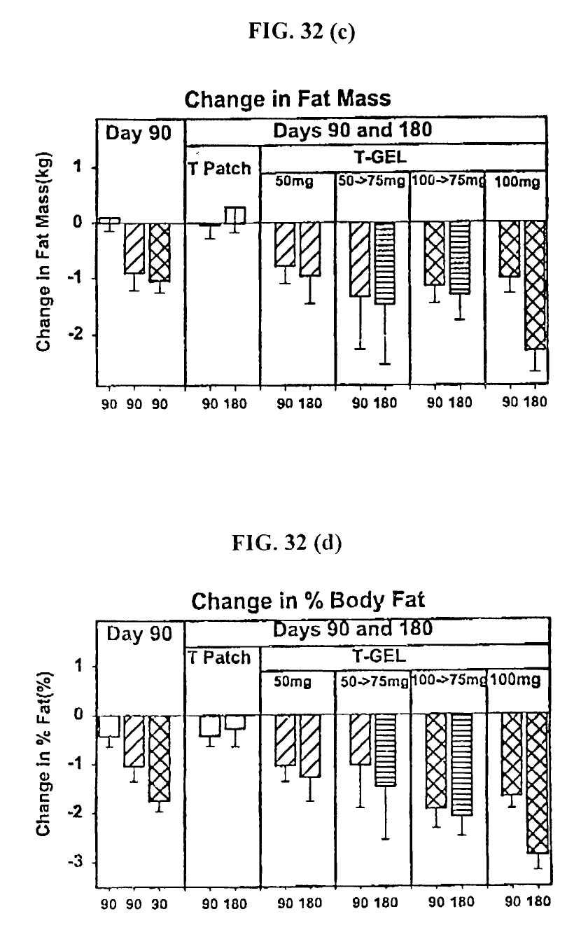

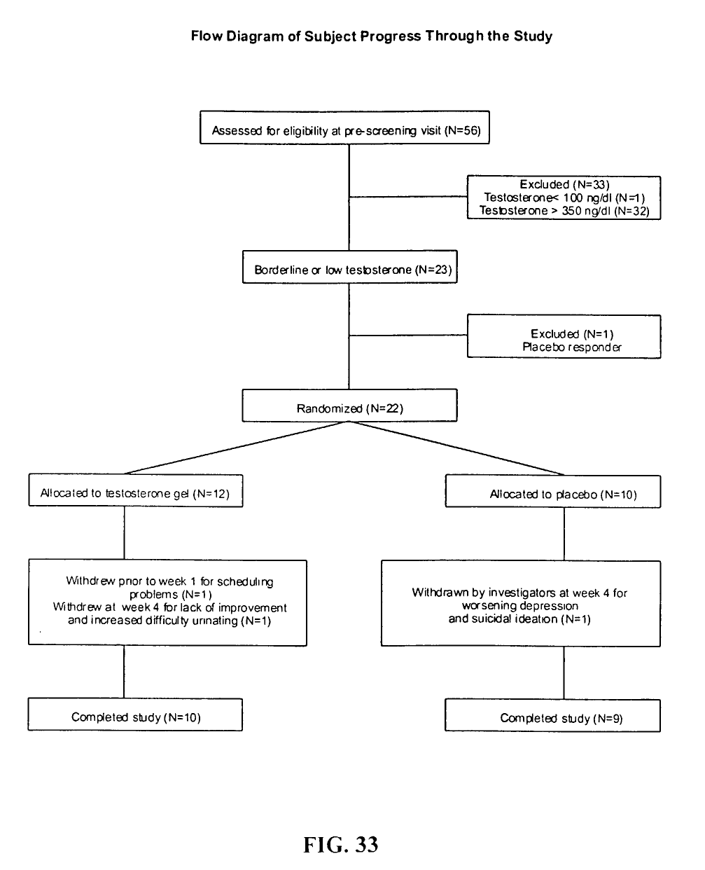

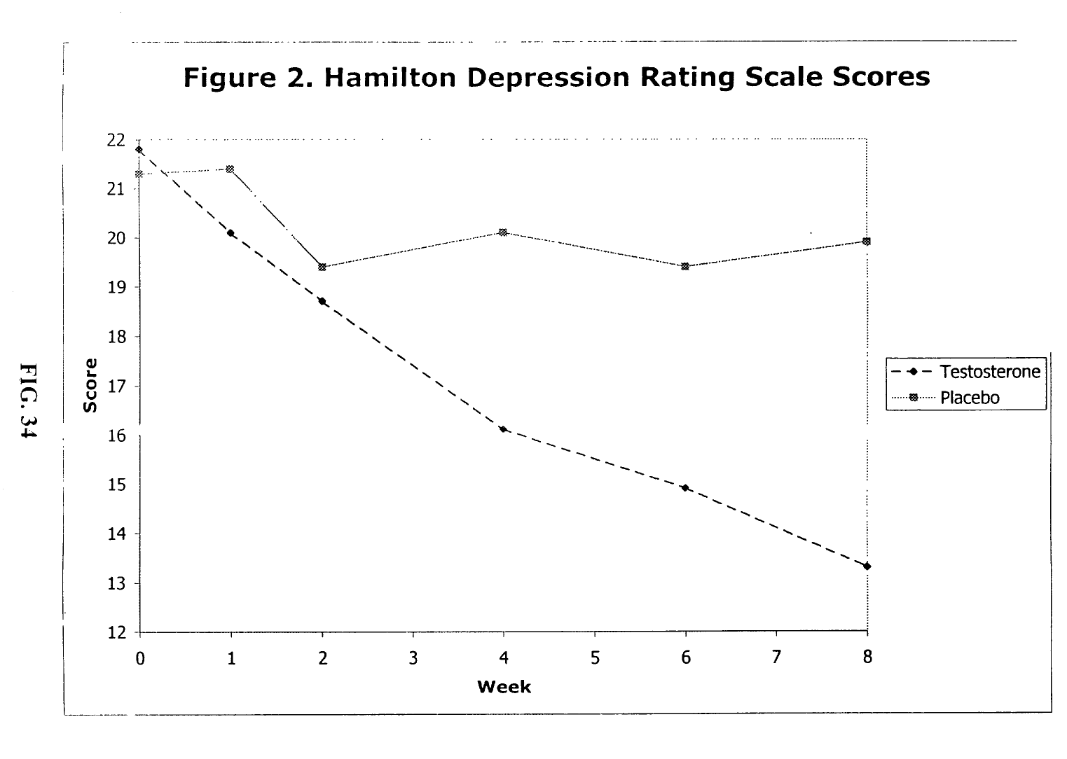

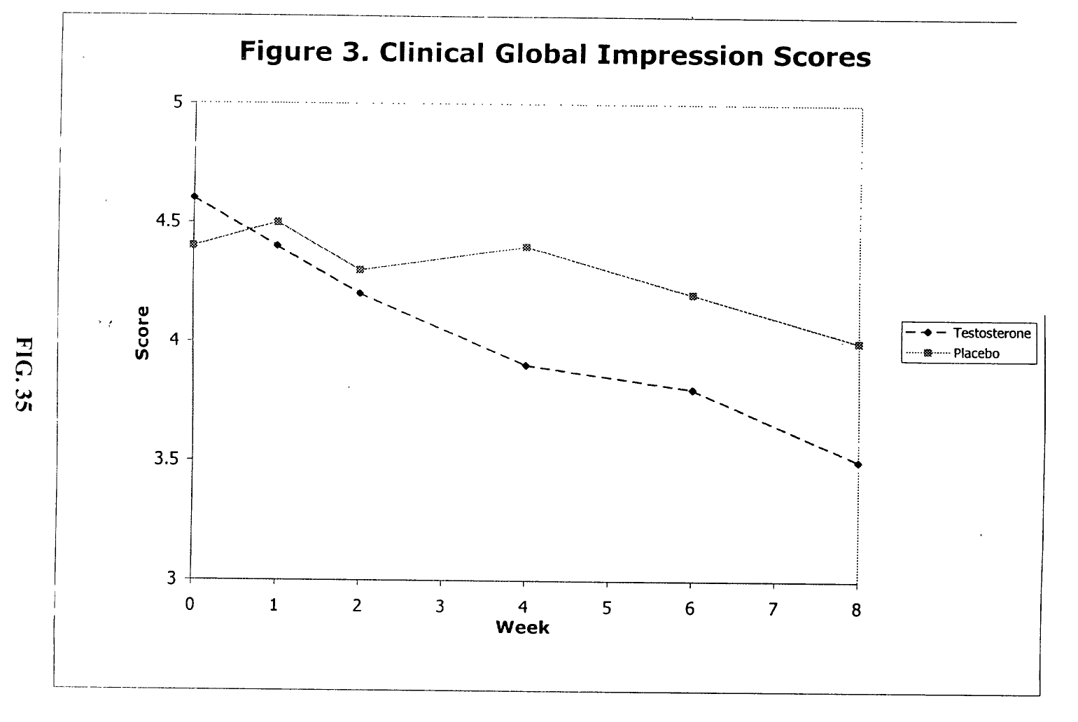

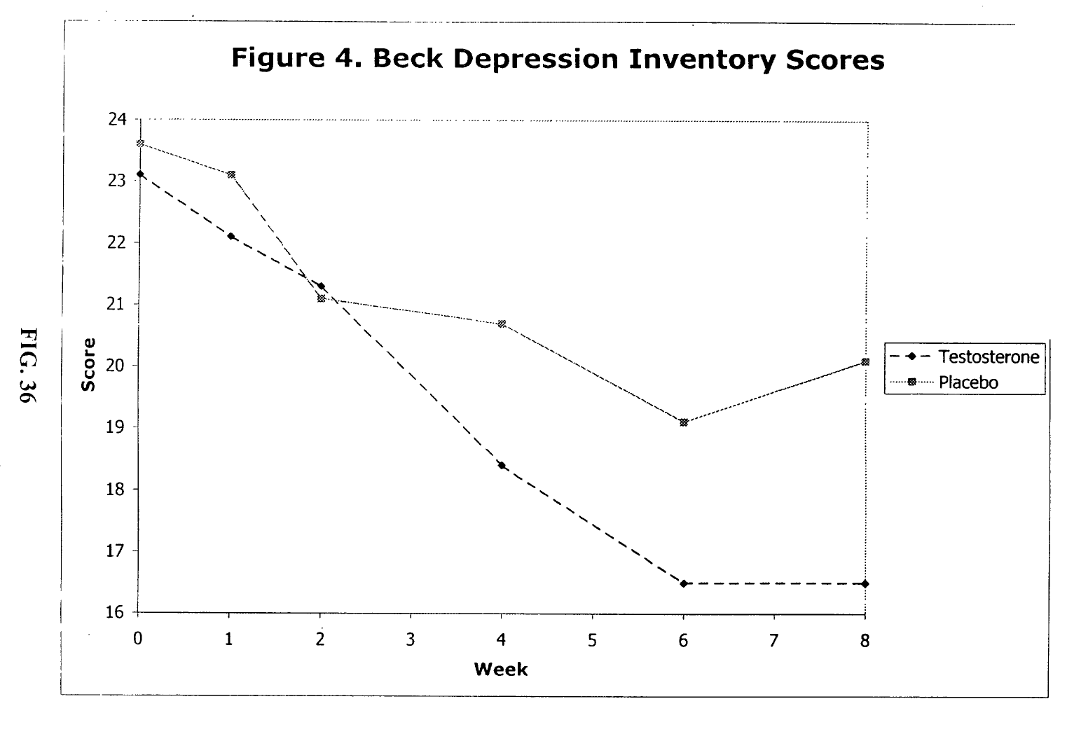

As shown in Tables 28(a) and (b) and FIG. 12(a), at baseline, the average serum testosterone concentrations over 24 hours (C_{avg}) were similar in the groups and below the adult normal range. Moreover the variations of the serum concentration (based on maximum and minimum concentrations during the 24-hour period, Cₘₐₓ and Cₘᵢₙ, respectively) during the day were also similar in the three groups. FIG. 12(a) shows that the mean testosterone levels had a the maximum level between 8 to 10 a.m. (i.e., at 0 to 2 hours) and the minimum 8 to 12 hours later, demonstrating a mild diurnal variation of serum testosterone. About one-third of the patients in each group had C_{avg} within the lower normal adult male range on day 0 (24/73 for the 5.0 g/day AndroGel® group, 26/78 for the 10.0 g/day AndroGel® group, and 25/76 for testosterone patch group). All except three of die subjects met the enrollment criterion of serum testosterone less than 300 ng/dL (10.4 nmol/L) on admission.

TABLE 28(a)
Baseline Phamacokinetic Parameters
by Initial Treatment Group (Mean ± SD)

| | 5.0 g/day T-Gel | 10.0 g/day T-gel | T-patch |
|---|---|---|---|
| N | 73 | 78 | 76 |
| C_{avg} (ng/dL) | 237 ± 130 | 248 ± 140 | 237 ± 139 |
| Cₘₐₓ (ng/dL) | 328 ± 178 | 333 ± 194 | 314 ± 179 |
| Tₘₐₓ* (hr) | 4.0 (0.0-24.5) | 7.9 (0.0-24.7) | 4.0 (0.0-24.3) |
| Cₘᵢₙ (ng/dL) | 175 ± 104 | 188 ± 112 | 181 ± 112 |
| Tₘᵢₙ* (hr) | 8.01 (0.0-24.1) | 8.0 (0.0-24.0) | 8.0 (0.0-23.9) |
| Fluc Index (ratio) | 0.627 ± 0.479 | 0.556 ± 0.384 | 0.576 ± 0.341 |

*Median (Range*)

TABLE 28(b)
Baseline Testosterone Pharmacokinetic Parameters
by Final Treatment Group (Mean ± SD)

| | Doses Received During Initial => Extended Treatment Phases | | | | |
|---|---|---|---|---|---|
| | 5.0 g/day | 5.0 => 7.5 g/day | 10.0 => 7.5 g/day | 10.0 g/day | |
| | T-gel | T-gel | T-gel | T-gel | T-patch |
| N | 53 | 20 | 20 | 58 | 76 |
| C_{avg} (ng/dL) | 247 ± 137 | 212 ± 109 | 282 ± 157 | 236 ± 133 | 237 ± 140 |
| Cₘₐₓ (ng/dL) | 333 ± 180 | 313 ± 174 | 408 ± 241 | 307 ± 170 | 314 ± 179 |
| Tₘₐₓ* (hr) | 4.0 (0.0-24.5) | 4.0 (0.0-24.0) | 19.7 (0.0-24.3) | 4.0 (0.0-24.7) | 4.0 (0.0-24.3) |
| Cₘᵢₙ (ng/dL) | 185 ± 111 | 150 ± 80 | 206 ± 130 | 182 ± 106 | 181 ± 112 |
| Tₘᵢₙ* (hr) | 8.0 (0.0-24.1) | 11.9 (0.0-24.0) | 8.0 (0.0-23.3) | 8.0 (0.0-24.0) | 8.0 (0.0-23.9) |
| Fluc Index (ratio) | 0.600 ± 0.471 | 0.699 ± 0.503 | 0.678 ± 0.580 | 0.514 ± 0.284 | 0.576 ± 0.341 |

*Median (range)

Day 1

FIG. 12(b) and Tables 28(c)-(d) show the pharmacokinetic profile for all three initial treatment groups after the first application of transdermal testosterone. In general, treatment with AndroGel® and the testosterone patch produced increases in testosterone concentrations sufficiently large to bring the patients into the normal range in just a few hours. However, even on day 1, the pharmacokinetic profiles were markedly different in the AndroGel® and patch groups. Serum testosterone rose most rapidly in the testosterone patch group reaching a maximum concentration (Cₘₐₓ) at about 12 hours (Tₘₐₓ). In contrast, serum testosterone rose steadily to the normal range after AndroGel® application with Cₘₐₓ levels achieved by 22 and 16 hours in the 5.0 g/day AndroGel® group and the 10.0 g/day AndroGel® group, respectively.

TABLE 28(c)
Testosterone Pharmacokinetic Parameters on Day 1
by Initial Treatment Group (Mean ± SD)

| | 5.0 g/day T-Gel | 10.0 g/day T-gel | T-patch |
|---|---|---|---|
| N | 73 | 76 | 74 |
| C_{avg} (ng/dL) | 398 ± 156 | 514 ± 227 | 482 ± 204 |
| Cₘₐₓ (ng/dL) | 560 ± 269 | 748 ± 349 | 645 ± 280 |
| Tₘₐₓ* (hr) | 22.1 (0.0-25.3) | 16.0 (0.0-24.3) | 11.8 (1.8-24.0) |
| Cₘᵢₙ (ng/dL) | 228 ± 122 | 250 ± 143 | 232 ± 132 |
| Tₘᵢₙ* (hr) | 1.9 (0.0-24.0) | 0.0 (0.0-24.2) | 1.5 (0.0-24.0) |

*Median (Range)

TABLE 28(d)
Testosterone Phamacokinetic Parameters on Day 1
by Final Treatment Group (Mean ± SD)

| | Doses Received During Initial => Extended Treatment Phases | | | | |
|---|---|---|---|---|---|
| | 5.0 g/day | 5.0 => 7.5 g/day | 10.0 => 7.5 g/day | 10.0 g/day | |
| | T-gel | T-gel | T-gel | T-gel | T-patch |
| N | 53 | 20 | 19 | 57 | 74 |
| C_{avg} (ng/dL) | 411 ± 160 | 363 ± 143 | 554 ± 243 | 500 ± 223 | 482 ± 204 |
| Cₘₐₓ (ng/dL) | 573 ± 285 | 525 ± 223 | 819 ± 359 | 724 ± 346 | 645 ± 280 |
| Tₘₐₓ* (hr) | 22.1 (0.0-25.3) | 19.5 (1.8-24.3) | 15.7 (3.9-24.0) | 23.0 (0.0-24.3) | 11.8 (1.8-24.0) |
| Cₘᵢₙ (ng/dL) | 237 ± 125 | 204 ± 112 | 265 ± 154 | 245 ± 140 | 232 ± 132 |
| Tₘᵢₙ* (hr) | 1.8 (0.0-24.0) | 3.5 (0.0-24.0) | 1.9 (0.0-24.2) | 0.0 (0.0-23.8) | 1.5 (0.0-24.0) |
| Fluc Index (ratio) | 0.600 ± 0.471 | 0.699 ± 0.503 | 0.678 ± 0.580 | 0.514 ± 0.284 | 0.576 ± 0.341 |

*Median (range)

Days 30, 90, and 180

FIG. Nos. 12(c) and 12(d) show the unique 24-hour pharmacokinetic profile of AndroGel®-treated patients on days 30 and 90. In the AndroGel® groups, serum testosterone levels showed small and variable increases shortly after dosing. The levels then returned to a relatively constant level. In contrast, in the testosterone patch group, patients exhibited a rise over the first 8 to 12 hours, a plateau for another 8 hours, and then a decline to the baseline of the prior day. Further, after gel application on both days 30 and 90, the C_{avg} in the 10.0 g/day AndroGel® group was 1.4 fold higher than in the 5.0 g/day AndroGel® group and 1.9 fold higher than the testosterone patch group. The testosterone patch group also had a Cₘᵢₙ substantially below the lower limit of the normal range. On day 30, the accumulation ratio was 0.94 for testosterone patch group, showing no accumulation. The accumulation ratios at 1.54 and 1.9 were significantly higher in the 5.0 g/day AndroGel® group and 10.0 g/day AndroGel® group, respectively. The differences in accumulation ratio among the groups persisted on day 90. This data indicates that the AndroGel® preparations had a longer effective half-life than testosterone patch.

FIG. 12(e) shows the 24-hour pharmacokinetic profile for the treatment groups on day 180. In general, as Table 28(e) shows, the serum testosterone concentrations achieved and the pharmacokinetic parameters were similar to those on days 30 and 90 in those patients who continued on their initial randomized treatment groups. Table 28(f) shows that the patients titrated to the 7.5 g/day AndroGel® group were not homogeneous. The patients that were previously in the 10.0 g/day group tended to have higher serum testosterone levels than those previously receiving 5.0 g/day. On day 180, the C_{avg} in the patients in the 10.0 g/day group who converted to 7.5 g/day on day 90 was 744 ng/dL, which was 1.7 fold higher than the. C_{avg} of 450 ng/dL in the patients titrated to 7.5 g/day from 5.0 g/day. Despite adjusting the dose up by 2.5 g/day in the 5.0 to 7.5 g/day group, the C_{avg} remained lower than those remaining in the 5.0 g/day group. In the 10.0 to 7.5 g/day group, the C_{avg} became similar to those achieved by patients remaining in the 10.0 g/day group without dose titration. These results suggest that many of the under-responders may actually be poorly compliant patients. For example, if a patient does not apply AndroGel® properly (e.g., preferentially from the placebo container or shortly before bathing), then increasing the dose will not provide any added benefit.

FIG. Nos. 12(f)-(h) compare the pharmacokinetic profiles for the 5.0 g/day AndroGel® group, the 10.0 AndroGel® g/day group, and the testosterone patch group at days 0, 1, 30, 90, and 180, respectively. In general, the mean serum testosterone levels in the testosterone patch group remained at the lower limit of the normal range throughout the treatment period. In contrast, the mean serum testosterone levels remained at about 490-570 ng/dL for the 5.0 g/day AndroGel® group and about 630-860 ng/dL AndroGel® for the 10.0 g/day group.

TABLE 28(e)
Testosterone Phamacokinetic Parameters on Day 1
by Initial Treatment Group (Mean ± SD)

| | 5.0 g/day T-Gel | 10.0 g/day T-gel | T-patch |
|---|---|---|---|
| Day 30 | N = 66 | N = 74 | N = 70 |
| C_{avg} (ng/dL) | 566 ± 262 | 792 ± 294 | 419 ± 163 |
| Cₘₐₓ (ng/dL) | 876 ± 466 | 1200 ± 482 | 576 ± 223 |
| Tₘₐₓ *(hr) | 7.9 (0.0-24.0) | 7.8 (0.0-24.3) | 11.3 (0.0-24.0) |
| Cₘᵢₙ (ng/dL) | 361 ± 149 | 505 ± 233 | 235 ± 122 |
| Tₘᵢₙ *(hr) | 8.0 (0.0-24.1) | 8.0 (0.0-25.8) | 2.0 (0.0-24.2) |
| Fluc Index | 0.857 ± 0.331 | 0.895 ± 0.434 | 0.823 ± 0.289 |
| (ratio) | | | |
| Accum Ratio | 1.529 ± 0.726 | 1.911 ± 1.588 | 0.937 ± 0.354 |
| (ratio) | | | |
| Day 90 | N = 65 | N = 73 | N = 64 |
| C_{avg} (ng/dL) | 553 ± 247 | 792 ± 276 | 417 ± 157 |
| Cₘₐₓ (ng/dL) | 846 ± 444 | 1204 ± 570 | 597 ± 242 |
| Tₘₐₓ *(hr) | 4.0 (0.0-24.1) | 7.9 (0.0-25.2) | 8.1 (0.0-25.0) |
| Cₘᵢₙ (ng/dL) | 354 ± 147 | 501 ± 193 | 213 ± 105 |
| Tₘᵢₙ *(hr) | 4.0 (0.0-25.3) | 8.0 (0.0-24.8) | 2.0 (0.0-24.0) |
| Fluc Index | 0.851 ± 0.402 | 0.859 ± 0.399 | 0.937 ± 0.442 |
| (ratio) | | | |
| Accum Ratio | 1.615 ± 0.859 | 1.927 ± 1.310 | 0.971 + 0.453 |
| (ratio) | | | |
| Day 180 | N = 63 | N = 68 | N = 45 |
| C_{avg} (ng/dL) | 520 ± 227 | 722 ± 242 | 403 ± 163 |
| Cₘₐₓ (ng/dL) | 779 ± 359 | 1091 ± 437 | 580 ± 240 |
| Tₘₐₓ *(hr) | 4.0 (0.0-24.0) | 7.9 (0.0-24.0) | 10.0 (0.0-24.0) |
| Cₘᵢₙ (ng/dL) | 348 ± 164 | 485 ± 184 | 223 ± 114 |
| Tₘᵢₙ *(hr) | 11.9 (0.0-24.0) | 11.8 (0.0-27.4) | 2.0 (0.0-25.7) |
| Fluc Index | 0.845 ± 0.379 | 0.829 ± 0.392 | 0.891 ± 0.319 |
| (ratio) | | | |
| Accum Ratio | 1.523 ± 1.024 | 1.897 ± 2.123 | 0.954 ± 0.4105 |
| (ratio) | | | |

*Median (Range)

TABLE 28(f)
Testosterone Phamacokinetic Parameters on Days 30, 90, 180
by Final Treatment Group (Mean ± SD)

| | Doses Received During Initial => Extended Treatment Phases | | | | |
|---|---|---|---|---|---|
| | 5.0 g/day | 5.0 => 7.5 g/day | 10.0 => 7.5 g/day | 10.0 g/day | |
| | T-gel | T-gel | T-gel | T-gel | T-patch |
| Day 30 | N = 47 | N = 19 | N = 19 | N = 55 | N = 70 |
| C_{avg} (ng/dL) | 604 ± 288 | 472 ± 148 | 946 ± 399 | 739 ± 230 | 419 ± 163 |
| Cₘₐₓ (ng/dL) | 941 ± 509 | 716 ± 294 | 1409 ± 556 | 1128 ± 436 | 576 ± 223 |
| Tₘₐₓ* (hr) | 7.9 (0.0-24.0) | 8.0 (0.0-24.0) | 8.0 (0.0-24.3) | 7.8 (0.0-24.3) | 11.3 (0.0-24.0) |
| Cₘᵢₙ (ng/dL) | 387 ± 159 | 296 ± 97 | 600 ± 339 | 471 ± 175 | 235 ± 122 |
| Tₘᵢₙ* (hr) | 8.1 (0.0-24.1) | 1.7 (0.0-24.1) | 11.4 (0.0-24.1) | 8.0 (0.0-25.8) | 2.0 (0.0-24.2) |
| Fluc Index (ratio) | 0.861 ± 0.341 | 0.846 ± 0.315 | 0.927 ± 0.409 | 0.884 ± 0.445 | 0.823 ± 0.289 |
| Accum Ratio (ratio) | 1.543 ± 0.747 | 1.494 ± 0.691 | 2.053 ± 1.393 | 1.864 ± 1.657 | 0.937 ± 0.354 |
| Day 90 | N = 45 | N = 20 | N = 18 | N = 55 | N = 64 |
| C_{avg} (ng/dL) | 596 ± 266 | 455 ± 164 | 859 ± 298 | 771 ± 268 | 417 ± 157 |
| Cₘₐₓ (ng/dL) | 931 ± 455 | 654 ± 359 | 1398 ± 733 | 1141 ± 498 | 597 ± 242 |
| Tₘₐₓ* (hr) | 3.8 (0.0-24.1) | 7.7 (0.0-24.0) | 7.9 (0.0-24.0) | 7.9 (0.0-25.2) | 8.1 (0.0-25.0) |
| Cₘᵢₙ (ng/dL) | 384 ± 147 | 286 ± 125 | 532 ± 181 | 492 ± 197 | 213 ± 105 |
| Tₘᵢₙ* (hr) | 7.9 (0.0-25.3) | 0.0 (0.0-24.0) | 12.0 (0.0-24.1) | 4.0 (0.0-24.8) | 2.0 (0.0-24.0) |
| Fluc Index (ratio) | 0.886 ± 0.391 | 0.771 ± 0.425 | 0.959 ± 0.490 | 0.826 ± 0.363 | 0.937 ± 0.442 |
| Accum Ratio (ratio) | 1.593 ± 0.813 | 1.737 ± 1.145 | 1.752 ± 0.700 | 1.952 ± 1.380 | 0.971 ± 0.453 |
| Day 180 | N = 44 | N = 18 | N = 19 | N = 48 | N = 41 |
| C_{avg} (ng/dL) | 555 ± 225 | 450 ± 219 | 744 ± 320 | 713 ± 209 | 408 ± 165 |
| Cₘₐₓ (ng/dL) | 803 ± 347 | 680 ± 369 | 1110 ± 468 | 1083 ± 434 | 578 ± 245 |
| Tₘₐₓ* (hr) | 5.8 (0.0-24.0) | 2.0 (0.0-24.0) | 7.8 (0.0-24.0) | 7.7 (0.0-24.0) | 10.6 (0.0-24.0) |
| Cₘᵢₙ (ng/dL) | 371 ± 165 | 302 ± 150 | 505 ± 233 | 485 ± 156 | 222 ± 116 |
| Tₘᵢₙ* (hr) | 11.9 (0.0-24.0) | 9.9 (0.0-24.0) | 12.0 (0.0-24.0) | 8.0 (0.0-27.4) | 2.0 (0.0-25.7) |
| Fluc Index (ratio) | 0.853 ± 0.402 | 0.833 ± 0.335 | 0.824 ± 0.298 | 0.818 ± 0.421 | 0.866 ± 0.311 |
| Accum Ratio (ratio) | 1.541 ± 0.917 | NA | NA | 2.061 ± 2.445 | 0.969 ± 0.415 |

*Median (range)

Dose Proportionality for AndroGel®

Table 28(g) shows the increase in AUC₀₋₂₄ on days 30, 90, and 180 from the pretreatment baseline (net AUC₀₋₂₄). In order to assess dose-proportionality, the bioequivalence assessment was performed on the log-transformed AUCs using “treatment” as the only factor. The AUCs were compared after subtracting away the AUC contribution from the endogenous secretion of testosterone (the AUC on day 0) and adjusting for the two-fold difference in applied doses. The AUC ratio on day 30 was 0.95 (90% C.I.:0.75-1.19) and on day 90 was 0.92 (90% C.I.:0.73-1.17). When the day 30 and day 90 data was combined, the AUC ratio was 0.93 (90% C.I.:0.79-1.10).

The data shows dose proportionality for AndroGel® treatment. The geometric mean for the increase in AUC₀₋₂₄ from day 0 to day 30 or day 90 was twice as great for the 10.0 g/day group as for the 5.0 g/day group. A 125 ng/dL mean increase in serum testosterone C_{avg} level was produced by each 2.5 g/day of AndroGel®. In other words, the data shows that 0.1 g/day of AndroGel® produced, on the average, a 5 ng/dL increase in serum testosterone concentration. This dose proportionality aids dosing adjustment by the physician. Because AndroGel® is provided in 2.5 g packets (containing 25 mg of testosterone), each 2.5 g packet will produce, on average, a 125 ng/dL increase in the C_{avg} for serum total testosterone.

TABLE 28(g)
Net AUC₀₋₂₄ (nmol*h/L) on Days 30, 90, and 180
after Transdermal Testosterone Application

| | T Patch | T gel 5.0 g/day | T gel 10.0 g/day |
|---|---|---|---|
| Day 30 | 154 ± 18 | 268 ± 28 | 446 ± 30 |
| Day 90 | 157 ± 20 | 263 ± 29 | 461 ± 28 |
| Day 180 | 160 ± 25 | 250 ± 32 | 401 ± 27 |

The increase in AUC₀₋₂₄ from pretreatment baseline achieved by the 10.0 g/day and the 5.0 g/day groups were approximately 2.7 and 1.7 fold higher than that resulting from application of the testosterone patch.

Pharmacokinetics of Serum Free Testosterone Concentration

Methods

Serum free testosterone was measured by RIA of the dialysate, after an overnight equilibrium dialysis, using the same RIA reagents as the testosterone assay. The LLQ of serum free testosterone, using the equilibrium dialysis method, was estimated to be 22 pmol/L. When steroid free serum was spiked with increasing doses of testosterone in the adult male range, increasing amounts of free testosterone were recovered with a coefficient of variation that ranged from 11.0-18.5%. The intra- and interassay coefficients of free testosterone were 15% and 16.8% for adult normal male values, respectively. As estimated by the UCLA-Harbor Medical Center, free testosterone concentrations range from 3.48-17.9 ng/dL (121-620 pmol/L) in normal adult men.

Pharmacokinetic Results

In general, as shown in Table 29, the pharmacokinetic parameters of serum free testosterone mirrored that of serum total testosterone as described above. At baseline (day 0), the mean serum free testosterone concentrations (C_{avg}) were similar in all three groups which were at the lower limit of the adult male range. The maximum serum free testosterone concentration occurred between 8 and 10 a.m., and the minimum about 8 to 16 hours later. This data is consistent with the mild diurnal variation of serum testosterone.

FIG. No. 13(a) shows the 24-hour pharmacokinetic profiles for the three treatment groups on day 1. After application of the testosterone patch, the serum free testosterone levels peaked at 12 hours about 4 hours earlier than those achieved by the AndroGel® groups The serum free testosterone levels then declined in the testosterone patch group whereas in the AndroGel® groups, the serum free testosterone levels continued to rise.

FIG. Nos. 13(b) and 6(c) show the pharmacokinetic profiles of free testosterone in the AndroGel®-treated groups resembled the unique testosterone profiles on days 30 and 90. After AndroGel® application, the mean serum free testosterone levels in the three groups were within normal range. Similar to the total testosterone results, the free testosterone C_{avg} achieved by the 10.0 g/day group was 1.4 fold higher than the 5.0 g/day group and 1.7 fold higher than the testosterone patch group. Moreover, the accumulation ratio for the testosterone patch was significantly less than that of the 5.0 g/day AndroGel® group and the 10.0 g/day AndroGel® group.

FIG. No. 13(d) shows the free testosterone concentrations by final treatment groups on day 180. In general, the free testosterone concentrations exhibited a similar pattern as serum testosterone. The 24-hour pharmacokinetic parameters were similar to those on days 30 and 90 in those subjects who remained in the three original randomized groups. Again, in the subjects titrated to receive 7.5 g/day of AndroGel®, the group was not homogenous. The free testosterone C_{avg} in the patients with doses adjusted upwards from 5.0 to 7.5 g/day remained 29% lower than those of subjects remaining in the 5.0 g/day group. The free testosterone C_{avg} in the patients whose doses were decreased from 10.0 to 7.5 g/day was 11% higher than those in remaining in the 10.0 g/day group.

FIG. Nos. 13(e)-(g) show the free testosterone concentrations in the three groups of subjects throughout the 180-day treatment period. Again, the free testosterone levels followed that of testosterone. The mean free testosterone levels in all three groups were within the normal range with the 10.0 g/day group maintaining higher free testosterone levels than both the 5.0 g/day and the testosterone patch groups.

TABLE 29
Free Testosterone Pharmacokinetic Parameters
by Final Treatment (Mean ± SD)

| | Doses Received During Initial => Extended Treatment Phases | | | | |
|---|---|---|---|---|---|
| | 5.0 g/day | 5.0 => 7.5 g/day | 10.0 => 7.5 g/day | 10/0 g/day | |
| | T-gel | T-gel | T-gel | T-gel | T-patch |
| Day 0 | N = 53 | N = 20 | N = 20 | N = 58 | N = 76 |
| C_{avg} (ng/dL) | 4.52 ± 3.35 | 4.27 ± 3.45 | 4.64 ± 3.10 | 4.20 ± 3.33 | 4.82 ± 3.64 |
| Cₘₐₓ (ng/dL) | 5.98 ± 4.25 | 6.06 ± 5.05 | 6.91 ± 4.66 | 5.84 ± 4.36 | 6.57 ± 4.90 |
| Tₘₐₓ* (hr) | 4.0 (0.0-24.5) | 2.0 (0.0-24.0) | 13.5 (0.0-24.2) | 2.1 (0.0-24.1) | 3.8 (0.0-24.0) |
| Cₘᵢₙ (ng/dL) | 3.23 ± 2.74 | 3.10 ± 2.62 | 3.14 ± 2.14 | 3.12 ± 2.68 | 3.56 ± 2.88 |
| Tₘᵢₙ* (hr) | 8.0 (0.0-24.2) | 9.9 (0.0-16.0) | 4.0 (0.0-23.3) | 8.0 (0.0-24.0) | 7.9 (0.0-24.0) |
| Fluc Index (ratio) | 0.604 ± 0.342 | 0.674 ± 0.512 | 0.756 ± 0.597 | 0.634 ± 0.420 | 0.614 ± 0.362 |
| Day 1 | N = 53 | N = 20 | N = 19 | N = 57 | N = 74 |
| C_{avg} (ng/dL) | 7.50 ± 4.83 | 6.80 ± 4.82 | 9.94 ± 5.04 | 8.93 ± 6.09 | 9.04 ± 4.81 |
| Cₘₐₓ (ng/dL) | 10.86 ± 7.45 | 10.10 ± 7.79 | 15.36 ± 7.31 | 13.20 ± 8.61 | 12.02 ± 6.14 |
| Tₘₐₓ* (hr) | 16.0 (0.0-25.3) | 13.9 (0.0-24.3) | 15.7 (2.0-24.0) | 23.5 (1.8-24.3) | 12.0 (1.8-24.0) |
| Cₘᵢₙ (ng/dL) | 4.30 ± 3.33 | 3.69 ± 3.24 | 3.88 ± 2.73 | 4.40 ± 3.94 | 4.67 ± 3.52 |
| Tₘᵢₙ* (hr) | 0.0 (0.0-24.1) | 1.8 (0.0-24.0) | 0.0 (0.0-24.2) | 0.0 (0.0-23.9) | 0.0 (0.0-24.0) |
| Day 30 | N = 47 | N = 19 | N = 19 | N = 55 | N = 70 |
| C_{avg} (ng/dL) | 11.12 ± 6.22 | 7.81 ± 3.94 | 16.18 ± 8.18 | 13.37 ± 7.13 | 8.12 ± 4.15 |
| Cₘₐₓ (ng/dL) | 16.93 ± 10.47 | 11.62 ± 6.34 | 25.14 ± 10.80 | 19.36 ± 9.75 | 11.48 ± 5.78 |
| Tₘₐₓ* (hr) | 8.0 (0.0-27.8) | 8.0 (0.0-26.3) | 8.0 (0.0-24.3) | 8.0 (0.0-24.3) | 8.0 (0.0-24.0) |
| Cₘᵢₙ (ng/dL) | 6.99 ± 3.82 | 4.78 ± 3.10 | 9.99 ± 7.19 | 8.25 ± 5.22 | 4.31 ± 3.20 |
| Tₘᵢₙ* (hr) | 4.0 (0.0-24.1) | 3.5 (0.0-24.1) | 11.4 (0.0-24.1) | 7.8 (0.0-25.8) | 2.0 (0.0-24.8) |
| Fluc Index (ratio) | 0.853 ± 0.331 | 0.872 ± 0.510 | 1.051 ± 0.449 | 0.861 ± 0.412 | 0.929 ± 0.311 |
| Accum Ratio (ratio) | 1.635 ± 0.820 | 1.479 ± 0.925 | 2.065 ± 1.523 | 1.953 ± 1.626 | 0.980 ± 0.387 |
| Day 90 | N = 45 | N = 20 | N = 18 | N = 55 | N = 64 |
| C_{avg} (ng/dL) | 12.12 ± 7.78 | 8.06 ± 3.78 | 17.65 ± 8.62 | 13.11 ± 5.97 | 8.50 ± 5.04 |
| Cₘₐₓ (ng/dL) | 18.75 ± 12.90 | 10.76 ± 4.48 | 25.29 ± 12.42 | 18.61 ± 8.20 | 12.04 ± 6.81 |
| Tₘₐₓ* (hr) | 4.0 (0.0-24.0) | 9.7 (0.0-24.0) | 8.0 (0.0-24.0) | 8.0 (0.0-25.2) | 11.6 (0.0-25.0) |
| Cₘᵢₙ (ng/dL) | 7.65 ± 4.74 | 4.75 ± 2.86 | 10.56 ± 6.07 | 8.40 ± 4.57 | 4.38 ± 3.70 |
| Tₘᵢₙ* (hr) | 8.0 (0.0-24.0) | 1.9 (0.0-24.0) | 5.9 (0.0-24.1) | 4.0 (0.0-24.8) | 2.0 (0.0-24.1) |
| Fluc Index (ratio) | 0.913 ± 0.492 | 0.815 ± 0.292 | 0.870 ± 0.401 | 0.812 ± 0.335 | 0.968 ± 0.402 |
| Accum Ratio (ratio) | 1.755 ± 0.983 | 1.916 ± 1.816 | 1.843 ± 0.742 | 2.075 ± 1.866 | 1.054 ± 0.498 |
| Day 180 | N = 44 | N = 18 | N = 19 | N = 48 | N = 41 |
| C_{avg} (ng/dL) | 11.01 ± 5.24 | 7.80 ± 4.63 | 14.14 ± 7.73 | 12.77 ± 5.70 | 7.25 ± 4.90 |
| Cₘₐₓ (ng/dL) | 16.21 ± 7.32 | 11.36 ± 6.36 | 22.56 ± 12.62 | 18.58 ± 9.31 | 10.17 ± 5.90 |
| Tₘₐₓ* (hr) | 7.9 (0.0-24.0) | 2.0 (0.0-23.9) | 7.8 (0.0-24.0) | 8.0 (0.0-24.0) | 11.1 (0.0-24.0) |
| Cₘᵢₙ (ng/dL) | 7.18 ± 3.96 | 5.32 ± 4.06 | 9.54 ± 6.45 | 8.23 ± 4.01 | 3.90 ± 4.20 |
| Tₘᵢₙ* (hr) | 9.9 (0.0-24.2) | 7.9 (0.0-24.0) | 8.0 (0.0-23.2) | 11.8 (0.0-27.4) | 2.5 (0.0-25.7) |
| Fluc Index (ratio) | 0.897 ± 0.502 | 0.838 ± 0.378 | 0.950 ± 0.501 | 0.815 ± 0.397 | 0.967 ± 0.370 |
| Accum Ratio (ratio) | 1.712 ± 1.071 | NA | NA | 2.134 ± 1.989 | 1.001 ± 0.580 |

*Median (Range)

Serum DHT Concentrations

Serum DHT was measured by RIA after potassium permanganate treatment of the sample followed by extraction. The methods and reagents of the DHT assay were provided by DSL (Webster, Tex.). The cross reactivities of the antiserum used in the RIA for DHT were 6.5% for 3-β-androstanediol, 1.2% for 3-α-androstanediol, 0.4% for 3-α-androstanediol glucuronide, and 0.4% for testosterone (after potassium permanganate treatment and extraction), and less than 0.01% for other steroids tested. This low cross-reactivity against testosterone was further confirmed by spiking steroid free serum with 35 nmol/L (1,000 pg/dL) of testosterone and taking the samples through the DHT assay. The results even on spiking with over 35 nmol/L of testosterone was measured as less than 0.1 nmol/L of DHT. The LLQ of serum DHT in the assay was 0.43 nmol/L. The mean accuracy (recovery) of the DHT assay determined by spiking steroid free serum with varying amounts of DHT from 0.43 nmol/L to 9 nmol/L was 101% and ranged from 83 to 114%. The intra-assay and inter-assay coefficients of variation for the DHT assay were 7.8 and 16.6%, respectively, for the normal adult male range. The normal adult male range of DHT was 30.7-193.2 ng/dL (1.06 to 6.66 nmol/L ) as determined by the UCLA-Harbor Medical Center.

As shown in Table 30, the pretreatment mean serum DHT concentrations were between 36 and 42 ng/dL, which were near the lower limit of the normal range in all three initial treatment groups. None of the patients had DHT concentrations above the upper limit of the normal range on the pretreatment day, although almost half (103 patients) had concentrations less than the lower limit.

FIG. No. 14 shows that after treatment, the differences between the mean DHT concentrations associated with the different treatment groups were statistically significant, with patients receiving AndroGel® having a higher mean DHT concentration than the patients using the patch and showing dose-dependence in the mean serum DHT concentrations. Specifically, after testosterone patch application mean serum DHT levels rose to about 1.3 fold above the baseline. In contrast, serum DHT increased to 3.6 and 4.8 fold above baseline after application of 5.0 g/day and 10.0 g/day of AndroGel®, respectively.

TABLE 30
DHT Concentrations (ng/dL)
on Each of the Observation Days
By Initial Treatment (Mean ± SD)

| | Day 0 | Day 30 | Day 60 | Day 90 | Day 120 | Day 150 | Day 180 |
|---|---|---|---|---|---|---|---|
| 5.0 g/day | N = 73 | N = 69 | N = 70 | N = 67 | N = 65 | N = 63 | N = 65 |
| T-gel | 36.0 ± 19.9 | 117.6 ± 74.9 | 122.4 ± 99.4 | 130.1 ± 99.2 | 121.8 ± 89.2 | 144.7 ± 110.5 | 143.7 ± 105.9 |
| 10.0 g/day | N = 78 | N = 78 | N = 74 | N = 75 | N = 68 | N = 67 | N = 71 |
| T-gel | 42.0 ± 29.4 | 200.4 ± 127.8 | 222.0 ± 126.6 | 207.7 ± 111.0 | 187.3 ± 97.3 | 189.1 ± 102.4 | 206.1 ± 105.9 |
| T-Patch | N = 76 | N = 73 | N = 68 | N = 66 | N = 49 | N = 46 | N = 49 |
| | 37.4 ± 21.4 | 50.8 ± 34.6 | 49.3 ± 27.2 | 43.6 ± 26.9 | 53.0 ± 52.8 | 54.0 ± 42.5 | 52.1 ± 34.3 |
| Across RX | 0.6041 | 0.0001 | 0.0001 | 0.0001 | 0.0001 | 0.0001 | 0.0001 |

The increase in DHT concentrations are likely attributed to the concentration and location of 5α-reductase in the skin. For example, the large amounts of 5α-reductase in the scrotal skin presumably causes an increase in DHT concentrations in the TESTODERM® patch. In contrast, the ANDRODERM® and TESTODERM TTS® patches create little change in DTH levels because the surface area of the patch is small and little 5α-reductase is located in nonscrotal skin. AndroGel® presumably causes an increase in DHT levels because the gel is applied to a relatively large skin area and thus exposes testosterone to greater amounts of the enzyme.

To date, elevated DHT levels have not been reported to have any adverse clinical effects. Moreover, there is some evidence to suggest that increased DHT levels may inhibit prostate cancer.

DHFT/T Ratio

The UCLA-Harbor Medical Center reports a DHT/T ratio of 0.052-0.328 for normal adult men. In this example, the mean ratios for all three treatments were within the normal range on day 0. As shown in FIG. No. 15 and Table 31, there were treatment and concentration-dependent increases observed over the 180-day period. Specifically, the AndroGel® treatment groups showed the largest increase in DHT/T ratio. However, the mean ratios for all of the treatment groups remained within the normal range on all observation days.

TABLE 31
DHT/T Ratio
on Each of the Observation Days
By Initial Treatment (Mean ± SD)

| | Day 0 | Day 30 | Day 60 | Day 90 | Day 120 | Day 150 | Day 180 |
|---|---|---|---|---|---|---|---|
| 5.0 g/day | N = 73 | N = 68 | N = 70 | N = 67 | N = 65 | N = 62 | N = 64 |
| T-gel | 0.198 ± 0.137 | 0.230 ± 0.104 | 0.256 ± 0.132 | 0.248 ± 0.121 | 0.266 ± 0.119 | 0.290 ± 0.145 | 0.273 ± 0.160 |
| 10.0 g/day | N = 78 | N = 77 | N = 74 | N = 74 | N = 68 | N = 67 | N = 71 |
| T-gel | 0.206 ± 0.163 | 0.266 ± 0.124 | 0.313 ± 0.160 | 0.300 ± 0.131 | 0.308 ± 0.145 | 0.325 ± 0.142 | 0.291 ± 0.124 |
| T-Patch | N = 76 | N = 73 | N = 68 | N = 65 | N = 49 | N = 46 | N = 46 |
| | 0.204 ± 0.135 | 0.192 ± 0.182 | 0.175 ± 0.102 | 0.175 ± 0.092 | 0.186 ± 0.134 | 0.223 ± 0.147 | 0.212 ± 0.160 |
| Across RX | 0.7922 | 0.0001 | 0.0001 | 0.0001 | 0.0001 | 0.0001 | 0.0002 |

Total Androgen (DHT+T)

The UCLA-Harbor Medical Center has determined that the normal total androgen concentration is 372 to 1,350 ng/dL. As shown in FIG. No. 16 and Table 32, the mean pre-dose total androgen concentrations for all three treatments were below the lower limit of the normal range on pretreatment day 0. The total androgen concentrations for both AndroGel® groups were within the normal range on all treatment observation days. In contrast, the mean concentrations for patients receiving the testosterone patch was barely within the normal range on day 60 and 120, but were below the lower normal limit on days 30, 90, 150, and 180.

TABLE 32
Total Androgen (DHT + T) (ng/dL)
on Each of the Observation Days
By Initial Treatment (Mean ± SD)

| | Day 0 | Day 30 | Day 60 | Day 90 | Day 120 | Day 150 | Day 180 |
|---|---|---|---|---|---|---|---|
| 5.0 g/day | N = 73 | N = 68 | N = 70 | N = 67 | N = 65 | N = 62 | N = 64 |
| T-gel | 281 ± 150 | 659 ± 398 | 617 ± 429 | 690 ± 431 | 574 ± 331 | 631 ± 384 | 694 ± 412 |
| 10.0 g/day | N = 78 | N = 77 | N = 74 | N = 74 | N = 68 | N = 67 | N = 71 |
| T-gel | 307 ± 180 | 974 ± 532 | 1052 ± 806 | 921 ± 420 | 827 ± 361 | 805 ± 383 | 944 ± 432 |
| T-Patch | N = 76 | N = 73 | N = 68 | N = 65 | N = 49 | N = 46 | N = 46 |
| | 282 ± 159 | 369 ± 206 | 392 ± 229 | 330 ± 173 | 378 ± 250 | 364 ± 220 | 355 ± 202 |
| Across RX | 0.7395 | 0.0001 | 0.0001 | 0.0001 | 0.0001 | 0.0001 | 0.0001 |

E₂ Concentrations

Serum E₂ levels were measured by a direct assay without extraction with reagents from ICN (Costa Mesa, Calif.). The intra-assay and inter-assay coefficients of variation of E₂ were 6.5 and 7.1% respectively. The UCLA-Harbor Medical Center reported an average E₂ concentration ranging from 7.1 to 46.1 pg/mL (63 to 169 pmol/L) for normal adult male range. The LLQ of the E₂ was 18 pmol/L. The cross reactivities of the E₂ antibody were 6.9% for estrone, 0.4% for equilenin, and less than 0.01% for all other steroids tested. The accuracy of the E₂ assay was assessed by spiking steroid free serum with increasing amount of E₂ (18 to 275 pmol/L). The mean recovery of E₂ compared to the amount added was 99.1% and ranged from 95 to 101%.

FIG. No. 17 depicts the E₂ concentrations throughout the 180-day study. The pretreatment mean E₂ concentrations for all three treatment groups were 23-24 pg/mL. During the study, the E₂ levels increased by an average 9.2% in the testosterone patch during the treatment period, 30.9% in the 5.0 g/day AndroGel® group, and 45.5% in the 10.0 g/day AndroGel® group. All of the mean concentrations fell within the normal range.

TABLE 33
Estradiol Concentration (pg/mL)
on Each of the Observation Days
By Initial Treatment (Mean ± SD)

| | Day 0 | Day 30 | Day 60 | Day 90 | Day 120 | Day 150 | Day 280 |
|---|---|---|---|---|---|---|---|
| 5.0 g/day T-gel | N = 73 | N = 69 | N = 68 | N = 67 | N = 64 | N = 65 | N = 65 |
| | 23.0 ± 9.2 | 29.2 ± 11.0 | 28.1 ± 10.0 | 31.4 ± 11.9 | 28.8 ± 9.9 | 30.8 ± 12.5 | 32.3 ± 13.8 |
| 10.0 g/day T-gel | N = 78 | N = 78 | N = 74 | N = 75 | N = 71 | N = 66 | N = 71 |
| | 24.5 ± 9.5 | 33.7 ± 11.5 | 36.5 ± 13.5 | 37.8 ± 13.3 | 34.6 ± 10.4 | 35.0 ± 11.1 | 36.3 ± 13.9 |
| T-Patch | N = 76 | N = 72 | N = 68 | N = 66 | N = 50 | N = 49 | N = 49 |
| | 23.8 ± 8.2 | 25.8 ± 9.8 | 24.8 ± 8.0 | 25.7 ± 9.8 | 25.7 ± 9.4 | 27.0 ± 9.2 | 26.9 ± 9.5 |
| Across RX | 0.6259 | 0.0001 | 0.0001 | 0.0001 | 0.0001 | 0.0009 | 0.0006 |

E₂ is believed to be important for the maintenance of normal bone. In addition, E₂ has a positive effect on serum lipid profiles.

Serum SHBG Concentrations

Serum SHBG levels were measured with a fluoroimmunometric assay (“FIA”) obtained from Delfia (Wallac, Gaithersberg, Md.). The intra- and interassay coefficients were 5% and 12% respectively. The LLQ was 0.5 nmol/L. The UCLA-Harbor Medical Center determined that the adult normal male range for the SHBG assay is 0.8 to 46.6 nmol/L.

As shown in FIG. No. 18 and Table 34, the serum SHBG levels were similar and within the normal adult male range in the three treatment groups at baseline. None of the treatment groups showed major changes from the baseline on any of the treatment visit days. After testosterone replacement, serum SHBG levels showed a small decrease in all three groups. The most marked change occurred in the 10.0 g/day AndroGel® group.

TABLE 34
SHBG Concentration (nmol/L)
on Each of the Observation Days
By Initial Treatment (Mean ± SD)

| | Day 0 | Day 30 | Day 60 | Day 90 | Day 120 | Day 150 | Day 180 |
|---|---|---|---|---|---|---|---|
| 5.0 g/day | N = 73 | N = 69 | N = 69 | N = 67 | N = 66 | N = 65 | N = 65 |
| T-gel | 26.2 ± 14.9 | 24.9 ± 14.0 | 25.9 ± 14.4 | 25.5 ± 14.7 | 25.2 ± 14.1 | 24.9 ± 12.9 | 24.2 ± 13.6 |
| 10.0 g/day | N = 78 | N = 78 | N = 75 | N = 75 | N = 72 | N = 68 | N = 71 |
| T-gel | 26.6 ± 17.8 | 24.8 ± 14.5 | 25.2 ± 15.5 | 23.6 ± 14.7 | 25.5 ± 16.5 | 23.8 ± 12.5 | 24.0 ± 14.5 |
| T-Patch | N = 76 | N = 72 | N = 68 | N = 66 | N = 50 | N = 49 | N = 49 |
| | 30.2 ± 22.6 | 28.4 ± 21.3 | 28.2 ± 23.8 | 28.0 ± 23.6 | 26.7 ± 16.0 | 26.7 ± 16.4 | 25.8 ± 15.1 |
| Across RX | 0.3565 | 0.3434 | 0.5933 | 0.3459 | 0.8578 | 0.5280 | 0.7668 |

Gonadotropins

Serum FSH and LH were measured by highly sensitive and specific solid-phase FIA assays with reagents provided by Delfia (Wallac, Gaithersburg, Md.). The intra-assay coefficient of variations for LH and FSH fluroimmunometric assays were 4.3 and 5.2%, respectively; and the interassay variations for LH and FSH were 11.0% and 12.0%, respectively. For both LH and FSH assays, the LLQ was determined to be 0.2 IU/L. All samples obtained from the same subject were measured in the same assay. The UCLA-Harbor Medical Center reports that the adult normal male range for LH is 1.0-8.1 U/L and for FSH is 1.0-6.9U/L.

FSH

Table 35(a)-(d) shows the concentrations of FSH throughout the 180-day treatment depending on the cause of hypogonadism: (1) primary, (2) secondary, (3) age-associated, or (4) unknown.

As discussed above, patients with primary hypogonadism have an intact feedback inhibition pathway, but the testes do not secrete testosterone. As a result, increasing serum testosterone levels should lead to a decrease in the serum FSH concentrations. In this example, a total of 94 patients were identified as having primary hypogonadism. For these patients, the mean FSH concentrations in the three treatment groups on day 0 were 21-26 mlU/mL, above the upper limit of the normal range. As shown in FIG. No. 19(a) and Table 35(a), the mean FSH concentrations decreased during treatment in all three treatment regimens. However, only the 10.0 g/day AndroGel® group reduced the mean concentrations to within the normal range during the first 90 days of treatment. Treatment with the 10.0 g/day AndroGel® group required approximately 120 days to reach steady state. The mean FSH concentration in patients applying 5.0 g/day of AndroGel® showed an initial decline that was completed by day 30 and another declining phase at day 120 and continuing until the end of treatment. Mean FSH concentrations in the patients receiving the testosterone patch appeared to reached steady state after 30 days but were significantly higher than the normal range.

TABLE 35(a)
FSH Concentrations (mlU/mL) on Each of the
Observation Days by Initial Treatment Group for Patients
Having Primary Hypogonadism (Mean ± SD)

| | N | 5 g/day | N | 10 g/day | N | T-patch |
|---|---|---|---|---|---|---|
| Day 0 | 26 | 21.6 ± 21.0 | 33 | 20.9 ± 15.9 | 34 | 25.5 ± 25.5 |
| Day 30 | 23 | 10.6 ± 15.0 | 34 | 10.6 ± 14.1 | 31 | 21.4 ± 24.6 |
| Day 60 | 24 | 10.8 ± 16.9 | 32 | 7.2 ± 12.6 | 31 | 21.7 ± 23.4 |
| Day 90 | 24 | 10.4 ± 19.7 | 31 | 5.7 ± 10.1 | 30 | 19.5 ± 20.0 |
| Day 120 | 24 | 8.1 ± 15.2 | 28 | 4.6 ± 10.2 | 21 | 25.3 ± 28.4 |
| Day 150 | 22 | 6.7 ± 15.0 | 29 | 5.3 ± 11.0 | 21 | 18.6 ± 24.0 |
| Day 180 | 24 | 6.2 ± 11.3 | 28 | 5.3 ± 11.2 | 22 | 24.5 ± 27.4 |

Patients with secondary hypogonadism have a deficient testosterone negative feedback system. As shown in FIG. No. 19(b), of 44 patients identified as having secondary hypogonadism, the mean FSH concentrations decreased during treatment, although the decrease over time was not statistically significant for the testosterone patch. The patients in the 5.0 g/day AndroGel® group showed a decrease in the mean FSH concentration by about 35% by day 30, with no further decrease evident by day 60. Beyond day 90, the mean FSH concentration in the patients appeared to slowly return toward the pretreatment value. By day 30, all of the 10.0 g/day AndroGel® group had FSH concentrations less than the lower limit.

TABLE 35(b)
FSH Concentrations (mlU/mL) on Each of the
Observation Days by Initial Treatment Group for Patients
Having Secondary Hypogonadism (Mean ± SD)

| | N | 5 g/day | N | 10 g/day | N | T-patch |
|---|---|---|---|---|---|---|
| Day 0 | 17 | 4.2 ± 6.6 | 12 | 2.1 ± 1.9 | 15 | 5.1 ± 9.0 |
| Day 30 | 16 | 2.8 ± 5.9 | 12 | 0.2 ± 0.1 | 14 | 4.2 ± 8.0 |
| Day 60 | 17 | 2.8 ± 6.1 | 12 | 0.2 ± 0.1 | 13 | 4.2 ± 7.4 |
| Day 90 | 15 | 2.9 ± 5.6 | 12 | 0.2 ± 0.1 | 14 | 4.9 ± 9.0 |
| Day 120 | 14 | 3.0 ± 6.1 | 12 | 0.1 ± 0.1 | 12 | 6.1 ± 10.7 |
| Day 150 | 14 | 3.5 ± 7.5 | 12 | 0.2 ± 0.2 | 11 | 4.6 ± 6.5 |
| Day 180 | 14 | 3.7 ± 8.6 | 12 | 0.1 ± 0.1 | 12 | 4.9 ± 7.4 |

Twenty-five patients were diagnosed with age-associated hypogonadism. As shown in FIG. No. 19(c), the 5.0 g/day AndroGel® group had a mean pretreatment FSH concentration above the normal range. The mean concentration for this group was within the normal range by day 30 and had decreased more than 50% on days 90 and 180. The decrease in FSH mean concentration in the 10.0 g/day AndroGel® group showed a more rapid response. The concentrations in all six patients decreased to below the lower normal limit by day 30 and remained there for the duration of the study. The six patients who received the testosterone patch exhibited no consistent pattern in the mean FSH level; however, there was an overall trend towards lower FHS levels with continued treatment.

TABLE 35(c)
FSH Concentrations (mlU/mL) on Each of the
Observation Days by Initial Treatment Group for Patients
Having Age-Related Hypogonadism (Mean ± SD)

| | N | 5 g/day | N | 10 g/day | N | T-patch |
|---|---|---|---|---|---|---|
| Day 0 | 13 | 8.0 ± 9.1 | 6 | 5.2 ± 1.9 | 6 | 4.7 ± 1.7 |
| Day 30 | 12 | 4.6 ± 7.4 | 6 | 0.4 ± 0.3 | 6 | 3.7 ± 2.0 |
| Day 60 | 12 | 3.9 ± 6.6 | 6 | 0.3 ± 0.3 | 4 | 4.3 ± 3.3 |
| Day 90 | 11 | 3.8 ± 7.0 | 6 | 0.4 ± 0.7 | 4 | 3.5 ± 1.9 |
| Day 120 | 11 | 4.2 ± 8.3 | 6 | 0.4 ± 0.7 | 4 | 4.2 ± 3.3 |
| Day 150 | 11 | 4.3 ± 8.1 | 5 | 0.2 ± 0.2 | 4 | 3.4 ± 2.7 |
| Day 180 | 11 | 4.0 ± 7.2 | 6 | 0.2 ± 0.2 | 4 | 2.7 ± 2.1 |

Sixty-four patients in the study suffered from unclassified hypogonadism. As shown in FIG. No. 19(d), the patients showed a marked and comparatively rapid FSH concentration decrease in all three groups, with the greatest decrease being in the 10.0 g/day AndroGel® group. The 10.0 g/day AndroGel® group produced nearly a 90% decrease in the mean FSH concentration by day 30 and maintained the effect to day 180. The 5.0 g/day AndroGel® group produced about a 75% drop in mean FSH concentration by day 30 and stayed at that level for the remainder of treatment. The 21 patients receiving the testosterone patch had a 50% decrease in the mean FSH concentration by day 30, a trend that continued to day 90 when the concentration was about one-third of its pretreatment value.

TABLE 35(d)
Concentrations (mlU/mL) for FSH on Each of
the Observation Days by Initial Treatment Group for
Patients Having Unknown-Related Hypogonadism (Mean ± SD)

| | N | 5 g/day | N | 10 g/day | N | T-patch |
|---|---|---|---|---|---|---|
| Day 0 | 17 | 4.0 ± 1.8 | 26 | 4.1 ± 1.6 | 21 | 3.7 ± 1.4 |
| Day 30 | 17 | 1.1 ± 1.0 | 26 | 0.5 ± 0.5 | 21 | 1.8 ± 0.8 |
| Day 60 | 16 | 1.1 ± 1.1 | 26 | 0.3 ± 0.3 | 18 | 1.6 ± 1.0 |
| Day 90 | 17 | 1.1 ± 1.1 | 25 | 0.4 ± 0.7 | 18 | 1.2 ± 0.9 |
| Day 120 | 16 | 1.2 ± 1.4 | 26 | 0.4 ± 0.6 | 12 | 1.4 ± 1.0 |
| Day 150 | 17 | 1.4 ± 1.4 | 23 | 0.3 ± 0.5 | 13 | 1.4 ± 1.2 |
| Day 180 | 16 | 1.0 ± 0.9 | 24 | 0.4 ± 0.4 | 11 | 1.3 ± 0.9 |

This data shows that feedback inhibition of FSH secretion functioned to some extent in all four subpopulations. The primary hypogonadal population showed a dose-dependency in both the extent and rate of the decline in FSH levels. The sensitivity of the feedback process appeared to be reduced in the secondary and age-associated groups in that only the highest testosterone doses had a significant and prolonged impact on FSH secretion. In contrast, the feedback inhibition pathway in the patients in the unclassified group was quite responsive at even the lowest dose of exogenous testosterone.

LH

The response of LH to testosterone was also examined separately for the same four subpopulations. Tables 36(a)-(d) shows the LH concentrations throughout the treatment period.

As shown in FIG. No. 20(a) and Table 36(a), the LH concentrations prior to treatment were about 175% of the upper limit of the normal range in primary hypogonadal patients. The mean LH concentrations decreased during treatment in all groups. However, only the AndroGel® groups decreased the mean LH concentrations enough to fall within the normal range. As with FSH, the primary hypogonadal men receiving AndroGel® showed dose-dependence in both the rate and extent of the LH response.

TABLE 36(a)
Concentrations for LH (mlU/mL) on Each of
the Observation Days for Patients Having Primary
Hypogonadism (Summary of Mean ± SD)

| | N | 5 g/day | N | 10 g/day | N | T-patch |
|---|---|---|---|---|---|---|
| Day 0 | 26 | 12.2 ± 12.1 | 33 | 13.9 ± 14.9 | 33 | 13.3 ± 14.3 |
| Day 30 | 23 | 5.6 ± 7.6 | 34 | 5.9 ± 8.1 | 31 | 10.9 ± 12.9 |
| Day 60 | 24 | 6.8 ± 9.0 | 32 | 4.8 ± 10.0 | 31 | 10.8 ± 11.8 |
| Day 90 | 24 | 5.9 ± 9.5 | 31 | 4.2 ± 11.0 | 30 | 10.0 ± 11.7 |
| Day 120 | 24 | 6.4 ± 11.9 | 28 | 3.8 ± 10.4 | 21 | 11.5 ± 11.5 |
| Day 150 | 22 | 4.4 ± 8.5 | 29 | 4.0 ± 11.3 | 21 | 7.4 ± 6.0 |
| Day 180 | 24 | 4.8 ± 6.8 | 28 | 4.0 ± 11.9 | 22 | 11.2 ± 10.5 |

The secondary hypogonadal men were less sensitive to exogenous testosterone. For the 44 patients identified as having secondary hypogonadism, the pretreatment mean concentrations were all within the lower limit normal range. The mean LH concentrations decreased during treatment with all three regimens as shown in FIG. No. 20(b) and Table 36(b).

TABLE 36(b)
Concentrations for LH (mlU/mL) on Each of
the Observation Days for Patients Having Secondary
Hypogonadism (Summary of Mean ± SD)

| | N | 5 g/day | N | 10 g/day | N | T-patch |
|---|---|---|---|---|---|---|
| Day 0 | 17 | 1.8 ± 2.6 | 12 | 1.4 ± 1.8 | 15 | 1.6 ± 3.1 |
| Day 30 | 16 | 1.1 ± 2.2 | 12 | 0.2 ± 0.2 | 14 | 0.4 ± 0.4 |
| Day 60 | 17 | 1.4 ± 3.8 | 12 | 0.2 ± 0.2 | 13 | 0.6 ± 0.5 |
| Day 90 | 15 | 1.2 ± 2.4 | 12 | 0.2 ± 0.2 | 14 | 0.7 ± 1.0 |
| Day 120 | 14 | 1.6 ± 4.0 | 12 | 0.2 ± 0.2 | 12 | 0.8 ± 0.8 |
| Day 150 | 14 | 1.6 ± 3.5 | 12 | 0.2 ± 0.2 | 11 | 1.2 ± 2.0 |
| Day 180 | 14 | 1.5 ± 3.7 | 12 | 0.2 ± 0.2 | 12 | 1.4 ± 2.1 |

None of the 25 patients suffering from age-associated hypogonadism had pretreatment LH concentrations outside of the normal range as shown in FIG. No. 20(c) and Table 36(c). The overall time and treatment effects were significant for the AndroGel® patients but not those patients using the testosterone patch.

TABLE 36(c)
Concentrations for LH (mlU/mL) on Each of
the Observation Days for Patients Having Age-Related
Hypogonadism (Summary of Mean ± SD)

| | N | 5 g/day | N | 10 g/day | N | T-patch |
|---|---|---|---|---|---|---|
| Day 0 | 13 | 3.2 ± 1.1 | 6 | 2.4 ± 1.8 | 6 | 2.9 ± 0.6 |
| Day 30 | 12 | 1.1 ± 1.0 | 6 | 0.1 ± 0.0 | 6 | 1.8 ± 1.1 |
| Day 60 | 12 | 0.8 ± 0.7 | 6 | 0.2 ± 0.3 | 5 | 3.4 ± 2.8 |
| Day 90 | 11 | 0.9 ± 1.2 | 6 | 0.1 ± 0.0 | 4 | 2.3 ± 1.4 |
| Day 120 | 11 | 1.0 ± 1.4 | 6 | 0.1 ± 0.0 | 4 | 2.2 ± 1.4 |
| Day 150 | 11 | 1.3 ± 1.5 | 5 | 0.1 ± 0.0 | 4 | 1.9 ± 1.2 |
| Day 180 | 11 | 1.8 ± 2.1 | 6 | 0.1 ± 0.0 | 4 | 1.4 ± 1.0 |

Of the 64 patients suffering from an unclassified hypogonadism, none of the patients had a pretreatment LH concentration above the upper limit. Fifteen percent, however, had pretreatment concentrations below the normal limit. The unclassified patients showed comparatively rapid LH concentration decreases in all treatment groups as shown in FIG. No. 20(d) and Table 36(d).

TABLE 36(d)
Concentrations for LH (mlU/mL) on Each of
the Observation Days for Patients Having Unknown-
Related Hypogonadism (Summary of Mean ± SD)

| | N | 5 g/day | N | 10 g/day | N | T-patch |
|---|---|---|---|---|---|---|
| Day 0 | 17 | 1.8 ± 1.2 | 26 | 2.5 ± 1.5 | 21 | 2.5 ± 1.5 |
| Day 30 | 17 | 0.3 ± 0.3 | 26 | 0.3 ± 0.3 | 21 | 1.3 ± 1.3 |
| Day 60 | 17 | 0.4 ± 0.5 | 26 | 0.3 ± 0.3 | 18 | 1.2 ± 1.4 |
| Day 90 | 17 | 0.5 ± 0.5 | 26 | 0.3 ± 0.4 | 18 | 1.0 ± 1.4 |
| Day 120 | 17 | 0.4 ± 0.4 | 26 | 0.4 ± 0.5 | 12 | 1.2 ± 1.1 |
| Day 150 | 17 | 0.8 ± 1.1 | 23 | 0.3 ± 0.4 | 13 | 1.1 ± 1.1 |
| Day 180 | 15 | 0.3 ± 0.4 | 25 | 0.4 ± 0.4 | 11 | 1.5 ± 1.3 |

Summary: LH and FSH

Patients receiving AndroGel® or the testosterone patch achieve “hormonal steady state” only after long-term treatment. Specifically, data involving FSH and LH show that these hormones do not achieve steady-state until many weeks after treatment. Because testosterone concentrations arc negatively inhibited by FSH and LG, testosterone levels do not achieve true steady state until these other hormones also achieve steady state. However, because these hormones regulate only endogenous testosterone (which is small to begin with in hypogonadal men) in an intact feedback mechanism (which may not be present depending on the cause of hypogonadism), the level of FSH and/or LH may have little effect on the actual testosterone levels achieved. The net result is that the patients do not achieve a “hormonal steady state” for testosterone even though the C_{avg} , Cₘᵢₙ, and Cₘₐₓ for testosterone remains relative constant after a few days of treatment.

Bone Mineral Density (“BMD”) and Similar Markers

BMD

BMD was assessed by dual energy X-ray absorptiometry (“DEXA”) using Hologic QDR 2000 or 4500 A (Hologic, Waltham, Mass.) on days 0 and 180 in the lumbar spine and left hip regions. BMD of spine was calculated as the average of BMD at L1 to L4. BMD of the left hip, which included Ward's triangle, was calculated by the average of BMD from neck, trochanter, and intertrochanter regions. The scans were centrally analyzed and processed at Hologic. BMD assessments were performed at 13 out of the 16 centers (206 out of 227 subjects) because of the lack of the specific DEXA equipment at certain sites.

Table 37 and FIG. Nos. 21(a)-14(b) show that before treatment, the BMD of the hip or the spine was not different among the three treatment groups. Significant increases in BMD occurred only in subjects in the AndroGel® 10.0 g/day group and those who switched from AndroGel® 10.0 to 7.5 g/day groups. The increases in BMD were about 1% in the hip and 2% in the spine during the six-month period. Average increases in BMD of 0.6% and 1% in the hip and spine were seen in those receiving 5.0 g/day of AndroGel® but no increase was observed in the testosterone patch group.

TABLE 37
BMD Concentrations on Day 0 and Day 180
by Final Treatment Group Mean (± SD)

| Final | | | | | | % Change from |
|---|---|---|---|---|---|---|
| Treatment Group | N | Day 0 | N | Day 180 | N | Day 0 to Day 180 |
| Hip | | | | | | |
| 5.0 g/day T-gel | 50 | 1.026 ± 0.145 | 41 | 1.022 ± 0.145 | 41 | 0.7 ± 2.1 |
| 5.0 to 7.5 g/day T-gel | 16 | 1.007 ± 0.233 | 15 | 1.011 ± 0.226 | 15 | 1.0 ± 4.9 |
| 10.0 to 7.5 g/day T-gel | 20 | 1.002 ± 0.135 | 19 | 1.026 ± 0.131 | 19 | 1.3 ± 2.4 |
| 100 g/day T-gel | 53 | 0.991 ± 0.115 | 44 | 0.995 ± 0.130 | 44 | 1.1 ± 1.9 |
| T-Patch | 67 | 0.982 ± 0.166 | 37 | 0.992 ± 0.149 | 37 | −0.2 ± 2.9 |
| Spine | | | | | | |
| 5.0 g/day T-gel | 50 | 1.066 ± 0.203 | 41 | 1.072 ± 0.212 | 41 | 1.0 ± 2.9 |
| 5.0 to 7.5 g/day T-gel | 16 | 1.060 ± 0.229 | 15 | 1.077 ± 0.217 | 15 | 0.4 ± 5.5 |
| 10.0 to 7.5 g/day T-gel | 19 | 1.049 ± 0.175 | 19 | 1.067 ± 0.175 | 18 | 1.4 ± 3.2 |
| 10.0 g/day T-gel | 53 | 1.037 ± 0.126 | 44 | 1.044 ± 0.124 | 44 | 2.2 ± 3.1 |
| T-Patch | 67 | 1.058 ± 0.199 | 36 | 1.064 ± 0.205 | 36 | −0.2 ± 3.4 |

Note:
Day 0 and Day 180 are arithmetic means, while percent change is a geometric mean.

The baseline hip and spine BMD and the change in BMD on day 180 were not significantly correlated with the average serum testosterone concentration on day 0. The changes in BMD in the hip or spine after testosterone replacement were not significantly different in subjects with hypogonadism due to primary, secondary, aging, or unclassified causes; nor were they different between naive and previously testosterone replaced subjects. The change in BMD in the spine was negatively correlated with baseline BMD values, indicating that the greatest increase in BMD occurred in subjects with the lowest initial BMD. The increase in BMD in the hip (but not in the spine) after testosterone treatment was correlated with the change in serum testosterone levels.

Bone Osteoblastic Activity Markers

The results described above are supported by measurements of a number of serum and urine markers of bone formation. Specifically, the mean concentrations of the serum markers (PTH, SALP, osteocalcin, type I procollagen) generally increase during treatment in all treatment groups. In addition, the ratios of two urine markers of bone formation (N-telopeptide/creatinine ratio and calcium/creatinine ratio) suggests a decrease in bone resorption.

PTH (Parathyroid or Calciotropic Hormone)

Serum intact PTH was measured by two site immunoradiometric assay (“IRMA”) kits from Nichol's Institute (San Juan Capistrano, Calif.). The LLC for the PTH assay was 12.5 ng/L. The intra- and inter-assay coefficients of variation were 6.9 and 9.6%, respectively. The UCLA-Flarbor Medical Center has reported previously that the normal male adult range of PTH is 6.8 to 66.4 ng/L.

Table 38 provides the PTH concentrations over the 180-day study. FIG. No. 22 shows that the mean serum PTH levels were within the normal male range in all treatment groups at baseline. Statistically significant increases in serum PTH were observed in all subjects as a group at day 90 without inter-group differences. These increases in serum PTH were maintained at day 180 in all three groups.

TABLE 38
PTH Concentrations on Each of the Observation Days
by Final Treatment Group (Mean ± SD)

| | | 5 g/day | | 5 => 7.5 g/day | | 10 => 7.5 g/day | | 10 g/day | | |
|---|---|---|---|---|---|---|---|---|---|---|
| | N | T-gel | N | T-gel | N | T-gel | N | T-gel | N | T-Patch |
| Day 0 | 53 | 16.31 ± 8.81 | 20 | 17.70 ± 9.66 | 20 | 18.02 ± 8.18 | 58 | 14.99 ± 6.11 | 75 | 15.60 ± 6.57 |
| Day 30 | 49 | 17.91 ± 10.36 | 20 | 18.33 ± 8.02 | 20 | 17.45 ± 5.67 | 58 | 18.04 ± 8.95 | 72 | 18.33 ± 10.92 |
| Day 90 | 47 | 21.32 ± 11.47 | 20 | 21.25 ± 10.96 | 19 | 17.10 ± 6.04 | 54 | 20.01 ± 9.77 | 66 | 21.45 ± 13.71 |
| Day 120 | 46 | 21.19 ± 11.42 | 19 | 21.42 ± 13.20 | 20 | 19.62 ± 9..96 | 50 | 22.93 ± 12.57 | 46 | 21.07 ± 11.44 |
| Day 180 | 46 | 22.85 ± 12.89 | 19 | 21.34 ± 11.08 | 19 | 21.02 ± 10.66 | 51 | 25.57 ± 15.59 | 46 | 25.45 ± 16.54 |

SALP

SALP was quantitated by IRMA using reagents supplied by Hybritech (San Diego, Calif.). The LLQ for the SALP assay was 3.8 [symbol] g/L.; and the intra- and inter-assay precision coefficients were 2.9 and 6.5%, respectively. The UCLA-Harbor Medical Center reported that the adult normal male concentration of SALP ranges from 2.4 to 16.6 [symbol] g/L.

The pretreatment SALP concentrations were within the normal range. FIG. No. 23 and Table 39 show that SALP levels increased with testosterone treatment in the first 90 days and reached statistical difference in the testosterone patch group. Thereafter serum SALP plateaued in all treatment groups.

TABLE 39
SALP Concentrations on Each of the Observation Days
by Final Treatment Group (Mean ± SD)

| | | 5 g/day | | 5 => 7.5 g/day | | 10 => 7.5 | | 10 g/day | | |
|---|---|---|---|---|---|---|---|---|---|---|
| | N | T-gel | N | T-gel | N | g/day T-gel | N | T-gel | N | T-Patch |
| Day 0 | 53 | 9.96 ± 5.61 | 20 | 12.36 ± 4.62 | 20 | 10.48 ± 3.68 | 58 | 9.80 ± 3.57 | 76 | 10.44 ± 3.77 |
| Day 30 | 49 | 10.20 ± 6.77 | 20 | 11.38 ± 4.09 | 20 | 11.83 ± 4.32 | 58 | 9.93 ± 3.88 | 71 | 10.86 ± 3.75 |
| Day 90 | 47 | 11.64 ± 7.98 | 20 | 11.97 ± 5.03 | 20 | 10.97 ± 3.18 | 55 | 9.56 ± 3.12 | 65 | 11.99 ± 9.36 |
| Day 120 | 46 | 11.71 ± 7.85 | 19 | 12.12 ± 5.25 | 20 | 11.61 ± 2.58 | 48 | 9.63 ± 3.58 | 45 | 11.63 ± 4.72 |
| Day 180 | 45 | 11.12 ± 7.58 | 19 | 11.67 ± 5.35 | 19 | 11.22 ± 3.44 | 51 | 9.19 ± 2.42 | 46 | 11.47 ± 3.77 |

Osteocalcin

Serum osteocalcin was measured by an IRMA from Immutopics (San Clemente, Calif.). The LLQ was 0.45 [symbol] g/L. The intra- and inter-assay coefficients were 5.6 and 4.4%, respectively. The UCLA-Harbor Medical Center reports that the normal male adult range for the osteocalcin assay ranges from 2.9 to 12.7 [symbol] g/L.

As shown in FIG. No. 24 and Table 40, the baseline mean serum osteocalcin levels were within the normal range in all treatment groups. During the first 90-day treatment, mean serum osteocalcin increased with testosterone replacement in all subjects as a group without significant differences between the groups. With continued treatment serum osteocalcin either plateaued or showed a decrease by day 180.

TABLE 40
Osteocalcin Concentrations on Each of the Observation Days
by Final Treatment Group (Mean ± SD)

| | | 5 g/day | | 5 => 7.5 g/day | | 10 => 7.5 g/day | | 10 g/day | | |
|---|---|---|---|---|---|---|---|---|---|---|
| | N | T-gel | N | T-gel | N | T-gel | N | T-gel | N | T-Patch |
| Day 0 | 53 | 4.62 ± 1.55 | 20 | 5.01 ± 2.03 | 20 | 4.30 ± 1.28 | 58 | 4.58 ± 1.92 | 76 | 4.53 ± 1.54 |
| Day 30 | 49 | 4.63 ± 1.65 | 20 | 5.35 ± 2.06 | 20 | 4.48 ± 1.72 | 58 | 4.91 ± 2.08 | 72 | 5.17 ± 1.61 |
| Day 90 | 47 | 4.91 ± 2.15 | 20 | 5.29 ± 1.87 | 19 | 4.76 ± 1.50 | 55 | 4.83 ± 2.13 | 66 | 5.18 ± 1.53 |
| Day 120 | 46 | 4.95 ± 1.97 | 18 | 4.97 ± 1.60 | 20 | 4.71 ± 1.39 | 49 | 4.61 ± 2.01 | 47 | 4.98 ± 1.87 |
| Day 180 | 45 | 4.79 ± 1.82 | 19 | 4.89 ± 1.54 | 19 | 4.47 ± 1.49 | 51 | 3.76 ± 1.60 | 46 | 5.15 ± 2.18 |

Type I Procollagen

Serum type I procollagen was measured using a RIA kit from Incstar Corp (Stillwater, Minn.). The LLQ of the procollagen assay was 5 μg/L, and the intra- and inter-assay precisions were 6.6 and 3.6%, respectively. The UCLA-Harbor Medical Center reports that the normal adult male concentration of type I procollagen ranges from 56 to 310 μg/L.

FIG. No. 25 and Table 41 show that serum procollagen generally followed the same pattern as serum osteocalcin. At baseline the mean levels were similar and within the normal range in all treatment groups. With transdermal treatment, serum procollagen increased significantly in all subjects as a group without treatment group differences. The increase in procollagen was highest on day 30 and then plateaued until day 120. By day 180, the serum procollagen levels returned to baseline levels.

TABLE 41
Procollagen Concentrations on Each of the Observation Days
by Final Treatment Group (Mean ± SD)

| | | 5 g/day | | 5 => 7.5 g/day | | 10 => 7.5 g/day | | 10 g/day | | |
|---|---|---|---|---|---|---|---|---|---|---|
| | N | T-gel | N | T-gel | N | T-gel | N | T-gel | N | T-Patch |
| Day 0 | 53 | 115.94 ± 43.68 | 20 | 109.27 ± 32.70 | 20 | 120.93 ± 28.16 | 58 | 125.33 ± 57.57 | 76 | 122.08 ± 51.74 |
| Day 30 | 49 | 141.09 ± 64.02 | 20 | 141.41 ± 77.35 | 20 | 147.25 ± 49.85 | 58 | 149.37 ± 60.61 | 71 | 139.26 ± 59.12 |
| Day 90 | 47 | 137.68 ± 68.51 | 20 | 129.02 ± 60.20 | 29 | 144.60 ± 58.20 | 55 | 135.59 ± 51.54 | 66 | 130.87 ± 49.91 |
| Day 120 | 46 | 140.07 ± 81.48 | 19 | 133.61 ± 54.09 | 20 | 139.00 ± 64.96 | 50 | 128.48 ± 45.56 | 46 | 130.39 ± 42.22 |
| Day 180 | 45 | 119.78 ± 49.02 | 19 | 108.78 ± 35.29 | 19 | 123.51 ± 39.30 | 51 | 108.52 ± 38.98 | 45 | 120.74 ± 56.10 |

Urine Bone Turnover Markers: N-telopeptide/Cr and Ca/Cr Ratios

Urine calcium and creatinine were estimated using standard clinical chemistry procedures by an autoanalyzer operated by the UCLA-Harbor Pathology Laboratory. The procedures were performed using the COBAS MIRA automated chemistry analyzer system manufactured by Roche Diagnostics Systems. The sensitivity of the assay for creatinine was 8.9 mg/dL and the LLQ was 8.9 mg/dL. According to the UCLA-Harbor Medical Center, creatinine levels in normal adult men range from 2.1 mM to 45.1 mM. The sensitivity of the assay for calcium was 0.7 mg/dL and the LLQ was 0.7 mg/dL. The normal range for urine calcium is 0.21 mM to 7.91 mM.

N-telopeptides were measured by an enzyme-linked immunosorbant assay (“ELISA”) from Ostex (Seattle, Wash.). The LLQ for the N-telopeptide assay was 5 nM bone collagen equivalent (“BCE”). The intra- and inter-assay had a precision of 4.6 and 8.9%, respectively. The normal range for the N-telopeptide assay was 48-2529 nM BCE. Samples containing low or high serum/urine bone marker levels were reassayed after adjusting sample volume or dilution to ensure all samples would be assayed within acceptable precision and accuracy.

The normal adult male range for the N-telopeptide/Cr ratio is 13 to 119 nM BCE/nM Cr. As shown in FIG. No. 26 and Table 42, urinary N-telopeptide/Cr ratios were similar in all three treatment groups at baseline but decreased significantly in the AndroGel® 10.0 g/day group but not in the AndroGel® 5.0 g/day or testosterone patch group during the first 90 days of treatment. The decrease was maintained such that urinary N-telopeptide/Cr ratio remained lower than baseline in AndroGel® 10.0 g/day and in those subjects adjusted to 7.5 g/day from 10.0 g/day group at day 180. This ratio also decreased in the testosterone patch treatment group by day 180.

TABLE 42
N-Telopeptide/Cr Ratio on Each of the Observation Days
by Initial Treatment Group (Mean ± SD)

| Initial Treatment | | 5.0 g/day | | 10.0 g/day | | | Across-group |
|---|---|---|---|---|---|---|---|
| Group | N | T-gel | N | T-gel | N | T-Patch | p-value |
| Day 0 | 71 | 90.3 ± 170.3 | 75 | 98.0 ± 128.2 | 75 | 78.5 ± 82.5 | 0.6986 |
| Day 30 | 65 | 74.6 ± 79.3 | 73 | 58.4 ± 66.4 | 66 | 91.6 ± 183.6 | 0.3273 |
| Day 90 | 62 | 70.4 ± 92.6 | 73 | 55.2 ± 49.1 | 63 | 75.0 ± 113.5 | 0.5348 |
| Day 120 | 35 | 78.8 ± 88.2 | 36 | 46.6 ± 36.4 | 21 | 71.2 ± 108.8 | 0.2866 |
| Day 180 | 64 | 68.2 ± 81.1 | 70 | 46.9 ± 43.1 | 47 | 49.4 ± 40.8 | 0.2285 |

The normal range for Ca/Cr ratio is 0.022 to 0.745 mM/mM. FIG. No. 27 shows no significant difference in baseline urinary Ca/Cr ratios in the three groups. With transdermal testosterone replacement therapy, urinary Ca/Cr ratios did not show a significant decrease in any treatment group at day 90. With continued testosterone replacement to day 180, urinary Ca/Cr showed marked variation without significant changes in any treatment groups.

TABLE 43
Ca/Cr Ratio on Each of the Observation Days
by Initial Treatment Group (Mean ± SD)

| Initial Treatment | | 5.0 g/day | | 10.0 g/day | | | Across-group |
|---|---|---|---|---|---|---|---|
| Group | N | T-gel | N | T-gel | N | T-Patch | p-value |
| Day 0 | 71 | 0.150 ± 0.113 | 75 | 0.174 ± 0.222 | 75 | 0.158 ± 0.137 | 0.6925 |
| Day 30 | 65 | 0.153 ± 0.182 | 73 | 0.128 ± 0.104 | 66 | 0.152 ± 0.098 | 0.3384 |
| Day 90 | 63 | 0.136 ± 0.122 | 73 | 0.113 ± 0.075 | 63 | 0.146 ± 0.099 | 0.2531 |
| Day 120 | 36 | 0.108 ± 0.073 | 36 | 0.117 ± 0.090 | 21 | 0.220 ± 0.194 | 0.0518 |
| Day 180 | 64 | 0.114 ± 0.088 | 70 | 0.144 ± 0.113 | 47 | 0.173 ± 0.108 | 0.0398 |

Interestingly, the change in Ca/Cr ratio from baseline at day 90 was inversely related to the baseline Ca/Cr ratios. Similarly, the change in urine N-telopeptide/Cr ratio was also inversely proportional to the baseline N-telopeptide/Cr ratio (r=0.80, p=0.0001). Thus subjects with the highest bone resorption markers at baseline showed the largest decreases of these markers during transdermal testosterone replacement. The decreases in urinary bone resorption markers were most prominent in subjects who had highest baseline values, suggesting that hypogonadal subjects with the most severe metabolic bone disease responded most to testosterone replacement therapy.

Serum Calcium

Serum calcium showed no significant inter-group differences at baseline, nor significant changes after testosterone replacement. Serum calcium levels showed insignificant changes during testosterone replacement.

Libido, Sexual Performance, and Mood

Sexual function and mood were assessed by questionnaires the patients answered daily for seven consecutive days before clinic visits on day 0 and on days 30, 60, 90, 120, 150, and 180 days during gel and patch application. The subjects recorded whether they had sexual day dreams, anticipation of sex, flirting, sexual interaction (e.g., sexual motivation parameters) and orgasm, erection, masturbation, ejaculation, intercourse (e.g., sexual performance parameters) on each of the seven days. The value was recorded as 0 (none) or 1 (any) for analyses and the number of days the subjects noted a parameter was summed for the seven-day period. The average of the four sexual motivation parameters was taken as the sexual motivation score and that of the five sexual motivation parameters as the sexual motivation mean score (0 to 7). The subjects also assessed their level of sexual desire, sexual enjoyment, and satisfaction of erection using a seven-point Likert-type scale (0 to 7) and the percent of full erection from 0 to 100%. The subjects rated their mood using a 0 to 7 score. The parameters assessed included positive mood responses: alert, friendly, full of energy, well/good feelings and negative mood responses: angry, irritable, sad, tired, nervous. Weekly average scores were calculated. The details of this questionnaire had been described previously and are fully incorporated by reference. See Wang et al., Testosterone Replacement Therapy Improves Mood in Hypogonadal Men—A Clinical Research Center Study, 81 J. CLINICAL ENDOCRINOLOGY & METABOLISM 3578-3583 (1996).

Libido

As shown in FIG. No. 28(a), at baseline, sexual motivation was the same in all treatment groups. After transdermal testosterone treatment, overall sexual motivation showed significant improvement. The change in the summary score from baseline, however, was not different among the three treatment groups.

Libido was assessed from responses on a linear scale of: (1) overall sexual desire, (2) enjoyment of sexual activity without a partner, and (3) enjoyment of sexual activity with a partner. As shown in FIG. No. 28(b) and Table 44, as a group, overall sexual desire increased after transdermal testosterone treatment without inter-group difference. Sexual enjoyment with and without a partner (FIG. No. 28(c) and Tables 45 and 26) also increased as a group.

Similarly the sexual performance score improved significantly in all subjects as a group. The improvement in sexual performance from baseline values was not different between transdermal preparations.

TABLE 44
Overall Sexual Desire
Changes From Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gel | 69 | 2.1 ± 1.6 | 63 | 3.5 ± 1.6 | 60 | 1.4 ± 1.9 | 0.0001 |
| 10.0 g/day T-gel | 77 | 2.0 ± 1.4 | 68 | 3.6 ± 1.6 | 67 | 1.5 ± 1.9 | 0.0001 |
| T-Patch | 72 | 2.0 ± 1.6 | 47 | 3.1 ± 1.9 | 45 | 1.6 ± 2.1 | 0.0001 |
| Across-Groups | | 0.8955 | | 0.2247 | | 0.8579 | |
| p-value | | | | | | | |

TABLE 45
Level of Sexual Enjoyment Without a Partner
Changes From Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gel | 60 | 1.5 ± 1.9 | 51 | 1.9 ± 1.9 | 44 | 0.8 ± 1.4 | 0.0051 |
| 10.0 g/day T-gel | 63 | 1.2 ± 1.4 | 53 | 2.2 ± 1.9 | 48 | 1.1 ± 1.6 | 0.0001 |
| T-Patch | 66 | 1.4 ± 1.8 | 44 | 2.2 ± 2.3 | 40 | 1.0 ± 1.9 | 0.0026 |
| Across-Groups | | 0.6506 | | 0.7461 | | 0.6126 | |
| p-value | | | | | | | |

TABLE 46
Level of Sexual Enjoyment With a Partner
Change from Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gel | 64 | 2.1 ± 2.1 | 55 | 2.6 ± 2.2 | 48 | 0.4 ± 2.2 | 0.0148 |
| 10.0 g/day T-gel | 66 | 1.8 ± 1.7 | 58 | 3.0 ± 2.2 | 52 | 1.0 ± 2.3 | 0.0053 |
| T-Patch | 61 | 1.5 ± 1.7 | 40 | 2.2 ± 2.4 | 35 | 0.7 ± 2.3 | 0.1170 |
| Across-Groups | | 0.2914 | | 0.1738 | | 0.3911 | |
| p-value | | | | | | | |

Sexual Performance

FIG. No. 29(a) shows that while all treatment groups had the same baseline sexual performance rating, the rating improved with transdermal testosterone treatment in all groups. In addition, as a group, the subjects' self-assessment of satisfaction of erection (FIG. No. 29(b) and Table 47) and percent full erection (FIG. No. 29(c) and Table 48) were also increased with testosterone replacement without significant differences between groups.

The improvement in sexual function was not related to the dose or the delivery method of testosterone. Nor was the improvement related to the serum testosterone levels achieved by the various testosterone preparations. The data suggest that once a threshold (serum testosterone level probably at the low normal range) is achieved, normalization of sexual function occurs. Increasing serum testosterone levels higher to the upper normal range does not further improve sexual motivation or performance.

TABLE 47
Satisfaction with Duration of Erection
Change from Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gel | 55 | 2.5 ± 2.1 | 57 | 4.3 ± 1.8 | 44 | 1.9 ± 2.0 | 0.0001 |
| 10/0 g/day T-gel | 64 | 2.9 ± 1.9 | 58 | 4.5 ± 1.7 | 53 | 1.5 ± 2.0 | 0.0001 |
| T-Patch | 45 | 3.4 ± 2.1 | 34 | 4.5 ± 2.0 | 20 | 1.3 ± 2.1 | 0.0524 |
| Across-Groups | | 0.1117 | | 0.7093 | | 0.5090 | |
| p-value | | | | | | | |

TABLE 48
Percentage of Full Erection
Change from Day 0 to Day 180
by Initial Treatment Group (Mean ± SD)

| Initial Treatment | | | | | | Change From | Within-Group |
|---|---|---|---|---|---|---|---|
| Group | N | Day 0 | N | Day 180 | N | Day 0 to Day 180 | p-value |
| 5.0 g/day T-gel | 53 | 53.1 ± 24.1 | 57 | 67.4 ± 22.5 | 43 | 18.7 ± 22.1 | 0.0001 |
| 10.0 g/day T-gel | 62 | 59.6 ± 22.1 | 59 | 72.0 ± 20.2 | 52 | 10.4 ± 23.4 | 0.0001 |
| T-Patch | 47 | 56.5 ± 24.7 | 33 | 66.7 ± 26.7 | 19 | 12.7 ± 20.3 | 0.0064 |
| Across-Groups | | 0.3360 | | 0.4360 | | 0.1947 | |
| p-value | | | | | | | |

Mood

The positive and negative mood summary responses to testosterone replacement therapy are shown in FIG. Nos. 30(a) and 30(b). All three treatment groups had similar scores at baseline and all subjects as a group showed improvement in positive mood. Similarly, the negative mood summary scores were similar in the three groups at baseline and as a group the responses to transdermal testosterone applications showed significant decreases without showing between group differences. Specifically, positive mood parameters, such as sense of well being and energy level, improved and negative mood parameters, such as sadness and irritability, decreased. The improvement in mood was observed at day 30 and was maintained with continued treatment. The improvement in mood parameters was not dependent on the magnitude of increase in the serum testosterone levels. Once the serum testosterone increased into the low normal range, maximal improvement in mood parameters occurred. Thus, the responsiveness in sexual function and mood in hypogonadal men in response to testosterone therapy appeared to be dependent on reaching a threshold of serum testosterone at the low normal range.

Muscle Strength

Muscle strength was assessed on days 0, 90, and 180. The one-repetitive maximum (“1-RM”) technique was used to measure muscle mass in bench press and seated leg press exercises. The muscle groups tested included those in the hips, legs, shoulders, arms, and chest. The 1-RM technique assesses the maximal force generating capacity of the muscles used to perform the test. After a 5-10 minute walking and stretching period, the test began with a weight believed likely to represent the patient's maximum strength. The test was repeated using increments of about 2-10 pounds until the patient was unable to lift additional weight with acceptable form Muscle strength was assessed in 167 out of the 227 patients. Four out of 16 centers did not participate in the muscle strength testing because of lack of the required equipment.

The responses of muscle strength testing by the arm/chest and leg press tests are shown in FIG. No. 31(a) and 31(b) and Table 49. There were no statistical significant differences in arm/chest or leg muscle strength among the three groups at baseline. In general, muscle strength improved in both the arms and legs in all three treatment groups without inter-group differences at both day 90 and 180. The results showed an improvement in muscle strength at 90 and 180 days, more in the legs than the arms, which was not different across treatment groups nor on the different days of assessment. Adjustment of the dose at day 90 did not significantly affect the muscle strength responses to transdermal testosterone preparations.

TABLE 49
Muscle Strength-Days 0, 90, and 180 Levels and Change (lbs.)
from Day 0 to Day 90 and from Day 0 to Day 180
by Final Treatment Group

| | | | | Arm/Chest | |
|---|---|---|---|---|---|
| | | Seated Leg Press | | (Bench Press) | |
| Final Treatment | | | Mean ± SD | | Mean ± SD |
| Group | Study Day | N | (lbs.) | N | (lbs.) |
| 5.0 g/day T-gel | 0 | 37 | 356.8 ± 170.0 | 37 | 100.5 ± 37.4 |
| | 90 | 30 | 396.4 ± 194.3 | 31 | 101.2 ± 30.7 |
| | 0-90 | 30 | 25.8 ± 49.2 | 31 | 4.0 ± 10.0 |
| | 180 | 31 | 393.4 ± 196.6 | 31 | 99.7 ± 31.4 |
| | 0-180 | 31 | 19.9 ± 62.4 | 31 | 1.3 ± 13.0 |
| 7.5 g/day T-gel | 0 | 16 | 302.8 ± 206.5 | 16 | 102.8 ± 48.9 |
| (from 5.0 g/day) | 90 | 15 | 299.8 ± 193.9 | 15 | 109.5 ± 47.6 |
| | 0-90 | 15 | 17.0 ± 88.4 | 15 | 5.0 ± 21.3 |
| | 180 | 14 | 300.6 ± 203.0 | 14 | 108.5 ± 49.3 |
| | 0-180 | 14 | −0.1 ± 110.2 | 14 | 5.6 ± 30.4 |
| 7.5 g/day T-gel | 0 | 14 | 363.4 ± 173.8 | 14 | 123.3 ± 54.7 |
| (From 10.0 g/day) | 90 | 14 | 401.6 ± 176.6 | 14 | 134.6 ± 57.5 |
| | 0-90 | 14 | 38.2 ± 42.9 | 14 | 11.3 ± 10.5 |
| | 180 | 12 | 409.9 ± 180.2 | 14 | 132.3 ± 61.5 |
| | 0-180 | 12 | 33.9 ± 67.3 | 14 | 9.0 ± 18.7 |
| 10.0 g/day T-gel | 0 | 45 | 345.9 ± 186.9 | 43 | 114.7 ± 55.1 |
| | 90 | 43 | 373.5 ± 194.8 | 41 | 119.8 ± 54.2 |
| | 0-90 | 43 | 27.6 ± 45.1 | 41 | 4.6 ± 12.8 |
| | 180 | 36 | 364.4 ± 189.1 | 34 | 112.0 ± 45.5 |
| | 0-180 | 36 | 32.2 ± 72.3 | 34 | 1.9 ± 14.8 |
| T-Patch | 0 | 55 | 310.4 ± 169.7 | 54 | 99.2 ± 43.1 |
| | 90 | 46 | 344.9 ± 183.9 | 46 | 106.2 ± 44.0 |
| | 0-90 | 46 | 25.4 ± 37.0 | 46 | 3.2 ± 12.0 |
| | 180 | 36 | 324.8 ± 199.0 | 35 | 104.8 ± 44.8 |
| | 0-180 | 36 | 15.2 ± 54.7 | 35 | 2.3 ± 15.7 |

Body Composition

Body composition was measured by DEXA with Hologic 2000 or 4500A series on days 0, 90, and 180. These assessments were done in 168 out of 227 subjects because the Hologic DEXA equipment was not available at 3 out of 16 study centers. All body composition measurements were centrally analyzed and processed by Hologic (Waltham, Mass.).

At baseline, there were no significant differences in total body mass (“TBM”), total body lean mass (“TLN”), percent fat (“PFT”), and total body fat mass (“TFT”) in the three treatment groups. As shown in FIG. Nos. 32(a) and Table 50, all treatment groups incurred an overall increase in TBM. The increase in TBM was mainly due to the increases in TLN. FIG. No. 32(b) and Table 50 show that after 90 days of testosterone replacement the increase in TLN was significantly higher in the 10.0 g/day AndroGel® group than in the other two groups. At day 180, the increases in TLN were further enhanced or maintained in all AndroGel® treated groups, as well as in the testosterone patch group.

FIG. Nos. 32(c) and (d) show that the TFT and the PFT decreased in all transdermal AndroGel® treatment groups. At 90 days of treatment, TFT was significantly reduced by [in] the 5.0 g/day and 10.0 g/day AndroGel® groups, but was not changed in the testosterone patch group. This decrease was maintained at day 180. Correspondingly, at day 90 and 180, the decrease in PFT remained significantly lower in all AndroGel® treated groups but not significantly reduced in the testosterone patch group.

The increase in TLN and the decrease in TFT associated with testosterone replacement therapy showed significant correlations with the serum testosterone level attained by the testosterone patch and the different doses of AndroGel®. Testosterone gel administered at 10.0 g/day increased lean mass more than the testosterone patch and the 5.0 g/day AndroGel® groups. The changes were apparent on day 90 after treatment and were maintained or enhanced at day 180. Such changes in body composition was significant even though the subjects were withdrawn from prior testosterone therapy for six weeks. The decrease in TFT and PFT was also related to the serum testosterone achieved and were different across the treatment groups. The testosterone patch group did not show a decrease in PFT or TFT after 180 days of treatment. Treatment with AndroGel® (5.0 to 10.0 g/day) for 90 days reduced PFT and TFT. This decrease was maintained in the 5.0 and 7.5 g/day groups at 180 days but were further lowered with continued treatment with the higher dose of the AndroGel®.

TABLE 50
Mean Change in Body Composition Parameters (DEXA)
From Baseline to Day 90 and Baseline to Day 180
By Final Treatment Groups

| Final Treatment Group | | N | TFT (g) | TLN (g) | TBM (g) | PFT |
|---|---|---|---|---|---|---|
| | | Mean Change from Day 0-Day 90 | | | | |
| 5.0 | g/day | 43 | −782 ± 2105 | 1218 ± 2114 | 447 ± 1971 | −1.0 ± 2.2 |
| | T-gel | | | | | |
| 7.5 | g/day | 12 | −1342 ± 3212 | 1562 ± 3321 | 241 ± 3545 | −1.0 ± 3.1 |
| | (from | | | | | |
| 5.0 | g/day) | | | | | |
| 7.5 | g/day | 16 | −1183 ± 1323 | 3359 ± 2425 | 2176 ± 2213 | −2.0 ± 1.5 |
| | (from | | | | | |
| 10.0 | g/day) | | | | | |
| 10.0 | g/day | 45 | −999 ± 1849 | 2517 ± 2042 | 1519 ± 2320 | −1.7 ± 1.8 |
| | T-gel | | | | | |
| | T-Patch | 52 | 11 ± 1769 | 1205 ± 1913 | 1222 ± 2290 | −0.4 ± 1.6 |
| | | Mean Change from Day 0-Day 180 | | | | |
| 5.0 | g/day | 38 | −972 ± 3191 | 1670 ± 2469 | 725 ± 2357 | −1.3 ± 3.1 |
| | T-gel | | | | | |
| 7.5 | g/day | 13 | −1467 ± 3851 | 2761 ± 3513 | 1303 ± 3202 | −1.5 ± 3.9 |
| | (from | | | | | |
| 5.0 | g/day) | | | | | |
| 7.5 | g/day | 16 | −1333 ± 1954 | 3503 ± 1726 | 2167 ± 1997 | −2.2 ± 1.7 |
| | (from | | | | | |
| 10.0 | g/day) | | | | | |
| 10.0 | g/day | 42 | −2293 ± 2509 | 3048 ± 2284 | 771 ± 3141 | −2.9 ± 2.1 |
| | T-gel | | | | | |
| | T-Patch | 34 | 293 ± 2695 | 997 ± 2224 | 1294 ± 2764 | −0.3 ± 2.2 |

Lipid Profile and Blood Chemistry

The serum total, HDL, and LDL cholesterol levels at baseline were not significantly different in all treatment groups. With transdermal testosterone replacement, there were no overall treatment effects nor inter-group differences in serum concentrations of total, HDL- and LDL-cholesterol (FIG. 12(d)) and triglycerides (data not shown). There was a significant change of serum total cholesterol concentrations as a group with time (p=0.0001), the concentrations on day 30, 90, and 180 were significantly lower than day 0.

Approximately 70 to 95% of the subjects had no significant change in their serum lipid profile during testosterone replacement therapy. Total cholesterol levels which were initially high were lowered into the normal range (of each center's laboratory) at day 180 in 17.2, 20.4, and 12.2% of subjects on testosterone patch, AndroGel® 5.0 g/day and AndroGel® 10.0 g/day, respectively. Serum HDL-cholesterol levels (initially normal) were reduced to below the normal range (of each center's laboratory) in 9.8, 4.0, 9. 1, and 12.5% of subjects at day 180 in the testosterone patch, AndroGel® 5.0, 7.5, and 10.0 g/day groups, respectively. There was no clinically significant changes in renal or liver function tests in any treatment group.

Skin Irritations

Skin irritation assessments were performed at every clinic visit using the following scale: 0=no erythema; 1=minimal erythema; 2=moderate erythema with sharply defined borders; 3=intense erythema with edema; and 4=intense erythema with edema and blistering/erosion.

Tolerability of the daily application of AndroGel® at the tested dosages was much better than with the permeation-enhanced testosterone patch. Minimal skin irritation (erythema) at the application site was noted in three patients in the AndroGel® 5.0 g/day group (5.7%) and another three in the AndroGel® 10.0 g/day group (5.3%). Skin irritation varying in intensity from minimal to severe (mild erythema to intense edema with blisters) occurred in 65.8% of patients in the patch group. Because of the skin irritation with the testosterone patch, 16 subjects discontinued the study; 14 of these had moderate to severe skin reactions at the medication sites. No patients who received AndroGel® discontinued the study because of adverse skin reactions. The open system and the lower concentration of alcohol in the AndroGel® formulation markedly reduced skin irritation resulting in better tolerability and continuation rate on testosterone replacement therapy.

Moreover, based on the difference in the weight of the dispensed and returned AndroGel® bottles, the mean compliance was 93.1% and 96.0% for the 5.0 g/day and 10.0 g/day AndroGel® groups during days 1-90, respectively. Compliance remained at over 93% for the three AndroGel® groups from days 91-180. In contrast, based on counting the patches returned by the subjects, the testosterone patch compliance was 65% during days 1-90 and 74% during days 91-180. The lower compliance in the testosterone patch group was mostly due to skin reactions from the subjects' records.

TABLE 51

Incidence of Skin-Associated Adverse Events: Day 1 to Day 180
in Patients Who Remained on Initial Treatment

| | 5.0 g/day T-gel | | 10.0 g/day T-gel | | T-Patch | |
|---|---|---|---|---|---|---|
| | N = 53 | | N = 57 | | N = 73 | |
| Total | 16 | (30.2%) | 18 | (31.6%) | 50 | (68.5%) |
| Application Site | 3 | (5.7% | 3 | (5.3%) | 48 | (65.8%) |
| Reaction | | | | | | |
| Acne | 1 | (1.9%) | 7 | (12.3%) | 3 | (4.1%) |
| Rash | 4 | (7.5%) | 4 | (7.0%) | 2 | (2.7%) |
| Skin Disorder | 2 | (3.8%) | 1 | (1.8%) | 1 | (1.4%) |
| Skin Dry | 2 | (3.8) | 0 | (0.0%) | 1 | (1.4%) |
| Sweat | 0 | (0.0%) | 2 | (3.5%) | 0 | (0.0%) |
| Reaction Unevaluable | 2 | (3.6%) | 1 | (1.7%) | 0 | (0.0%) |
| Cyst | 0 | (0.0%) | 0 | (0.0%) | 2 | (2.7%) |

Glucose Serum Concentration

Table 52 shows the glucose concentration of patients whose serum glucose concentration was greater than 100 mg/dl at the beginning of the study for each of the observation days by the final treatment group.

TABLE 52
Glucose Concentrations of Patients (Mean; mg/dL)

| | | 5 g/day | | 5 => 7.5 g/day | | 10 => 7.5 g/day | | 10 g/day | | |
|---|---|---|---|---|---|---|---|---|---|---|
| | N | T-gel | N | T-gel | N | T-gel | N | T-gel | N | T-Patch |
| Day 1 | 14 | 161.9 | 5 | 208.6 | 4 | 172 | 18 | 158.3 | 20 | 148.6 |
| Day 30 | 14 | 148.7 | 5 | 223.4 | 4 | 108.3 | 18 | 123.5 | 20 | 129.4 |
| Day 90 | 14 | 145.1 | 5 | 197.0 | 4 | 111.8 | 18 | 119.1 | 20 | 141.1 |
| Day 120 | 14 | 147.0 | 5 | 187.0 | 4 | 156.5 | 18 | 131.6 | 13 | 146.5 |
| Day 180 | 14 | 154.4 | 5 | 214.6 | 4 | 134.8 | 18 | 132.0 | 13 | 134.1 |

Table 53 shows overall glucose change of patients whose serum glucose concentration was greater than 110 mg/dl at the start of the study from day 0 to day 180 by initial treatment group.

TABLE 53
Overall Glucose Changes of Patients
(Mean; mg/dL)

| Initial Treatment | | | | | Change From |
|---|---|---|---|---|---|
| Group | N | Day 1 | N | Day 180 | Day 0 to Day 180 |
| 5.0 g/day T-gcl | 19 | 174.2 | 19 | 170.3 | −3.9 |
| 10.0 g/day T-gel | 22 | 160.8 | 22 | 132.5 | −28.3 |
| T-Patch | 20 | 148.6 | 13 | 146.5 | −2.1 |

Table 54 shows the mean overall glucose change for patients from day 0 to day 180.

TABLE 54
Mean Overall Glucose
(Mean; mg/dL)

| Initial Treatment | | | | | | |
|---|---|---|---|---|---|---|
| Group | N | Day 1 | N | Day 90 | N | Day 180 |
| 5.0 g/day T-gcl | 69 | 119.8 | 69 | 115.1 | 54 | 111.7 |
| 7.5 g/day T-gel | NA | NA | NA | NA | 40 | 121.3 |
| 10.0 g/day T-gel | 75 | 111.4 | 75 | 99.0 | 56 | 100.3 |
| T-Patch | 71 | 110.3 | 68 | 108.2 | 71 | 107.8 |

### Example 2

### Gel Delivery Dosage Forms and Devices

The present invention is also directed to a method for dispensing and packaging the gel. In one embodiment, the invention comprises a hand-held pump capable of delivering about 2.5 g of testosterone gel with each actuation. In another embodiment, the gel is packaged in foil packets comprising a polyethylene liner. Each packet holds about 2.5 g of testosterone gel. The patient simply tears the packet along a perforated edge to remove the gel. However, because isopropyl myristate binds to the polyethylene liner, additional isopropyl myristate is added to the gel in order to obtain a pharmaceutically effective gel when using this delivery embodiment. Specifically, when dispensing the gel via the foil packet, about 41% more isopropyl myristate is used in the gel composition (i.e., about 0.705 g instead of about 0.5 g in Table 5), to compensate for this phenomenon.

### Example 11

### Method of Treating Men Having Erectile Dysfunction in Conjunction with other Pharmaceuticals

As discussed above, transdermal application of testosterone using AndroGel® to hypogonadal men results in improved libido and sexual performance. This example is directed use of AndroGel in combination with pharmaceuticals useful for treating erectile dysfunction. Such pharmaceuticals include any agent that is effective to inhibit the activity of a phosphodiesterase. Suitable phosphodiesterase inhibitors include, but are not limited to, inhibitors of the type III phosphodiesterase (cAMP-specific-cGMP inhibitable form), the type IV phospodiesterase (high affinity-high specificity cAMP form) and the type V phosphodiesterase (the cGMP specific form). Additional inhibitors that may be used in conjunction with the present invention are cGMP-specific phosphodiesterase inhibitors other than type V inhibitors.

Examples of type III phospodiesterase inhibitors that may be administered include, but are not limited to, bypyridines such as milrinone and amirinone, imidazolones such as piroximone and enoximone, dihydropyridazinones such as imazodan, 5-methyl-imazodan, indolidan and ICI1118233, quinolinone compounds such as cilostamide, cilostazol and vesnarinone, and other molecules such as bemoradan, anergrelide, siguazodan, trequinsin, pimobendan, SKF-94120, SKF-95654, lixazinone and isomazole.

Examples of type IV phosphodiesterase inhibitors suitable herein include, but are not limited to, rolipram and rolipram derivatives such as RO20-1724, nitraquazone and nitraquazone derivatives such as CP-77059 and RS-25344-00, xanthine derivatives such as denbufylline and ICI63197, and other compounds such as EMD54622, LAS-31025 and etazolate.

Examples of type V phosphodiesterase inhibitors include, but are not limited to, zaprinast, MY5445, dipyridamole, and sildenafil. Other type V phosphodiesterase inhibitors are disclosed in PCT Publication Nos. WO 94/28902 and WO 96/16644. In the preferred embodiment, an inhibitor of phosphodiesterase type 5 (“PDE5”), such as VIAGRA® (sildenafil citrate USP) is used.

The compounds described in PCT Publication No. WO 94/28902 are pyrazolopyrimidinones. Examples of the inhibitor compounds include 5-(2-ethoxy-5-morpholinoacetylphenyl)-1-methyl-3-n-propyl-1,6-dihydro-7H-p yrazolo[4,3-d]pyrimidin-7-one, 5-(5-morpholinoacetyl-2-n-propoxyphenyl)-1-methyl-3-n-propyl-1,6-dihydro-7-H-pyrazolo[4,3-d]pyrimidin-7-one, 5-[2-ethoxy-5-(4-methyl-1-piperazinylsulfonyl)-phenyl]1-methyl-3-n-propyl-1,6-dihydro-7H-pyrazolo[4,3-d]pyrimidin-7-one, 5-[2-allyloxy-5-(4-methyl-1-piperazinylsulfonyl)-phenyl]-1-methyl-3-n-propyl-1,6-dihydro-7H-pyrazolo[4,3-d]pyrimidin-7-one, 5-[2-ethoxy-5-[4-(2-propyl)-1-piperazinylsulfonyl)-phenyl]-1-methyl-3-n-propyl-1,6-dihydro-7H-pyrazolo[4,3-d]pyrimidin-7-one, 5-[2-ethoxy-5-[4-(2-hydroxyethyl)-1-piperazinylsulfonyl)phenyl]-1-methyl-3-n-propyl-1,6-dihydro-7H-pyrazolo[4,3-d]pyrimidin-7-one, 5-[5-[4-(2-hydroxyethyl)-1-piperazinylsulfonyl]-2-n-propoxyphenyl]-1-methy 1-3-n-propyl-1,6-dihydro-7H-pyrazolo[4,3-d]pyrimidin-7-one, 5[2-ethoxy-5-(4-methyl-1-piperazinylcarbonyl)phenyl]-1-methyl-3-n-propyl-1,6-dihydro-7H-pyrazolo[4,3-d]pyrimidin-7-one, and 5-[2-ethoxy-5-(1-methyl-2-imidazolyl)phenyl]-1-methyl-3-n-propyl-1,6-dihyd ro-7H-pyrazolo[4,3-d]pyrimidin-7-one.

The phosphodiesterase inhibitors described in PCT Publication No. WO 96/16644 include griseolic acid derivatives, 2-phenylpurinone derivatives, phenylpyridone derivatives, fused and condensed pyrimidines, pyrimidopyrimidine derivatives, purine compounds, quinazoline compounds, phenylpyrimidinone derivative, imidazoquinoxalinone derivatives or aza analogues thereof, phenylpyridone derivatives, and others. Specific examples of the phosphodiesterase inhibitors disclosed in WO 96/16644 include 1,3-dimethyl-5-benzylpyrazolo[4,3-d]pyrimidine-7-one, 2-(2-propoxyphenyl)-6-purinone, 6-(2-propoxyphenyl)-1,2-dihydro-2-oxypyridine-3-carboxamide, 2-(2-propoxyphenyl)-pyrido[2,3-d]pyrimid-4(3H)-one, 7-methylthio-4-oxo-2-(2-propoxyphenyl)-3,4-dihydro-pyrimido[4,5-d]pyrimidi ne, 6-hydroxy-2-(2-propoxyphenyl)pyrimidine-4-carboxamide, 1-ethyl-3-methylimidazo[1,5a]quinoxalin-4(5H)-one, 4-phenylmethylamino-6-chloro-2-(1-imidazoloyl)quinazoline, 5-ethyl-8-[3-(N-cyclohexyl-N-methylcarbamoyl)-propyloxy]-4,5-dihydro-4-oxo-pyrido[3,2-e]-pyrrolo[1,2-a]pyrazine, 5′-methyl-3′-(phenylmethyl)-spiro[cyclopentane-1,7′(8′H)-(3′H)-imidazo[2,1 b]purin]4′(5′H)-one, 1-[6-chloro-4-(3,4-methylenedioxybenzyl)-aminoquinazolin-2-yl)piperidine-4-carboxylic acid, (6R, 9S)-2-(4-trifluoromethyl-phenyl)methyl-5-methyl-3,4,5,6a,7,8,9,9a-octahydr ocyclopent[4,5]-midazo[2,1-b]-purin-4-one, 1t-butyl-3-phenylmethyl-6-(4-pyridyl)pyrazolo[3,4-d]-pyrimid-4-one, 1-cyclopentyl-3 -methyl-6-(4-pyridyl)-4,5-dihydro-1H-pyrazolo[3,4-d]pyrimid-4-one, 2-butyl-1-(2-chlorobenzyl)6-ethoxy-carbonylbenzimidaole, and 2-(4-carboxypiperidino)-4-(3,4-methylenedioxy-benzyl)amino-6-nitroquinazol ine, and 2-phenyl-8-ethoxycycloheptimidazole.

Still other type V phosphodiesterase inhibitors useful in conjunction with the present invention include: IC-351 (ICOS); 4-bromo-5-(pyridylmethylamino)-6-[3-(4-chlorophenyl)propoxy]-3(2H)pyridazi none; 1-[4-[(1,3-benzodioxol-5-ylmethyl)amiono]-6-chloro-2-quinazolinyl]-4-piper idine-carboxylic acid, monosodium salt; (+)-cis-5,6a,7,9,9,9a-hexahydro-2-[4-(trifluoromethyl)-phenymmethyl-5-meth yl-cyclopent-4,5]imidazo[2,1-9 purin-4(3H)one; furazlocillin; cis-2-hexyl-5-methyl-3,4,5,6a,7,8,9,9a-octahydrocyclopent[4,5]imidazo[2,1-b]purin-4-one; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; 1-methyl-5 -(5 -morpholinoacetyl-2-n-propoxypheniyl)-3-ni-propyl-1 ,6-dihydro-7 H-pyrazolo(4,3-d)pyrimidin-7-one; 1-[4-[(1,3-benzodioxol-5-ylmethyl)amino]-6-chloro-2-quinazolinyl]-4-piperi dinecarboxylic acid, monosodium salt; Pharmaprojects No. 4516 (Glaxo Wellcome); Pharmaprojects No. 5051 (Bayer); Pharniaprojects No. 5064 (Kyowa Hakko; see WO 96/26940); Pharmaprojects No. 5069 (Schering Plough); GF-196960 (Glaxo Wellcome); and Sch-51866.

Other phosphodiesterase inhibitors that may be used in the method of this invention include nonspecific phosphodiesterase inhibitors such as theophylline, IBMX, pentoxifylline and papaverine, and direct vasodilators such as hydralazine.

The active agents may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by J. March, Advanced Organic Chemistry, Reactions, Mechanisms and Structure, 4th Ed. (New York: Wiley-Interscience, 1992). For example, acid addition salts are prepared from the free base using conventional methodology, and involves reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of acid moieties which may be present on a phosphodiesterase inhibitor molecule are prepared in a similar manner using a pharmaceutically acceptable base such as sodium hydroxide, potassium hydroxide, ammonium hydroxide, calcium hydroxide, trimethylamine, or the like. Particularly preferred basic salts herem are alkali metal salts, e.g., the sodium salt, and copper salts. Preparation of esters involves functionalization of hydroxyl and/or carboxyl groups which may be present within the molecular structure of the drug. The esters are typically acyl-substituted derivatives of free alcohol groups, i.e., moieties which are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

Other compounds useful for treating erectile dysfunciton may also be used. These include: (a) pentoxifylline (TRENTAL®); (b) yohimbine hydrocholoride (ACTIBINE®, YOCON®, YOHIMEX®); (c) apomorphine (UPRIMA®); (d) alprostadil (the MUSE® system, TOPIGLAN®, CAVERJECT®); (e) papavaerine (PAVABID®, CERESPAN®); (f) phentolamine (VASOMAX®, REGITINE®), and combinations, salts, derivatives and enantiomers of all of the above.

A testosterone containing gel, such as AndroGel® is administered to increase and enhance the therapeutic effectiveness of such drugs, in either hypogonadal or eugonadal men having erectile dysfunction. While phamiaceuticals such as VIAGRA® work principally by various physiological mechanisms of erection initiation and maintenance, the testosterone gel used in accordance with the present invention plays a beneficial role physiologically, and stimulates both sexual motivation (i.e., libido) and sexual performance. Testosterone controls the expression of the nitric oxide synthase gene. See Reilly et al., Androgenic Regulation of NO Availability in Rat Penile Erection, 18 J. ANDROLOGY 110 (1997); Park et al., Effects of Androgens on the Expression of Nitric Oxide Synthase mRNAs in Rat Corpous Cavernosum, 83 BJU INT'L. 327 (1999). Thus, testosterone and other androgens clearly play a role in erectile dysfunction. See Lugg et al., The Role of Nitric Oxide in Erectile Function, 16 J. ANDROLOGY 2 (1995); Penson et al., Androgen and Pituitary Control of Penile Nitric Oxide Synthase and Erectile Function In the Rat, 55 BIOLOGY OF REPRODUCTION 576 (1996); Traish et al., Effects of Castration and Androgen Replacement on Erectile Function in a Rabbit Model, 140 ENDOCRINOLOGY 1861 (1999). Moreover, testosterone replacement restores nitric oxide activity. See Baba et al. Delayed Testosterone Replacement Restores Nitric Oxide Synthase Containing Nerve Fibres and the Erectile Response in Rat Penis, BJU INT'L 953 (2000); Garban et al., Restoration of Normal Adult Penile Erectile Response in Aged Rats by Long-Term Treatment with Androgens, 53 BIOLOGY OF REPRODUCTION 1365 (1995); Marin et al., Androgen-dependent Nitric Oxide Release in Rat Penis Correlates with Levels of Constitutive Nitric Oxide Synthase Isoenzymes, 61 BIOLOGY OF REPRODUCTION 1012 (1999).

As disclosed herein, adequate blood levels of testosterone are important to erection. In one embodiment, AndroGel® is applied to the body in accordance with the protocol summarized in Example 1. The pharmaceutical(s) for erectile dysfunction is taken in accordance with the prescription requirements. For example, VIAGRA® is generally taken 20-40 minutes before sexual intercourse in 50 mg doses. This combination of therapy is particularly useful in hypogonadal men who need increased testosterone levels in order to optimize the effects of VIAGRA® and the sexual experience as a whole. In essence, a synergistic effect is obtained. AndroGel® is preferably applied to the body for a sufficient number of days so that the steady-state levels of testosterone are achieved.

In a prophetic example, 10 males age 18 and older will be randomized to receive: (a) 5.0 g/day of AndroGel® (delivering 50 mg/day of testosterone to the skin of which about 10% or 5 mg is absorbed) for 30 days plus 50 mg of sildenafil citrate 1 hour before intercourse after at least 1 day of AndroGel® therapy; or (b) 10.0 g/day of AndroGel® (delivering 100 mg/day of testosterone to the skin of which about 10% or 10 mg is absorbed) for 30 days plus 50 mg of sildenafil citrate 1 hour before intercourse after at least 1 day of AndroGel® therapy; or (c) 5.0 g/day of AndroGel® (delivering 50 mg/day of testosterone) for 30 days and nothing before intercourse. Libido, erections and sexual performance will be studied as in the previous Examples. Applicant expects that all test parameters will show improvement with the combination.

### Example 12

### Method of Treating a Depressive Disorder in a Subject

An eight-week randomized placebo-controlled trial of testosterone transdermal gel (AndroGel®) was conducted with 22 treatment-refractory depressed men with low or borderline total testosterone levels (≦350 ng/dl). Testosterone gel, added to the subjects' existing antidepressant regimens, proved significantly superior to placebo in antidepressant response on the HAM-D and the CGI-severity scales, although not on the BDI.

Men age 30-65 years, presently taking an adequate dose of antidepressant medication (as defined by the manufacturer's published product information) for at least the last four weeks, but still complaining of depressive symptoms sufficient to meet DSM-IV criteria for current major depressive disorder. Subjects were initially screened. The screening was scheduled so as to have testosterone at the diurnal maximum level (prior to 10 am). Subjects were then administered the depression module the Structured Clinical Interview for DSM-IV (SCID) to confirm the diagnosis of current major depressive disorder. Subjects were next administered the American Urological Association (AUA) Symptom Index for benign prostatic hyperplasia (B PH), subjects scoring higher than 14 on this index were excluded. Blood was then collected for total testosterone and PSA levels.

Men who displayed low or borderline morning testosterone levels (100-350 ng/dl; normal range, 270-1070 ng/dl) and normal PSA levels (<1.5 ng/ml in men age 30-39, <2.5 ng/ml in men 40-49, <3.5 ng/ml in men 50-59, and <4.0 ng/ml in men 60-64) were chosen for a second screening evaluation. Next, the subjects were administered: 1) basic demographic questions; 2) the remainder of the SCID; 3) questions regarding history of previous antidepressant drug treatment; 4) the HAM-D; 5) the BDI; 6) the Clinical Global Impression Scale (CGI); 7) medical history questions; 8) physical examination, including vital signs, height, weight, and digital rectal examination of the prostate; 9) laboratory tests for standard chemistries, hematology, urinalysis, and HIV serology; 10) electrocardiogram (EKG); and 11) determination of body fat with calipers, together with calculation of fat-free mass index (FFMI), a measure of muscularity previously developed in our laboratory. Subjects were excluded if they exhibited 1) any substance use disorder within the past year (or illicit anabolic steroid use at any time in their lives); 2) current or past psychotic symptoms; 3) a history of bipolar disorder; 4) any abnormality on digital rectal examination; or 5) evidence of other clinically significant medical disease on the basis of medical history and physical examination.

Qualifying subjects were then started on a one-week single-blind placebo washout with placebo gel. All subjects were asked to continue taking their existing antidepressant medications, together with any other medications that they were prescribed, at their present dose throughout the study.

Baseline (Week 0): Subjects were assessed for scores on the HAM-D, BDI, and CGI-Severity of Illness; adverse events; and vital signs. Results from the laboratory tests drawn at screen and from EKG readings were also reviewed. Subjects were eliminated if they: a) displayed more than 50% improvement on the HAM-D or BDI after the placebo treatment; or b) were found to have a clinically significant abnormality on the laboratory tests or EKG. Subjects were then randomized to receive either 10 grams of 1% testosterone gel or placebo daily for 7 days. Drug and placebo were supplied in identical-appearing packets that contained either 2.5 g of AndroGel or a placebo gel.

Week 1: Subjects were assessed for scores on the HAM-D, BDI, and CGI (both Severity of Illness and Improvement as compared to Baseline); adverse events; and vital signs. Subjects provided blood for a total testosterone level, drawn at least four hours after the morning application of the gel.

Weeks 2, 4, 6, and 8: Subjects were assessed at weeks 2, 4,6 and 8 for HAM-D, BDI, CGI, adverse events, and vital signs. Week 8, subjects received an additional determination of PSA and measurement of weight and body fat. The blind was then broken and the correct identity of treatment assignment determined.

Subjects were eliminated prior to week 8 if they: 1) voluntarily elected to withdraw for any reason; 2) displayed an adverse event judged clinically significant by the investigators; or 3) failed to comply with the requirements of the protocol.

Statistical Analysis: Baseline characteristics of each group were compared using Fisher's exact test for categorical variables and the 1-test for continuous variables. Two populations of patients were defined: (1) an intent-to-treat group of patients with at least one available efficacy measure, and (2) a completers group, defined as patients who completed the 8-week treatment period.

The primary protocol-defined analysis of efficacy was a repeated measures random regression analysis comparing the rate of change of scores on the HAM-D, BDI, and CGI-severity during the treatment period between groups, using methods described by Diggle et al. and Gibbons et al. A model was used for the mean of the outcome variable that included terms for treatment, time, and treatment-by-time interaction. Time as a continuous variable was modeled, with weeks ranging from 0 (Baseline) to 8 (after randomization). The measure of effect was the treatment-by-time interaction (or the difference in the rate of change per unit of time, or the difference in slope with respect to time) of the efficacy measure. To account for the correlation of observations within individuals, the standard errors of the parameter estimates were calculated using generalized estimating equations, with compound symmetry as the working covariance, as implemented by the PROC GENMOD command in SAS software.

As secondary analyses of the outcome measures, two analyses of change from baseline to endpoint were used: 1) an intent-to-treat analysis, using the last observation carried forward for all subjects completing at least one post-baseline assessment; and 2) a completers analysis, using all subjects who completed 8 weeks of randomized treatment. The t-test was used to compare the difference between groups in change from baseline to endpoint on the HAM-D, BDI, and CGI-severity.

For laboratory measures, including body fat and FFMI, the mean difference between endpoint and baseline measures were used, and then compared the treatment groups using the t-test. The correlation coefficients were calculated by using rank-transformed data (Spearman rank correlation). All statistical tests were two-sided with alpha=0.05.

Recruitment and participant flow: The mean (SD) age of the subjects was 46.9 (9.2) years (range 30-65); all 56 subjects met the PSA and BPII criteria for the study described above. The men's total testosterone levels, despite being measured near their diurnal maximum, were remarkably low for their age range (1.27), with a median (interquartile range) of only 376 (301,477) ng/dl. Total testosterone levels were inversely correlated with age, but only weakly so (Spearman p=−0.25; P=0.06). Twenty-four (43.6%) of the subjects displayed levels of 350 ng/dl or less. Their median baseline total testosterone level was 292 (266,309) ng/dl. All of the remaining 22 subjects were randomized at Week 0. Of these, 3 (14%) withdrew during the follow-up period and 19 (86%) completed the full 8 weeks of the study. (FIG. No. 33)

TABLE 55
Demographic and Clinical Characteristics of Subjects at Screen

| | Randomized to | Randomized to |
|---|---|---|
| | Testosterone | Placebo |
| | (N = 12) | (N = 10) |
| Characteristic | N | N |
| Ethnicity | | |
| Caucasian | 11 | 10 |
| African-American | 1 | 0 |
| Marital Status | | |
| Married | 8 | 8 |
| Single | 2 | 1 |
| Divorced | 2 | 1 |
| Sexual Orientation | | |
| Heterosexual | 11 | 10 |
| Homosexual | 1 | 0 |
| | Mean | Mean |
| Age (years) | 48.9 | 49.5 |
| Height (cm) | 177 | 181 |
| Weight (kg) | 93.3 | 104.5 |
| Body Fat Percentage | 28.5 | 30.4 |
| Fat-Free Mass Index (kg/m2) | 21.2 | 22 |
| Prostate-Specific Antigen (ng/ml) | 0.8 | 0.8 |
| Total Testosterone Level (ng/dl) | 293 | 267 |
| Hamilton Depression Rating | 21.8 | 21.3 |
| Scale^{b} | | |
| Beck Depression Inventory^{b} | 23.1 | 23.6 |
| Clinical Global Impression - | 4.7 | 4.3 |
| Severity^{b} | | |

^{b}Represents score at baseline; all other variables are at screen

Baseline characteristics of subjects: The 12 subjects randomized to testerone did not differ significantly from the 10 randomized to placebo on attributes at screen (Table 55), except that the placebo subjects were slightly heavier than testerone subjects. The antidepressant regimens of the subjects were SSRI's (5 testerone subjects, 8 placebo subjects), buproprion (2 testosterone), bupropion plus SSRI's (2 placebo), venlafaxine (3 testosterone), nefazodone (1 testosterone), and methylphenidate (1 testosterone).

Efficacy analyses: The primary analysis of efficacy, involving all 22 subjects with at least one rating of outcome measures, revealed that testosterone-treated patients had a significantly greater rate of decrease in HAM-D scores than placebo-treated patients (FIG. No. 34). This improvement was evident on both the vegetative and affective symptoms subscales of the HAM-D (Table 28). Testosterone was also associated with significantly greater rates of decrease in CGI-severity scores (FIG. No. 35), although not BDI scores (FIG. No. 36). All rate-of-change data are summarized in Table 56. The endpoint analyses produced similar results, but with slightly less statistical power than the longitudinal analysis .

TABLE 56
Mean change (SD) on outcome measures from
baseline to endpoint, by treatment group

| | Intent to Treat^{a} | | | | Completers^{b} | | |
|---|---|---|---|---|---|---|---|
| Outcome | Placebo | | Testosterone | | Placebo | Testosterone | |
| Measure | N = 10 | | N = 11 | | N = 9 | N = 10 | |
| HAM-D, | −0.3 | (4.0) | −7.4 | (7.1) | −1.1 | −8.8 | (6.0) |
| Total score | | | | | (3.2) | | |
| HAM-D, | 0.0 | (1.5) | −2.1 | (3.4) | −0.2 | −2.7 | (2.9) |
| Affective | | | | | (1.4) | | |
| Subscale | | | | | | | |
| HAM-D, | −0.7 | (2.5) | −3.2 | (2.0) | −0.9 | −3.5 | (1.8) |
| Vegetative | | | | | (2.5) | | |
| Subscale | | | | | | | |
| BDI, | −2.0 | (5.2) | −5.5 | (8.7) | −2.4 | −6.8 | (7.8) |
| Total Score | | | | | | | |
| | | | | | (5.3) | | |
| CGI-Severity | −0.2 | (0.6) | −0.9 | (1.4) | −0.3 | −1.2 | (1.0) |
| | | | | | (0.5) | | |
| CGI- | 3.90 | | 3.09 | (1.14) | 3.67 | 2.9 | (0.99) |
| Improvement | (0.88) | | | | (0.50) | | |

^{a}Last observation carried forward as endpoint; includes all subjects who completed at least one post-baseline visit
^{b}Week 8 as endpoint

Among study completers, there were no significant differences between subjects receiving testosterone and those receiving placebo on change in percent body fat [−2.8 (1.7)% vs. −1.9 (2.6)%; t=0.90, df=17, p=0.38] or change in muscle mass as expressed by FFMI [1.1 (0.9) vs. 0.6 (1.2) kg/meter²; t=1.03, df=17, p=0.32].

Mean testosterone levels at Week 1 were 789 (519) ng/dl in the testosterone group vs. 249 (68) ng/dl in the placebo group (t=3.26, df=19, p=0.004). Notably, 3 of 11 testosterone subjects displayed ≦70 ng/dl increase in their total testosterone levels with the gel; these same subjects also displayed little improvement in depressive symptoms (changes of 0, 0, and 1, respectively on CGI-severity at termination). The remaining 8 subjects all achieved ≧200 ng/dl increase in testosterone levels at Week 1; 4 (50%) of these subjects improved by 2 points or more on CGI-severity, as compared to none of the 10 subjects receiving placebo (p=0.023 by Fisher's exact test, two-tailed). Testosterone gel benefited psychological aspects of depression (such as the depressed mood, guilt, and psychological anxiety items on the HAM-D) to nearly the same degree as the somatic aspects of depression (such as the HAM-D items involving sleep, appetite, libido, and somatic symptoms). Preliminary data suggest that in much lower doses, testosterone may exhibit antidepressant effects in women as well.

The contents of all cited references throughout this application are hereby expressly incorporated by reference. The practice of the present invention will employ, unless otherwise indicated, conventional techniques of pharmacology and pharmaceutics, which are within the skill of the art.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

What is claimed is

1. A method of treating, preventing or reducing the risk of developing a depressive disorder in a subject in need thereof, comprising: administering a depressive-disorder-effective amount of a composition to an area of skin of the subject for delivery of a steroid in the testosterone synthetic pathway to blood serum of the subject, wherein the composition comprises:
(a) about 0.01% to about 70% of the steroid in the testosterone synthetic pathway;
(b) about 0.01% to about 50% penetration enhancing agent;
(c) about 0.01% to about 50% thickening agent; and
(d) about 30% to about 98% lower alcohol;
wherein the composition is capable of releasing the steroid after applying the composition to the skin at a rate and duration that delivers at least about 10 μg per day of the steroid to the blood serum of the subject; and the percentages are on a weight to weight basis of the composition.

2. The method of claim 1, wherein the steroid in the testosterone synthetic pathway comprises about 0.1% to about 10% testosterone, or a salt, ester, amide, enantiomer, isomer, tautomer, prodrug, or derivative thereof.

3. The method of claim 1, wherein the steroid in the testosterone synthetic pathway comprises about 1% testosterone, or a salt, ester, amide, enantiomer, isomer, tautomer, prodrug, or derivative thereof.

4. The method of claim 2, wherein the penetration enhancing agent comprises about 0.1% to about 5% of isostearic acid, octanoic acid, lauryl alcohol, ethyl oleate, isopropyl myristate, butyl stearate, methyl laurate, diusopropyl adipate, glyceryl monolaurate, tetrahydrofurfuryl alcohol, polyethylene glycol ether, polyethylene glycol, propylene glycol, 2-(2-ethoxyethoxy) ethanol, diethylene glycol monomethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, or terpene.

5. The method of claim 4, wherein the penetration enhancing agent is isopropyl myristate.

6. The method of claim 2, wherein the thickening agent comprises about 0.1% to about 5% polyacrylic acid.

7. The method of claim 6, wherein the thickening agent comprises about 0.9% polyacrylic acid.

8. The method of claim 6, wherein the polyacrylic acid is carboxypolymethylene.

9. The method of claim 2, wherein the lower alcohol comprises about 45% to about 90% ethanol or isopropanol.

10. The method of claim 2, wherein the composition further comprises about 0.1% to about 10% sodium hydroxide.

11. The method of claim 2, wherein the composition weighs equal to or less than about 100 grams.

12. The method of claim 2, wherein the composition weighs about 1.0 grams to about 10 grams.

13. The method of claim 2, wherein the composition weighs about 2.5 grams to about 7.5 grams.

14. The method of claim 2, wherein the composition weighs about 5.0 grams.

15. The method of claim 2, wherein the composition is capable of releasing the testosterone after applying the composition to the skin at a rate and duration that achieves circulating serum concentration of the testosterone greater than about 400 ng testosterone per dl serum during a time period beginning about 2 hours after administration and ending about 24 hours after administration.

16. The method of claim 15, wherein the serum testosterone concentration is maintained between about 400 ng testosterone per dl serum to about 1050 ng testosterone per dl serum.

17. The method of claim 2, wherein for each about 0.1 gram per day application of the composition to the skin, an increase of at least about 5 ng/dl in serum testosterone concentration results in the subject.

18. The method of claim 2, wherein the composition is provided to the subject for daily administration in about a 0.1 g to about a 10 g dose.

19. The method of claim 2, wherein the amount of the composition is a 5 g dose delivering about 5 mg to about 500 mg of testosterone to the skin.

20. The method of claim 2, wherein the amount of the composition is a 7.5 g dose delivering about 7.5 mg to about 750 mg of testosterone to the skin.

21. The method of claim 2, wherein the amount of the composition is a 10 g dose delivering 10 mg to about 1000 mg of testosterone to the skin.

22. The method of claim 2, wherein the composition is provided to the subject in one or more packets.

23. The method of claim 22, wherein the packet comprises a polyethylene liner between the composition and inner surface of the packet.

24. The method of claim 2, wherein the composition is provided as a separate component to a kit.

25. The method of claim 2, wherein the subject has a pretreatment serum testosterone concentration less than about 300 ng/dl.

26. The method of claim 25, wherein after at least about 30 days of daily administration serum testosterone concentration in the subject is at least about 490 ng/dl to about 860 ng/dl.

27. The method of claim 25, wherein after at least about 30 days of daily administration total serum androgen concentration in the subject is greater than about 372 ng/dl.

28. The method of claim 2, wherein the composition is administered once, twice, or three times daily for at least about 7 days.

29. A method of treating, preventing or reducing the risk of developing a depressive disorder in a subject in need thereof, comprising: administering to the subject:
(a) an amount of a composition comprising:
(i) about 0.01% to about 70% steroid in the testosterone synthetic pathway;
(ii) about 0.01% to about 50% penetration enhancing agent;
(iii) about 0.01% to about 50% thickening agent; and
(iv) about 30% to about 98% lower alcohol; and
(b) an amount of a therapeutic agent comprising an antidepressant agent, an inhibitor of the synthesis of sex hormone binding globulin, or an estrogenic hormone;
wherein
the composition is administered to an area of skin of the subject for delivery of the steroid to blood serum of the subject, and is capable of releasing the steroid after applying the composition to the skin at a rate and duration that delivers at least about 10 μg per day of the steroid to the blood serum of the subject, and the percentages are on a weight to weight basis of the composition; and
the amount of the composition and the amount of the therapeutic agent together make a depressive-disorder-effective amount.

30. The method of claim 29, wherein the steroid in the testosterone synthetic pathway comprises about 0.1% to about 10% testosterone, or a salt, ester, amide, enantiomer, isomer, tautomer, prodrug, or derivative thereof.

31. The method of claim 29, wherein the steroid in the testosterone synthetic pathway comprises about 1% testosterone, or a salt, ester, amide, enantiomer, isomer, tautomer, prodrug, or derivative thereof.

32. The method of claim 29, wherein the penetration enhancing agent comprises about 0.1% to about 5% of isostearic acid, octanoic acid, lauryl alcohol, ethyl oleate, isopropyl myristate, butyl stearate, methyl laurate, diisopropyl adipate, glyceryl monolaurate, tetrahydrofurfuryl alcohol, polyethylene glycol ether, polyethylene glycol, propylene glycol, 2-(2-ethoxyethoxy) ethanol, diethylene glycol monomethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, or terpene.

33. The method of claim 32, wherein the penetration enhancing agent is isopropyl myristate.

34. The method of claim 29, wherein the thickening agent comprises about 0.1% to about 5% polyacrylic acid.

35. The method of claim 34, wherein the thickening agent comprises about 0.9% polyacrylic acid.

36. The method of claim 34, wherein the polyacrylic acid is carboxypolymethylene.

37. The method of claim 29, wherein the lower alcohol comprises about 45% to about 90% ethanol or isopropanol.

38. The method of claim 29, wherein the composition further comprises about 0.1% to about 10% sodium hydroxide.

39. The method of claim 29, wherein the composition and the therapeutic agent are provided as separate components to a kit.

40. The method of claim 29, wherein the composition and the therapeutic agent are administered substantially simultaneously, or sequentially.

41. The method of claim 29, wherein the therapeutic agent is administered orally, percutaneously, intravenously, intramuscularly, or by direct absorption through mucous membrane tissue.

42. A pharmaceutical composition for administration to skin of a subject, comprising:
(i) about 0.01% to about 70% steroid in the testosterone synthetic pathway;
(ii) about 0.01% to about 50% penetration enhancing agent;
(iii) about 0.01% to about 50% thickening agent;
(iv) about 30% to about 98% lower alcohol; and
(v) a therapeutic agent comprising an antidepressant agent, an inhibitor of the synthesis of sex hormone binding globulin, or an estrogenic hormone; and
wherein
the composition is capable of being administered to an area of skin of the subject for delivery of the steroid in the testosterone synthetic pathway and the therapeutic agent to blood serum of the subject, and is capable of releasing the steroid after applying the composition to the skin at a rate and duration that delivers at least about 10 μg per day of the steroid to the blood serum of the subject, and the percentages are on a weight to weight basis of the composition; and
the amount of the steroid and the amount of the therapeutic agent together make a depressive-disorder-effective amount.
